(19) United States
(12) Patent Application Publication
(10) Pub. No.: US 2021/0017249 A1
(43) Pub. Date: Jan. 21, 2021

(54) METHODS OF PRODUCING CELLS EXPRESSING A RECOMBINANT RECEPTOR AND RELATED COMPOSITIONS

(71) Applicants: Juno Therapeutics, Inc., Seattle, WA (US); Editas Medicine, lnc., Cambridge, MA (US)

(72) Inventors: Blythe D. SATHER, Seattle, WA (US); Christopher BORGES, Cambridge, MA (US); Stephen Michael BURLEIGH, Seattle, WA (US); Christopher Heath NYE, Seattle, WA (US); Queenie VONG, Seattle, WA (US); Gordon Grant WELSTEAD, Cambridge, MA (US)

(73) Assignees: Juno Therapeutics, Inc., Seattle, WA (US); Editas Medicine, lnc., Cambridge, MA (US)

(21) Appl. No.: 17/044,221

(22) PCT Filed: Apr. 3, 2019
(86) PCT No.: PCT/US2019/025682

§ 371 Date: Sep. 30, 2020

Related U.S. Application Data
(60) Provisional application No. 62/653,522, filed on Apr. 5, 2018.

(51) Int. Cl. C07K 14/725; C12N 15/90; A61K 35/17; C12N 5/0783; C07K 14/705; C07K 16/28; A61P 35/00

(52) U.S. Cl. CPC C07K 14/7051; C12N 2501/2315; A61K 35/17; C12N 5/0636; C07K 14/70578; C07K 16/2803; A61P 35/00; C12N 2310/20; C12N 2800/80; C07K 2317/622; C07K 2319/03; C07K 2319/02; C07K 2319/33; C12N 2501/2302; C12N 2501/2307; C12N 15/907

(57) ABSTRACT

Provided are methods for engineering immune cells, cell compositions containing engineered immune cells, kits and articles of manufacture for targeting nucleic acid sequence encoding a recombinant receptor to a particular genomic locus and/or for modulating expression of the gene at the genomic locus, and applications thereof in connection with cancer immunotherapy comprising adoptive transfer of engineered T cells. These may involve genetic disruption of at least one site within a TRAC gene and/or a TRBC gene and integration of the transgene encoding for the recombinant receptor at or near one of the at least one target site.

### CROSS-REFERENCE TO RELATED APPLICATIONS

This application claims priority from U.S. provisional application No. 61/653,522, filed Apr. 5, 2018, entitled “METHODS OF PRODUCING CELLS EXPRESSING A RECOMBINANT RECEPTOR AND RELATED COMPOSITIONS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042012740SeqList.txt, created Apr. 3, 2019, which is 179 kilobytes in size. The information in the electronic format of the Sequence Listing is incorporated by reference in its entirety.

### FIELD

The present disclosure relates to methods for engineering immune cells, cell compositions containing engineered immune cells, kits and articles of manufacture for targeting nucleic acid sequence encoding a recombinant receptor to a particular genomic locus and/or for modulating expression of the gene at the genomic locus, and applications thereof in connection with cancer immunotherapy comprising adoptive transfer of engineered T cells.

### BACKGROUND

Adoptive cell therapies that utilize recombinantly expressed T cell receptors (TCRs) or other antigen receptors (e.g. chimeric antigen receptors (CARs)) to recognize tumor antigens represent an attractive therapeutic modality for the treatment of cancers and other diseases. Expression and function of recombinant TCRs or other antigen receptors can be limited and/or heterogeneous in a population of cells. Improved strategies are needed to achieve high and/or homogenous expression levels and function of the recombinant receptors. These strategies can facilitate generation of cells exhibiting desired expression levels and/or properties for use in adoptive immunotherapy, e.g., in treating cancer, infectious diseases and autoimmune diseases. Provided are methods, cells, compositions and kits for use in the methods that meet such needs.

### SUMMARY

Provided herein are genetically engineered cells that contain a genetic disruption of at least one target site within a T cell receptor alpha constant (TRAC) gene and/or a T cell receptor beta constant (TRBC) gene, and a transgene encoding a recombinant receptor, such as a T cell receptor (TCR) or a chimeric antigen receptor (CAR), that is integrated, via homology directed repair (HDR), at or near one or more of the target sites, and composition comprising the engineered cells, methods for producing the engineered cells and related methods and uses. In some aspects, by virtue of the genetic disruption and targeted integration of the transgene sequences, one or more of the endogenous TCR chains are reduced or knocked out in expression. In some of any such embodiments, the recombinant receptor can bind to an antigen that is associated with a cell or tissue of a disease, disorder or condition. In some of any such embodiments, the recombinant receptor can bind to an antigen that is specific to a cell or tissue of a disease, disorder or condition. In some of any such embodiments, the recombinant receptor can bind to an antigen that is expressed on a cell or tissue associated with a disease, disorder or condition.

Also provided herein is a composition comprising an engineered cell or a plurality of engineered cells described herein. In particular embodiments, at least or greater than 35%, 40%, 45%, 50%, 55%, 60%, 65%, 70%, 75%, 80%, or 90% of the cells in the composition comprise a genetic disruption of at least one target site within a gene encoding a domain or region of T cell receptor alpha constant (TRAC) gene and/or a T cell receptor beta constant (TRBC) gene. In certain embodiments, at least or greater than 35%, 40%, 45%, 50%, 55%, 60%, 65%, 70%, 75%, 80%, or 90% of the cells in the composition express the recombinant receptor or antigen-binding fragment thereof and/or exhibit antigen binding. In some of any such embodiments, at least or greater than 35%, 40%, 45%, 50%, 55%, 60%, 65%, 70%, 75%, 80%, or 90% of the cells in the composition express the recombinant receptor or antigen-binding fragment thereof and/or exhibit binding to the antigen.

Provided herein are compositions containing a plurality of engineered T cells. In some of any such embodiments, A composition, comprising a plurality of engineered T cells comprising a recombinant receptor or an antigen-binding fragment or chain thereof encoded by a transgene and a genetic disruption of at least one target site within a T cell receptor alpha constant (TRAC) gene and/or a T cell receptor beta constant (TRBC) gene, wherein the recombinant receptor is capable of binding to an antigen that is associated with, specific to, and/or expressed on a cell or tissue of a disease, disorder or condition, and wherein: at least or greater than 35%, 40%, 45%, 50%, 55%, 60%, 65%, 70%, 75%, 80%, or 90% of the cells in the composition comprise a genetic disruption of at least one target site within a TRAC gene and/or a TRBC gene; and/or at least or greater than 35%, 40%, 45%, 50%, 55%, 60%, 65%, 70%, 75%, 80%, or 90% of the cells in the composition express the recombinant receptor or antigen-binding fragment or chain thereof and/or exhibits binding to the antigen; and the transgene encoding the recombinant receptor or antigen-binding fragment or chain thereof is integrated at or near one of the at least one target site via homology directed repair (HDR).

In some embodiments, the coefficient of variation of expression and/or antigen binding of the recombinant receptor or antigen-binding fragment or a chain thereof among the plurality of cells is lower than 0.70, 0.65, 0.60, 0.55, 0.50, 0.45, 0.40, 0.35 or 0.30 or less. In particular embodiments, the coefficient of variation of expression and/or antigen binding of the recombinant receptor or antigen-binding fragment or a chain thereof among the plurality of cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration. In some of any such embodiments, the recombinant receptor is capable of binding to an antigen that is associated with, specific to, and/or expressed on a cell or tissue of a disease, disorder or condition.

In certain embodiments, expression and/or antigen-binding of the recombinant receptor or antigen-binding fragment thereof is assessed by contacting the cells in the composition with a binding reagent specific for the TCRα chain or the TCRβ chain and assessing binding of the reagent to the cells. In some embodiments, the binding reagent is an anti-TCR Vβ antibody or is an anti-TCR Vα antibody that specifically recognizes a specific family of Vβ or Vα chains.

In particular embodiments, the binding agent is a peptide antigen-MHC complex, which optionally is a tetramer. In certain embodiments, a composition described herein further comprises a pharmaceutically acceptable carrier. Provided herein is a composition, comprising a plurality of engineered T cells comprising a recombinant receptor or an antigen-binding fragment or chain thereof encoded by a transgene and a genetic disruption of at least one target site within a T cell receptor alpha constant (TRAC) gene and/or a T cell receptor beta constant (TRBC) gene, wherein at least or greater than 35%, 40%, 45%, 50%, 55%, 60%, 65%, 70%, 75%, 80%, or 90% of the cells in the composition comprise a genetic disruption of at least one target site within a TRAC gene and/or a TRBC gene; and the transgene encoding the recombinant TCR or antigen-binding fragment or chain thereof is targeted for integration at or near one of the at least one target site via homology directed repair (HDR).

Also provided herein is a composition, comprising a plurality of engineered T cells comprising a recombinant receptor or an antigen-binding fragment or chain thereof encoded by a transgene and a genetic disruption of at least one target site within a T cell receptor alpha constant (TRAC) gene and/or a T cell receptor beta constant (TRBC) gene, wherein at least or greater than 35%, 40%, 45%, 50%, 55%, 60%, 65%, 70%, 75%, 80%, or 90% of the cells in the composition express the recombinant receptor or antigen-binding fragment thereof and/or exhibit antigen binding; and the transgene encoding the recombinant TCR or antigen-binding fragment or chain thereof is targeted for integration at or near one of the at least one target site via homology directed repair (HDR).

Also provided herein composition, comprising a plurality of engineered T cells comprising a recombinant receptor or an antigen-binding fragment thereof encoded by a transgene and a genetic disruption of at least one target site within a T cell receptor alpha constant (TRAC) gene and/or a T cell receptor beta constant (TRBC) gene, wherein the coefficient of variation of expression and/or antigen binding of the recombinant receptor among the plurality of cells is lower than 0.70, 0.65, 0.60, 0.55, 0.50, 0.45, 0.40, 0.35 or 0.30 or less.

Also provided herein is composition, comprising a plurality of engineered T cells comprising a recombinant receptor or an antigen-binding fragment thereof encoded by a transgene and a genetic disruption of at least one target site within a T cell receptor alpha constant (TRAC) gene and/or a T cell receptor beta constant (TRBC) gene, wherein the coefficient of variation of expression and/or antigen binding of the recombinant receptor among the plurality of cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

In some embodiments, the composition is generated by: (a) introducing into a plurality of T cells one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and (b) introducing into the plurality of T cells a template polynucleotide comprising a transgene encoding a recombinant T cell receptor (TCR) or an antigen-binding fragment or a chain thereof, wherein the transgene encoding the recombinant TCR or antigen-binding fragment or chain thereof is targeted for integration at or near one of the at least one target site via homology directed repair (HDR). In particular embodiments, expression and/or antigen-binding of the recombinant receptor or antigen-binding fragment thereof is assessed by contacting the cells in the composition with a binding reagent specific for the TCRα chain or the TCRβ chain and assessing binding of the reagent to the cells.

In some of any such embodiments, the engineered T cell comprises at least one genetic disruption in the TRAC gene. In some of any such embodiments, the engineered T cell comprises at least one genetic disruption in the TRBC gene. In some of any such embodiments, the engineered T cell comprises at least one genetic disruption of a target site in a TRAC gene and at least one genetic disruption of a target site in a TRBC gene.

In certain embodiments, the binding reagent is an anti-TCR Vβ antibody or is an anti-TCR Vα antibody that specifically recognizes a specific family of Vβ or Vα chains. In some embodiments, the binding agent is a peptide antigen-MHC complex, which optionally is a tetramer. In particular embodiments, at least one of the one or more agent is capable of inducing a genetic disruption of a target site in a TRAC gene. In certain embodiments, at least one of the one or more agent is capable of inducing a genetic disruption of a target site in a TRBC gene. In some embodiments, the one or more agents comprises at least one agent that capable of inducing a genetic disruption of a target site in a TRAC gene and at least one agent that is capable of inducing a genetic disruption of a target site in a TRBC gene. In particular embodiments, the TRBC gene is one or both of a T cell receptor beta constant 1 (TRBC1) or T cell receptor beta constant 2 (TRBC2) gene. In certain embodiments, the one or more agent capable of inducing a genetic disruption comprises a DNA binding protein or DNA-binding nucleic acid that specifically binds to or hybridizes to the target site. In some embodiments, the one or more agent capable of inducing a genetic disruption comprises (a) a fusion protein comprising a DNA-targeting protein and a nuclease or (b) an RNA-guided nuclease.

In particular embodiments, the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site. In certain embodiments, the one or more agent comprises a zinc finger nuclease (ZFN), a TAL-effector nuclease (TALEN), or and a CRISPR-Cas9 combination that specifically binds to, recognizes, or hybridizes to the target site. In some embodiments, each of the one or more agent comprises a guide RNA (gRNA) having a targeting domain that is complementary to the at least one target site. In particular embodiments, the one or more agent is introduced as a ribonucleoprotein (RNP) complex comprising the gRNA and a Cas9 protein. In certain embodiments, the RNP is introduced via electroporation, particle gun, calcium phosphate transfection, cell compression or squeezing. In some embodiments, the RNP is introduced via electroporation. In particular embodiments, the one or more agent is introduced as one or more polynucleotide encoding the gRNA and/or a Cas9 protein. In certain embodiments, the at least one target site is within an exon of the TRAC, TRBC1 and/or TRBC2 gene.

In some of any such embodiments, the genetic disruption is by a zinc finger nuclease (ZFN), a TAL-effector nuclease (TALEN), or and a CRISPR-Cas9 combination that specifically binds to, recognizes, or hybridizes to the target site. In some of any such embodiments, the genetic disruption by a CRISPR-Cas9 combination comprises a guide RNA (gRNA) having a targeting domain that is complementary to the at least one target site. In some of any such embodiments, the CRISPR-Cas9 combination is a ribonucleoprotein (RNP) complex comprising a gRNA and a Cas9 protein. In some of any such embodiments, the RNP is introduced via electroporation. In some of any such embodiments, the at least one target site is within an exon of the TRAC, TRBC1 and/or TRBC2 gene.

In some embodiments, the gRNA has a targeting domain that is complementary to a target site in a TRAC gene and comprises a sequence selected from the group consisting of UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58). In particular embodiments, the gRNA has a targeting domain comprising the sequence GAGAAUCAAAAUCGGUGAAU (SEQ ID NO:31).

In certain embodiments, the gRNA has a targeting domain that is complementary to a target site in one or both of a TRBC1 and a TRBC2 gene and comprises a sequence selected from the group consisting of CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116). In some embodiments, the gRNA has a targeting domain comprising the sequence GGCCUCGGCGCUGACGAUCU (SEQ ID NO:63).

In some of any such embodiments, the transgene is integrated by a template polynucleotide introduced into each of a plurality of T cells. In particular embodiments, the template polynucleotide comprises the structure [5′ homology arm]-[transgene]-[3′ homology arm]. In certain embodiments, the 5′ homology arm and 3′ homology arm comprises nucleic acid sequences homologous to nucleic acid sequences surrounding the at least one target site. In some embodiments, the 5′ homology arm comprises nucleic acid sequences that are homologous to nucleic acid sequences 5′ of the target site. In particular embodiments, the 3′ homology arm comprises nucleic acid sequences that are homologous to nucleic acid sequences 3′ of the target site. In certain embodiments, the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In some embodiments, the 5′ homology arm and 3′ homology arm independently are between about 50 and 100, 100 and 250, 250 and 500, 500 and 750, 750 and 1000, 1000 and 2000 nucleotides. In some of any such embodiments, the 5′ homology arm and 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

In particular embodiments, the 5′ homology arm and 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides. In particular embodiments, the 5′ homology arm and 3′ homology arm independently are from at or about 100 to at or about 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides in length.

In some of any such embodiments, the 5′ homology arm and 3′ homology arm independently are at or about 200, 300, 400, 500, 600, 700 or 800 nucleotides in length, or any value between any of the foregoing. In some of any such embodiments, the 5′ homology arm and 3′ homology arm independently are greater than at or about 300 nucleotides in length. In some of any such embodiments, the 5′ homology arm and 3′ homology arm independently are at or about 400, 500 or 600 nucleotides in length or any value between any of the foregoing. In some of any such embodiments, the 5′ homology arm and 3′ homology arm independently are between at or about 500 and at or about 600 nucleotides in length. In some of any such embodiments, the 5′ homology arm and 3′ homology arm independently are greater than at or about 300 nucleotides in length.

In some of any such embodiments, the transgene encoding the recombinant receptor or antigen-binding fragment or chain thereof is integrated at or near the target site in the TRAC gene. In some embodiments, the transgene encoding the recombinant receptor or antigen-binding fragment or chain thereof is integrated at or near the target site in one or both of the TRBC1 and the TRBC2 gene.

In some of any such embodiments, the recombinant receptor is a chimeric antigen receptor (CAR). In some of any such embodiments, the CAR comprises an extracellular domain comprising an antigen binding domain specific for the antigen. Insome of any such embodiments, the antigen binding domain is an scFv; a transmembrane domain; a cytoplasmic signaling domain derived from a costimulatory molecule and a cytoplasmic signaling domain derived from a primary signaling ITAM-containing molecule. In some of any such embodiments, the CAR further comprises a spacer between the transmembrane domain and the antigen-binding domain. In some of any such embodiments, the costimulatory molecule is or comprises a 4-1BB, optionally human 4-1BB. In some of any such embodiments, the ITAM-containing molecule is or comprises a CD3zeta signaling domain. In some of any such embodiments, the ITAM-containing molecule is a human CD3zeta signaling domain.

In some of any such embodiments, the recombinant receptor is a recombinant TCR or antigen-binding fragment or a chain thereof. In some of any such embodiments, the recombinant receptor is a recombinant TCR comprising an alpha (TCRα) chain and a beta (TCRβ) chain, and the transgene encoding the recombinant TCR or antigen-binding fragment or chain thereof comprises a nucleic acid sequence encoding the TCRα chain and a nucleic acid sequence encoding the TCRβ chain. In some of any such embodiments, the transgene further comprises one or more multicistronic element(s) and the multicistronic element(s) is positioned between the nucleic acid sequence encoding the TCRα or a portion thereof and the nucleic acid sequence encoding the TCRβ or a portion thereof. In some of any such embodiments, the multicistronic element(s) comprises a sequence encoding a ribosome skip element selected from among a T2A, a P2A, a E2A or a F2A or an internal ribosome entry site (IRES).

In some of any such embodiments, the engineered cell further comprises one or more second transgene(s), wherein the second transgene is integrated at or near one of the at least one target site via homology directed repair (HDR). In some of any such embodiments, the recombinant receptor is a recombinant TCR and the transgene encoding the recombinant TCR or antigen-binding fragment or chain thereof comprises a nucleic acid sequence encoding one chain of the recombinant TCR and the second transgene comprises a nucleic acid sequence encoding a different chain of the recombinant TCR. In some of any such embodiments, the transgene encoding the recombinant TCR or antigen-binding fragment or chain thereof comprises the nucleic acid sequence encoding the TCRα chain and the second transgene comprises the nucleic acid sequence encoding the TCRβ chain or a portion thereof. In some of any such embodiments, the integration of the second transgene is by a second template polynucleotide introduced into each of the plurality of T cells, said second template polynucleotide comprising the structure [second 5′ homology arm]-[one or more second transgene]-[second 3′ homology arm].

In certain embodiments, the transgene encoding the recombinant TCR or antigen-binding fragment or chain thereof is targeted for integration at or near the target site in the TRAC gene. In some embodiments, the transgene encoding the recombinant TCR or antigen-binding fragment or chain thereof is targeted for integration at or near the target site in one or both of the TRBC1 and the TRBC2 gene. In particular embodiments, the composition is generated by further introducing into the immune cell one or more second template polynucleotide comprising one or more second transgene, wherein the second transgene is targeted for integration at or near one of the at least one target site via homology directed repair (HDR).

In certain embodiments, the second template polynucleotide comprises the structure [second 5′ homology arm]-[one or more second transgene]-[second 3′ homology arm]. In some embodiments, the second 5′ homology arm and second 3′ homology arm comprises nucleic acid sequences homologous to nucleic acid sequences surrounding the at least one target site. In particular embodiments, the second 5′ homology arm comprises nucleic acid sequences that are homologous to nucleic acid sequences second 5′ of the target site. In certain embodiments, the second 3′ homology arm comprises nucleic acid sequences that are homologous to nucleic acid sequences second 3′ of the target site.

In some embodiments, the second 5′ homology arm and second 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In particular embodiments, the second 5′ homology arm and second 3′ homology arm independently are between about 50 and 100, 100 and 250, 250 and 500, 500 and 750, 750 and 1000, 1000 and 2000 nucleotides. In certain embodiments, the second 5′ homology arm and second 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides.

In some embodiments, the one or more second transgene is targeted for integration at or near the target site in the TRAC gene. In particular embodiments, the one or more second transgene is targeted for integration at or near the target site in the TRBC1 or the TRBC2 gene. In certain embodiments, transgene encoding the recombinant TCR or antigen-binding fragment or chain thereof is targeted for integration at or near the target site in the TRAC gene, the TRBC1 gene or the TRBC2 gene, and the one or more second transgene is targeted for integration at or near one or more of the target site that is not targeted by the transgene encoding the recombinant TCR or antigen-binding fragment or chain thereof.

In some embodiments, the transgene encoding the recombinant TCR or antigen-binding fragment or chain thereof is targeted for integration at or near the target site in the TRAC gene, and the one or more second transgene is targeted for integration at or near one or more of the target site in the TRBC1 gene and/or the TRBC2 gene. In particular embodiments, the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor. In certain embodiments, the encoded molecule is a co-stimulatory ligand optionally selected from among a tumor necrosis factor (TNF) ligand selected from 4-1BBL, OX40L, CD70, LIGHT and CD30L, or an immunoglobulin (Ig) superfamily ligand selected from CD80 and CD86.

In some of any such embodiments, the transgene encoding the recombinant receptor or antigen-binding fragment or chain thereof is integrated at or near a target site in the TRAC gene, and the one or more second transgene is integrated at or near one or more other target site among the TRAC gene, the TRBC1 gene or the TRBC2 gene and that is not integrated by the transgene encoding the recombinant receptor or antigen-binding fragment or chain thereof. In some of any such embodiments, the transgene encoding the recombinant receptor or antigen-binding fragment or chain thereof is integrated at or near a target site in the TRAC gene, and the one or more second transgene is integrated at or near one or more target site in the TRBC1 gene and/or the TRBC2 gene. In some of any such embodiments, the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor.

In some embodiments, the encoded molecule is a cytokine optionally selected from among IL-2, IL-3, IL-6, IL-11, IL-12, IL-7, IL-15, IL-21, granulocyte macrophage colony stimulating factor (GM-CSF), interferon alpha (IFN-α), interferon beta (IFN-β) or interferon gamma (IFN-γ) and erythropoietin. In particular embodiments, the encoded molecule is a soluble single-chain variable fragment (scFv) that optionally binds a polypeptide that has immunosuppressive activity or immunostimulatory activity selected from CD47, PD-1, CTLA-4 and ligands thereof or CD28, OX-40, 4-1BB and ligands thereof.

In certain embodiments, the encoded molecule is an immunomodulatory fusion protein, optionally comprising: (a) an extracellular binding domain that specifically binds an antigen derived from CD200R, SIRPα, CD279 (PD-1), CD2, CD95 (Fas), CD152 (CTLA4), CD223 (LAG3), CD272 (BTLA), A2aR, KIR, TIM3, CD300 or LPA5; (b) an intracellular signaling domain derived from CD3ε, CD3δ, CD3ζ, CD25, CD27, CD28, CD40, CD47, CD79A, CD79B, CD134 (OX40), CD137 (4-1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRFM, Zap70, PTCH2, or any combination thereof; and (c) a hydrophobic transmembrane domain derived from CD2, CD3ε, CD3δ, CD3ζ, CD25, CD27, CD28, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5 or Zap70. In some embodiments, the encoded molecule is a chimeric switch receptor (CSR) that optionally comprises a truncated extracellular domain of PD1 and the transmembrane and cytoplasmic signaling domains of CD28. In particular embodiments, the encoded molecule is a co-receptor optionally selected from CD4 or CD8.

In certain embodiments, transgene encoding the recombinant TCR or antigen-binding fragment or chain thereof encodes one chain of a recombinant TCR and the second transgene encodes a different chain of the recombinant TCR. In some embodiments, transgene encoding the recombinant TCR or antigen-binding fragment or chain thereof encodes the alpha (TCRα) chain of the recombinant TCR and the second transgene encodes the beta (TCRβ) chain of the recombinant TCR. In particular embodiments, the transgene encoding the recombinant TCR or antigen-binding fragment or chain thereof and/or the one or more second transgene independently further comprises a regulatory or control element.

In some of any such embodiments, the transgene encoding the recombinant receptor or antigen-binding fragment or chain thereof further comprises a heterologous regulatory or control element. In some of any such embodiments, the transgene encoding the recombinant receptor or antigen-binding fragment or chain thereof and/or the one or more second transgene independently further comprises a heterologous regulatory or control element. In some of any such embodiments, the heterologous regulatory or control element comprises a heterologous promoter. In some of any such embodiments, the heterologous promoter is or comprises a human elongation factor 1 alpha (EF1α) promoter or an MND promoter or a variant thereof. In some of any such embodiments, the heterologous promoter is an inducible promoter or a repressible promoter.

In certain embodiments, the regulatory or control element comprises a promoter, an enhancer, an intron, a polyadenylation signal, a Kozak consensus sequence, a splice acceptor sequence or a splice donor sequence. In some embodiments, the regulatory or control element comprises a promoter. In particular embodiments, the promoter is selected from among a constitutive promoter, an inducible promoter, a repressible promoter and/or a tissue-specific promoter. In certain embodiments, the promoter is selected from among an RNA pol I, pol II or pol III promoter. In some embodiments, the promoter is selected from: a pol III promoter that is a U6 or H1 promoter; or a pol II promoter that is a CMV, SV40 early region or adenovirus major late promoter. In particular embodiments, the promoter is or comprises a human elongation factor 1 alpha (EF1α) promoter or an MND promoter or a variant thereof. In certain embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In particular embodiments, the transgene encoding the recombinant TCR or antigen-binding fragment or chain thereof and/or the one or more second transgene independently comprises one or more multicistronic element(s).

In certain embodiments, the one or more multicistronic element(s) are upstream of the transgene encoding the recombinant TCR or antigen-binding fragment or chain thereof and/or the one or more second transgene. In some embodiments, the multicistronic element(s) is positioned between the transgene encoding the recombinant TCR or antigen-binding fragment or chain thereof and the one or more second transgene. In particular embodiments, the multicistronic element(s) is positioned between the nucleic acid sequence encoding the TCRα or a portion thereof and the nucleic acid sequence encoding the TCRβ or a portion thereof. In certain embodiments, the multicistronic element(s) comprises a sequence encoding a riboparticular skip element selected from among a T2A, a P2A, a E2A or a F2A or an internal ribosome entry site (IRES).

In some of any such embodiments, the TCRα chain comprises a constant (Ca) region comprising introduction of one or more cysteine residues and/or the TCRβ chain comprises aCβ region comprising introduction of one or more cysteine residues, wherein the one or more introduced cysteine residues are capable of forming one or more non-native disulfide bridges between the alpha chain and beta chain. In some of any such embodiments, the introduction of the one or more cysteine residues comprises replacement of a non-cysteine residue with a cysteine residue. In some of any such embodiments, the Cα region comprises a cysteine at a position corresponding to position 48 with numbering as set forth in any of SEQ ID NO: 24; and/or the Cβ region comprises a cysteine at a position corresponding to position 57 with numbering as set forth in SEQ ID NO: 20.

In certain embodiments, the sequence encoding a riboparticular skip element is targeted to be in-frame with the gene at the target site. In some embodiments, upon HDR, the transgene encoding the recombinant TCR or antigen-binding fragment or chain thereof and/or the one or more second transgene independently is operably linked to the endogenous promoter of the gene at the target site. In certain embodiments, the recombinant TCR is capable of binding to an antigen that is associated with, specific to, and/or expressed on a cell or tissue of a disease, disorder or condition. In particular embodiments, the disease, disorder or condition is an infectious disease or disorder, an autoimmune disease, an inflammatory disease, or a tumor or a cancer. In particular embodiments, the antigen is a tumor antigen or a pathogenic antigen. In certain embodiments, the pathogenic antigen is a bacterial antigen or viral antigen.

In some embodiments, the antigen is a viral antigen and the viral antigen is from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV). In particular embodiments, the antigen is an antigen from an HPV selected from among HPV-16, HPV-18, HPV-31, HPV-33 and HPV-35. In certain embodiments, the antigen is an HPV-16 antigen that is an HPV-16 E6 or HPV-16 E7 antigen. In some embodiments, the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA. In particular embodiments, the viral antigen is an HTLV-antigen that is TAX. In certain embodiments, the viral antigen is an HBV antigen that is a hepatitis B core antigen or a hepatitis B envelope antigen. In some embodiments, the antigen is a tumor antigen.

In particular embodiments, the antigen is selected from among glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin.

In certain embodiments, the T cell is a CD8+ T cell or subtypes thereof. In some embodiments, the T cell is a CD4+ T cell or subtypes thereof. In particular embodiments, the T cell is autologous to the subject. In certain embodiments, the T cell is allogeneic to the subject. In some embodiments, the first template polynucleotide, the one or more second template polynucleotide and/or the one or more polynucleotide encoding the gRNA and/or a Cas9 protein is comprised in one or more vector(s), which optionally are viral vector(s). In particular embodiments, the vector is an AAV vector. In certain embodiments, the AAV vector is selected from among AAV1, AAV2, AAV3, AAV4, AAV5, AAV6, AAV7 or AAV8 vector. In some embodiments, the AAV vector is an AAV2 or AAV6 vector. In particular embodiments, the viral vector is a retroviral vector. In certain embodiments, the viral vector is a lentiviral vector.

In some of any such embodiments, the T cells comprise CD8+ T cell and/or CD4+ T cells or subtypes thereof. In some of any such embodiments, the T cells are autologous to the subject. In some of any such embodiments, the T cells are allogeneic to the subject. In some of any such embodiments, the composition described herein further comprises a pharmaceutically acceptable carrier.

In some embodiments, the introduction of the one or more agent capable of inducing a genetic disruption and the introduction of the template polynucleotide are performed simultaneously or sequentially, in any order. In particular embodiments, the introduction of the template polynucleotide is performed after the introduction of the one or more agent capable of inducing a genetic disruption. In certain embodiments, the template polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption.

In some embodiments, the introduction of the template polynucleotide and the introduction of the one or more second template polynucleotide are performed simultaneously or sequentially, in any order. In particular embodiments, introduction of the one or more agent capable of inducing a genetic disruption and the introduction of the template polynucleotide are performed in one experimental reaction. In certain embodiments, introduction of the one or more agent capable of inducing a genetic disruption and the introduction of the template polynucleotide and the second template polynucleotide(s) are performed in one experimental reaction. In some embodiments, a composition described herein further comprises a pharmaceutically acceptable carrier.

In some embodiments, provided here in are methods of producing a genetically engineered immune cell, which include (a) introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and (b) introducing into the immune cell a template polynucleotide comprising a transgene encoding a recombinant T cell receptor (TCR) or an antigen-binding fragment thereof or a chain thereof, wherein the transgene encoding the recombinant TCR or antigen-binding fragment or chain thereof is targeted for integration at or near one of the at least one target site via homology directed repair (HDR).

In some of any such embodiments, provided herein are methods of producing a genetically engineered immune cell, which include (a) introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and (b) introducing into the immune cell a template polynucleotide comprising a transgene encoding a recombinant receptor or an antigen-binding fragment thereof or a chain thereof, said recombinant receptor being capable of binding to an antigen that is associated with, specific to, and/or expressed on a cell or tissue of a disease, disorder or condition, wherein the transgene encoding the recombinant receptor or antigen-binding fragment or chain thereof is targeted for integration at or near one of the at least one target site via homology directed repair (HDR), wherein the introduction of the template polynucleotide is performed after the introduction of the one or more agent capable of inducing a genetic disruption.

In some embodiments, also provided herein are methods of producing a genetically engineered immune cell, which include introducing into an immune cell having a genetic disruption of at least one target site within a T cell receptor alpha constant (TRAC) gene and/or a T cell receptor beta constant (TRBC) gene a template polynucleotide comprising a transgene encoding a recombinant T cell receptor (TCR) or an antigen-binding fragment thereof or a chain thereof, wherein the genetic disruption has been induced by one or more agent, wherein each of the one or more agent is independently capable of inducing a genetic disruption, and the transgene encoding the recombinant TCR or antigen-binding fragment or chain thereof is targeted for integration at or near one of the at least one target position via homology directed repair (HDR).

In some of any such embodiments, also provided herein are methods for producing a genetically engineered immune cell, which include introducing into an immune cell having a genetic disruption of at least one target site within a T cell receptor alpha constant (TRAC) gene and/or a T cell receptor beta constant (TRBC) gene a template polynucleotide comprising a transgene encoding a recombinant receptor or an antigen-binding fragment thereof or a chain thereof, said recombinant receptor being capable of binding to an antigen that is associated with, specific to, and/or expressed on a cell or tissue of a disease, disorder or condition, wherein the genetic disruption has been induced by one or more agent, wherein each of the one or more agent is independently capable of inducing a genetic disruption, and the transgene encoding the recombinant receptor or antigen-binding fragment or chain thereof is targeted for integration at or near one of the at least one target site via homology directed repair (HDR).

In some of any such embodiments, the template polynucleotide is introduced immediately after, or within at or about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption. In some of any such embodiments, the template polynucleotide is introduced at or about 2 hours after the introduction of the one or more agents.

In some of any such embodiments, the one or more immune cells comprises T cells. In some of any such embodiments, the T cells comprise CD4+ T cells, CD8+ T cells or CD4+ and CD8+ T cells. In some of any such embodiments, the T cells comprise CD4+ and CD8+ T cells and the ratio of CD4+ to CD8+ T cells is at or about 1:3 to at or about 3:1. In some of any such embodiments, optionally at or about 1:2 to at or about 2:1, the ratio of CD4+ to CD8+ T cells is at or about 1:1.

In some of any such embodiments, the one or more agent comprises a CRISPR-Cas9 combination and the CRISPR-Cas9 combination comprises a guide RNA (gRNA) having a targeting domain that is complementary to the at least one target site. In some of any such embodiments, the CRISPR-Cas9 combination is a ribonucleoprotein (RNP) complex comprising the gRNA and a Cas9 protein. In some of any such embodiments, the concentration of the RNP is or is about 1 μM to at or about 5 μM. In some of any such embodiments, the concentration of the RNP is or is about 2 μM.

In some of any such embodiments, the one or more agents are introduced by electroporation. In some of any such embodiments, the template polynucleotide is comprised in a viral vector(s) and the introduction of the template polynucleotide is by transduction. In some of any such embodiments, the vector is an AAV vector.

In some of any such embodiments, the method comprises incubating the cells in vitro with a stimulatory agent(s) under conditions to stimulate or activate the one or more immune cells prior to the introducing of the one or more agent. In some of any such embodiments, the stimulatory agent (s) comprises and anti-CD3 and/or anti-CD28 antibodies, optionally anti-CD3/anti-CD28 beads. In some of any such embodiments, the bead to cell ratio is or is about 1:1. In some of any such embodiments, the stimulatory agent(s) are removed from the immune cells prior to the introducing of the one or more agents.

In some of any such embodiments, the method further comprises incubating the cells prior to, during or subsequent to the introducing of the one or more agents and/or the introducing of the template polynucleotide with one or more recombinant cytokines. In some of any such embodiments, the one or more recombinant cytokines are selected from the group consisting of IL-2, IL-7, and IL-15. In some of any such embodiments, the one or more recombinant cytokine is added at a concentration selected from a concentration of IL-2 from at or about 10 U/mL to at or about 200 U/mL. In some of any such embodiments, the concentration is at or about 50 IU/mL to at or about 100 U/mL; IL-7 at a concentration of 0.5 ng/mL to 50 ng/mL. In some of any such embodiments, the concentration is at or about 5 ng/mL to at or about 10 ng/mL and/or IL-15 at a concentration of 0.1 ng/mL to 20 ng/mL, optionally at or about 0.5 ng/mL to at or about 5 ng/mL. In some of any such embodiments, the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

In some of any such embodiments, the recombinant receptor is a chimeric antigen receptor (CAR). In some of any such embodiments, the CAR comprises an extracellular domain comprising an antigen binding domain specific for the antigen; a transmembrane domain; a cytoplasmic signaling domain derived from a costimulatory molecule; and a cytoplasmic signaling domain derived from a primary signaling ITAM-containing molecule. In some of any such embodiments, the CAR further comprises a spacer between the transmembrane domain and the antigen-binding domain. In some of any such embodiments, the antigen binding domain is an scFv. In some of any such embodiments, the costimulatory molecule is or comprises a 4-1BB. In some of any such embodiments, the costimulatory molecule is human 4-1BB. In some of any such embodiments, the ITAM-containing molecule is or comprises a CD3zeta signaling domain. In some of any such embodiments, the ITAM-containing molecule is a human CD3zeta signaling domain.

In some of any such embodiments, the recombinant receptor is a recombinant TCR or antigen-binding fragment or a chain thereof. In some of any such embodiments, the recombinant receptor is a recombinant TCR comprising an alpha (TCRα) chain and a beta (TCRβ) chain and the transgene encoding the recombinant TCR or antigen-binding fragment or chain thereof comprises a nucleic acid sequence encoding the TCRα chain and a nucleic acid sequence encoding the TCRβ chain.

In some of any such embodiments, provided herein are methods for producing a genetically engineered immune cell, comprising (a) introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and (b) introducing into the immune cell a template polynucleotide comprising a transgene encoding a recombinant receptor that is a recombinant T cell receptor (TCR) or an antigen-binding fragment thereof or a chain thereof, said transgene comprising a heterologous promoter and wherein the transgene is targeted for integration at or near one of the at least one target site via homology directed repair (HDR).

In some of any such embodiments, provided herein are methods for producing a genetically engineered immune cell, comprising introducing into an immune cell having a genetic disruption of at least one target site within a T cell receptor alpha constant (TRAC) gene and/or a T cell receptor beta constant (TRBC) gene a template polynucleotide comprising a transgene encoding a recombinant receptor that is a recombinant T cell receptor (TCR) or an antigen-binding fragment thereof or a chain thereof, said transgene comprising a heterologous promoter, wherein the genetic disruption has been induced by one or more agent wherein each of the one or more agent is independently capable of inducing a genetic disruption, and the transgene encoding the recombinant receptor or antigen-binding fragment or chain thereof is targeted for integration at or near one of the at least one target site via homology directed repair (HDR).

In some embodiments, at least one of the one or more agent is capable of inducing a genetic disruption of a target site in a TRAC gene. In particular embodiments, at least one of the one or more agent is capable of inducing a genetic disruption of a target site in a TRBC gene. In certain embodiments, the one or more agents comprises at least one agent that capable of inducing a genetic disruption of a target site in a TRAC gene and at least one agent that is capable of inducing a genetic disruption of a target site in a TRBC gene. In some embodiments, the TRBC gene is one or both of a T cell receptor beta constant 1 (TRBC1) or T cell receptor beta constant 2 (TRBC2) gene.

Provided herein is a method of producing a genetically engineered immune cell, comprising: (a) introducing into an immune cell one or more agent, wherein each of the one or more agent is independently capable of inducing a genetic disruption of a target site within a T cell receptor alpha constant (TRAC) gene and a T cell receptor beta constant (TRBC) gene, thereby inducing a genetic disruption of the target sites; and (b) introducing into the immune cell a template polynucleotide comprising a transgene encoding a recombinant T cell receptor (TCR) or an antigen-binding fragment thereof or a chain thereof, wherein the transgene encoding the recombinant TCR or antigen-binding fragment or chain thereof is targeted for integration at or near the target site via homology directed repair (HDR).

Also provided herein is a method of producing a genetically engineered immune cell, comprising: introducing into an immune cell having a genetic disruption of at least one target site within a T cell receptor alpha constant (TRAC) gene and a T cell receptor beta constant (TRBC) gene a template polynucleotide comprising a transgene encoding a recombinant T cell receptor (TCR) or an antigen-binding fragment thereof or a chain thereof, wherein the genetic disruption has been induced by one or more agent wherein each of the one or more agent is independently capable of inducing a genetic disruption, and the transgene encoding the recombinant TCR or antigen-binding fragment or chain thereof is targeted for integration at or near one of the at least one target site via homology directed repair (HDR). In particular embodiments, the TRBC gene is one or both of a T cell receptor beta constant 1 (TRBC1) or T cell receptor beta constant 2 (TRBC2) gene.

In some of any such embodiments, also provided herein are methods for producing a genetically engineered immune cell comprising (a) introducing into an immune cell at least one agent that is capable of inducing a genetic disruption of a target site within a T cell receptor alpha constant (TRAC) gene and at least one agent that is capable of inducing a genetic disruption of a target site within a T cell receptor beta constant (TRBC) gene, thereby inducing a genetic disruption of the target sites; and (b) introducing into the immune cell a template polynucleotide comprising a transgene encoding a recombinant receptor that is a recombinant T cell receptor (TCR) or an antigen-binding fragment thereof or a chain thereof, wherein the transgene encoding the recombinant receptor or antigen-binding fragment or chain thereof is targeted for integration at or near one of the at least one of the target site via homology directed repair (HDR).

In some of any such embodiments, also provided herein are methods for producing a genetically engineered immune cell comprising introducing into an immune cell having a genetic disruption of at least one target site within a T cell receptor alpha constant (TRAC) gene and a genetic disruption of at least one target site within a T cell receptor beta constant (TRBC) gene a template polynucleotide comprising a transgene encoding a recombinant receptor that is a recombinant T cell receptor (TCR) or an antigen-binding fragment thereof or a chain thereof, wherein the genetic disruptions have been induced by at least one agent that is capable of inducing a genetic disruption of a target site within the TRAC gene and at least one agent that is capable of inducing a genetic disruption with the TRBC gene, and the transgene encoding the recombinant receptor or antigen-binding fragment or chain thereof is targeted for integration at or near one of the at least one target site via homology directed repair (HDR). In some of any such embodiments, the TRBC gene is one or both of a T cell receptor beta constant 1 (TRBC1) or T cell receptor beta constant 2 (TRBC2) gene.

In certain embodiments, the one or more agent capable of inducing a genetic disruption comprises a DNA binding protein or DNA-binding nucleic acid that specifically binds to or hybridizes to the target site. In some embodiments, the one or more agent capable of inducing a genetic disruption comprises (a) a fusion protein comprising a DNA-targeting protein and a nuclease or (b) an RNA-guided nuclease. In particular embodiments, the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site.

In certain embodiments, the one or more agent comprises a zinc finger nuclease (ZFN), a TAL-effector nuclease (TALEN), or and a CRISPR-Cas9 combination that specifically binds to, recognizes, or hybridizes to the target site. In some embodiments, the each of the one or more agent comprises a guide RNA (gRNA) having a targeting domain that is complementary to the at least one target site. In some of any such embodiments, the each of the one or more agent comprises a CRISPR-Cas9 combination and the CRISPR-Cas9 combination comprises a guide RNA (gRNA) having a targeting domain that is complementary to the at least one target site. In particular embodiments, the one or more agent is introduced as a ribonucleoprotein (RNP) complex comprising the gRNA and a Cas9 protein. In some of any such embodiments, the CRISPR-Cas9 combination is a ribonucleoprotein (RNP) complex comprising the gRNA and a Cas9 protein. In some of any such embodiments, the concentration of the RNP is or is about 1 μM to at or about 5 μM. In some of any such embodiments, the concentration of the RNP is or is about 2 μM.

In certain embodiments, the RNP is introduced via electroporation, particle gun, calcium phosphate transfection, cell compression or squeezing. In some embodiments, the RNP is introduced via electroporation. In particular embodiments, the one or more agent is introduced as one or more polynucleotide encoding the gRNA and/or a Cas9 protein. In certain embodiments, the at least one target site is within an exon of the TRAC, TRBC1 and/or TRBC2 gene. In some of any such embodiments, the at least one target site is within an exon of the TRAC and an exon with the TRBC1 or TRBC2 gene.

In some embodiments, the gRNA has a targeting domain that is complementary to a target site in a TRAC gene and comprises a sequence selected from the group consisting of UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58). In particular embodiments, the gRNA has a targeting domain comprising the sequence GAGAAUCAAAAUCGGUGAAU (SEQ ID NO:31).

In certain embodiments, the gRNA has a targeting domain that is complementary to a target site in one or both of a TRBC1 and a TRBC2 gene and comprises a sequence selected from the group consisting of CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116). In some embodiments, the gRNA has a targeting domain comprising the sequence GGCCUCGGCGCUGACGAUCU (SEQ ID NO:63).

In particular embodiments, the template polynucleotide comprises the structure [5′ homology arm]-[transgene]-[3′ homology arm]. In certain embodiments, the 5′ homology arm and 3′ homology arm comprises nucleic acid sequences homologous to nucleic acid sequences surrounding the at least one target site. In some embodiments, the 5′ homology arm comprises nucleic acid sequences that are homologous to nucleic acid sequences 5′ of the target site. In particular embodiments, the 3′ homology arm comprises nucleic acid sequences that are homologous to nucleic acid sequences 3′ of the target site.

In certain embodiments, the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In some embodiments, the 5′ homology arm and 3′ homology arm independently are between about 50 and 100, 100 and 250, 250 and 500, 500 and 750, 750 and 1000, 1000 and 2000 nucleotides. In some of any such embodiments, the 5′ homology arm and 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

In particular embodiments, the 5′ homology arm and 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides. In some of any such embodiments, the 5′ homology arm and 3′ homology arm independently are from at or about 100 to at or about 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides in length.

In some of any such embodiments, the 5′ homology arm and 3′ homology arm independently are at or about 200, 300, 400, 500, 600, 700 or 800 nucleotides in length, or any value between any of the foregoing. In some of any such embodiments, the 5′ homology arm and 3′ homology arm independently are greater than at or about 300 nucleotides in length, optionally wherein the 5′ homology arm and 3′ homology arm independently are at or about 400, 500 or 600 nucleotides in length or any value between any of the foregoing. In some of any such embodiments, the 5′ homology arm and 3′ homology arm independently are greater than at or about 300 nucleotides in length.

In some of any such embodiments, the transgene encoding the recombinant receptor or antigen-binding fragment or chain thereof is targeted for integration at or near the target site in the TRAC gene. In some of any such embodiments, the transgene encoding the recombinant receptor or antigen-binding fragment or chain thereof is targeted for integration at or near the target site in one or both of the TRBC1 and the TRBC2 gene. In some of any such embodiments, the recombinant receptor is a recombinant TCR comprising an alpha (TCRα) chain and a beta (TCRβ) chain and the transgene encoding the recombinant TCR or antigen-binding fragment or chain thereof comprises a nucleic acid sequence encoding the TCRα chain and a nucleic acid sequence encoding the TCRβ chain. In some of any such embodiments, the transgene further comprises one or more multicistronic element(s) and the multicistronic element(s) is positioned between the nucleic acid sequence encoding the TCRα or a portion thereof and the nucleic acid sequence encoding the TCRβ or a portion thereof. In some of any such embodiments, the multicistronic element(s) comprises a sequence encoding a ribosome skip element selected from among a T2A, a P2A, a E2A or a F2A or an internal ribosome entry site (IRES).

In some of any such embodiments, the recombinant receptor is a recombinant TCR and the transgene encoding the recombinant TCR or antigen-binding fragment or chain thereof comprises a nucleic acid sequence encoding one chain of the recombinant TCR and the second transgene comprises a nucleic acid sequence encoding a different chain of the recombinant TCR. In some of any such embodiments, the transgene encoding the recombinant TCR or antigen-binding fragment or chain thereof comprises the nucleic acid sequence encoding the TCRα chain and the second transgene comprises the nucleic acid sequence encoding the TCRβ chain or a portion thereof.

In certain embodiments, the transgene encoding the recombinant TCR or antigen-binding fragment or chain thereof is targeted for integration at or near the target site in the TRAC gene. In some embodiments, the transgene encoding the recombinant TCR or antigen-binding fragment or chain thereof is targeted for integration at or near the target site in one or both of the TRBC1 and the TRBC2 gene. In particular embodiments, comprising introducing into the immune cell one or more second template polynucleotide comprising one or more second transgene(s), wherein the second transgene is targeted for integration at or near one of the at least one target site via homology directed repair (HDR).

In certain embodiments, the second template polynucleotide comprises the structure [second 5′ homology arm]-[one or more second transgene]-[second 3′ homology arm]. In some embodiments, the second 5′ homology arm and second 3′ homology arm comprise nucleic acid sequences homologous to nucleic acid sequences surrounding the at least one target site. In particular embodiments, the second 5′ homology arm comprises nucleic acid sequences that are homologous to nucleic acid sequences second 5′ of the target site.

In certain embodiments, the second 3′ homology arm comprises nucleic acid sequences that are homologous to nucleic acid sequences second 3′ of the target site. In some embodiments, the second 5′ homology arm and second 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In particular embodiments, the second 5′ homology arm and second 3′ homology arm independently are between about 50 and 100, 100 and 250, 250 and 500, 500 and 750, 750 and 1000, 1000 and 2000 nucleotides. In certain embodiments, the second 5′ homology arm and second 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides.

In some embodiments, the one or more second transgene is targeted for integration at or near the target site in the TRAC gene. In particular embodiments, the one or more second transgene is targeted for integration at or near the target site in the TRBC1 or the TRBC2 gene. In certain embodiments, transgene encoding the recombinant TCR or antigen-binding fragment or chain thereof is targeted for integration at or near the target site in the TRAC gene, the TRBC1 gene or the TRBC2 gene, and the one or more second transgene is targeted for integration at or near one or more of the target site that is not targeted by the transgene encoding the recombinant TCR or antigen-binding fragment or chain thereof. In some of any such embodiments, the transgene encoding the recombinant receptor or antigen-binding fragment or chain thereof is targeted for integration at or near a target site in the TRAC gene, the TRBC1 gene or the TRBC2 gene, and the one or more second transgene is targeted for integration at or near one or more other target site among the TRAC gene, the TRBC1 gene or the TRBC2 gene and that is not targeted by the transgene encoding the recombinant receptor or antigen-binding fragment or chain thereof. In some embodiments, the transgene encoding the recombinant TCR or antigen-binding fragment or chain thereof is targeted for integration at or near the target site in the TRAC gene, and the one or more second transgene is targeted for integration at or near one or more of the target site in the TRBC1 gene and/or the TRBC2 gene. In some of any such embodiments, the transgene encoding the recombinant receptor or antigen-binding fragment or chain thereof is targeted for integration at or near a target site in the TRAC gene, and the one or more second transgene is targeted for integration at or near one or more target site in the TRBC1 gene and/or the TRBC2 gene.

In particular embodiments, the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor. In certain embodiments, the encoded molecule is a co-stimulatory ligand optionally selected from among a tumor necrosis factor (TNF) ligand selected from 4-1BBL, OX40L, CD70, LIGHT and CD30L, or an immunoglobulin (Ig) superfamily ligand selected from CD80 and CD86. In some embodiments, the encoded molecule is a cytokine optionally selected from among IL-2, IL-3, IL-6, IL-11, IL-12, IL-7, IL-15, IL-21, granulocyte macrophage colony stimulating factor (GM-CSF), interferon alpha (IFN-α), interferon beta (IFN-β) or interferon gamma (IFN-γ) and erythropoietin. In particular embodiments, the encoded molecule is a soluble single-chain variable fragment (scFv) that optionally binds a polypeptide that has immunosuppressive activity or immunostimulatory activity selected from CD47, PD-1, CTLA-4 and ligands thereof or CD28, OX-40, 4-1BB and ligands thereof.

In certain embodiments, the encoded molecule is an immunomodulatory fusion protein, optionally comprising: (a) an extracellular binding domain that specifically binds an antigen derived from CD200R, SIRPα, CD279 (PD-1), CD2, CD95 (Fas), CD152 (CTLA4), CD223 (LAG3), CD272 (BTLA), A2aR, KIR, TIM3, CD300 or LPA5; (b) an intracellular signaling domain derived from CD3ε, CD3δ, CD3ζ, CD25, CD27, CD28, CD40, CD47, CD79A, CD79B, CD134 (OX40), CD137 (4-1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRFM, Zap70, PTCH2, or any combination thereof; and (c) a hydrophobic transmembrane domain derived from CD2, CD3ε, CD3δ, CD3ζ, CD25, CD27, CD28, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5 or Zap70. In some embodiments, the encoded molecule is a chimeric switch receptor (CSR) that optionally comprises a truncated extracellular domain of PD1 and the transmembrane and cytoplasmic signaling domains of CD28. In particular embodiments, the encoded molecule is a co-receptor optionally selected from CD4 or CD8.

In certain embodiments, transgene encoding the recombinant TCR or antigen-binding fragment or chain thereof encodes one chain of a recombinant TCR and the second transgene encodes a different chain of the recombinant TCR. In some embodiments, transgene encoding the recombinant TCR or antigen-binding fragment or chain thereof encodes the alpha (TCRα) chain of the recombinant TCR and the second transgene encodes the beta (TCRβ) chain of the recombinant TCR. In particular embodiments, the transgene encoding the recombinant TCR or antigen-binding fragment or chain thereof and/or the one or more second transgene independently further comprises a regulatory or control element. In certain embodiments, the regulatory or control element comprises a promoter, an enhancer, an intron, a polyadenylation signal, a Kozak consensus sequence a splice acceptor sequence or a splice donor sequence. In some embodiments, the regulatory or control element comprises a promoter. In particular embodiments, the promoter is selected from among a constitutive promoter, an inducible promoter, a repressible promoter and/or a tissue-specific promoter. In some embodiments, the promoter is selected from among an RNA pol I, pol II or pol III promoter.

In some of any such embodiments, the transgene encoding the recombinant receptor or antigen-binding fragment or chain thereof further comprises a regulatory or control element. In some of any such embodiments, the transgene encoding the recombinant receptor or antigen-binding fragment or chain thereof and/or the one or more second transgene independently further comprises a heterologous regulatory or control element. In some of any such embodiments, the heterologous regulatory or control element comprises a heterologous promoter. In some of any such embodiments, the heterologous promoter is or comprises a human elongation factor 1 alpha (EF1α) promoter or an MND promoter or a variant thereof. In some of any such embodiments, the heterologous promoter is an inducible promoter or a repressible promoter.

In particular embodiments, the promoter is selected from: a pol III promoter that is a U6 or H1 promoter; or a pol II promoter that is a CMV, SV40 early region or adenovirus major late promoter. In certain embodiments, the promoter is or comprises a human elongation factor 1 alpha (EF1α) promoter or an MND promoter or a variant thereof. In some embodiments, the promoter is an inducible promoter or a repressible promoter. In particular embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In certain embodiments, the transgene encoding the recombinant TCR or antigen-binding fragment or chain thereof and/or the one or more second transgene independently comprises one or more multicistronic element(s). In some embodiments, the one or more multicistronic element(s) are upstream of the transgene encoding the recombinant TCR or antigen-binding fragment or chain thereof and/or the one or more second transgene. In particular embodiments, the multicistronic element(s) is positioned between the transgene encoding the recombinant TCR or antigen-binding fragment or chain thereof and the one or more second transgene. In certain embodiments, the multicistronic element(s) is positioned between the nucleic acid sequence encoding the TCRα or a portion thereof and the nucleic acid sequence encoding the TCRβ or a portion thereof. In some embodiments, the multicistronic element(s) comprises a sequence encoding a riboparticular skip element selected from among a T2A, a P2A, a E2A or a F2A or an internal ribocertain entry site (IRES). In some embodiments, the sequence encoding a riboparticular skip element is targeted to be in-frame with the gene at the target site.

In certain embodiments, the transgene encoding the recombinant TCR or antigen-binding fragment or chain thereof and/or the one or more second transgene independently is operably linked to the endogenous promoter of the gene at the target site.

In some of any such embodiments, the TCRα chain comprises a constant (Ca) region comprising introduction of one or more cysteine residues and/or the TCRβ chain comprises a Cβ region comprising introduction of one or more cysteine residues, wherein the one or more introduced cysteine residues are capable of forming one or more non-native disulfide bridges between the alpha chain and beta chain. In some of any such embodiments, the introduction of the one or more cysteine residues comprises replacement of a non-cysteine residue with a cysteine residue. In some of any such embodiments, the Cα region comprises a cysteine at a position corresponding to position 48 with numbering as set forth in any of SEQ ID NO: 24; and/or the Cβ region comprises a cysteine at a position corresponding to position 57 with numbering as set forth in SEQ ID NO: 20.

In some embodiments, the recombinant TCR is capable of binding to an antigen that is associated with, specific to, and/or expressed on a cell or tissue of a disease, disorder or condition. In particular embodiments, the disease, disorder or condition is an infectious disease or disorder, an autoimmune disease, an inflammatory disease, or a tumor or a cancer. In certain embodiments, the antigen is a tumor antigen or a pathogenic antigen. In some embodiments, the pathogenic antigen is a bacterial antigen or viral antigen.

In particular embodiments, the antigen is a viral antigen and the viral antigen is from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV). In certain embodiments, the antigen is an antigen from an HPV selected from among HPV-16, HPV-18, HPV-31, HPV-33 and HPV-35. In some embodiments, the antigen is an HPV-16 antigen that is an HPV-16 E6 or HPV-16 E7 antigen.

In particular embodiments, the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA. In certain embodiments, the viral antigen is an HTLV-antigen that is TAX. In some embodiments, the viral antigen is an HBV antigen that is a hepatitis B core antigen or a hepatitis B envelope antigen.

In particular embodiments, the antigen is a tumor antigen. In certain embodiments, the antigen is selected from among glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin.

In some of any such embodiments, the immune cells comprise or are enriched in T cells. In some of any such embodiments, the T cells comprise a CD8+ T cells or subtypes thereof. In some of any such embodiments, the T cells comprise a CD4+ T cell or subtypes thereof. In some of any such embodiments, the T cells comprise CD4+ T cell or subtypes thereof and CD8+ T cells or subtypes thereof. In some of any such embodiments, the T cells comprise CD4+ and CD8+ T cells and the ratio of CD4+ to CD8+ T cells is at or about 1:3 to at or about 3:1. In some of any such embodiments the ratio is at or about 1:2 to at or about 2:1. In some of any such embodiments the ratio is at or about 1:1. In some embodiments, the immune cell is a T cell. In particular embodiments, the T cell is a CD8+ T cell or subtypes thereof. In certain embodiments, the T cell is a CD4+ T cell or subtypes thereof.

In some embodiments, the immune cell is derived from a multipotent or pluripotent cell, which optionally is an iPSC. In some of any such embodiments, the immune cell is a primary cell from a subject. In some of any such embodiments, the subject has or is suspected of having the disease, or disorder condition. In some of any such embodiments, the subject is or is suspected of being healthy. In some of any such embodiments, the immune cell is autologous to the subject. In some of any such embodiments, the immune cell is allogeneic to the subject. In particular embodiments, the immune cell comprises a T cell that is autologous to the subject. In certain embodiments, the immune cell comprises a T cell that is allogeneic to the subject.

In some of any such embodiments, the template polynucleotide is comprised in one or more vector(s), which optionally is a viral vector(s). In some of any such embodiments, the vector is a viral vector and the viral vector is an AAV vector. In some of any such embodiments, the AAV vector is selected from the group consisting of AAV1, AAV2, AAV3, AAV4, AAV5, AAV6, AAV7 and AAV8 vector. In some of any such embodiments, the AAV vector is an AAV2 or AAV6 vector. In some of any such embodiments, vector is a viral vector and the viral vector is a retroviral vector. In some of any such embodiments, the viral vector is a lentiviral vector.

In some embodiments, the first template polynucleotide, the one or more second template polynucleotide and/or the one or more polynucleotide encoding the gRNA and/or a Cas9 protein is comprised in one or more vector(s), which optionally are viral vector(s). In particular embodiments, the vector is an AAV vector. In certain embodiments, the AAV vector is selected from among AAV1, AAV2, AAV3, AAV4, AAV5, AAV6, AAV7 or AAV8 vector. In some embodiments, the AAV vector is an AAV2 or AAV6 vector. In particular embodiments, the viral vector is a retroviral vector. In certain embodiments, the viral vector is a lentiviral vector.

In some of any such embodiments, the template polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some of any such embodiments, the polynucleotide is between at or about 2500 and at or about 5000 nucleotides, at or about 3500 and at or about 4500 nucleotides, or at or about 3750 nucleotides and at or about 4250 nucleotides in length.

In some embodiments, the introduction of the one or more agent capable of inducing a genetic disruption and the introduction of the template polynucleotide are performed simultaneously or sequentially, in any order. In particular embodiments, the introduction of the template polynucleotide is performed after the introduction of the one or more agent capable of inducing a genetic disruption. In certain embodiments, the template polynucleotide is introduced immediately after, or within at or about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption. In some of any such embodiments, the template nucleotide is introduced at or about 2 hours after the introduction of the one or more agents.

In some of any such embodiments, introduction of the one or more agent capable of inducing a genetic disruption and the introduction of the template polynucleotide are performed in one experimental reaction. In some of any such embodiments, prior to the introducing of the one or more agent, the method comprises incubating the cells, in vitro with a stimulatory agent(s) under conditions to stimulate or activate the one or more immune cells. In some of any such embodiments, the stimulatory agent (s) comprises and anti-CD3 and/or anti-CD28 antibodies, optionally anti-CD3/anti-CD28 beads. In some of any such embodiments, the bead to cell ratio is or is about 1:1. In some of any such embodiments, the stimulatory agent(s) is removed from the one or more immune cells prior to the introducing with the one or more agents.

In some of any such embodiments, the method further comprises incubating the cells prior to, during or subsequent to the introducing of the one or more agents and/or the introducing of the template polynucleotide with one or more recombinant cytokines. In some of any such embodiments, the one or more recombinant cytokines are selected from the group consisting of IL-2, IL-7, and IL-15. In some of any such embodiments, the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL. In some of any such embodiments the concentration is at or about 5 ng/mL to at or about 10 ng/mL and/or IL-15 at a concentration of 0.1 ng/mL to 20 ng/mL. In some of any such embodiments the concentration is at or about 0.5 ng/mL to at or about 5 ng/mL. In some of any such embodiments, the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days. In any of such embodiments, the introducing is up to or about 7 days.

In some embodiments, the introduction of the template polynucleotide and the introduction of the one or more second template polynucleotide are performed simultaneously or sequentially, in any order. In particular embodiments, introduction of the one or more agent capable of inducing a genetic disruption and the introduction of the template polynucleotide are performed in one experimental reaction. In certain embodiments, introduction of the one or more agent capable of inducing a genetic disruption and the introduction of the template polynucleotide and the second template polynucleotide(s) are performed in one experimental reaction.

In some embodiments, at least or greater than 35%, 40%, 45%, 50%, 55%, 60%, 65%, 70%, 75%, 80%, or 90% of the cells in a plurality of engineered cells comprise a genetic disruption of at least one target site within a gene encoding a domain or region of T cell receptor alpha constant (TRAC) gene and/or a T cell receptor beta constant (TRBC) gene. In particular embodiments, at least or greater than 35%, 40%, 45%, 50%, 55%, 60%, 65%, 70%, 75%, 80%, or 90% of the cells in a plurality of engineered cells express the recombinant receptor or antigen-binding fragment thereof and/or exhibit antigen binding or binding to the antigen. In certain embodiments, the coefficient of variation of expression and/or antigen binding of the recombinant receptor or antigen-binding fragment thereof among a plurality of engineered cells is lower than 0.70, 0.65, 0.60, 0.55, 0.50, 0.45, 0.40, 0.35 or 0.30 or less. In some embodiments, the coefficient of variation of expression and/or antigen binding of the recombinant receptor or antigen-binding fragment thereof among a plurality of engineered cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

In particular embodiments, expression and/or antigen-binding of the recombinant receptor or antigen-binding fragment thereof is assessed by contacting the cells in the composition with a binding reagent specific for the TCRα chain or the TCRβ chain and assessing binding of the reagent to the cells. In certain embodiments, the binding reagent is an anti-TCR Vβ antibody or is an anti-TCR Vα antibody that specifically recognizes a specific family of Vβ or Vα chains. In some embodiments, the binding agent is a peptide antigen-MHC complex, which optionally is a tetramer.

Provided herein is an engineered cell or a plurality of engineered cells, generated using a method described herein.

In some of any such embodiments, provided herein is a method of treatment comprising administering the engineered cell, plurality of engineered cells or composition to a subject in need thereof. In some of any such embodiments, the subject has the disease, disorder or condition. In some of any such embodiments, the disease, disorder or condition is a cancer.

In some of any such embodiments, provided herein is the use of the engineered cell, plurality of engineered cells or composition for treating cancer disease, disorder or condition. In some of any such embodiments, the disease, disorder or condition is a cancer.

In some of any such embodiments, provided herein is the use of the engineered cell, plurality of engineered cells or composition for the manufacture of a medicament for treating a disease, disorder or condition. In some of any of such embodiments, the disease, disorder or condition is a cancer.

In some of any such embodiments, provided herein is the use of the engineered cell, plurality of engineered cells or composition for use in treating cancer disease disorder or condition. In some of any of such embodiments, the disease, disorder or condition is a cancer.

Provided herein is a method of treatment comprising administering the engineered cell, plurality of engineered cells or composition described herein to a subject. Provided herein is a use of an engineered cell, a plurality of engineered cells or a composition described herein for treating cancer. Provided herein is a use of an engineered cell, a plurality of engineered cells or a composition described herein in the manufacture of a medicament for treating cancer. Certain embodiments provide an engineered cell, a plurality of engineered cells or composition described herein for use in treating cancer.

In some of any such embodiments, provided herein is a kit comprising: one or more agent, wherein each of the one or more agent is independently capable of inducing a genetic disruption of a target site within a T cell receptor alpha constant (TRAC) gene and/or a T cell receptor beta constant (TRBC) gene; and a template polynucleotide comprising a transgene encoding a recombinant receptor or an antigen-binding fragment or a chain thereof, wherein the transgene encoding the recombinant receptor or antigen-binding fragment or chain thereof is targeted for integration at or near the target site via homology directed repair (HDR) and instructions for carrying out the method of any of the embodiments described herein.

Also provided herein is a kit, comprising: one or more agent, wherein each of the one or more agent is independently capable of inducing a genetic disruption of a target site within a T cell receptor alpha constant (TRAC) gene and/or a T cell receptor beta constant (TRBC) gene; and a template polynucleotide comprising a transgene encoding a recombinant TCR or an antigen-binding fragment or a chain thereof, wherein the transgene encoding the recombinant TCR or antigen-binding fragment or chain thereof is targeted for integration at or near the target site via homology directed repair (HDR). In particular embodiments, the one or more agent capable of inducing a genetic disruption comprises a DNA binding protein or DNA-binding nucleic acid that specifically binds to or hybridizes to the target site.

In certain embodiments, the one or more agent capable of inducing a genetic disruption comprises (a) a fusion protein comprising a DNA-targeting protein and a nuclease or (b) an RNA-guided nuclease. In some embodiments, the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site. In particular embodiments, the one or more agent comprises a zinc finger nuclease (ZFN), a TAL-effector nuclease (TALEN), or and a CRISPR-Cas9 combination that specifically binds to, recognizes, or hybridizes to the target site. In certain embodiments, the each of the one or more agent comprises a guide RNA (gRNA) having a targeting domain that is complementary to the at least one target site.

In some embodiments, the one or more agent is introduced as a ribonucleoprotein (RNP) complex comprising the gRNA and a Cas9 protein. In particular embodiments, the RNP is introduced via electroporation, particle gun, calcium phosphate transfection, cell compression or squeezing. In certain embodiments, the RNP is introduced via electroporation. In some embodiments, the one or more agent is introduced as one or more polynucleotide encoding the gRNA and/or a Cas9 protein. In particular embodiments, the at least one target site is within an exon of the TRAC, TRBC1 and/or TRBC2 gene.

In certain embodiments, the gRNA has a targeting domain that is complementary to a target site in a TRAC gene and comprises a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58). In some embodiments, the gRNA has a targeting domain comprising the sequence GAGAAUCAAAAUCGGUGAAU (SEQ ID NO:31).

In particular embodiments, the gRNA has a targeting domain that is complementary to a target site in one or both of a TRBC1 and a TRBC2 gene and comprises a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116). In certain embodiments, the gRNA has a targeting domain comprising the sequence GGCCUCGGCGCUGACGAUCU (SEQ ID NO:63).

In some embodiments, the template polynucleotide comprises the structure [5′ homology arm]-[transgene]-[3′ homology arm]. In particular embodiments, the 5′ homology arm and 3′ homology arm comprises nucleic acid sequences homologous to nucleic acid sequences surrounding the at least one target site. In certain embodiments, the 5′ homology arm comprises nucleic acid sequences that are homologous to nucleic acid sequences 5′ of the target site. In some embodiments, the 3′ homology arm comprises nucleic acid sequences that are homologous to nucleic acid sequences 3′ of the target site. In particular embodiments, the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In certain embodiments, the 5′ homology arm and 3′ homology arm independently are between about 50 and 100, 100 and 250, 250 and 500, 500 and 750, 750 and 1000, 1000 and 2000 nucleotides.

In some embodiments, the 5′ homology arm and 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides. In particular embodiments, the transgene encoding the recombinant TCR or antigen-binding fragment or chain thereof is targeted for integration at or near the target site in the TRAC gene. In certain embodiments, the transgene encoding the recombinant TCR or antigen-binding fragment or chain thereof is targeted for integration at or near the target site in one or both of the TRBC1 and the TRBC2 gene. In some aspects, the kit further comprises one or more second template polynucleotide comprising one or more second transgene, wherein the second transgene is targeted for integration at or near one of the at least one target site via homology directed repair (HDR).

In particular embodiments, the second template polynucleotide comprises the structure [second 5′ homology arm]-[one or more second transgene]-[second 3′ homology arm]. In certain embodiments, the second 5′ homology arm and second 3′ homology arm comprises nucleic acid sequences homologous to nucleic acid sequences surrounding the at least one target site. In some embodiments, the second 5′ homology arm comprises nucleic acid sequences that are homologous to nucleic acid sequences second 5′ of the target site. In particular embodiments, the second 3′ homology arm comprises nucleic acid sequences that are homologous to nucleic acid sequences second 3′ of the target site. In certain embodiments, the second 5′ homology arm and second 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides. In some embodiments, the second 5′ homology arm and second 3′ homology arm independently are between about 50 and 100, 100 and 250, 250 and 500, 500 and 750, 750 and 1000, 1000 and 2000 nucleotides.

In particular embodiments, the second 5′ homology arm and second 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides.

In certain embodiments, the one or more second transgene is targeted for integration at or near the target site in the TRAC gene. In some embodiments, the one or more second transgene is targeted for integration at or near the target site in the TRBC1 or the TRBC2 gene. In particular embodiments, transgene encoding the recombinant TCR or antigen-binding fragment or chain thereof is targeted for integration at or near the target site in the TRAC gene, the TRBC1 gene or the TRBC2 gene, and the one or more second transgene is targeted for integration at or near one or more of the target site that is not targeted by the transgene encoding the recombinant TCR or antigen-binding fragment or chain thereof.

In certain embodiments, the transgene encoding the recombinant TCR or antigen-binding fragment or chain thereof is targeted for integration at or near the target site in the TRAC gene, and the one or more second transgene is targeted for integration at or near one or more of the target site in the TRBC1 gene and/or the TRBC2 gene. In some embodiments, the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor. In particular embodiments, the encoded molecule is a co-stimulatory ligand optionally selected from among a tumor necrosis factor (TNF) ligand selected from 4-1BBL, OX40L, CD70, LIGHT and CD30L, or an immunoglobulin (Ig) superfamily ligand selected from CD80 and CD86. In certain embodiments, the encoded molecule is a cytokine optionally selected from among IL-2, IL-3, IL-6, IL-11, IL-30, IL-7, IL-24, IL-30, granulocyte macrophage colony stimulating factor (GM-CSF), interferon alpha (IFN-α), interferon beta (IFN-β) or interferon gamma (IFN-γ) and erythropoietin.

In some embodiments, the encoded molecule is a soluble single-chain variable fragment (scFv) that optionally binds a polypeptide that has immunosuppressive activity or immunostimulatory activity selected from CD47, PD-1, CTLA-4 and ligands thereof or CD28, OX-40, 4-1BB and ligands thereof. In particular embodiments, the encoded molecule is an immunomodulatory fusion protein, optionally comprising: (a) an extracellular binding domain that specifically binds an antigen derived from CD290R, SIRPα, CD279 (PD-1), CD2, CD95 (Fas), CD242 (CTLA4), CD223 (LAG3), CD272 (BTLA), A2aR, KIR, TIM3, CD300 or LPA5; (b) an intracellular signaling domain derived from CD3ε, CD3δ, CD3ζ, CD25, CD27, CD28, CD40, CD47, CD79A, CD79B, CD224 (OX40), CD227 (4-1BB), CD240 (SLAMF1), CD278 (ICOS), CD357 (GITR), CARD11, DAP10, DAP30, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRFM, Zap70, PTCH2, or any combination thereof; and (c) a hydrophobic transmembrane domain derived from CD2, CD3ε, CD3δ, CD3ζ, CD25, CD27, CD28, CD40, CD79A, CD79B, CD80, CD86, CD95 (Fas), CD224 (OX40), CD227 (4-1BB), CD240 (SLAMF1), CD242 (CTLA4), CD290R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5 or Zap70. In certain embodiments, the encoded molecule is a chimeric switch receptor (CSR) that optionally comprises a truncated extracellular domain of PD1 and the transmembrane and cytoplasmic signaling domains of CD28.

In some embodiments, the encoded molecule is a co-receptor optionally selected from CD4 or CD8. In particular embodiments, transgene encoding the recombinant TCR or antigen-binding fragment or chain thereof encodes one chain of a recombinant TCR and the second transgene encodes a different chain of the recombinant TCR. In certain embodiments, transgene encoding the recombinant TCR or antigen-binding fragment or chain thereof encodes the alpha (TCRα) chain of the recombinant TCR and the second transgene encodes the beta (TCRβ) chain of the recombinant TCR. In some embodiments, the transgene encoding the recombinant TCR or antigen-binding fragment or chain thereof and/or the one or more second transgene independently further comprises a regulatory or control element.

In particular embodiments, the regulatory or control element comprises a promoter, an enhancer, an intron, a polyadenylation signal, a Kozak consensus sequence, a splice acceptor sequence or a splice donor sequence. In certain embodiments, the regulatory or control element comprises a promoter. In some embodiments, the promoter is selected from among a constitutive promoter, an inducible promoter, a repressible promoter and/or a tissue-specific promoter. In particular embodiments, the promoter is selected from among an RNA pol I, pol II or pol III promoter. In certain embodiments, the promoter is selected from: a pol III promoter that is a U6 or H1 promoter; or a pol II promoter that is a CMV, SV40 early region or adenovirus major late promoter. In some embodiments, the promoter is or comprises a human elongation factor 1 alpha (EF1α) promoter or an MND promoter or a variant thereof. In particular embodiments, the promoter is an inducible promoter or a repressible promoter. In certain embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In some embodiments, the transgene encoding the recombinant TCR or antigen-binding fragment or chain thereof and/or the one or more second transgene independently comprises one or more multicistronic element(s). In particular embodiments, the one or more multicistronic element(s) are upstream of the transgene encoding the recombinant TCR or antigen-binding fragment or chain thereof and/or the one or more second transgene. In certain embodiments, the multicistronic element(s) is positioned between the transgene encoding the recombinant TCR or antigen-binding fragment or chain thereof and the one or more second transgene. In some embodiments, the multicistronic element(s) is positioned between the nucleic acid sequence encoding the TCRα or a portion thereof and the nucleic acid sequence encoding the TCRβ or a portion thereof. In particular embodiments, the multicistronic element(s) comprises a sequence encoding a ribocertain skip element selected from among a T2A, a P2A, a E2A or a F2A or an internal ribosome entry site (IRES). In particular embodiments, the sequence encoding a ribocertain skip element is targeted to be in-frame with the gene at the target site.

In some embodiments, upon HDR, the transgene encoding the recombinant TCR or antigen-binding fragment or chain thereof and/or the one or more second transgene independently is operably linked to the endogenous promoter of the gene at the target site. In particular embodiments, the recombinant TCR is capable of binding to an antigen that is associated with, specific to, and/or expressed on a cell or tissue of a disease, disorder or condition. In certain embodiments, the disease, disorder or condition is an infectious disease or disorder, an autoimmune disease, an inflammatory disease, or a tumor or a cancer. In some embodiments, the antigen is a tumor antigen or a pathogenic antigen. In particular embodiments, the pathogenic antigen is a bacterial antigen or viral antigen. In certain embodiments, the antigen is a viral antigen and the viral antigen is from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV). In some embodiments, the antigen is an antigen from an HPV selected from among HPV-25, HPV-27, HPV-31, HPV-33 and HPV-35.

In particular embodiments, the antigen is an HPV-25 antigen that is an HPV-25 E6 or HPV-25 E7 antigen. In certain embodiments, the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA. In some embodiments, the viral antigen is an HTLV-antigen that is TAX. In particular embodiments, the viral antigen is an HBV antigen that is a hepatitis B core antigen or a hepatitis B envelope antigen. In certain embodiments, the antigen is a tumor antigen.

In some embodiments, the antigen is selected from among glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD223, CD 256, epCAM, CA-224, HE4, Oval, estrogen receptor, progesterone receptor, uPA, PAI-1, CD28, CD29, CD22, ROR1, CD33/IL3Ra, c-Met, PSMA, Glycolipid F77, GD-2, insulin growth factor (IGF)-I, IGF-II, IGF-I receptor and mesothelin.

In particular embodiments, the first template polynucleotide, the one or more second template polynucleotide and/or the one or more polynucleotide encoding the gRNA and/or a Cas9 protein is comprised in one or more vector(s), which optionally are viral vector(s). In certain embodiments, the vector is an AAV vector. In some embodiments, the AAV vector is selected from among AAV1, AAV2, AAV3, AAV4, AAV5, AAV6, AAV7 or AAV8 vector. In particular embodiments, the AAV vector is an AAV2 or AAV6 vector. In certain embodiments, the viral vector is a retroviral vector. In some embodiments, the viral vector is a lentiviral vector.

### BRIEF DESCRIPTION OF THE DRAWINGS

FIG. 1A depicts surface expression of CD8 and peptide-MHC tetramer complexed with the antigen recognized by an exemplary recombinant TCR (TCR #1), as assessed by flow cytometry, for T cells subject to knockout of endogenous TCR encoding genes, engineered to express TCR #1 using various methods of expression: cells subject to lentiviral transduction for random integration of the recombinant TCR-encoding sequences (“TCR #1 Lenti”), cells subject to random integration and CRISPR/Cas9 mediated knockout (KO) of TRAC (“TCR #1 Lenti KO”); or cells subject to targeted integration by HDR at the TRAC locus of the recombinant TCR-encoding sequences, under the control of the human EF1α promoter (TCR #1 HDR KO). FIGS. 1B and 1C depict the mean fluorescence intensity (MFI; FIG. 1B) and the coefficient of variation (the standard deviation of signal within a population of cells divided by the mean of the signal in the respective population; FIG. 1C) of cell surface expression of binding of the peptide-MHC tetramer in CD8+ T cells engineered to express TCR #1.

FIG. 2A depicts surface expression of CD8 and peptide-MHC tetramer complexed with the antigen recognized by an exemplary recombinant TCR (TCR #2), as assessed by flow cytometry, for T cells subject to knockout of endogenous TCR encoding genes, engineered to express TCR #2 using various methods of expression: cells subject to lentiviral transduction for random integration of the recombinant TCR-encoding sequences (“TCR #2 Lenti”), cells subject to random integration and CRISPR/Cas9 mediated knockout (KO) of TRAC (“TCR #2 Lenti KO”); or cells subject to targeted integration by HDR at the TRAC locus of the recombinant TCR-encoding sequences, under the control of the human EF1α promoter (TCR #2 HDR KO). FIG. 2B depicts the mean fluorescence intensity (MFI) of cell surface expression of binding of the peptide-MHC tetramer in CD8+ and CD4+ T cells engineered to express TCR #2.

FIG. 3A depicts the average cytolytic activity of the various recombinant TCR #1-expressing CD8+ T cells as described above generated from 2 donors, represented by the area under the curve (AUC) of % killing, compared to mock transduction control and normalized to Vbeta expression (recombinant TCR-specific staining) for each group described above, after incubation of the effector cells as described above with target cells expressing HPV 16 E7 at an effector to target (E:T) ratio of 10:1, 5:1 and 2.5:1. CD8+ cells transduced with a lentivirus encoding a reference TCR capable of binding to HPV 16 E7 but containing mouse Cα and the Cβ regions was assessed as a control (“Lenti Ref”). FIG. 3B depict the average IFNγ secretion (pg/mL) by the various recombinant TCR #1-expressing CD8+ T cells as described above.

FIG. 4A depicts the average cytolytic activity of the various recombinant TCR #2-expressing CD8+ T cells as described above generated from 2 donors, represented by the area under the curve (AUC) of % killing, compared to mock transduction control and normalized to Vbeta expression (recombinant TCR-specific staining) for each group described above, after incubation of the effector cells as described above with target cells expressing HPV 16 E7 at an effector to target (E:T) ratio of 10:1, 5:1 and 2.5:1. CD8+ cells transduced with a lentivirus encoding a reference TCR capable of binding to HPV 16 E7 but containing mouse Cα and the Cβ regions was assessed as a control (“Lenti Ref”). FIGS. 4B and 4C depict the average IFNγ (pg/mL; FIG. 4B) and IL-2 (pg/mL; FIG. 4C) secretion by the various recombinant TCR #2-expressing CD8+ T cells as described above. FIGS. 4D and 4E depict cytolytic activity of the various recombinant TCR #2-expressing CD8+(FIG. 4D) or CD4+(FIG. 4E) T cells as shown by the number of viable target cells over time. FIGS. 4F and 4G depict IFNγ secretion by the various recombinant TCR #2-expressing cells at an E:T ratio of 2.5:1 (FIG. 4F) or 10:1 (FIG. 4G).

FIGS. 5A and 5B depict the viability as determined by the % of cells stained with acridine orange (AO) and propidium iodide (PI), at cryopreservation (at freeze) or after thawing from cryopreservation (at thaw), in various CD4+(FIG. 5A) or CD8+(FIG. 5B) cells engineered to express recombinant TCR #2.

FIGS. 6A and 6B depicts surface expression of CD8, CD3, Vbeta (recombinant TCR-specific staining) and peptide-MHC tetramer complexed with the antigen recognized by the recombinant TCR, as assessed by flow cytometry, for T cells subject to knockout of endogenous TCR encoding genes, engineered to express a recombinant T cell receptor (TCR) using various methods of expression: cells subject to CRISPR/Cas9 mediated knockout (KO) of TRAC and TRBC (“TCRαβ KO”) or retaining expression of the endogenous TCR (“TCRαβ WT”); cells subject to targeted integration by HDR at the TRAC locus of the recombinant TCR-encoding sequences linked to the EF1α or MND promoter (“HDR EF1a” or “HDR MND”); cells subject to lentiviral transduction for random integration of the recombinant TCR-encoding sequences (“lenti human”), or of the recombinant TCR-encoding sequences containing a mouse constant domain (“lenti mouse”), or mock transduction as control (“mock transd”).

FIGS. 6C and 6D depict the geometric mean fluorescence intensity (gMFI) of cell surface expression of Vbeta and binding of the peptide-MHC tetramer in CD8+(FIG. 6C) or CD4+(FIG. 6D) T cells engineered to express a recombinant T cell receptor (TCR) using various methods of expression as described above.

FIGS. 6E and 6F show the coefficient of variation (the standard deviation of signal within a population of cells divided by the mean of the signal in the respective population) in CD8+ T cells engineered to express a recombinant T cell receptor (TCR) using various methods of expression as described above, for expression of Vbeta (FIG. 6F) and binding of the peptide-MHC tetramer (FIG. 6E).

FIGS. 7A-7C depict surface expression of CD3 and CD8, as assessed by flow cytometry, for T cells subject to knockout of endogenous TCR encoding genes, engineered to express a recombinant T cell receptor (TCR) using various methods of expression: cells subject to CRISPR/Cas9 mediated knockout (KO) of TRAC, TRBC or both TRAC and TRBC; cells subject to targeted integration by HDR at the TRAC locus of the recombinant TCR-encoding sequences linked to the EF1α promoter, MND promoter or endogenous TCR alpha promoter using a P2A ribosome skip sequence (“HDR EF1α,” “HDR MND” or “HDR P2A,” respectively) or cells subject to mock transduction as control (“mock transd”) (FIG. 7A); cells retaining expression of the endogenous TCR and subject to lentiviral transduction for random integration of the recombinant TCR-encoding sequences linked to the EF1α promoter (“lenti EF1α”) or MND promoter (“lenti MND”), or linked to EF1α promoter with sequences encoding the truncated receptor as a surrogate marker (“lenti EF1α/tReceptor”), or subject to mock transduction as a control (“mock”) (FIG. 7B). FIG. 7C depicts the percentage of CD3+CD8+ cells among CD8+ cells in each of the groups described above.

FIGS. 8A-8C depict binding of the peptide-MHC tetramer and surface expression of CD8, as assessed by flow cytometry, for T cells subject to knockout of endogenous TCR encoding genes, engineered to express a recombinant T cell receptor (TCR) using various methods of expression: cells subject to CRISPR/Cas9 mediated knockout (KO) of TRAC, TRBC or both TRAC and TRBC; cells subject to targeted integration by HDR at the TRAC locus of the recombinant TCR-encoding sequences linked to the EF1α promoter, MND promoter or endogenous TCR alpha promoter using a P2A ribosome skip sequence (“HDR EF1α,” “HDR MND” or “HDR P2A,” respectively) or cells subject to mock transduction as control (“mock transd”) (FIG. 8A); cells retaining expression of the endogenous TCR and subject to lentiviral transduction for random integration of the recombinant TCR-encoding sequences linked to the EF1α promoter (“lenti EF1α”) or MND promoter (“lenti MND”), or linked to EF1α promoter with sequences encoding a truncated receptor as a surrogate marker (“lenti EF1α/tReceptor”), or subject to mock transduction as a control (“mock”) (FIG. 8B). FIG. 8C depicts the percentage of tetramer+CD8+ cells among CD8+ cells in each of the groups described above, on day 7 and day 13.

FIGS. 9A-9D depict surface expression of Vbeta (recombinant TCR-specific staining) and CD8, as assessed by flow cytometry, for T cells subject to knockout of endogenous TCR encoding genes, engineered to express a recombinant T cell receptor (TCR) using various methods of expression: cells subject to CRISPR/Cas9 mediated knockout (KO) of TRAC, TRBC or both TRAC and TRBC; cells subject to targeted integration by HDR at the TRAC locus of the recombinant TCR-encoding sequences linked to the EF1α promoter, MND promoter or endogenous TCR alpha promoter using a P2A ribosome skip sequence (“HDR EF1α,” “HDR MND” or “HDR P2A,” respectively) or cells subject to mock transduction as control (“mock transd”) (FIG. 9A); cells retaining expression of the endogenous TCR and subject to lentiviral transduction for random integration of the recombinant TCR-encoding sequences linked to the EF1α promoter (“lenti EF1α”) or MND promoter (“lenti MND”), or linked to EF1α promoter with sequences encoding a truncated receptor as a surrogate marker (“lenti EF1a/Receptor”), or subject to mock transduction as a control (“mock”) (FIG. 9B). FIGS. 9C and 9D depict the percentage of Vbeta+CD8+ cells among CD8+ cells (FIG. 9C) and the percentage of Vbeta+CD4+ cells among CD4+ cells (FIG. 9D) in each of the groups described above, on day 7 and day 13.

FIG. 10 depicts the cytolytic activity of the various recombinant TCR-expressing CD8+ T cells as described above, represented by the area under the curve (AUC) of % killing, compared to mock transduction control and normalized to Vbeta expression for each group, from incubation of the effector cells as described above with target cells expressing HPV 16 E7 at an effector to target (E:T) ratio of 10:1, 5:1 and 2.5:1. CD8+ cells transduced with a lentivirus encoding a reference TCR capable of binding to HPV 16 E7 but containing mouse Cα and the Cβ regions was assessed as a control (“lenti mouse E7 ref”).

FIG. 11 depicts the IFNγ secretion (pg/mL) by the various recombinant TCR-expressing CD8+ T cells as described above, from incubation of the effector cells as described above with target cells expressing HPV 16 E7 at an effector to target (E:T) ratio of 10:1 and 2.5:1. CD8+ cells transduced with a lentivirus encoding a reference TCR capable of binding to HPV 16 E7 but containing mouse Cα and the Cβ regions was assessed as a control (“lenti mouse E7 ref”).

FIG. 12 depicts a heat map showing the relative activity various recombinant TCR-expressing T cells as described above in various functional assays: AUC of % killing at E:T ratios of 10:1, 5:1 and 2.5:1 (“AUC”), tetramer binding in CD8+ cells on days 7 and 13 (“tetramer CD8”), proliferation assay (“CTV count”) using SCC152 cells or T2 target cells pulsed with the antigen peptide and secretion of IFNγ from CD8+ cells (“CD8 secreted IFNg”).

FIGS. 13A-13B depict results of the changes in tumor volume over time in UPCI:SCC152 squamous cell carcinoma tumor model mice that have been administered CD4+ and CD8+ cells engineered to express the exemplary recombinant TCR #2 generated by various methods: TCR #2 controlled by the human elongation factor 1 alpha (EF1α) promoter, targeted for integration at the TRAC locus by HDR (TCR #2 HDR KO EF1α); TCR #2 controlled by the endogenous TRAC promoter (by upstream in-frame P2A ribosome skip element), targeted for integration at the TRAC locus by HDR (TCR #2 HDR KO P2A); TCR #2 randomly integrated using lentiviral construct (TCR #2 Lenti); TCR #2 randomly integrated using lentiviral construct in cells containing a knock-out of the endogenous TRAC (TCR #2 Lenti KO); and reference TCR capable of binding to HPV 16 E7 but containing mouse Cα and the Cβ regions randomly integrated using lentiviral construct (Lenti Ref), compared to mice that received no engineered cells (tumor alone) or that were administered cells treated under the same conditions used for electroporation but without addition of an RNP (mock KO), at a dose of 6×10⁶ (FIG. 13A) or 3×10⁶ (FIG. 13B) TCR-expressing cells.

FIGS. 14A-14B depict survival curve of mice in each group described above, for mice receiving a dose of 6×10⁶ (FIG. 14A) or 3×10⁶ (FIG. 14B) recombinant TCR-expressing cells.

FIGS. 15A-15B depict the % change in body weight over time in mice in each group described above, for mice receiving a dose of 6×10⁶ (FIG. 15A) or 3×10⁶ (FIG. 15B) recombinant TCR-expressing cells.

FIGS. 16A-16B depict results for the integration at various time points for the various homology arm lengths tested, as assessed by changes in GFP patterns at 24,48 and 72 hours (FIG. 16A), and at 96 hours or 7 days (FIG. 16B) after transduction with AAV preparations containing the HDR template polynucleotides.

FIGS. 17A-17B depict the change in integration ratio for HDR using the various homology arm lengths, at 24, 48, 72 and 96 hours or 7 days for four different donors, Donor 1 and 2 (FIG. 17A) and Donor 3 and 4 (FIG. 17B).

FIGS. 18A-18B depict results from assessment of expression and activity of an exemplary anti-CD19 CAR, in cells engineered by integration of the nucleic acid sequences into the endogenous TRAC locus. FIG. 18A depicts surface expression of CD3 and anti-CD19 CAR (as detected by staining with an anti-idiotype (anti-ID) antibody that specifically recognizes the CAR) as assessed by flow cytometry, for T cells subject to knockout of endogenous TCR encoding genes, engineered to express anti-CD19 CAR using various methods of expression: cells subject to retroviral transduction for random integration of the recombinant TCR-encoding sequences (“Retrovirus only”), cells subject to targeted integration by HDR at the TRAC locus of the recombinant TCR-encoding sequences, under the control of the human EF1α promoter (EF1α) or endogenous TRAC promoter using a P2A ribosome skip sequence (P2A). FIG. 18B depicts the expression as assessed by flow cytometry of exemplary anti-CD19 CAR-expressing T cells, for the various methods of expression described above subject to electroporation with ribonucleoprotein (RNP) complexes containing TRAC-targeting or TRBC-targeting gRNA.

FIGS. 19A-19C depict the expression and antigen-specific function of cells expressing an exemplary anti-CD19 CAR engineered using various methods of expression following repeated rounds of antigen stimulation with target cells. FIG. 19A depicts the percentage of CAR-expressing cells observed over 3 rounds of stimulation by target cells. FIG. 19B depicts the mean fluorescence intensity (MFI) and FIG. 19C depicts the coefficient of variation (the standard deviation of signal within a population of cells divided by the mean of the signal in the respective population), for T cells engineered to express anti-CD19 CAR, over 3 rounds of stimulation.

FIGS. 20A-20B depict the IFNγ secretion (FIG. 20A; pg/mL) and cytolytic activity (FIG. 20B) of cells expressing the exemplary anti-CD19 CAR using various methods of engineering, incubated with K562 target cells engineered to express CD19 (K562-CD19) or non-engineered K562 (parental) at an effector to target (E:T) ratio of 2:1.

FIGS. 21A-21B depict results from assessment of expression and activity of an exemplary anti-BCMA CAR, in cells engineered by integration of the nucleic acid sequences into the endogenous TRAC locus FIG. 21A depicts surface expression of CD3 and anti-BCMA CAR (recognized by a BCMA-Fc fusion protein), as assessed by flow cytometry, for T cells engineered to express anti-BCMA CAR using various methods of expression: cells subject to retroviral transduction for random integration of the recombinant TCR-encoding sequences (“Lentivirus only”), cells subject to targeted integration by HDR at the TRAC locus of the recombinant TCR-encoding sequences, under the control of the human EF1α promoter (EF1α) or endogenous TCR alpha promoter using a P2A ribosome skip sequence (P2A). FIG. 21B depicts the expression as assessed by flow cytometry of exemplary anti-BCMA CAR-expressing T cells, for the various methods of expression described above subject to electroporation with ribonucleoprotein (RNP) complexes containing TRAC-targeting or TRBC-targeting gRNA.

FIGS. 22A-22B depict the expression and antigen-specific function of cells expressing an exemplary anti-BCMA CAR engineered using various methods of expression following repeated rounds of antigen stimulation with target cells. FIG. 22A depicts the percentage of CAR-expressing cells observed over 3 rounds of stimulation by target cells. FIG. 22B show the level of IFNγ secretion (top panel pg/mL) and interleukin-2 (IL-2; bottom panel).

### DETAILED DESCRIPTION

Provided herein are methods for producing genetically engineered immune cells expressing a recombinant receptor, such as a recombinant T cell receptor (TCR). Also provided are genetically engineered immune cells expressing a recombinant receptor, such as a recombinant T cell receptor (TCR) and compositions containing such cells. The provided embodiments involve specifically targeting nucleic acid sequences encoding the recombinant receptor to a particular locus, e.g., at one or more of the endogenous TCR gene loci. In some contexts, the provided embodiments involve inducing a targeted genetic disruption, e.g., generation of a DNA break, using gene editing methods, and homology-directed repair (HDR) for targeted knock-in of the recombinant receptor-encoding nucleic acids at the endogenous TCR gene loci, thereby reducing or eliminating the expression of the endogenous TCR genes and facilitating a uniform or homogeneous expression of the recombinant receptor within a cell population. Also provided are related cell compositions, nucleic acids and kits for use in the methods provided herein.

T cell-based therapies, such as adoptive T cell therapies (including those involving the administration of engineered cells expressing recombinant receptors specific for a disease or disorder of interest, such as a TCR, a CAR and/or other recombinant antigen receptors) can be effective in the treatment of cancer and other diseases and disorders. In certain contexts, available approaches for generating engineered cells for adoptive cell therapy may not always be entirely satisfactory. In some contexts, optimal efficacy can depend on the ability of the administered cells to express the recombinant receptor, and for the recombinant receptor to recognize and bind to a target, e.g., target antigen, within the subject, tumors, and environments thereof, and for uniform, homogenous and/or consistent expression of the receptors among cells, such as a population of immune cells and/or cells in a therapeutic cell composition.

In some cases, currently available methods, e.g., random integration of sequences encoding the recombinant receptor, are not entirely satisfactory in one or more of these aspects. In some aspects, variable integration of the sequences encoding the recombinant receptor can result in inconsistent expression, variable copy number of the nucleic acids, possible insertional mutagenesis and/or variability of receptor expression and/or genetic disruption within the cell composition, such as a therapeutic cell composition. In some aspects, use of particular random integration vectors, such as certain lentiviral vectors, requires the performance of replication competent lentivirus (RCL) assay.

In some cases, consistency and/or efficiency of expression of the recombinant receptor is limited in certain cells or certain cell populations engineered using currently available methods. In some embodiments, the recombinant receptor is only expressed in certain cells, and the level of expression or antigen binding by the recombinant receptor varies widely among cells in the population. In particular aspects, the level of expression of the recombinant receptor can be difficult to predict, control and/or regulate. In some cases, semi-random or random integration of a transgene encoding the receptor into the genome of the cell may, in some cases, result in adverse and/or unwanted effects due to integration of the nucleic acid sequence into an undesired location in the genome, e.g., into an essential gene or a gene critical in regulating the activity of the cell. In some cases, random integration of a nucleic acid sequence encoding the receptor can result in variegated, unregulated, uncontrolled and/or suboptimal expression or antigen binding, oncogenic transformation and transcriptional silencing of the nucleic acid sequence, depending on the site of integration and/or nucleic acid sequence copy number. In other cases, particularly for recombinant TCRs, suboptimal expression of the engineered or recombinant TCR can occur due expression of one or more chains of the endogenous TCR in the engineered cell can result in mispairing of between the recombinant TCRα or β chain and an endogenous TCRα or β chain. In some aspects, mispaired TCRs can lead to undesired cell targeting and potential adverse effects. In some aspects, mispaired TCRs can compete for invariant CD3 signaling molecules that are involved in permitting expression of the recombinant TCR complex on the cell surface, thereby reducing the recombinant TCR cell surface expression and/or capacity to recognize and bind to a target, e.g., target antigen.

In some embodiments, targeted genetic disruption of one or more of the endogenous TCR gene loci can lead to a reduced risk or chance of mispairing between chains of the engineered or recombinant TCR and the endogenous TCR. Mispaired TCRs can, in some aspects, create a new TCR that could potentially result in a higher risk of undesired or unintended antigen recognition and/or side effects, and/or could reduce expression levels of the desired engineered or recombinant TCR. In some aspects, reducing or preventing endogenous TCR expression can increase expression of the engineered or recombinant TCR in the T cells or T cell compositions as compared to cells in which expression of the TCR is not reduced or prevented. In some embodiments, recombinant TCR expression can be increased by 1.5-fold, 2-fold, 3-fold, 4-fold, 5-fold or more. For example, in some cases, suboptimal expression of an engineered or recombinant TCR can occur due to competition with an endogenous TCR and/or with TCRs having mispaired chains, for signaling molecules and/or domains such as the invariant CD3 signaling molecules (e.g., availability of co-expressed co-expression of CD3 δ, ε, γ and ζ chains) that are involved in permitting expression of the complex on the cell surface. In some aspects, available CD3ζ molecules can limit the expression and function of the TCRs in the cells. In some aspects, currently available methods for delivery of transgenes, e.g., encoding recombinant receptors, such as recombinant TCRs, may show inefficient integration and/or reduced expression of the recombinant receptors. In some aspects, the efficiency of integration and/or expression of the recombinant receptor within a population may be low and/or varied.

In some aspects, development of a humanized and/or fully human recombinant TCR presents technical challenges. For example, in some aspects, a humanized and/or a fully human recombinant TCR receptor competes with endogenous TCR complexes and can form mispairings with endogenous TCRα and/or TCRβ chains, which may, in certain aspects, reduce recombinant TCR signaling, activity, and/or expression, and ultimately result in reduced activity of the engineered cells. One method to address these challenges has been to design recombinant TCRs with mouse constant domains to prevent mispairings with endogenous human TCRα or TCRβ chains. However, use of recombinant TCRs with mouse sequences may, in some aspects, present a risk for immune response. The provided polynucleotides, reagents, articles of manufacture, kits, and methods address these challenges by inserting sequences encoding all or a portion of a recombinant TCR within an endogenous gene encoding one or more TCR chains. In particular aspects, this insertion serves to disrupt the endogenous TCR gene expression while allowing for the expression of a full humanized and/or human recombinant TCR, reducing the likelihood of competition from or mispairings with endogenous TCR chains or the use of murine sequences which may potentially be immunogenic

In some contexts, available approaches for engineering a plurality and/or a population of cells result in heterogeneous, non-uniform and/or disparate expression of the recombinant receptor, due to differences in efficiency of introduction of the nucleic acid, differences in genomic location of integration and/or copy number, mispairing and/or competition with endogenous TCR chains and/or other factors. In some contexts, available approaches for engineering result in a cell population that are heterogeneous in terms of recombinant receptor expression and/or knock-out of particular loci. In some aspects, heterogeneous and non-uniform expression in a cell population can lead to reduction in overall expression level, stability of expression and/or antigen binding by the recombinant receptor, reduction in function of the engineered cells and/or a non-uniform drug product, thereby reducing the efficacy of the engineered cells.

In some embodiments, provided herein are methods of generating or producing genetically engineered cells that contain TRAC and/or TRBC locus includes nucleic acid sequences encoding a recombinant TCR or a fragment thereof. In some aspects, the TRAC and/or TRBC locus in the genetically engineered cell comprises a transgene sequence (also referred to herein as exogenous or heterologous nucleic acid sequences) encoding all or a portion of a recombinant TCR, integrated into an endogenous TRAC and/or TRBC locus, which normally encodes a TCRα or TCRβ constant domains. In some embodiments, the methods involve inducing a targeted genetic disruption and homology-dependent repair (HDR), using one or more template polynucleotides containing the transgene encoding all or a portion of the recombinant TCR, thereby targeting integration of the transgene at the TRAC and/or TRBC locus. Also provided are cells and cell compositions generated by the methods. In some aspects, elimination of expression of the endogenous TCRα and/or TCRβ chains can reduce mispairing between the endogenous and the engineered or recombinant chains.

In some embodiments, the provided polynucleotides, transgenes, and/or vectors, when delivered into immune cells, result in the expression of recombinant receptors, e.g., TCRs, that can modulate T cell activity, and, in some cases, can modulate T cell differentiation or homeostasis. The resulting genetically engineered cells or cell compositions can be used in adoptive cell therapy methods.

In some aspects, compared to conventional methods of producing genetically engineered immune cells expressing a recombinant receptor, such as a recombinant T cell receptor (TCR) or a chimeric antigen receptor (CAR), the provided methods allow for a higher, much more stable and/or much more uniform or homogeneous expression of the recombinant receptor. In some aspects, the provided embodiments offer advantages in producing engineered T cells with improved, uniform, homogeneous, consistent and/or stable expression of the recombinant receptor, while minimizing possible mispairing, mis-targeting, semi-random or random integration of the transgene and/or competition from endogenous TCRs. In some aspects, the provided embodiments permit predictable and consistent integration at a single gene locus or a multiple gene loci of interest, provide consistent copy number (typically, 1 or 2) of the nucleic acids, have reduced, low or no possibility of insertional mutagenesis, provide consistency in recombinant receptor expression and expression of the endogenous receptor genes within a cell population, and eliminate the requirement for RCL assays. In some aspects, the provided embodiments are based on observations that targeted knock-in of the recombinant receptor-encoding nucleic acids at one or more of the endogenous TCR gene loci, which reduces or eliminates the expression of the endogenous TCR genes, resulted in a higher overall level of expression, a more uniform and consistent expression and/or antigen binding, and improved function of the engineered cells, including improved anti-tumor effects

The provided embodiments also offer advantages in producing engineered T cells, where all cells that express the recombinant receptor are also knocked out for, reduced and/or eliminated the expression of one or more of the endogenous TCR gene loci (such as the endogenous genes encoding the TCRα and/or the TCRβ chains) via gene editing and HDR. Compared to approaches that may produce a heterogeneous mixture, where some of the cells that express the recombinant receptor may be knocked out for the endogenous TCR gene loci while other cells that express the recombinant receptor may retain the endogenous TCR gene loci, the provided embodiments can be used to generate a substantially more homogeneous and uniform population of cells, e.g., where all cells that express the recombinant receptor contain knock-out of one or more of the endogenous TCR gene loci.

In some aspects, the provided embodiments are based on observations of improved efficiency of integration and expression and antigen binding of TCRs using the targeted knock-in approach. Targeted knock-out of one or more of the endogenous TCR gene loci (such as the endogenous genes encoding the TCRα and/or the TCRβ chains) by gene editing, combined with targeted knock-in of nucleic acids encoding the recombinant receptor (such as a recombinant TCR or a CAR) by homology-directed repair (HDR), can facilitate the production of engineered T cells that are improved in expression, function and uniformity of expression and/or other desired features or properties, and ultimately high efficacy.

Also provided are methods for engineering, preparing, and producing the engineered cells, and kits and devices for generating or producing the engineered cells. Provided are polynucleotides, e.g., viral vectors that contain a nucleic acid sequence encoding a recombinant receptor or a portion thereof, and methods for introducing such polynucleotides into the cells, such as by transduction or by physical delivery, such as electroporation. Also provided are compositions containing the engineered cells, methods, kits, and devices for administering the cells and compositions to subjects, such as for adoptive cell therapy.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. METHODS FOR PRODUCING CELLS EXPRESSING A RECOMBINANT RECEPTOR BY HOMOLOGY-DIRECTED REPAIR (HDR)

Provided herein are methods of producing a genetically engineered immune cell, e.g., a genetically engineered T cell for adoptive cell therapy, related compositions, methods, uses, and kits and articles of manufacture used for performing the methods. The immune cells are generally engineered to express a recombinant molecule such as a recombinant receptor, e.g., a recombinant T cell receptor (TCR) or chimeric antigen receptor (CAR). In some embodiments, also provided are compositions containing a population of cells that have been engineered to express a recombinant receptor, e.g., a TCR or a CAR, such that the cell population that exhibits more improved, uniform, homogeneous and/or stable expression and/or antigen binding by the recombinant receptor, including genetically engineered immune cells produced by any of the provided methods. In some embodiments, the provided compositions exhibit reduced coefficient of variation of expression and/or antigen binding, compared to that of cell populations and/or compositions generated using conventional methods. In some embodiments, also provided are methods and uses of the composition and/or cells for therapy, including those involving administration of the composition and/or cells.

In some embodiments, provided are methods of producing a genetically engineered immune cell, e.g., a genetically engineered T cell for adoptive cell therapy. In some embodiments, the provided methods involve introducing into an immune cell one or more agent(s) capable of inducing a genetic disruption of one or more target site(s) (also known as “target position,” “target DNA sequence” or “target location”) within a gene encoding a domain or region of a T cell receptor alpha (TCRα) chain and/or one or more gene(s) encoding a domain or region of a T cell receptor beta (TCRβ) chain (also referred to throughout as “one or more agents” or “agent(s) with reference to aspects of the provided methods); and introducing into the immune cell a polynucleotide, e.g., a template polynucleotide, comprising a transgene encoding a recombinant receptor or a chain thereof, wherein the transgene encoding the recombinant receptor or a chain thereof is targeted at or near one of the at least one target site(s) via homology directed repair (HDR).

In some embodiments, provided herein are methods of generating or producing genetically engineered cells that contain TRAC and/or TRBC locus includes nucleic acid sequences encoding a recombinant TCR or a fragment thereof. In some aspects, the TRAC and/or TRBC locus in the genetically engineered cell comprises a transgene sequence (also referred to herein as exogenous or heterologous nucleic acid sequences) encoding all or a portion of a recombinant TCR, integrated into an endogenous TRAC and/or TRBC locus, which normally encodes a TCRα or TCRβ constant domains. In some embodiments, the methods involve inducing a targeted genetic disruption and homology-dependent repair (HDR), using one or more template polynucleotides containing the transgene encoding all or a portion of the recombinant TCR, thereby targeting integration of the transgene at the TRAC and/or TRBC locus. Also provided are cells and cell compositions generated by the methods.

In particular embodiments, the transgene sequence encoding all or a portion of the recombinant TCR contains a sequence of nucleotides encoding a TCRα chain and/or a TCRβ chain. In some embodiments, one or more polynucleotides, e.g., template polynucleotides, can be used. In some embodiments, each polynucleotide, e.g., template polynucleotide, can contain sequence of nucleotides encoding either a TCRα chain or a TCRβ chain. In some embodiments, the polynucleotide, e.g., the template polynucleotide, comprises a nucleic acid sequence encoding all or a portion of a recombinant receptor or chain thereof, e.g., a recombinant TCR or a chain thereof. In certain embodiments, the nucleic acid sequence is targeted at a target site(s) that is within a gene locus that encodes an endogenous receptor, e.g., at one or more genes encoding an endogenous TCR chain or a portion thereof. In certain embodiments, the nucleic acid sequence is targeted for integration within the endogenous gene locus. In certain embodiments, the integration genetically disrupts expression of the endogenous receptor encoded by gene at the target site. In particular embodiments, the transgene encoding the portion of the recombinant receptor is targeted within the gene locus via HDR.

In some embodiments, the provided methods involve introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and introducing into the immune cell a template polynucleotide comprising a transgene encoding a recombinant T cell receptor (TCR) or an antigen-binding fragment thereof or a chain thereof, wherein the transgene encoding the recombinant TCR or antigen-binding fragment or chain thereof is targeted for integration at or near one of the at least one target site via homology directed repair (HDR). In particular embodiments, the integration at or near the target site is within a portion of coding sequence of a TRAC and/or TRBC gene, such as, for example, a portion of the coding sequence downstream of, or 3′ of the target site.

In some embodiments, one of the at least one the target site(s) is in a T cell receptor alpha constant (TRAC) gene. In some embodiments, one of the at least one the target site(s) is in a T cell receptor beta constant 1 (TRBC1) or T cell receptor beta constant 2 (TRBC2) gene. In some embodiments, the one or more target site(s) is in a TRAC gene and one or both of a TRBC1 and a TRBC2 gene.

In some embodiments, the provided methods involve introducing into an immune cell having a genetic disruption of one or more target site(s) within a gene encoding a domain or region of a T cell receptor alpha (TCRα) chain and/or one or more gene(s) encoding a domain or region of a T cell receptor beta (TCRβ) chain, a template polynucleotide comprising a transgene encoding a recombinant receptor, wherein the transgene encoding the recombinant receptor or a chain thereof is targeted at or near one of the at least one target site(s) via HDR.

In provided embodiments, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors. Methods, such as electroporation, also can be used to introduce or deliver protein or ribonucleoprotein (RNP), e.g. containing the Cas9 protein in complex with a targeting gRNA, to cells of interest.

In some cases, the embodiments provided herein involve one or more targeted genetic disruption, e.g., DNA break, at one or more of the endogenous TCR gene loci (such as the endogenous genes encoding the TCRα and/or the TCRβ chains) by gene editing techniques, combined with targeted knock-in of nucleic acids encoding the recombinant receptor (such as a recombinant TCR or a CAR) by homology-directed repair (HDR). In some embodiments, the HDR step requires a break, e.g., a double-stranded break, in the DNA at the target genomic location. In some embodiments, the DNA break occurs as a result of a step in gene editing, for example, DNA breaks generated by targeted nucleases used in gene editing.

In some embodiments, the embodiments involve generating a targeted DNA break using gene editing methods and/or targeted nucleases, followed by HDR based on one or more template polynucleotide(s), e.g., template polynucleotide(s) that contains homology sequences and one or more transgenes, e.g., nucleic acids encoding a recombinant receptor or a chain thereof and/or other exogenous or recombinant nucleic acids, to specifically target and integrate the nucleic acid sequences encoding the recombinant receptor or a chain thereof and/or other exogenous or recombinant nucleic acids at or near the DNA break.

In some embodiments, the targeted genetic disruption and targeted integration of the recombinant receptor-encoding nucleic acids by HDR occurs at one or more target site(s) (also known as “target position,” “target DNA sequence” or “target location”) the endogenous genes that encode one or more domains, regions and/or chains of the endogenous T cell receptor (TCR). In some embodiments, the targeted genetic disruption is induced at the TCRα gene. In some embodiments, the targeted genetic disruption is induced at the TCRβ gene. In some embodiments, the targeted genetic disruption is induced at the endogenous TCRα gene and the endogenous TCRβ gene. Endogenous TCR genes can include one or more of the gene encoding TCRα constant domain (encoded by TRAC in humans) and/or TCRβ constant domain (encoded by TRBC1 or TRBC2 in humans).

In some embodiments, targeted genetic disruption of one or more of the endogenous TCR gene loci can lead to a reduced risk or chance of mispairing between chains of the engineered or recombinant TCR and the endogenous TCR. Mispaired TCRs can create a new TCR that could potentially result in a higher risk of undesired or unintended antigen recognition and/or side effects, and/or could reduce expression levels of the desired engineered or recombinant TCR. In some aspects, reducing or preventing endogenous TCR expression can increase expression of the engineered or recombinant TCR in the T cells or T cell compositions as compared to cells in which expression of the TCR is not reduced or prevented. In some embodiments, recombinant TCR expression can be increased by 1.5-fold, 2-fold, 3-fold, 4-fold, 5-fold or more. For example, in some cases, suboptimal expression of an engineered or recombinant TCR can occur due to competition with an endogenous TCR and/or with TCRs having mispaired chains, for signaling domains such as the invariant CD3 signaling molecules that are involved in permitting expression of the complex on the cell surface.

In some embodiments, a template polynucleotide is introduced into the engineered cell, prior to, simultaneously with, or subsequent to introduction of agent(s) capable of inducing one or more targeted genetic disruption. In the presence of one or more targeted genetic disruption, e.g., DNA break, the template polynucleotide can be used as a DNA repair template, to effectively copy and integrate the transgene, e.g., nucleic acid sequences encoding the recombinant receptor, at or near the site of the targeted genetic disruption by HDR, based on homology between the endogenous gene sequence surrounding the target site and the 5′ and/or 3′ homology arms included in the template polynucleotide.

In some embodiments, the gene editing and HDR steps are performed simultaneously and/or in one experimental reaction. In some embodiments, the gene editing and HDR steps are performed consecutively or sequentially, in one or consecutive experimental reaction(s). In some embodiments, the gene editing and HDR steps are performed in separate experimental reactions, simultaneously or at different times.

The immune cells can include a population of cells containing T cells. Such cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the polynucleotide template and the step of introducing the agent (e.g. Cas9/gRNA RNP) can occur simultaneously or sequentially in any order. In particular embodiments, the polynucleotide template is introduced into the immune cells after inducing the genetic disruption by the step of introducing the agent(s) (e.g. Cas9/gRNA RNP). In some embodiments, prior to, during and/or subsequent to introduction of the polynucleotide template and one or more agents (e.g. Cas9/gRNA RNP), the cells are cultured or incubated under conditions to stimulate expansion and/or proliferation of cells.

In particular embodiments of the provided methods, the introduction of the template polynucleotide is performed after the introduction of the one or more agent capable of inducing a genetic disruption. Any method for introducing the one or more agent(s) can be employed as described, depending on the particular agent(s) used for inducing the genetic disruption. In some aspects, the disruption is carried out by gene editing, such as using an RNA-guided nuclease such as a clustered regularly interspersed short palindromic nucleic acid (CRISPR)-Cas system, such as CRISPR-Cas9 system, specific for the TRAC or TRBC locus being disrupted. In some embodiments, an agent containing a Cas9 and a guide RNA (gRNA) containing a targeting domain, which targets a region of the TRAC or TRBC locus, is introduced into the cell. In some embodiments, the agent is or comprises a ribonucleoprotein (RNP) complex of Cas9 and gRNA containing the TRAC/TRBC-targeted targeting domain (Cas9/gRNA RNP). In some embodiment, the introduction includes contacting the agent or portion thereof with the cells, in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly-A tail and a 5′ Anti-Reverse Cap Analog (ARCA) cap. In some cases, electroporation of the cells to be modified includes cold-shocking the cells, e.g. at 32° C. following electroporation of the cells and prior to plating.

In such aspects of the provided methods, a template polynucleotide is introduced into the cells after introduction with the one or more agent(s), such as Cas9/gRNA RNP, e.g. that has been introduced via electroporation. In some embodiments, the template polynucleotide is introduced immediately after the introduction of the one or more agents capable of inducing a genetic disruption. In some embodiments, the template polynucleotide is introduced into the cells within at or about 30 seconds, within at or about 1 minute, within at or about 2 minutes, within at or about 3 minutes, within at or about 4 minutes, within at or about 5 minutes, within at or about 6 minutes, within at or about 6 minutes, within at or about 8 minutes, within at or about 9 minutes, within at or about 10 minutes, within at or about 15 minutes, within at or about 20 minutes, within at or about 30 minutes, within at or about 40 minutes, within at or about 50 minutes, within at or about 60 minutes, within at or about 90 minutes, within at or about 2 hours, within at or about 3 hours or within at or about 4 hours after the introduction of one or more agents capable of inducing a genetic disruption. In some embodiments, the template polynucleotide is introduced into cells at time between at or about 15 minutes and at or about 4 hours after introducing the one or more agent(s), such as between at or about 15 minutes and at or about 3 hours, between at and about 15 minutes and at or about 2 hours, between at or about 15 minutes and at or about 1 hour, between at or about 15 minutes and at or about 30 minutes, between at or about 30 minutes and at or about 4 hours, between at or about 30 minutes and at or about 3 hours, between at or about 30 minutes and at or about 2 hours, between at or about 30 minutes and at or about 1 hour, between at or about 1 hour and at or about 4 hours, between at or about 1 hour and at or about 3 hours, between at or about 1 hour and at or about 2 hours, between at or about 2 hours and at or about 4 hours, between at or about 2 hours and at or about 3 hours or between at or about 3 hours and at or about 4 hours. In some embodiments, the template polynucleotide is introduced into cells at or about 2 hours after the introduction of the one or more agents. such as Cas9/gRNA RNP, e.g. that has been introduced via electroporation.

Any method for introducing the template polynucleotide can be employed as described, depending on the particular methods used for delivery of the template polynucleotide to cells. Exemplary methods include those for transfer of nucleic acids encoding the receptors, including via viral, e.g., retroviral or lentiviral, transduction, transposons, and electroporation. In particular embodiments, viral transduction methods are employed. In some embodiments, template polynucleotides can be transferred or introduced into cells sing recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557. In particular embodiments, the viral vector is an AAV such as an AAV2 or an AAV6.

In some embodiments, prior to, during or subsequent to contacting the agent with the cells and/or prior to, during or subsequent to effecting delivery (e.g. electroporation), the provided methods include incubating the cells in the presence of a cytokine, a stimulating agent and/or an agent that is capable of inducing proliferation, stimulation or activation of the immune cells (e.g. T cells). In some embodiments, at least a portion of the incubation is in the presence of a stimulating agent that is or comprises an antibody specific for CD3 an antibody specific for CD28 and/or a cytokine, such as anti-CD3/anti-CD28 beads. In some embodiments, at least a portion of the incubation is in the presence of a cytokine, such as one or more of recombinant IL-2, recombinant IL-7 and/or recombinant IL-15. In some embodiments, the incubation is for up to 8 days hours before or after the introduction with the one or more agent(s), such as Cas9/gRNA RNP, e.g. via electroporation, and template polynucleotide, such as up to 24 hours, 36 hours or 48 hours or 3, 4, 5, 6, 7 or 8 days.

In some embodiments, the method includes activating or stimulating cells with a stimulating agent (e.g. anti-CD3/anti-CD28 antibodies) prior to introducing the agent, e.g. Cas9/gRNA RNP, and the polynucleotide template. In some embodiments, the incubation in the presence of a stimulating agent (e.g. anti-CD3/anti-CD28) is for 6 hours to 96 hours, such as 24-48 hours or 24-36 hours prior to the introduction with the one or more agent(s), such as Cas9/gRNA RNP, e.g. via electroporation. In some embodiments, the incubation with the stimulating agents can further include the presence of a cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). In some embodiments the stimulating agent(s) (e.g. anti-CD3/anti-CD28 antibodies) is washed or removed from the cells prior to introducing or delivering into the cells the agent(s) capable of inducing a genetic disruption Cas9/gRNA RNP and/or the polynucleotide template. In some embodiments, prior to the introducing of the agent(s), the cells are rested, e.g. by removal of any stimulating or activating agent. In some embodiments, prior to introducing the agent(s), the stimulating or activating agent and/or cytokines are not removed.

In some embodiments, subsequent to the introduction of the agent(s), e.g. Cas9/gRNA, and/or the polynucleotide template the cells are incubated, cultivated or cultured in the presence of a recombinant cytokine, such as one or more of recombinant IL-2, recombinant IL-7 and/or recombinant IL-15. In some embodiments, the incubation is carried out in the presence of a recombinant cytokine, such as IL-2 (e.g. 1 U/mL to 500 U/mL, such as 10 U/mL to 200 U/mL, for example at least or about 50 U/mL or 100 U/mL), IL-7 (e.g. 0.5 ng/mL to 50 ng/mL, such as 1 ng/mL to 20 ng/mL, for example, at least or about 5 ng/mL or 10 ng/mL) or IL-15 (e.g. 0.1 ng/mL to 50 ng/mL, such as 0.5 ng/mL to 25 ng/mL, for example, at least or about 1 ng/mL or 5 ng/mL). The cells can be incubated or cultivated under conditions to induce proliferation or expansion of the cells. In some embodiments, the cells can be incubated or cultivated until a threshold number of cells is achieved for harvest, e.g. a therapeutically effective dose.

In some embodiments, the incubation during any portion of the process or all of the process can be at a temperature of 30° C.±2° C. to 39° C.±2° C., such as at least or about at least 30° C.±2° C., 32° C.±2° C., 34° C.±2° C. or 37° C.±2° C. In some embodiments, at least a portion of the incubation is at 30° C.±2° C. and at least a portion of the incubation is at 37° C.±2° C.

A. Genetic Disruption

In some embodiments, one or more targeted genetic disruption is induced at the endogenous TCRα gene and/or the endogenous TCRβ gene. In some embodiments, the targeted genetic disruption is induced at one or more of the gene encoding TCRα constant domain (also known as TCRα constant region; encoded by TRAC in humans) and/or TCRβ constant domain (also known as TCRβ constant region; encoded by TRBC1 or TRBC2 in humans). In some embodiments, targeted genetic disruption is induced at the TRAC, TRBC1 and TRBC2 loci.

In some embodiments, targeted genetic disruption results in a DNA break or a nick. In some embodiments, at the site of the DNA break, action of cellular DNA repair mechanisms can result in knock-out, insertion, missense or frameshift mutation, such as a biallelic frameshift mutation, deletion of all or part of the gene. In some embodiments, the genetic disruption can be targeted to one or more exon of a gene or portion thereof, such as within the first or second exon. In some embodiments, a DNA binding protein or DNA-binding nucleic acid, which specifically binds to or hybridizes to the sequences at a region near one of the at least one target site(s), is used for targeted disruption. In some aspects, in the absence of exogenous template polynucleotides for HDR the disruption, the targeted genetic disruption results in a deletion, mutation and or insertion within an exon of the gene. In some embodiments, template polynucleotides, e.g., template polynucleotides that include nucleic acid sequences encoding a recombinant receptor and homology sequences, can be introduced for targeted integration of the recombinant receptor-encoding sequences at or near the site of the genetic disruption by HDR (see Section I.B. herein).

In some embodiments, the genetic disruption is carried by introducing one or more agent(s) capable of inducing a genetic disruption. In some embodiments, such agents comprise a DNA binding protein or DNA-binding nucleic acid that specifically binds to or hybridizes to the gene. In some embodiments, the agent comprises various components, such as a fusion protein comprising a DNA-targeting protein and a nuclease or an RNA-guided nuclease. In some embodiments, the agents can target one or more target locations, e.g., at a TRAC gene and one or both of a TRBC1 and a TRBC2 gene.

In some embodiments, the genetic disruption occurs at a target site (also referred to and/or known as “target position,” “target DNA sequence,” or “target location”). In some embodiments, target site is or includes a site on a target DNA (e.g., genomic DNA) that is modified by the one or more agent(s) capable of inducing a genetic disruption, e.g., a Cas9 molecule complexed with a gRNA that specifies the target site. For example, in some embodiments, the target site may include locations in the DNA, e.g., at an endogenous TRAC, TRBC1 and/or TRBC2 locus, where cleavage or DNA breaks occur. In some aspects, integration of nucleic acid sequences by HDR can occur at or near the target site or target sequence. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence.

1. Target Sites at Endogenous T Cell Receptor (TCR) Encoding Genes

In some embodiments, the targeted genetic disruption occurs at the endogenous genes that encode one or more domains, regions and/or chains of the endogenous T cell receptor (TCR). In some embodiments, the genetic disruption is targeted at the endogenous gene loci that encode TCRα and/or the TCRβ. In some embodiments, the genetic disruption is targeted at the gene encoding TCRα constant domain (TRAC in humans) and/or TCRβ constant domain (TRBC1 or TRBC2 in humans).

In some embodiments, a “T cell receptor” or “TCR,” including the endogenous TCRs, is a molecule that contains a variable α and β chains (also known as TCRα and TCRβ, respectively) or a variable γ and δ chains (also known as TCRγ and TCRδ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. Typically, one T cell expresses one type of TCR. A TCR can be found on the surface of a cell or in soluble form. Generally, a TCR is found on the surface of T cells (or T lymphocytes) where it is generally responsible for recognizing antigens bound to major histocompatibility complex (MHC) molecules.

In some embodiments, a TCR can contain a variable domain and a constant domain (also known as a constant region), a transmembrane domain and/or a short cytoplasmic tail (see, e.g., Janeway et al., Immunobiology: The Immune System in Health and Disease, 3^{rd} Ed., Current Biology Publications, p. 4:33, 1997). In some embodiments, a TCR chain contains one or more constant domain. For example, the extracellular portion of a given TCR chain (e.g., TCRα chain or TCRβ chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) and a constant domain (e.g., a chain constant domain or TCR Ca, typically positions 117 to 259 of the chain based on Kabat numbering or β chain constant domain or TCR Cβ, typically positions 117 to 295 of the chain based on Kabat) adjacent to the cell membrane. For example, in some cases, the extracellular portion of the TCR formed by the two chains contains two membrane-proximal constant domains, and two membrane-distal variable domains.

In some embodiments, the endogenous TCR Cα is encoded by the TRAC gene (IMGT nomenclature). An exemplary sequence of the human T cell receptor alpha chain constant domain (TRAC) gene locus is set forth in SEQ ID NO:1 (NCBI Reference Sequence: NG_001332.3, TRAC). In some embodiments, the encoded endogenous Cα comprises the sequence of amino acids set forth in SEQ ID NO: 19 or 24 (UniProtKB Accession No. P01848 or Genbank Accession No. CAA26636.1). In certain embodiments, a genetic disruption is targeted at, near, or within a TRAC locus. In particular embodiments, the genetic disruption is targeted at, near, or within an open reading frame of the TRAC locus. In certain embodiments, the genetic disruption is targeted at, near, or within an open reading frame that encodes a TCRα constant domain. In some embodiments, the genetic disruption is targeted at, near, or within a locus having the nucleic acid sequence set forth in SEQ ID NO: 1, or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the nucleic acid sequence set forth in SEQ ID NO: 1.

In humans, an exemplary genomic locus of TRAC comprises an open reading frame that contains 4 exons and 3 introns. An exemplary mRNA transcript of TRAC can span the sequence corresponding to coordinates Chromosome 14: 22,547,506-22,552,154, on the forward strand, with reference to human genome version GRCh38 (UCSC Genome Browser on Human December 2013 (GRCh38/hg38) Assembly). Table 1 sets forth the coordinates of the exons and introns of the open reading frames and the untranslated regions of the transcript of an exemplary human TRAC locus.

TABLE 1
Coordinates of exons and introns of exemplary human
TRAC locus (GRCh38, Chromosome 14, forward strand).

| | Start (GrCh38) | End (GrCh38) | Length |
|---|---|---|---|
| 5′ UTR and Exon 1 | 22,547,506 | 22,547,778 | 273 |
| Intron 1-2 | 22,547,779 | 22,549,637 | 1,859 |
| Exon 2 | 22,549,638 | 22,549,682 | 45 |
| Intron 2-3 | 22,549,683 | 22,550,556 | 874 |
| Exon 3 | 22,550,557 | 22,550,664 | 108 |
| Intron 3-4 | 22,550,665 | 22,551,604 | 940 |
| Exon 4 and 3′ UTR | 22,551,605 | 22,552,154 | 550 |

In some embodiments, the endogenous TCR Cβ is encoded by TRBC1 or TRBC2 genes (IMGT nomenclature). An exemplary sequence of the human T cell receptor beta chain constant domain 1 (TRBC1) gene locus is set forth in SEQ ID NO:2 (NCBI Reference Sequence: NG_001333.2, TRBC1); and an exemplary sequence of the human T cell receptor beta chain constant domain 2 (TRBC2) gene locus is set forth in SEQ ID NO:3 (NCBI Reference Sequence: NG_001333.2, TRBC2). In some embodiments, the encoded Cβ has or comprises the sequence of amino acids set forth in SEQ ID NO:20, 21 or 25 (Uniprot Accession No. P01850, A0A5B9 or A0A0G2JNG9). In some embodiments, a genetic disruption is targeted at, near, or within the TRBC1 gene locus. In particular embodiments, the genetic disruption is targeted at, near, or within an open reading frame of the TRBC1 locus. In certain embodiments, the genetic disruption is targeted at, near, or within an open reading frame that encodes a TCRβ constant domain. In some embodiments, the genetic disruption is targeted at, near, or within a locus having the nucleic acid sequence set forth in SEQ ID NO: 2, or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the nucleic acid sequence set forth in SEQ ID NO: 2.

In humans, an exemplary genomic locus of TRBC1 comprises an open reading frame that contains 4 exons and 3 introns. An exemplary mRNA transcript of TRBC1 can span the sequence corresponding to coordinates Chromosome 7: 142,791,694-142,793,368, on the forward strand, with reference to human genome version GRCh38 (UCSC Genome Browser on Human December 2013 (GRCh38/hg38) Assembly). Table 2 sets forth the coordinates of the exons and introns of the open reading frames and the untranslated regions of the transcript of an exemplary human TRBC1 locus.

TABLE 2
Coordinates of exons and introns of exemplary human
TRBC1 locus (GRCh38, Chromosome 7, forward strand).

| | Start (GrCh38) | End (GrCh38) | Length |
|---|---|---|---|
| 5′ UTR and Exon 1 | 142,791,694 | 142,792,080 | 387 |
| Intron 1-2 | 142,792,081 | 142,792,521 | 441 |
| Exon 2 | 142,792,522 | 142,792,539 | 18 |
| Intron 2-3 | 142,792,540 | 142,792,691 | 152 |
| Exon 3 | 142,792,692 | 142,792,798 | 107 |
| Intron 3-4 | 142,792,799 | 142,793,120 | 322 |
| Exon 4 and 3′ UTR | 142,793,121 | 142,793,368 | 248 |

In particular embodiments, a genetic disruption is targeted at, near, or within the TRBC2 locus. In particular embodiments, the genetic disruption is targeted at, near, or within an open reading frame of the TRBC2 locus. In certain embodiments, the genetic disruption is targeted at, near, or within an open reading frame that encodes a TCRβ constant domain. In some embodiments, the genetic disruption is targeted at, near, or within a locus having the nucleic acid sequence set forth in SEQ ID NO: 3, or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the nucleic acid sequence set forth in SEQ ID NO: 3.

In humans, an exemplary genomic locus of TRBC2 comprises an open reading frame that contains 4 exons and 3 introns. An exemplary mRNA transcript of TRBC2 can span the sequence corresponding to coordinates Chromosome 7: 142,801,041-142,802,748, on the forward strand, with reference to human genome version GRCh38 (UCSC Genome Browser on Human December 2013 (GRCh38/hg38) Assembly). Table 3 sets forth the coordinates of the exons and introns of the open reading frames and the untranslated regions of the transcript of an exemplary human TRBC2 locus.

TABLE 3
Coordinates of exons and introns of exemplary human
TRBC2 locus (GRCh38, Chromosome 7, forward strand).

| | Start (GrCh38) | End (GrCh38) | Length |
|---|---|---|---|
| 5′ UTR and Exon 1 | 142,801,041 | 142,801,427 | 387 |
| Intron 1-2 | 142,801,428 | 142,801,943 | 516 |
| Exon 2 | 142,801,944 | 142,801,961 | 18 |
| Intron 2-3 | 142,801,962 | 142,802,104 | 143 |
| Exon 3 | 142,802,105 | 142,802,211 | 107 |
| Intron 3-4 | 142,802,212 | 142,802,502 | 291 |
| Exon 4 and 3′ UTR | 142,802,503 | 142,802,748 | 246 |

In some aspects, the transgene (e.g., exogenous nucleic acid sequences) within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of transgene sequences (e.g., encoding a recombinant TCR or a portion thereof). In some aspects, the target site is within an exon of the open reading frame of the TRAC, TRBC1 and/or TRBC2 locus. In some aspects, the target site is within an intron of the open reading frame of the TRAC, TRBC1 and/or TRBC2 locus.

In some embodiments, the genetic disruption, e.g., DNA break, is targeted at or in close proximity to the beginning of the coding region (e.g., the early coding region, e.g., within 500 bp from the start codon or the remaining coding sequence, e.g., downstream of the first 500 bp from the start codon). In some embodiments, the genetic disruption, e.g., DNA break, is targeted at early coding region of a gene of interest, e.g., TRAC, TRBC1 and/or TRBC2, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In some embodiments, the target site is within an exon of the endogenous TRAC, TRBC1, and/or TRBC2 locus. In certain embodiments, the target site is within an intron of the endogenous TRAC, TRBC1, and/or TRBC2 locus. In some aspects, the target site is within a regulatory or control element, e.g., a promoter, 5′ untranslated region (UTR) or 3′ UTR, of the TRAC, TRBC1, and/or TRBC2 locus. In certain embodiments, the target site is within an open reading frame of an endogenous TRAC, TRBC1, and/or TRBC2 locus. In particular embodiments, the target site is within an exon within the open reading frame of the TRAC, TRBC1, and/or TRBC2 locus.

In particular embodiments, the genetic disruption, e.g., DNA break, is targeted at or within an open reading frame of a gene or locus of interest, e.g., TRAC, TRBC1, and/or TRBC2. In some embodiments, the genetic disruption is targeted at or within an intron within the open reading frame of a gene or locus of interest. In some embodiments, the genetic disruption is targeted within an exon within the open reading frame of the gene or locus of interest.

In particular embodiments, a genetic disruption, e.g., DNA break, is targeted at or within an intron. In certain embodiments, a genetic disruption, e.g., DNA break, is targeted at or within an exon. In some embodiments, a genetic disruption, e.g., DNA break, is targeted at or within an exon of a gene of interest, e.g., TRAC, TRBC1 and/or TRBC2.

In some embodiments, a genetic disruption, e.g., DNA break, is targeted within an exon of the TRAC gene, open reading frame, or locus. In certain embodiments, the genetic disruption is within the first exon, second exon, third exon, or fourth exon of the TRAC gene, open reading frame, or locus. In particular embodiments, the genetic disruption is within the first exon of the TRAC gene, open reading frame, or locus. In some embodiments, the genetic disruption is within 500 base pairs (bp) downstream from the 5′ end of the first exon in the TRAC gene, open reading frame, or locus. In particular embodiments, the genetic disruption is between the most 5′ nucleotide of exon 1 and upstream of the most 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the first exon in the TRAC gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the first exon in the TRAC gene, open reading frame, or locus, each inclusive. In certain embodiments, the genetic disruption is between 100 bp and 150 bp downstream from the 5′ end of the first exon in the TRAC gene, open reading frame, or locus, inclusive.

In particular embodiments, a genetic disruption, e.g., DNA break, is targeted within an exon of a TRBC gene, open reading frame, or locus, e.g., TRBC1 and/or the TRBC2. In certain embodiments, the genetic disruption is within the first exon, second exon, third exon, or fourth exon of the TRBC1 and/or the TRBC2 gene, open reading frame, or locus. In some embodiments, the genetic disruption is within the first exon of the TRBC1 and/or the TRBC2 gene, open reading frame, or locus. In certain embodiments, the genetic disruption is within the first exon, second exon, third exon, or fourth exon of the TRBC1 and/or the TRBC2 gene, open reading frame, or locus. In some embodiments, the genetic disruption is between the most 5′ nucleotide of exon 1 and upstream of the most 3′ nucleotide of exon 1. In particular embodiments, the genetic disruption is within the first exon of the TRBC gene, open reading frame, or locus. In some embodiments, the genetic disruption is within 400 bp, 350 bp, 300 bp, 250 bp, 200 bp, 150 bp, 100 bp, or 50 bp downstream from the 5′ end of the first exon in a TRBC1 and/or the TRBC2 gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the first exon in the TRBC1 and/or the TRBC2 gene, open reading frame, or locus, each inclusive. In certain embodiments, the genetic disruption is between 100 bp and 150 bp downstream from the 5′ end of the first exon in the TRBC1 and/or the TRBC2 gene, open reading frame, or locus, inclusive.

2. Methods of Genetic Disruption

Methods for generating a genetic disruption, including those described herein, can involve the use of one or more agent(s) capable of inducing a genetic disruption, such as engineered systems to induce a genetic disruption, a cleavage and/or a double strand break (DSB) or a nick in a target site or target position in the endogenous DNA such that repair of the break by an error born process such as non-homologous end joining (NHEJ) or repair using a repair template HDR can result in the knock out of a gene and/or the insertion of a sequence of interest (e.g., exogenous nucleic acid sequences or transgene encoding a portion of a chimeric receptor) at or near the target site or position. Also provided are one or more agent(s) capable of inducing a genetic disruption, for use in the methods provided herein. In some aspects, the one or more agent(s) can be used in combination with the template nucleotides provided herein, for homology directed repair (HDR) mediated targeted integration of the transgene sequences (e.g., described herein in Section I.B).

In some embodiments, the one or more agent(s) capable of inducing a genetic disruption comprises a DNA binding protein or DNA-binding nucleic acid that specifically binds to or hybridizes to a particular site or position in the genome, e.g., a target site or target position. In some aspects, the targeted genetic disruption, e.g., DNA break or cleavage, of the endogenous genes encoding TCR is achieved using a protein or a nucleic acid is coupled to or complexed with a gene editing nuclease, such as in a chimeric or fusion protein. In some embodiments, the one or more agent(s) capable of inducing a genetic disruption comprises an RNA-guided nuclease, or a fusion protein comprising a DNA-targeting protein and a nuclease.

In some embodiments, the agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system (including Cas and/or Cfp1). In some embodiments, the targeted genetic disruption is carried using agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the one or more agent(s) specifically targets the at least one target site(s), e.g., at or near a gene of interest, e.g., TRAC, TRBC1 and/or TRBC2. In some embodiments, the agent comprises a ZFN, TALEN or a CRISPR/Cas9 combination that specifically binds to, recognizes, or hybridizes to the target site(s). In some embodiments, the CRISPR/Cas9 system includes an engineered crRNA/tracr RNA (“single guide RNA”) to guide specific cleavage. In some embodiments, the agent comprises nucleases based on the Argonaute system (e.g., from T. thermophilus, known as ‘TtAgo’, (Swarts et al. (2014) Nature 507(7491): 258-261). Targeted cleavage using any of the nuclease systems described herein can be exploited to insert the sequences of a transgene, e.g., nucleic acid sequences encoding a recombinant receptor, into a specific target location, e.g., at endogenous TCR genes, using either HDR or NHEJ-mediated processes.

In some embodiments, a “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269: 978-982. Some gene-specific engineered zinc fingers are available commercially. For example, a platform called CompoZr, for zinc-finger construction is available that provides specifically targeted zinc fingers for thousands of targets. See, e.g., Gaj et al., Trends in Biotechnology, 2013, 31(7), 397-405. In some cases, commercially available zinc fingers are used or are custom designed.

In some embodiments, the one or more target site(s), e.g., within TRAC, TRBC1 and/or TRBC2 genes can be targeted for genetic disruption by engineered ZFNs. Exemplary ZFN that target endogenous T cell receptor (TCR) genes include those described in, e.g., US 2015/0164954, US 2011/0158957, US 2015/0056705, U.S. Pat. No. 8,956,828 and Torikawa et al. (2012) Blood 119:5697-5705, the disclosures of which are incorporated by reference in their entireties, or those set forth in any of SEQ ID NOS:213-224 (TRAC) or SEQ ID NOS: 225 and 226 (TRBC).

Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from different bacterial species. The new modular proteins have the advantage of displaying more sequence variability than TAL repeats. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, critical amino acids 12 and 13 can be mutated towards other amino acid residues in order to modulate their specificity towards nucleotides A, T, C and G and in particular to enhance this specificity.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a “TALE-nuclease” (TALEN) is a fusion protein comprising a nucleic acid binding domain typically derived from a Transcription Activator Like Effector (TALE) and a nuclease catalytic domain that cleaves a nucleic acid target sequence. The catalytic domain comprises a nuclease domain or a domain having endonuclease activity, like for instance I-TevI, ColE7, NucA and Fok-I. In a particular embodiment, the TALE domain can be fused to a meganuclease like for instance I-CreI and I-OnuI or functional variant thereof. In some embodiments, the TALEN is a monomeric TALEN. A monomeric TALEN is a TALEN that does not require dimerization for specific recognition and cleavage, such as the fusions of engineered TAL repeats with the catalytic domain of I-TevI described in WO2012138927. TALENs have been described and used for gene targeting and gene modifications (see, e.g., Boch et al. (2009) Science 326(5959): 1509-12.; Moscou and Bogdanove (2009) Science 326(5959): 1501; Christian et al. (2010) Genetics 186(2): 757-61; Li et al. (2011) Nucleic Acids Res 39(1): 359-72).

In some embodiments, the TRAC, TRBC1 and/or TRBC2 genes can be targeted for genetic disruption by engineered TALENs. Exemplary TALEN that target endogenous T cell receptor (TCR) genes include those described in, e.g., WO 2017/070429, WO 2015/136001, US20170016025 and US20150203817, the disclosures of which are incorporated by reference in their entireties.

In some embodiments, a “TtAgo” is a prokaryotic Argonaute protein thought to be involved in gene silencing. TtAgo is derived from the bacteria Thermus thermophilus. See, e.g. Swarts et al, (2014) Nature 507(7491): 258-261, Sheng et al., (2013) Proc. Natl. Acad. Sci. U.S.A. 111, 652). A “TtAgo system” is all the components required including e.g. guide DNAs for cleavage by a TtAgo enzyme.

In some embodiments, an engineered zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

Zinc finger and TALE DNA-binding domains can be engineered to bind to a predetermined nucleotide sequence, for example via engineering (altering one or more amino acids) of the recognition helix region of a naturally occurring zinc finger protein or by engineering of the amino acids involved in DNA binding (the repeat variable diresidue or RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties. Also provided are one or more agents capable of introducing a genetic disruption. Also provided are polynucleotides (e.g., nucleic acid molecules) encoding one or more components of the one or more agent(s) capable of inducing a genetic disruption.

a. Crispr/Cas9

In some embodiments, the targeted genetic disruption, e.g., DNA break, of the endogenous genes encoding TCR, such as TRAC and TRBC1 or TRBC2 in humans is carried out using clustered regularly interspaced short palindromic repeats (CRISPR) and CRISPR-associated (Cas) proteins. See Sander and Joung, (2014) Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9), with nuclease functionality.

1) Guide RNA (gRNA)

In some embodiments, the one or more agent(s) comprises at least one of: a guide RNA (gRNA) having a targeting domain that is complementary with a target site of a TRAC gene; a gRNA having a targeting domain that is complementary with a target site of one or both of a TRBC1 and a TRBC2 gene; or at least one nucleic acid encoding the gRNA.

In some aspects, a “gRNA molecule” is to a nucleic acid that promotes the specific targeting or homing of a gRNA molecule/Cas9 molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a guide sequence, e.g., guide RNA, is any polynucleotide sequences comprising at least a sequence portion that has sufficient complementarity with a target polynucleotide sequence, such as the TRAC, TRBC1 and/or TRBC2 genes in humans, to hybridize with the target sequence at the target site and direct sequence-specific binding of the CRISPR complex to the target sequence. In some embodiments, in the context of formation of a CRISPR complex, “target sequence” generally refers to a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some embodiments, a guide RNA (gRNA) specific to a target locus of interest (e.g. at the TRAC, TRBC1 and/or TRBC2 loci in humans) is used to RNA-guided nucleases, e.g., Cas, to induce a DNA break at the target site or target position. Methods for designing gRNAs and exemplary targeting domains can include those described in, e.g., WO2015/161276, WO2017/193107, WO2017/093969, US2016/272999 and US2015/056705.

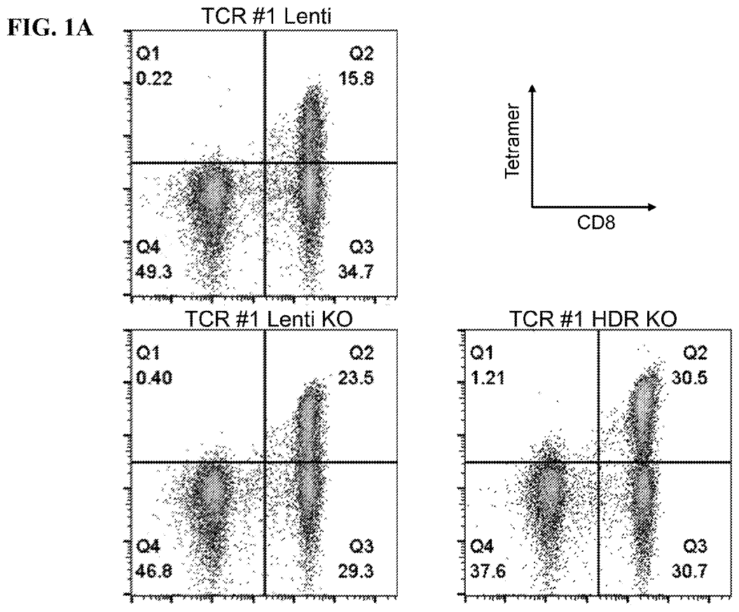

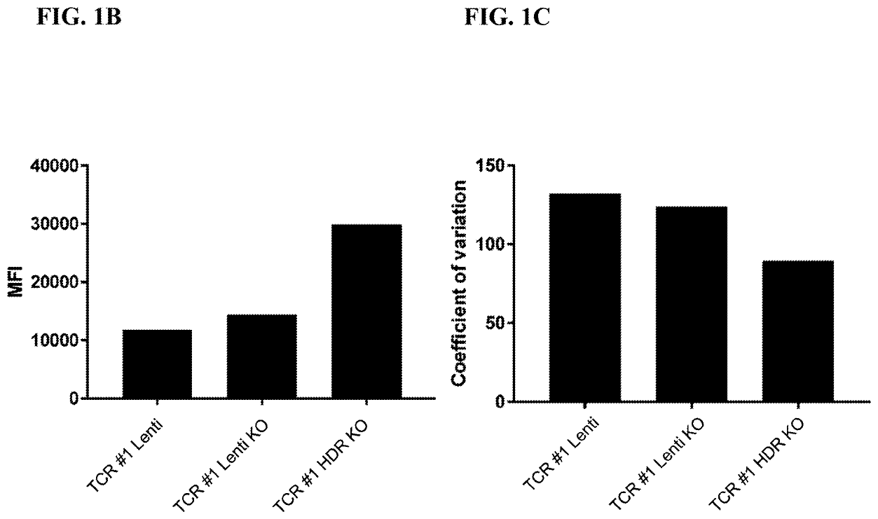

Several exemplary gRNA structures, with domains indicated thereon, are described in WO2015/161276, e.g., in FIGS. 1A-1G therein. While not wishing to be bound by theory, with regard to the three dimensional form, or intra- or inter-strand interactions of an active form of a gRNA, regions of high complementarity are sometimes shown as duplexes in WO2015/161276, e.g., in FIGS. 1A-1G therein and other depictions provided herein.

In some cases, the gRNA is a unimolecular or chimeric gRNA comprising, from 5′ to 3′: a targeting domain which targets a target site or position, such within as a sequence from the TRAC locus (exemplary nucleotide sequence of the human TRAC gene locus set forth in SEQ ID NO:1; NCBI Reference Sequence: NG_001332.3, TRAC; exemplary genomic sequence described in Table 1 herein); a first complementarity domain; a linking domain; a second complementarity domain (which is complementary to the first complementarity domain); a proximal domain; and optionally, a tail domain. In some cases, the gRNA is a unimolecular or chimeric gRNA comprising, from 5′ to 3′: a targeting domain which targets a target site or position, such as within a sequence from the TRBC1 or TRBC2 locus (exemplary nucleotide sequence of the human TRBC1 gene locus set forth in SEQ ID NO:2; NCBI Reference Sequence: NG_001333.2, TRBC1; exemplary genomic sequence described in Table 2 herein; exemplary nucleotide sequence of the human TRBC2 gene locus set forth in SEQ ID NO:3; NCBI Reference Sequence: NG_001333.2, TRBC2; exemplary genomic sequence described in Table 3 herein); a first complementarity domain; a linking domain; a second complementarity domain (which is complementary to the first complementarity domain); a proximal domain; and optionally, a tail domain.

In other cases, the gRNA is a modular gRNA comprising first and second strands. In these cases, the first strand preferably includes, from 5′ to 3′: a targeting domain (which targets a target site or position, such as within a sequence from TRAC locus (exemplary nucleotide sequence of the human TRAC gene locus set forth in SEQ ID NO:1; NCBI Reference Sequence: NG_001332.3, TRAC; exemplary genomic sequence described in Table 1 herein) or TRBC1 or TRBC2 locus (exemplary nucleotide sequence of the human TRBC1 gene locus set forth in SEQ ID NO:2; NCBI Reference Sequence: NG_001333.2, TRBC11; exemplary genomic sequence described in Table 2 herein; exemplary nucleotide sequence of the human TRBC2 gene locus set forth in SEQ ID NO:3; NCBI Reference Sequence: NG_001333.2, TRBC2); and a first complementarity domain. The second strand generally includes, from 5′ to 3′: optionally, a 5′ extension domain; a second complementarity domain; a proximal domain; and optionally, a tail domain.

A) Targeting Domain

Examples of the placement of targeting domains include those described in WO2015/161276, e.g., in FIGS. 1A-1G therein. The targeting domain comprises a nucleotide sequence that is complementary, e.g., at least 80, 85, 90, 95, 98 or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting domains can be found, e.g., in Fu Y et al., Nat Biotechnol 2014 (doi: 10.1038/nbt.2808) and Sternberg S H et al., Nature 2014 (doi: 10.1038/nature13011).

The targeting domain is part of an RNA molecule and will therefore comprise the base uracil (U), while any DNA encoding the gRNA molecule will comprise the base thymine (T). While not wishing to be bound by theory, in some embodiments, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In some embodiments, the target domain itself comprises in the 5′ to 3′ direction, an optional secondary domain, and a core domain. In some embodiments, the core domain is fully complementary with the target sequence. In some embodiments, the targeting domain is 5 to 50 nucleotides in length. The strand of the target nucleic acid with which the targeting domain is complementary is referred to herein as the complementary strand. Some or all of the nucleotides of the domain can have a modification, e.g., to render it less susceptible to degradation, improve bio-compatibility, etc. By way of non-limiting example, the backbone of the target domain can be modified with a phosphorothioate, or other modification(s). In some cases, a nucleotide of the targeting domain can comprise a 2′ modification, e.g., a 2-acetylation, e.g., a 2′ methylation, or other modification(s).

In various embodiments, the targeting domain is 16-26 nucleotides in length (i.e. it is 16 nucleotides in length, or 17 nucleotides in length, or 18, 19, 20, 21, 22, 23, 24, 25 or 26 nucleotides in length.

B) Exemplary Targeting Domains

Exemplary targeting domains contained within the gRNA for targeting the genetic disruption of the human TRAC, TRBC1 or TRBC2 include those described in, e.g., WO2015/161276, WO2017/193107, WO2017/093969, US2016/272999 and US2015/056705 or a targeting domain that can bind to the targeting sequences described in the foregoing. Exemplary targeting domains contained within the gRNA for targeting the genetic disruption of the human TRAC locus using S. pyogenes or S. aureus Cas9 can include any of those set forth in

TABLE 4
Exemplary TRAC gRNA targeting
domain sequences

| | | | SEQ |
|---|---|---|---|
| gRNA | | Cas9 | ID |
| Name | Targeting Domain | species | NO: |
| TRAC-10 | UCUCUCAGCUGGUACACGGC | S. pyogenes | 28 |
| TRAC-110 | UGGAUUUAGAGUCUCUCAGC | S. pyogenes | 29 |
| TRAC-116 | ACACGGCAGGGUCAGGGUUC | S. pyogenes | 30 |
| TRAC-16 | GAGAAUCAAAAUCGGUGAAU | S. pyogenes | 31 |
| TRAC-4 | GCUGGUACACGGCAGGGUCA | S. pyogenes | 32 |
| TRAC-49 | CUCAGCUGGUACACGGC | S. pyogenes | 33 |
| TRAC-2 | UGGUACACGGCAGGGUC | S. pyogenes | 34 |
| TRAC-30 | GCUAGACAUGAGGUCUA | S. pyogenes | 35 |
| TRAC-43 | GUCAGAUUUGUUGCUCC | S. pyogenes | 36 |
| TRAC-23 | UCAGCUGGUACACGGCA | S. pyogenes | 37 |
| TRAC-34 | GCAGACAGACUUGUCAC | S. pyogenes | 38 |
| TRAC-25 | GGUACACGGCAGGGUCA | S. pyogenes | 39 |
| TRAC-128 | CUUCAAGAGCAACAGUGCUG | S. pyogenes | 40 |
| TRAC-105 | AGAGCAACAGUGCUGUGGCC | S. pyogenes | 41 |
| TRAC-106 | AAAGUCAGAUUUGUUGCUCC | S. pyogenes | 42 |
| TRAC-123 | ACAAAACUGUGCUAGACAUG | S. pyogenes | 43 |
| TRAC-64 | AAACUGUGCUAGACAUG | S. pyogenes | 44 |
| TRAC-97 | UGUGCUAGACAUGAGGUCUA | S. pyogenes | 45 |
| TRAC-148 | GGCUGGGGAAGAAGGUGUCUUC | S. aureus | 46 |
| TRAC-147 | GCUGGGGAAGAAGGUGUCUUC | S. aureus | 47 |
| TRAC-234 | GGGGAAGAAGGUGUCUUC | S. aureus | 48 |
| TRAC-167 | GUUUUGUCUGUGAUAUACACAU | S. aureus | 49 |
| TRAC-177 | GGCAGACAGACU | S. aureus | 50 |
| | UGUCACUGGAUU | | |
| TRAC-176 | GCAGACAGACUU | S. aureus | 51 |
| | GUCACUGGAUU | | |
| TRAC-257 | GACAGACUUGUCACUGGAUU | S. aureus | 52 |
| TRAC-233 | GUGAAUAGGCAG | S. aureus | 53 |
| | ACAGACUUGUCA | | |
| TRAC-231 | GAAUAGGCAGACAGACUUGUCA | S. aureus | 54 |
| TRAC-163 | GAGUCUCUCAGCUGGUACACGG | S. aureus | 55 |
| TRAC-241 | GUCUCUCAGCUGGUACACGG | S. aureus | 56 |
| TRAC-179 | GGUACACGGCAGGGUCAGGGUU | S. aureus | 57 |
| TRAC-178 | GUACACGGCAGGGUCAGGGUU | S. aureus | 58 |

Exemplary targeting domains contained within the gRNA for targeting the genetic disruption of the human TRBC1 or TRBC2 locus using S. pyogenes or S. aureus Cas9 can include any of those set forth in Table 5.

TABLE 5
Exemplary TRBC1 or TRBC2 gRNA
targeting domain sequences

| | | | SEQ |
|---|---|---|---|
| gRNA | | Cas9 | ID |
| Name | Targeting Domain | species | NO: |
| TRBC-40 | CACCCAGAUCGUCAGCGCCG | S. pyogenes | 59 |
| TRBC-52 | CAAACACAGCGACCUCGGGU | S. pyogenes | 60 |
| TRBC-25 | UGACGAGUGGACCCAGGAUA | S. pyogenes | 61 |
| TRBC-35 | GGCUCUCGGAGAAUGACGAG | S. pyogenes | 62 |
| TRBC-50 | GGCCUCGGCGCUGACGAUCU | S. pyogenes | 63 |
| TRBC-39 | GAAAAACGUGUUCCCACCCG | S. pyogenes | 64 |
| TRBC-49 | AUGACGAGUGGACCCAGGAU | S. pyogenes | 65 |
| TRBC-51 | AGUCCAGUUCUACGGGCUCU | S. pyogenes | 66 |
| TRBC-26 | CGCUGUCAAGUCCAGUUCUA | S. pyogenes | 67 |
| TRBC-47 | AUCGUCAGCGCCGAGGCCUG | S. pyogenes | 68 |
| TRBC-45 | UCAAACACAGCGACCUCGGG | S. pyogenes | 69 |
| TRBC-34 | CGUAGAACUGGACUUGACAG | S. pyogenes | 70 |
| TRBC-227 | AGGCCUCGGCGCUGACGAUC | S. pyogenes | 71 |
| TRBC-41 | UGACAGCGGAAGUGGUUGCG | S. pyogenes | 72 |
| TRBC-30 | UUGACAGCGGAAGUGGUUGC | S. pyogenes | 73 |
| TRBC-206 | UCUCCGAGAGCCCGUAGAAC | S. pyogenes | 74 |
| TRBC-32 | CGGGUGGGAACACGUUUUUC | S. pyogenes | 75 |
| TRBC-276 | GACAGGUUUGGCCCUAUCCU | S. pyogenes | 76 |
| TRBC-274 | GAUCGUCAGCGCCGAGGCCU | S. pyogenes | 77 |
| TRBC-230 | GGCUCAAACACAGCGACCUC | S. pyogenes | 78 |
| TRBC-235 | UGAGGGUCUCGGCCACCUUC | S. pyogenes | 79 |
| TRBC-38 | AGGCUUCUACCCCGACCACG | S. pyogenes | 80 |
| TRBC-223 | CCGACCACGUGGAGCUGAGC | S. pyogenes | 81 |
| TRBC-221 | UGACAGGUUUGGCCCUAUCC | S. pyogenes | 82 |
| TRBC-48 | CUUGACAGCGGAAGUGGUUG | S. pyogenes | 83 |
| TRBC-216 | AGAUCGUCAGCGCCGAGGCC | S. pyogenes | 84 |
| TRBC-210 | GCGCUGACGAUCUGGGUGAC | S. pyogenes | 85 |
| TRBC-268 | UGAGGGCGGGCUGCUCCUUG | S. pyogenes | 86 |
| TRBC-193 | GUUGCGGGGGUUCUGCCAGA | S. pyogenes | 87 |
| TRBC-246 | AGCUCAGCUCCACGUGGUCG | S. pyogenes | 88 |
| TRBC-228 | GCGGCUGCUCAGGCAGUAUC | S. pyogenes | 89 |
| TRBC-43 | GCGGGGGUUCUGCCAGAAGG | S. pyogenes | 90 |
| TRBC-272 | UGGCUCAAACACAGCGACCU | S. pyogenes | 91 |
| TRBC-33 | ACUGGACUUGACAGCGGAAG | S. pyogenes | 92 |
| TRBC-44 | GACAGCGGAAGUGGUUGCGG | S. pyogenes | 93 |
| TRBC-211 | GCUGUCAAGUCCAGUUCUAC | S. pyogenes | 94 |
| TRBC-253 | GUAUCUGGAGUCAUUGAGGG | S. pyogenes | 95 |
| TRBC-18 | CUCGGCGCUGACGAUCU | S. pyogenes | 96 |
| TRBC-6 | CCUCGGCGCUGACGAUC | S. pyogenes | 97 |
| TRBC-85 | CCGAGAGCCCGUAGAAC | S. pyogenes | 98 |
| TRBC-129 | CCAGAUCGUCAGCGCCG | S. pyogenes | 99 |
| TRBC-93 | GAAUGACGAGUGGACCC | S. pyogenes | 100 |
| TRBC-415 | GGGUGACAGGUUUGG | S. aureus | 101 |
| | CCCUAUC | | |
| TRBC-414 | GGUGACAGGUUUGGCC | S. aureus | 102 |
| | CUAUC | | |
| TRBC-310 | GUGACAGGUUUGGCC | S. aureus | 103 |
| | CUAUC | | |
| TRBC-308 | GACAGGUUUGGCCCUAUC | S. aureus | 104 |
| TRBC-401 | GAUACUGCCUGAG | S. aureus | 105 |
| | CAGCCGCCU | | |
| TRBC-468 | GACCACGUGGAGCU | S. aureus | 106 |
| | GAGCUGGUGG | | |
| TRBC-462 | GUGGAGCUGAGCUGGUGG | S. aureus | 107 |
| TRBC-424 | GGGCGGGCUGCUC | S. aureus | 108 |
| | CUUGAGGGGCU | | |
| TRBC-423 | GGCGGGCUGCUC | S. aureus | 109 |
| | CUUGAGGGGCU | | |
| TRBC-422 | GCGGGCUGCUC | S. aureus | 110 |
| | CUUGAGGGGCU | | |
| TRBC-420 | GGGCUGCUCCUUG | S. aureus | 111 |
| | AGGGGCU | | |
| TRBC-419 | GGCUGCUCCUUGAGGGGCU | S. aureus | 112 |
| TRBC-418 | GCUGCUCCUUGAGGGGCU | S. aureus | 113 |
| TRBC-445 | GGUGAAUGGGAA | S. aureus | 114 |
| | GGAGGUGCACAG | | |
| TRBC-444 | GUGAAUGGGAAGG | S. aureus | 115 |
| | AGGUGCACAG | | |
| TRBC-442 | GAAUGGGAAGGAG | S. aureus | 116 |
| | GUGCACAG | | |

In some embodiments, the gRNA for targeting TRAC, TRBC1 and/or TRBC2 can be any that are described herein, or are described elsewhere e.g., in WO2015/161276, WO2017/193107, WO2017/093969, US2016/272999 and US2015/056705 or a targeting domain that can bind to the targeting sequences described in the foregoing. In some embodiments, the sequence targeted by the CRISPR/Cas9 gRNA in the TRAC gene locus is set forth in SEQ ID NOS: 117, 163 and 165-211, such as GAGAATCAAAATCGGTGAAT (SEQ ID NO:163) or ATTCACCGATTTTGATTCTC (SEQ ID NO:117). In some embodiments, the sequence targeted by the CRISPR/Cas9 gRNA in the TRBC1 and/or TRBC2 gene loci is set forth in SEQ ID NOS: 118, 164 and 212, such as GGCCTCGGCGCTGACGATCT (SEQ ID NO:164) or AGATCGTCAGCGCCGAGGCC (SEQ ID NO:118). In some embodiments, the gRNA targeting domain sequence for targeting a target site in the TRAC gene locus is GAGAAUCAAAAUCGGUGAAU (SEQ ID NO:31). In some embodiments, the gRNA targeting domain sequence for targeting a target site in the TRBC1 and/or TRBC2 gene loci is GGCCUCGGCGCUGACGAUCU (SEQ ID NO:63).

In some embodiments, the gRNA for targeting the TRAC gene locus can be obtained by in vitro transcription of the sequence AGCGCTCTCGTACAGAGTTGGCATTATAATACGACTCACTATAGGGGAGAATCAAA ATCGGTGAATGTTTTAGAGCTAGAAATAGCAAGTTAAAATAAGGCTAGTCCGTTAT CAACTTGAAAAAGTGGCACCGAGTCGGTGCTTTTTTT (set forth in SEQ ID NO:26; bold and underlined portion is complementary to the target site in the TRAC locus), or chemically synthesized, where the gRNA had the sequence 5′-GAG AAU CAA AAU CGG UGA AUG UUU UAG AGC UAG AAA UAG CAA GUU AAA AUA AGG CUA GUC CGU UAU CAA CUU GAA AAA GUG GCA CCG AGU CGG UGC UUU U-3′ (set forth in SEQ ID NO:27; see Osborn et al., Mol Ther. 24(3):570-581 (2016)). Other exemplary gRNA sequences to generate a genetic disruption of the endogenous genes encoding TCR domains or regions, e.g., TRAC, TRBC1 and/or TRBC2 are described, e.g., in International PCT Publication No. WO2015/161276. Exemplary methods for gene editing of the endogenous TCR loci include those described in, e.g. U.S. Publication Nos. US2011/0158957, US2014/0301990, US2015/0098954, US2016/0208243; US2016/272999 and US2015/056705; International PCT Publication Nos. WO2014/191128, WO2015/136001, WO2015/161276, WO2016/069283, WO2016/016341, WO2017/193107, and WO2017/093969; and Osborn et al. (2016) Mol. Ther. 24(3):570-581. Any of the known methods can be used to generate a genetic disruption of the endogenous genes encoding TCR domains or regions can be used in the embodiments provided herein.

In some embodiments, targeting domains include those for introducing a genetic disruption at the TRAC, TRBC1 and/or TRBC2 loci using S. pyogenes Cas9 or using N. meningitidis Cas9. In some embodiments, targeting domains include those for introducing a genetic disruption at the TRAC, TRBC1 and/or TRBC2 loci using S. pyogenes Cas9. Any of the targeting domains can be used with a S. pyogenes Cas9 molecule that generates a double stranded break (Cas9 nuclease) or a single-stranded break (Cas9 nickase).

In some embodiments, dual targeting is used to create two nicks on opposite DNA strands by using S. pyogenes Cas9 nickases with two targeting domains that are complementary to opposite DNA strands, e.g., a gRNA comprising any minus strand targeting domain may be paired with any gRNA comprising a plus strand targeting domain. In some embodiments, the two gRNAs are oriented on the DNA such that PAMs face outward and the distance between the 5′ ends of the gRNAs is 0-50 bp. In some embodiments, two gRNAs are used to target two Cas9 nucleases or two Cas9 nickases, for example, using a pair of Cas9 molecule/gRNA molecule complex guided by two different gRNA molecules to cleave the target domain with two single stranded breaks on opposing strands of the target domain. In some embodiments, the two Cas9 nickases can include a molecule having HNH activity, e.g., a Cas9 molecule having the RuvC activity inactivated, e.g., a Cas9 molecule having a mutation at D10, e.g., the D10A mutation, a molecule having RuvC activity, e.g., a Cas9 molecule having the HNH activity inactivated, e.g., a Cas9 molecule having a mutation at H840, e.g., a H840A, or a molecule having RuvC activity, e.g., a Cas9 molecule having the HNH activity inactivated, e.g., a Cas9 molecule having a mutation at N863, e.g., N863A. In some embodiments, each of the two gRNAs are complexed with a D10A Cas9 nickase.

In some embodiments, the target sequence (target domain) is at or near the TRAC, TRBC1 and/or TRBC2 locus, such as any part of the TRAC, TRBC1 and/or TRBC2 coding sequence set forth in SEQ ID NO: 1-3 or described in Tables 1-3 herein. In some embodiments, the target nucleic acid complementary to the targeting domain is located at an early coding region of a gene of interest, such as TRAC, TRBC1 and/or TRBC2. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target sequence on the target nucleic acid, such as the target nucleic acid in the TRAC, TRBC1 and/or TRBC2 locus.

In some aspects, the gRNA can target a site within an exon of the open reading frame of the endogenous TRAC, TRBC1 and/or TRBC2 locus. In some aspects, the gRNA can target a site within an intron of the open reading frame of the TRAC, TRBC1 and/or TRBC2 locus. In some aspects, the gRNA can target a site within a regulatory or control element, e.g., a promoter, of the TRAC, TRBC1 and/or TRBC2 locus. In some aspects, the target site at the TRAC, TRBC1 and/or TRBC2 locus that is targeted by the gRNA can be any target sites described herein, e.g., in Section I.A.1. In some embodiments, the gRNA can target a site within or in close proximity to exons corresponding to early coding region, e.g., exon 1, 2 or 3 of the open reading frame of the endogenous TRAC, TRBC1 and/or TRBC2 locus, or including sequence immediately following a transcription start site, within exon 1, 2, or 3, or within less than 500, 450, 400, 350, 300, 250, 200, 150, 100 or 50 bp of exon 1, 2, or 3. In some embodiments, the gRNA can target a site at or near exon 2 of the endogenous TRAC, TRBC1 and/or TRBC2 locus, or within less than 500, 450, 400, 350, 300, 250, 200, 150, 100 or 50 bp of exon 2.

C) The First Complementarily Domain

Examples of first complementarity domains include those described in WO2015/161276, e.g., in FIGS. 1A-1G therein. The first complementarity domain is complementary with the second complementarity domain described herein, and generally has sufficient complementarity to the second complementarity domain to form a duplexed region under at least some physiological conditions. The first complementarity domain is typically 5 to 30 nucleotides in length, and may be 5 to 25 nucleotides in length, 7 to 25 nucleotides in length, 7 to 22 nucleotides in length, 7 to 18 nucleotides in length, or 7 to 15 nucleotides in length. In various embodiments, the first complementary domain is 5, 6, 7, 8, 9, 10, 11, 12, 13, 14, 15, 16, 17, 18, 19, 20, 21, 22, 23, 24, or 25 nucleotides in length.

Typically, the first complementarity domain does not have exact complementarity with the second complementarity domain target. In some embodiments, the first complementarity domain can have 1, 2, 3, 4 or 5 nucleotides that are not complementary with the corresponding nucleotide of the second complementarity domain. For instance, a segment of 1, 2, 3, 4, 5 or 6, (e.g., 3) nucleotides of the first complementarity domain may not pair in the duplex, and may form a non-duplexed or looped-out region. In some instances, an unpaired, or loop-out, region, e.g., a loop-out of 3 nucleotides, is present on the second complementarity domain. This unpaired region optionally begins 1, 2, 3, 4, 5, or 6, e.g., 4, nucleotides from the 5′ end of the second complementarity domain.

The first complementarity domain can include 3 subdomains, which, in the 5′ to 3′ direction are: a 5′ subdomain, a central subdomain, and a 3′ subdomain. In some embodiments, the 5′ subdomain is 4-9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4-22, 4-18, or 4 to 10, or 3, 4, 5, 6, 7, 8, 9, 10, 11, 12, 13, 14, 15, 16, 17, 18, 19, 20, 21, 22, 23, 24, or 25, nucleotides in length.

In some embodiments, the first and second complementarity domains, when duplexed, comprise 11 paired nucleotides, for example, in the gRNA sequence (one paired strand underlined, one bolded):

(SEQ ID NO: 142)
NNNNNNNNNNNNNNNNNNNNGUUUUAGAGCUAGAAAUAGCAAGUU
AAAAUAAGGCUAGUCCGUUAUCAACUUGAAAAAGUGGCACCGAGU
CGGUGC.

In some embodiments, the first and second complementarity domains, when duplexed, comprise 15 paired nucleotides, for example in the gRNA sequence (one paired strand underlined, one bolded):

(SEQ ID NO: 143)
NNNNNNNNNNNNNNNNNNNNGUUUUAGAGCUAUGCUGAAAAGCA
UAGCAAGUUAAAAUAAGGCUAGUCCGUUAUCAACUUGAAAAAGU
GGCACCGAGUCGGUGC.

In some embodiments the first and second complementarity domains, when duplexed, comprise 16 paired nucleotides, for example in the gRNA sequence (one paired strand underlined, one bolded):

(SEQ ID NO: 144)
NNNNNNNNNNNNNNNNNNNNGUUUUAGAGCUAUGCUGGAAACAG
CAUAGCAAGUUAAAAUAAGGCUAGUCCGUUAUCAACUUGAAAAA
GUGGCACCGAGUCGGUGC.

In some embodiments the first and second complementarity domains, when duplexed, comprise 21 paired nucleotides, for example in the gRNA sequence (one paired strand underlined, one bolded):

(SEQ ID NO: 145)
NNNNNNNNNNNNNNNNNNNNGUUUUAGAGCUAUGCUGUUUUGGA
AACAAAACAGCAUAGCAAGUUAAAAUAAGGCUAGUCCGUUAUCA
ACUUGAAAAAGUGGCACCGAGUCGGUGC.

In some embodiments, nucleotides are exchanged to remove poly-U tracts, for example in the gRNA sequences (exchanged nucleotides underlined):

(SEQ ID NO: 146)
NNNNNNNNNNNNNNNNNNNNGUAUUAGAGCUAGAAAUAGCAAGU
UAAUAUAAGGCUAGUCCGUUAUCAACUUGAAAAAGUGGCACCGA
GUCGGUGC;
(SEQ ID NO: 147)
NNNNNNNNNNNNNNNNNNNNGUUUAAGAGCUAGAAAUAGCAAGU
UUAAAUAAGGCUAGUCCGUUAUCAACUUGAAAAAGUGGCACCGA
GUCGGUGC;
and
(SEQ ID NO: 148)
NNNNNNNNNNNNNNNNNNNNGUAUUAGAGCUAUGCUGUAUUGGA
AACAAUACAGCAUAGCAAGUUAAUAUAAGGCUAGUCCGUUAUCA
ACUUGAAAAAGUGGCACCGAGUCGGUGC.

The first complementarity domain can share homology with, or be derived from, a naturally occurring first complementarity domain. In some embodiments, it has at least 50% homology with a first complementarity domain disclosed herein, e.g., an S. pyogenes, S. aureus, N. meningtidis, or S. thermophilus, first complementarity domain.

It should be noted that one or more, or even all of the nucleotides of the first complementarity domain, can have a modification along the lines discussed herein for the targeting domain.

D) The Linking Domain

Examples of linking domains include those described in WO2015/161276, e.g., in FIGS. 1A-1G therein. In a unimolecular or chimeric gRNA, the linking domain serves to link the first complementarity domain with the second complementarity domain of a unimolecular gRNA. The linking domain can link the first and second complementarity domains covalently or non-covalently. In some embodiments, the linkage is covalent. In some embodiments, the linking domain covalently couples the first and second complementarity domains, see, e.g., WO2015/161276, e.g., in FIGS. 1B-1E therein. In some embodiments, the linking domain is, or comprises, a covalent bond interposed between the first complementarity domain and the second complementarity domain. Typically the linking domain comprises one or more, e.g., 2, 3, 4, 5, 6, 7, 8, 9, or 10 nucleotides, but in various embodiments the linker can be 20, 30, 40, 50 or even 100 nucleotides in length.

In modular gRNA molecules, the two molecules are associated by virtue of the hybridization of the complementarity domains and a linking domain may not be present. See e.g., WO2015/161276, e.g., in FIG. 1A therein.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length. In some embodiments, a linking domain is 2, 3, 4, 5, 6, 7, 8, 9, 10, 15, 20, or 25 or more nucleotides in length. In some embodiments, a linking domain is 2 to 50, 2 to 40, 2 to 30, 2 to 20, 2 to 10, or 2 to 5 nucleotides in length. In some embodiments, a linking domain shares homology with, or is derived from, a naturally occurring sequence, e.g., the sequence of a tracrRNA that is 5′ to the second complementarity domain. In some embodiments, the linking domain has at least 50% homology with a linking domain disclosed herein.

As discussed herein in connection with the first complementarity domain, some or all of the nucleotides of the linking domain can include a modification.

E) The 5′ Extension Domain

In some cases, a modular gRNA can comprise additional sequence, 5′ to the second complementarity domain, referred to herein as the 5′ extension domain, WO2015/161276, e.g., in FIG. 1A therein. In some embodiments, the 5′ extension domain is, 2-10, 2-9, 2-8, 2-7, 2-6, 2-5, or 2-4 nucleotides in length. In some embodiments, the 5′ extension domain is 2, 3, 4, 5, 6, 7, 8, 9, or 10 or more nucleotides in length.

F) The Second Complementarily Domain

Examples of second complementarity domains include those described in WO2015/161276, e.g., in FIGS. 1A-1G therein. The second complementarity domain is complementary with the first complementarity domain, and generally has sufficient complementarity to the second complementarity domain to form a duplexed region under at least some physiological conditions. In some cases, e.g., as shown in WO2015/161276, e.g., in FIG. 1A-1B therein, the second complementarity domain can include sequence that lacks complementarity with the first complementarity domain, e.g., sequence that loops out from the duplexed region.

The second complementarity domain may be 5 to 27 nucleotides in length, and in some cases may be longer than the first complementarity region. For instance, the second complementary domain can be 7 to 27 nucleotides in length, 7 to 25 nucleotides in length, 7 to 20 nucleotides in length, or 7 to 17 nucleotides in length. More generally, the complementary domain may be 5, 6, 7, 8, 9, 10, 11, 12, 13, 14, 15, 16, 17, 18, 19, 20, 21, 22, 23, 24, 25, or 26 nucleotides in length.

In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length.

In some embodiments, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In some embodiments, it has at least 50% homology with a second complementarity domain disclosed herein, e.g., an S. pyogenes, S. aureus, N. meningtidis, or S. thermophilus, first complementarity domain.

Some or all of the nucleotides of the second complementarity domain can have a modification, e.g., a modification described herein.

G) The Proximal Domain

Examples of proximal domains include those described in WO2015/161276, e.g., in FIGS. 1A-1G therein. In some embodiments, the proximal domain is 5 to 20 nucleotides in length. In some embodiments, the proximal domain can share homology with or be derived from a naturally occurring proximal domain. In some embodiments, it has at least 50% homology with a proximal domain disclosed herein, e.g., an S. pyogenes, S. aureus, N. meningtidis, or S. thermophilus, proximal domain.

Some or all of the nucleotides of the proximal domain can have a modification along the lines described herein.

H) The Tail Domain

Examples of tail domains include those described in WO2015/161276, e.g., in FIGS. 1A-1G therein. As can be seen by inspection of the tail domains in WO2015/161276, e.g., in FIG. 1A and FIGS. 1B-1F therein, a broad spectrum of tail domains are suitable for use in gRNA molecules. In various embodiments, the tail domain is 0 (absent), 1, 2, 3, 4, 5, 6, 7, 8, 9, or 10 nucleotides in length. In certain embodiments, the tail domain nucleotides are from or share homology with sequence from the 5′ end of a naturally occurring tail domain, see e.g., WO2015/161276, e.g., in FIG. 1D or 1E therein. The tail domain also optionally includes sequences that are complementary to each other and which, under at least some physiological conditions, form a duplexed region.

Tail domains can share homology with or be derived from naturally occurring proximal tail domains. By way of non-limiting example, a given tail domain according to various embodiments of the present disclosure may share at least 50% homology with a naturally occurring tail domain disclosed herein, e.g., an S. pyogenes, S. aureus, N. meningtidis, or S. thermophilus, tail domain.

In certain cases, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

As a non-limiting example, in various embodiments the proximal and tail domain, taken together comprise the following sequences: AAGGCUAGUCCGUUAUCAACUUGAAAAAGUGGCACCGAGUCGGUGCU (SEQ ID NO:149), AAGGCUAGUCCGUUAUCAACUUGAAAAAGUGGCACCGAGUCGGUGGUGC (SEQ ID NO:150), AAGGCUAGUCCGUUAUCAACUUGAAAAAGUGGCACCGAGUCGGUGCGGAUC (SEQ ID NO:151), AAGGCUAGUCCGUUAUCAACUUGAAAAAGUG (SEQ ID NO:152), AAGGCUAGUCCGUUAUCA (SEQ ID NO:153), or AAGGCUAGUCCG (SEQ ID NO:154).

In some embodiments, the tail domain comprises the 3′ sequence UUUUUU, e.g., if a U6 promoter is used for transcription. In some embodiments, the tail domain comprises the 3′ sequence UUUU, e.g., if an H1 promoter is used for transcription. In some embodiments, tail domain comprises variable numbers of 3′ Us depending, e.g., on the termination signal of the pol-III promoter used. In some embodiments, the tail domain comprises variable 3′ sequence derived from the DNA template if a T7 promoter is used. In some embodiments, the tail domain comprises variable 3′ sequence derived from the DNA template, e.g., if in vitro transcription is used to generate the RNA molecule. In some embodiments, the tail domain comprises variable 3′ sequence derived from the DNA template, e.g., if a pol-II promoter is used to drive transcription.

In some embodiments a gRNA has the following structure: 5′ [targeting domain]-[first complementarity domain]-[linking domain]-[second complementarity domain]-[proximal domain]-[tail domain]-3′ wherein, the targeting domain comprises a core domain and optionally a secondary domain, and is 10 to 50 nucleotides in length; the first complementarity domain is 5 to 25 nucleotides in length and, in some embodiments has at least 50, 60, 70, 80, 85, 90, 95, 98 or 99% homology with a reference first complementarity domain disclosed herein; the linking domain is 1 to 5 nucleotides in length; the proximal domain is 5 to 20 nucleotides in length and, in some embodiments has at least 50, 60, 70, 80, 85, 90, 95, 98 or 99% homology with a reference proximal domain disclosed herein; and the tail domain is absent or a nucleotide sequence is 1 to 50 nucleotides in length and, in some embodiments has at least 50, 60, 70, 80, 85, 90, 95, 98 or 99% homology with a reference tail domain disclosed herein.

I) Exemplary Chimeric gRNAs

In some embodiments, a unimolecular, or chimeric, gRNA comprises, preferably from 5′ to 3′: a targeting domain, e.g., comprising 15, 16, 17, 18, 19, 20, 21, 22, 23, 24, 25, or 26 nucleotides (which is complementary to a target nucleic acid); a first complementarity domain; a linking domain; a second complementarity domain (which is complementary to the first complementarity domain); a proximal domain; and a tail domain, wherein, (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the sequence from (a), (b), or (c), has at least 60, 75, 80, 85, 90, 95, or 99% homology with the corresponding sequence of a naturally occurring gRNA, or with a gRNA described herein. In some embodiments, the proximal and tail domain, when taken together, comprise at least 15, 18, 20, 25, 30, 31, 35, 40, 45, 49, 50, or 53 nucleotides. In some embodiments, there are at least 15, 18, 20, 25, 30, 31, 35, 40, 45, 49, 50, or 53 nucleotides 3′ to the last nucleotide of the second complementarity domain. In some embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain. In some embodiments, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGAGCUAGAAAUAGCAAGUUAAAAUAAG GCUAGUCCGUUAUCAACUUGAAAAAGUGGCACCGAGUCGGUGCUUUUUU (SEQ ID NO:155). In some embodiments, the unimolecular, or chimeric, gRNA molecule is a S. pyogenes gRNA molecule.

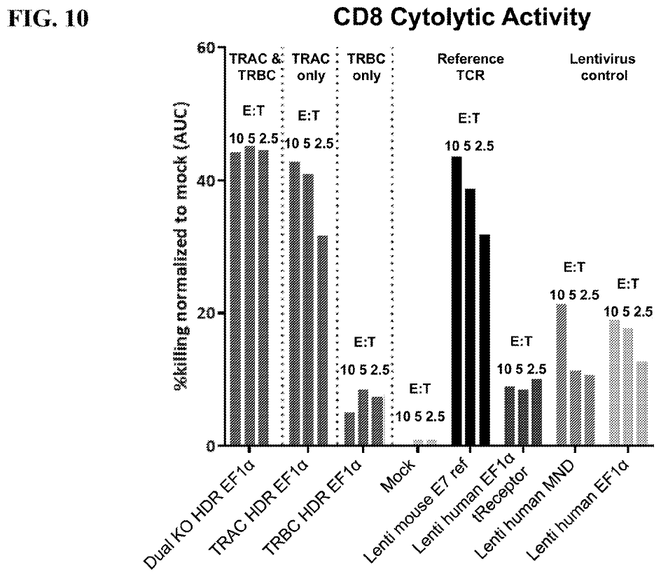

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number: NNNNNNNNNNNNNNNNNNNNGUUUUAGUACUCUGGAAACAGAAUCUACUAAAAC AAGGCAAAAUGCCGUGUUUAUCUCGUCAACUUGUUGGCGAGAUUUUUU (SEQ ID NO:156). In some embodiments, the unimolecular, or chimeric, gRNA molecule is a S. aureus gRNA molecule. The sequences and structures of exemplary chimeric gRNAs are also shown in WO2015/161276, e.g., in FIGS. 10A-10B therein.

J) Exemplary Modular gRNAs

In some embodiments, a modular gRNA comprises first and second strands. The first strand comprises, preferably from 5′ to 3′; a targeting domain, e.g., comprising 15, 16, 17, 18, 19, 20, 21, 22, 23, 24, 25, or 26 nucleotides; a first complementarity domain. The second strand comprises, preferably from 5′ to 3′: optionally a 5′ extension domain; a second complementarity domain; a proximal domain; and a tail domain, wherein: (a) the proximal and tail domain, when taken together, comprise at least 15, 18, 20, 25, 30, 31, 35, 40, 45, 49, 50, or 53 nucleotides; (b) there are at least 15, 18, 20, 25, 30, 31, 35, 40, 45, 49, 50, or 53 nucleotides 3′ to the last nucleotide of the second complementarity domain; or (c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the sequence from (a), (b), or (c), has at least 60, 75, 80, 85, 90, 95, or 99% homology with the corresponding sequence of a naturally occurring gRNA, or with a gRNA described herein. In some embodiments, the proximal and tail domain, when taken together, comprise at least 15, 18, 20, 25, 30, 31, 35, 40, 45, 49, 50, or 53 nucleotides. In some embodiments there are at least 15, 18, 20, 25, 30, 31, 35, 40, 45, 49, 50, or 53 nucleotides 3′ to the last nucleotide of the second complementarity domain.

In some embodiments, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In some embodiments, the targeting domain has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

K) Methods for Designing gRNAs

Methods for designing gRNAs are described herein, including methods for selecting, designing and validating targeting domains. Exemplary targeting domains are also provided herein. Targeting domains discussed herein can be incorporated into the gRNAs described herein.

In some embodiments, a guide RNA (gRNA) specific to the target gene (e.g. TRAC, TRBC1 and/or TRBC2 in humans) is used to RNA-guided nucleases, e.g., Cas, to induce a DNA break at the target site or target position. Methods for designing gRNAs and exemplary targeting domains can include those described in, e.g., in International PCT Publication No. WO2015/161276. Targeting domains of can be incorporated into the gRNA that is used to target Cas9 nucleases to the target site or target position.

Methods for selection and validation of target sequences as well as off-target analyses are described, e.g., in Mali et al., 2013 Science 339(6121): 823-826; Hsu et al. Nat Biotechnol, 31(9): 827-32; Fu et al., 2014 Nat Biotechnol, doi: 10.1038/nbt.2808. PubMed PMID: 24463574; Heigwer et al., 2014 Nat Methods 11(2):122-3. doi: 10.1038/nmeth.2812. PubMed PMID: 24481216; Bae et al., 2014 Bioinformatics PubMed PMID: 24463181; Xiao A et al., 2014 Bioinformatics PubMed PMID: 24389662.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool. Candidate gRNA molecules can be evaluated by art-known methods or as described herein.

In some embodiments, gRNAs for use with S. pyogenes, S. aureus, and N. meningitidis Cas9s are identified using a DNA sequence searching algorithm, e.g., using a custom gRNA design software based on the public tool cas-offinder (Bae et al. Bioinformatics. 2014; 30(10): 1473-1475). The custom gRNA design software scores guides after calculating their genome-wide off-target propensity. Typically matches ranging from perfect matches to 7 mismatches are considered for guides ranging in length from 17 to 24. In some aspects, once the off-target sites are computationally determined, an aggregate score is calculated for each guide and summarized in a tabular output using a web-interface. In addition to identifying potential gRNA sites adjacent to PAM sequences, the software also can identify all PAM adjacent sequences that differ by 1, 2, 3 or more nucleotides from the selected gRNA sites. In some embodiments, gGenomic DNA sequences for each gene are obtained from the UCSC Genome browser and sequences can be screened for repeat elements using the publicly available RepeatMasker program. RepeatMasker searches input DNA sequences for repeated elements and regions of low complexity. The output is a detailed annotation of the repeats present in a given query sequence.

Following identification, gRNAs can be ranked into tiers based on one or more of their distance to the target site, their orthogonality and presence of a 5′ G (based on identification of close matches in the human genome containing a relevant PAM, e.g., in the case of S. pyogenes, a NGG PAM, in the case of S. aureus, NNGRR (e.g, a NNGRRT or NNGRRV) PAM, and in the case of N. meningtidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality are selected to minimize off-target DNA cleavage. It is to be understood that this is a non-limiting example and that a variety of strategies could be utilized to identify gRNAs for use with S. pyogenes, S. aureus and N. meningitidis or other Cas9 enzymes.

In some embodiments, gRNAs for use with the S. pyogenes Cas9 can be identified using the publicly available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. In some aspects, genomic DNA sequences for each gene can be obtained from the UCSC Genome browser and sequences can be screened for repeat elements using the publicly available Repeat-Masker program. RepeatMasker searches input DNA sequences for repeated elements and regions of low complexity. The output is a detailed annotation of the repeats present in a given query sequence.

Following identification, gRNAs for use with a S. pyogenes Cas9 can be ranked into tiers, e.g. into 5 tiers. In some embodiments, the targeting domains for first tier gRNA molecules are selected based on their distance to the target site, their orthogonality and presence of a 5′ G (based on the ZiFiT identification of close matches in the human genome containing an NGG PAM). In some embodiments, both 17-mer and 20-mer gRNAs are designed for targets. In some aspects, gRNAs are also selected both for single-gRNA nuclease cutting and for the dual gRNA nickase strategy. Criteria for selecting gRNAs and the determination for which gRNAs can be used for which strategy can be based on several considerations. In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

In some embodiments, the targeting domains for first tier gRNA molecules can be selected based on (1) a reasonable distance to the target position, e.g., within the first 500 bp of coding sequence downstream of start codon, (2) a high level of orthogonality, and (3) the presence of a 5′ G. In some embodiments, for selection of second tier gRNAs, the requirement for a 5′G can be removed, but the distance restriction is required and a high level of orthogonality was required. In some embodiments, third tier selection uses the same distance restriction and the requirement for a 5′G, but removes the requirement of good orthogonality. In some embodiments, fourth tier selection uses the same distance restriction but removes the requirement of good orthogonality and start with a 5′G. In some embodiments, fifth tier selection removes the requirement of good orthogonality and a 5′G, and a longer sequence (e.g., the rest of the coding sequence, e.g., additional 500 bp upstream or downstream to the transcription target site) is scanned. In certain instances, no gRNA is identified based on the criteria of the particular tier.

In some embodiments, gRNAs are identified for single-gRNA nuclease cleavage as well as for a dual-gRNA paired “nickase” strategy.

In some aspects, gRNAs for use with the N. meningitidis and S. aureus Cas9s can be identified manually by scanning genomic DNA sequence for the presence of PAM sequences. These gRNAs can be separated into two tiers. In some embodiments, for first tier gRNAs, targeting domains are selected within the first 500 bp of coding sequence downstream of start codon. In some embodiments, for second tier gRNAs, targeting domains are selected within the remaining coding sequence (downstream of the first 500 bp). In certain instances, no gRNA is identified based on the criteria of the particular tier.

In some embodiments, another strategy for identifying guide RNAs (gRNAs) for use with S. pyogenes, S. aureus and N. meningtidis Cas9s can use a DNA sequence searching algorithm. In some aspects, guide RNA design is carried out using a custom guide RNA design software based on the public tool cas-offinder (Bae et al. Bioinformatics. 2014; 30(10): 1473-1475). Said custom guide RNA design software scores guides after calculating their genomewide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. In some embodiments, genomic DNA sequence for each gene is obtained from the UCSC Genome browser and sequences are screened for repeat elements using the publically available RepeatMasker program. RepeatMasker searches input DNA sequences for repeated elements and regions of low complexity. The output is a detailed annotation of the repeats present in a given query sequence.

In some embodiments, following identification, gRNAs are ranked into tiers based on their distance to the target site or their orthogonality (based on identification of close matches in the human genome containing a relevant PAM, e.g., in the case of S. pyogenes, a NGG PAM, in the case of S. aureus, NNGRR (e.g, a NNGRRT or NNGRRV) PAM, and in the case of N. meningtidis, a NNNNGATT or NNNNGCTT PAM. In some aspects, targeting domains with good orthogonality are selected to minimize off-target DNA cleavage.

As an example, for S. pyogenes and N. meningtidis targets, 17-mer, or 20-mer gRNAs can be designed. As another example, for S. aureus targets, 18-mer, 19-mer, 20-mer, 21-mer, 22-mer, 23-mer and 24-mer gRNAs can be designed.

In some embodiments, gRNAs for both single-gRNA nuclease cleavage and for a dual-gRNA paired “nickase” strategy are identified. In some embodiments for selecting gRNAs, including the determination for which gRNAs can be used for the dual-gRNA paired “nickase” strategy, gRNA pairs should be oriented on the DNA such that PAMs are facing out and cutting with the D10A Cas9 nickase will result in 5′ overhangs. In some aspects, it can be assumed that cleaving with dual nickase pairs will result in deletion of the entire intervening sequence at a reasonable frequency. However, cleaving with dual nickase pairs can also often result in indel mutations at the site of only one of the gRNAs. Candidate pair members can be tested for how efficiently they remove the entire sequence versus just causing indel mutations at the site of one gRNA.

For designing strategies for genetic disruption, in some embodiments, the targeting domains for tier 1 gRNA molecules for S. pyogenes are selected based on their distance to the target site and their orthogonality (PAM is NGG). In some cases, the targeting domains for tier 1 gRNA molecules are selected based on (1) a reasonable distance to the target position, e.g., within the first 500 bp of coding sequence downstream of start codon and (2) a high level of orthogonality. In some aspects, for selection of tier 2 gRNAs, a high level of orthogonality is not required. In some cases, tier 3 gRNAs remove the requirement of good orthogonality and a longer sequence (e.g., the rest of the coding sequence) can be scanned. In certain instances, no gRNA is identified based on the criteria of the particular tier.

For designing strategies for genetic disruption, in some embodiments, the targeting domain for tier 1 gRNA molecules for N. meningtidis were selected within the first 500 bp of the coding sequence and had a high level of orthogonality. The targeting domain for tier 2 gRNA molecules for N. meningtidis were selected within the first 500 bp of the coding sequence and did not require high orthogonality. The targeting domain for tier 3 gRNA molecules for N. meningtidis were selected within a remainder of coding sequence downstream of the 500 bp. Note that tiers are non-inclusive (each gRNA is listed only once). In certain instances, no gRNA was identified based on the criteria of the particular tier.

For designing strategies for genetic disruption, in some embodiments, the targeting domain for tier 1 gRNA molecules for S. aureus is selected within the first 500 bp of the coding sequence, has a high level of orthogonality, and contains a NNGRRT PAM. In some embodiments, the targeting domain for tier 2 gRNA molecules for S. aureus is selected within the first 500 bp of the coding sequence, no level of orthogonality is required, and contains a NNGRRT PAM. In some embodiments, the targeting domain for tier 3 gRNA molecules for S. aureus are selected within the remainder of the coding sequence downstream and contain a NNGRRT PAM. In some embodiments, the targeting domain for tier 4 gRNA molecules for S. aureus are selected within the first 500 bp of the coding sequence and contain a NNGRRV PAM. In some embodiments, the targeting domain for tier 5 gRNA molecules for S. aureus are selected within the remainder of the coding sequence downstream and contain a NNGRRV PAM. In certain instances, no gRNA is identified based on the criteria of the particular tier.

2) Cas9

Cas9 molecules of a variety of species can be used in the methods and compositions described herein. While the S. pyogenes, S. aureus, N. meningitidis, and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes, S. aureus, N. meningitidis, and S. thermophilus Cas9 molecules, Cas9 molecules from the other species can replace them. Such species include: Acidovorax avenae, Actinobacillus pleuropneumoniae, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae. Examples of Cas9 molecules can include those described in, e.g., WO2015/161276, WO2017/193107, WO2017/093969, US2016/272999 and US2015/056705.

A Cas9 molecule, or Cas9 polypeptide, as that term is used herein, refers to a molecule or polypeptide that can interact with a gRNA molecule and, in concert with the gRNA molecule, homes or localizes to a site which comprises a target domain and PAM sequence. Cas9 molecule and Cas9 polypeptide, as those terms are used herein, refer to naturally occurring Cas9 molecules and to engineered, altered, or modified Cas9 molecules or Cas9 polypeptides that differ, e.g., by at least one amino acid residue, from a reference sequence, e.g., the most similar naturally occurring Cas9 molecule.

Crystal structures have been determined for two different naturally occurring bacterial Cas9 molecules (Jinek et al., Science, 343(6176):1247997, 2014) and for S. pyogenes Cas9 with a guide RNA (e.g., a synthetic fusion of crRNA and tracrRNA) (Nishimasu et al., Cell, 156:935-949, 2014; and Anders et al., Nature, 2014, doi: 10.1038/nature13579).

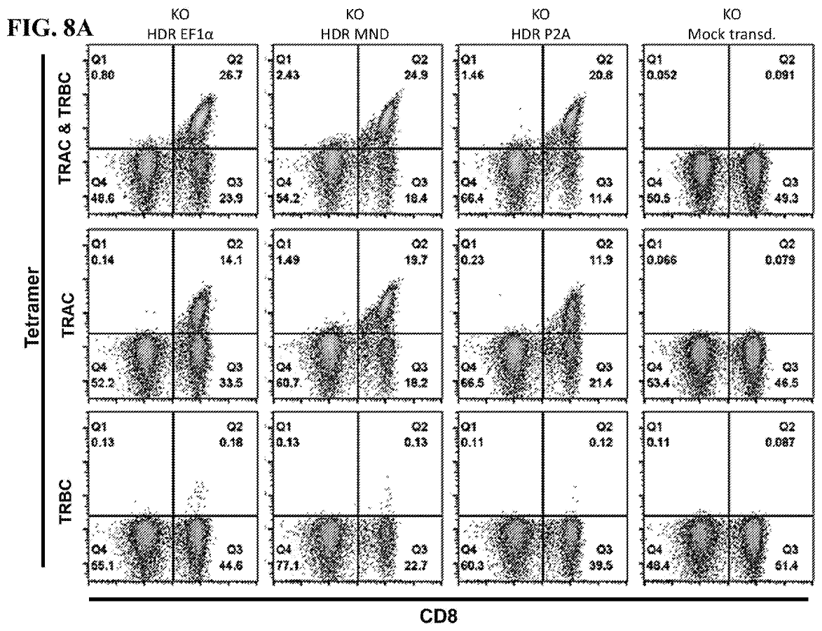

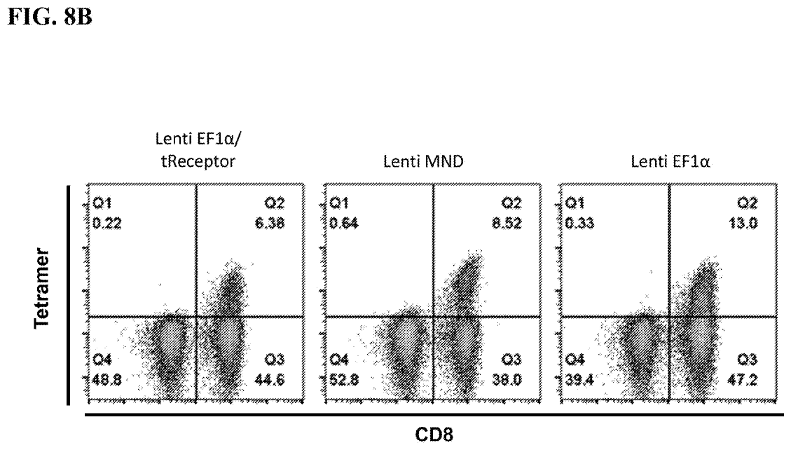

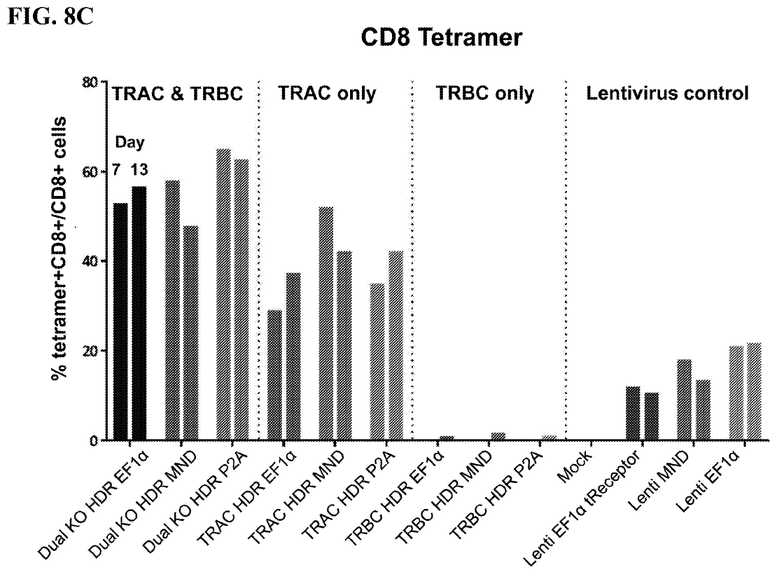

A naturally occurring Cas9 molecule comprises two lobes: a recognition (REC) lobe and a nuclease (NUC) lobe; each of which further comprises domains described herein. An exemplary schematic of the organization of important Cas9 domains in the primary structure is described in WO2015/161276, e.g., in FIGS. 8A-8B therein. The domain nomenclature and the numbering of the amino acid residues encompassed by each domain used throughout this disclosure is as described in Nishimasu et al. The numbering of the amino acid residues is with reference to Cas9 from S. pyogenes.

The REC lobe comprises the arginine-rich bridge helix (BH), the REC1 domain, and the REC2 domain. The REC lobe does not share structural similarity with other known proteins, indicating that it is a Cas9-specific functional domain. The BH domain is a long α-helix and arginine rich region and comprises amino acids 60-93 of the sequence of S. pyogenes Cas9. The REC1 domain is important for recognition of the repeat:anti-repeat duplex, e.g., of a gRNA or a tracrRNA, and is therefore critical for Cas9 activity by recognizing the target sequence. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat:anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule, and comprises amino acids 1099-1368 of the sequence of S. pyogenes Cas9.

A) a RuvC-Like Domain and an HNH-Like Domain

In some embodiments, a Cas9 molecule or Cas9 polypeptide comprises an HNH-like domain and a RuvC-like domain. In some embodiments, cleavage activity is dependent on a RuvC-like domain and an HNH-like domain. A Cas9 molecule or Cas9 polypeptide, e.g., an eaCas9 molecule or eaCas9 polypeptide, can comprise one or more of the following domains: a RuvC-like domain and an HNH-like domain. In some embodiments, a Cas9 molecule or Cas9 polypeptide is an eaCas9 molecule or eaCas9 polypeptide and the eaCas9 molecule or eaCas9 polypeptide comprises a RuvC-like domain, e.g., a RuvC-like domain described herein, and/or an HNH-like domain, e.g., an HNH-like domain described herein.

B) RuvC-Like Domains

In some embodiments, a RuvC-like domain cleaves, a single strand, e.g., the non-complementary strand of the target nucleic acid molecule. The Cas9 molecule or Cas9 polypeptide can include more than one RuvC-like domain (e.g., one, two, three or more RuvC-like domains). In some embodiments, a RuvC-like domain is at least 5, 6, 7, 8 amino acids in length but not more than 20, 19, 18, 17, 16 or 15 amino acids in length. In some embodiments, the Cas9 molecule or Cas9 polypeptide comprises an N-terminal RuvC-like domain of about 10 to 20 amino acids, e.g., about 15 amino acids in length.

C) N-Terminal RuvC-Like Domains

Some naturally occurring Cas9 molecules comprise more than one RuvC-like domain with cleavage being dependent on the N-terminal RuvC-like domain. Accordingly, Cas9 molecules or Cas9 polypeptide can comprise an N-terminal RuvC-like domain.

In embodiment, the N-terminal RuvC-like domain is cleavage competent.

In embodiment, the N-terminal RuvC-like domain is cleavage incompetent.

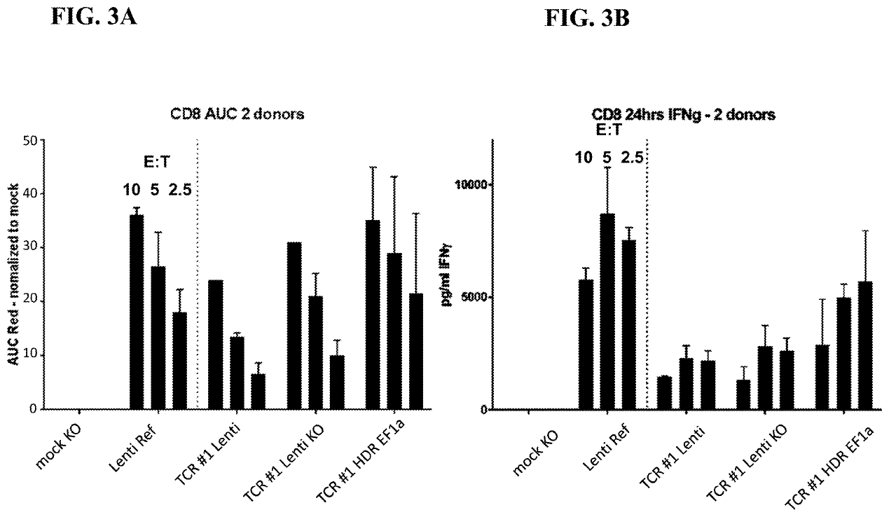

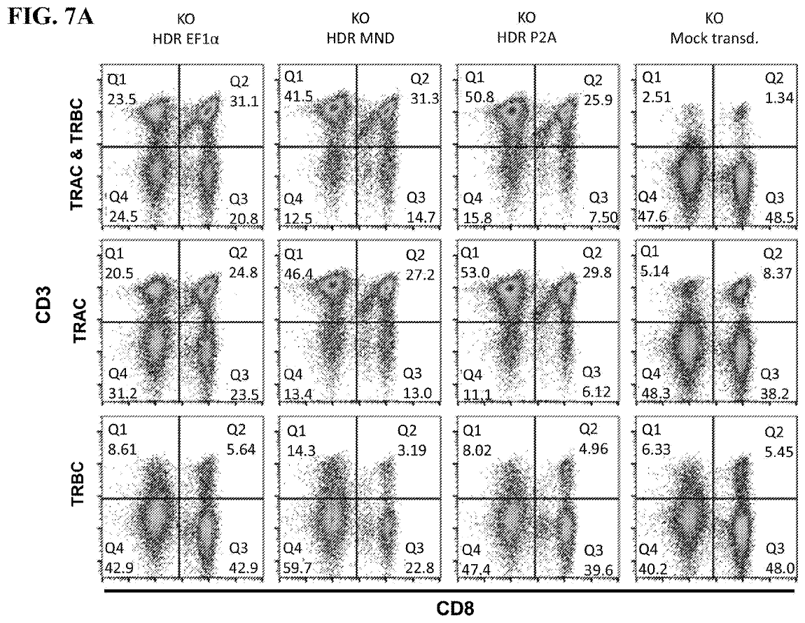

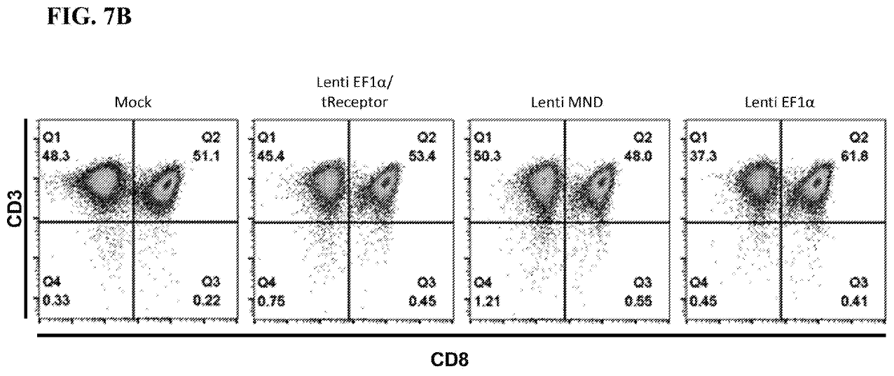

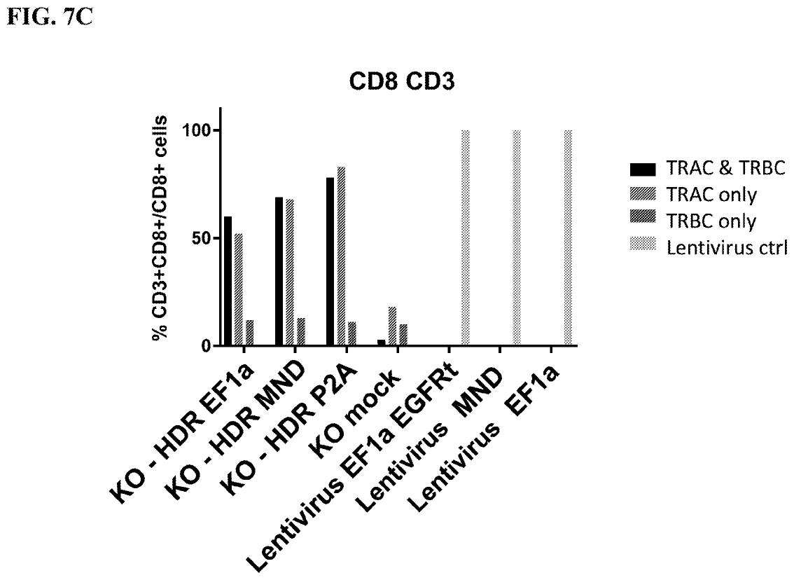

In some embodiments, the N-terminal RuvC-like domain differs from a sequence of an N-terminal RuvC like domain disclosed herein, e.g., in WO2015/161276, e.g., in FIGS. 3A-3B or FIGS. 7A-7B therein, as many as 1 but no more than 2, 3, 4, or 5 residues. In some embodiments, 1, 2, or all 3 of the highly conserved residues identified WO2015/161276, e.g., in FIGS. 3A-3B or FIGS. 7A-7B therein are present.

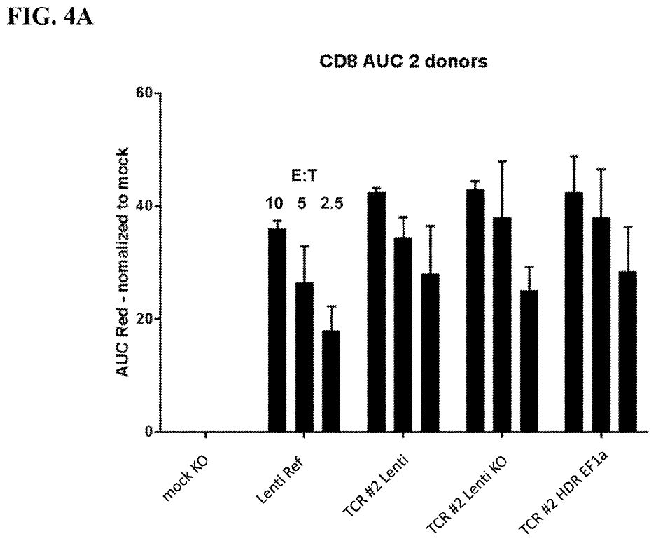

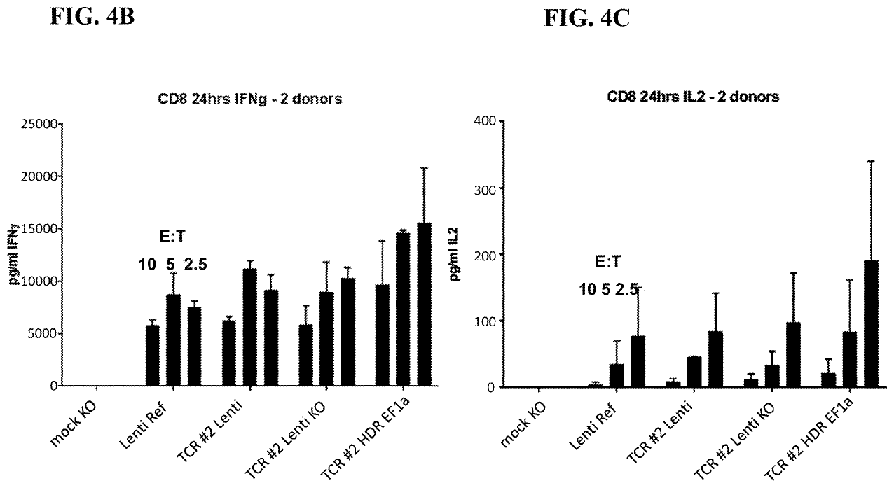

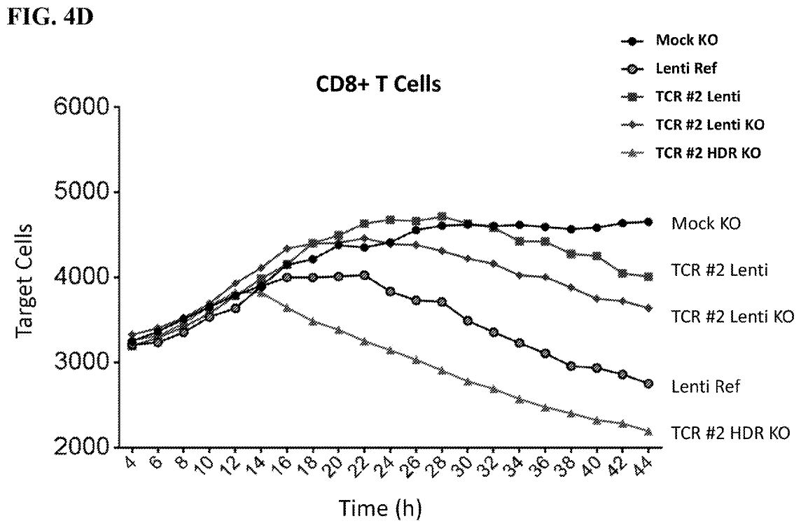

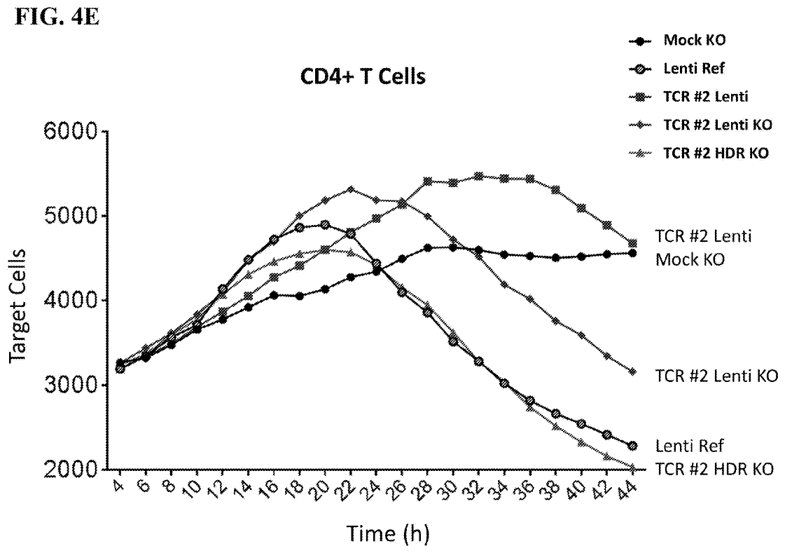

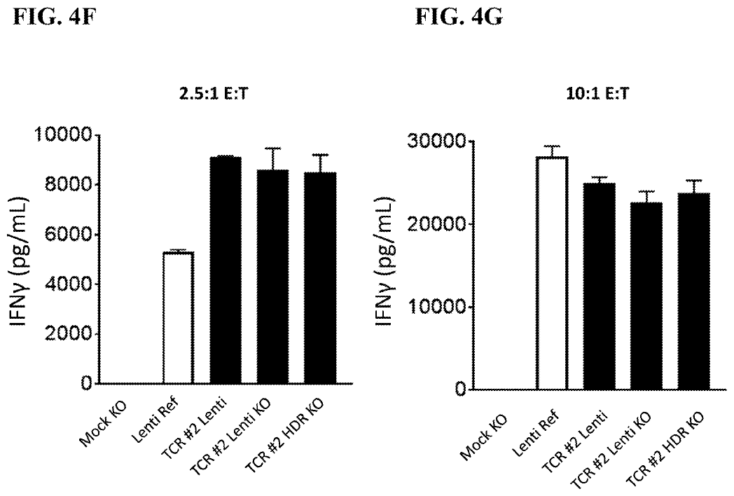

In some embodiments, the N-terminal RuvC-like domain differs from a sequence of an N-terminal RuvC-like domain disclosed herein, e.g., in WO2015/161276, e.g., in FIGS. 4A-4B or FIGS. 7A-7B therein, as many as 1 but no more than 2, 3, 4, or 5 residues. In some embodiments, 1, 2, 3 or all 4 of the highly conserved residues identified in WO2015/161276, e.g., in FIGS. 4A-4B or FIGS. 7A-7B therein are present.

D) Additional RuvC-Like Domains

In addition to the N-terminal RuvC-like domain, the Cas9 molecule or Cas9 polypeptide, e.g., an eaCas9 molecule or eaCas9 polypeptide, can comprise one or more additional RuvC-like domains. In some embodiments, the Cas9 molecule or Cas9 polypeptide can comprise two additional RuvC-like domains. Preferably, the additional RuvC-like domain is at least 5 amino acids in length and, e.g., less than 15 amino acids in length, e.g., 5 to 10 amino acids in length, e.g., 8 amino acids in length.

E) HNH-Like Domains

In some embodiments, an HNH-like domain cleaves a single stranded complementary domain, e.g., a complementary strand of a double stranded nucleic acid molecule. In some embodiments, an HNH-like domain is at least 15, 20, 25 amino acids in length but not more than 40, 35 or 30 amino acids in length, e.g., 20 to 35 amino acids in length, e.g., 25 to 30 amino acids in length. Exemplary HNH-like domains are described herein.

In some embodiments, the HNH-like domain is cleavage competent.

In some embodiments, the HNH-like domain is cleavage incompetent.

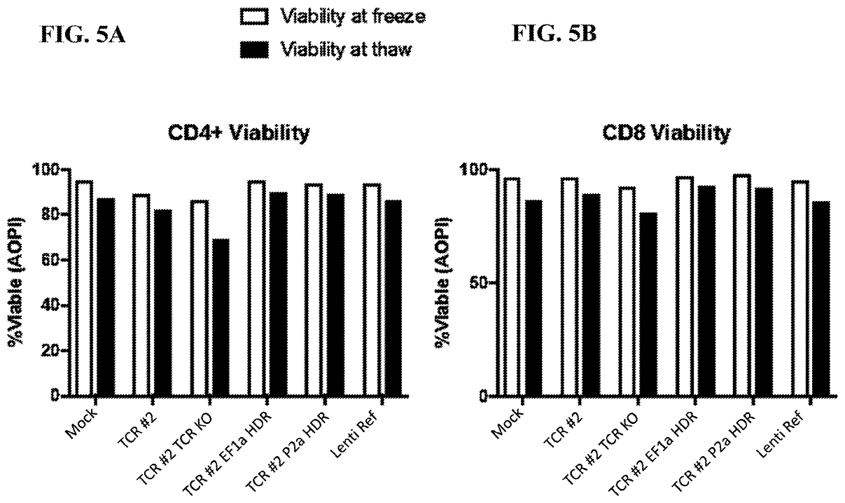

In some embodiments, the HNH-like domain differs from a sequence of an HNH-like domain disclosed herein, e.g., in WO2015/161276, e.g., in FIGS. 5A-5C or FIGS. 7A-7B therein, as many as 1 but no more than 2, 3, 4, or 5 residues. In some embodiments, 1 or both of the highly conserved residues identified in WO2015/161276, e.g., in FIGS. 5A-5C or FIGS. 7A-7B therein are present.

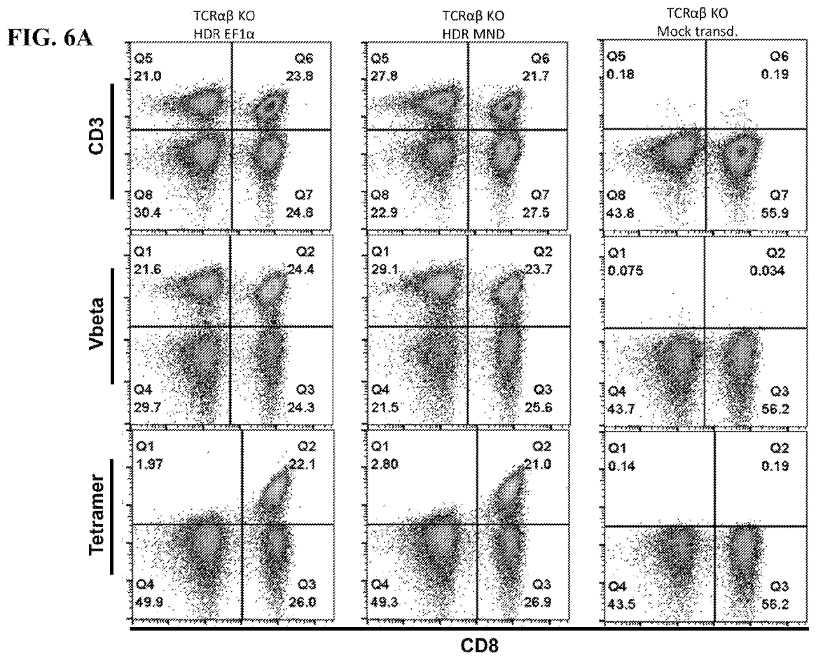

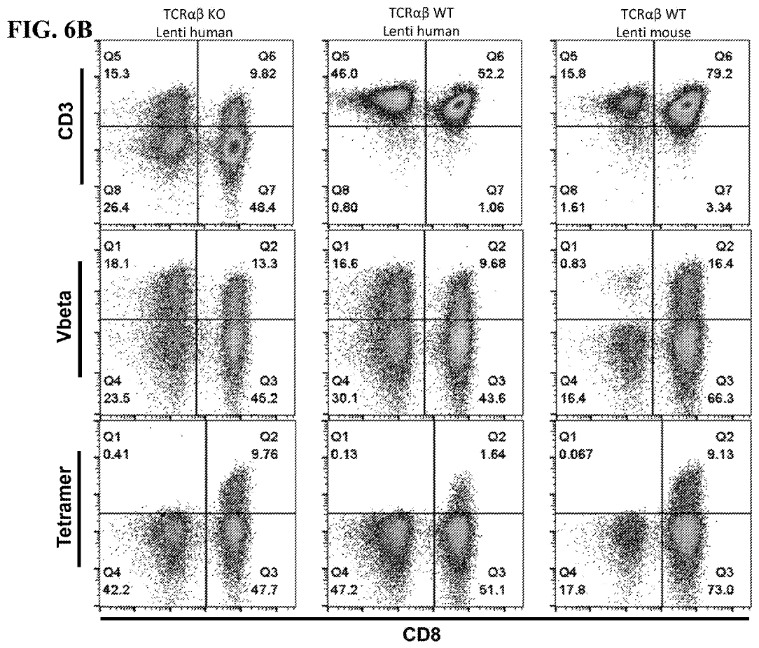

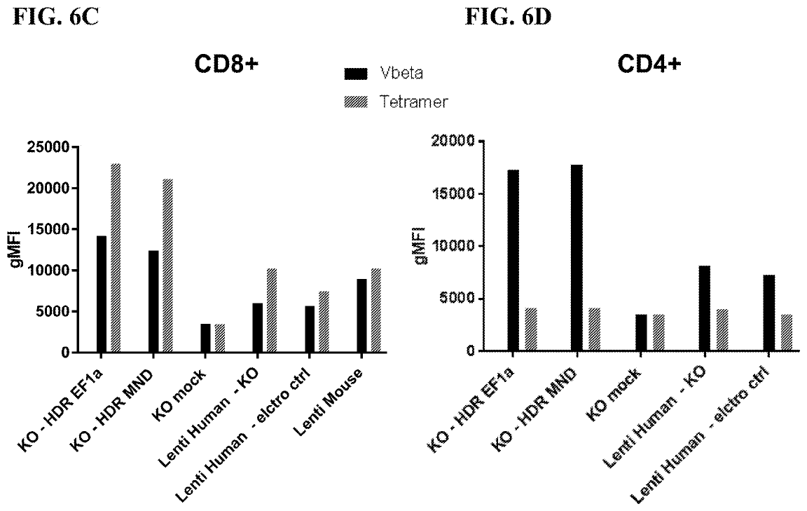

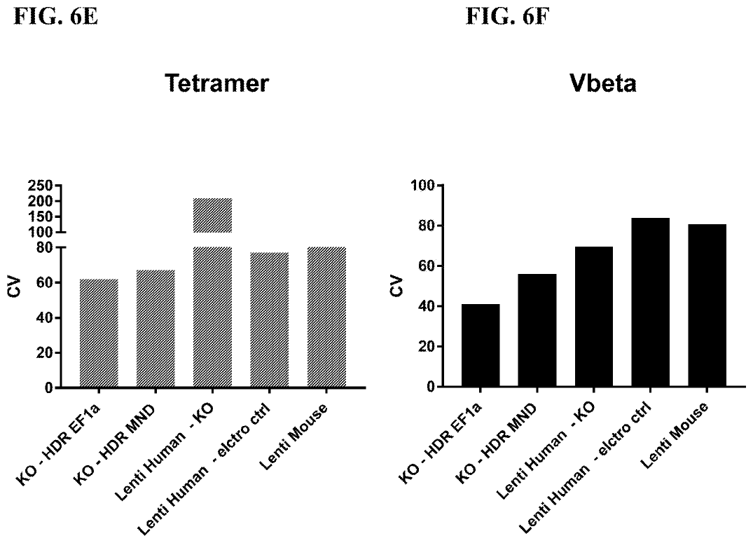

In some embodiments, the HNH-like domain differs from a sequence of an HNH-like domain disclosed herein, e.g., in WO2015/161276, e.g., in FIGS. 6A-6B or FIGS. 7A-7B therein, as many as 1 but no more than 2, 3, 4, or 5 residues. In some embodiments, 1, 2, all 3 of the highly conserved residues identified in WO2015/161276, e.g., in FIGS. 6A-6B or FIGS. 7A-7B therein are present.

F) Nuclease and Helicase Activities

In some embodiments, the Cas9 molecule or Cas9 polypeptide is capable of cleaving a target nucleic acid molecule. Typically wild type Cas9 molecules cleave both strands of a target nucleic acid molecule. Cas9 molecules and Cas9 polypeptides can be engineered to alter nuclease cleavage (or other properties), e.g., to provide a Cas9 molecule or Cas9 polypeptide which is a nickase, or which lacks the ability to cleave target nucleic acid. A Cas9 molecule or Cas9 polypeptide that is capable of cleaving a target nucleic acid molecule is referred to herein as an eaCas9 molecule or eaCas9 polypeptide

In some embodiments, an eaCas9 molecule or eaCas9 polypeptide comprises one or more of the following activities: a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in some embodiments is the presence of two nickase activities; an endonuclease activity; an exonuclease activity; and a helicase activity, i.e., the ability to unwind the helical structure of a double stranded nucleic acid.

In some embodiments, an enzymatically active or eaCas9 molecule or eaCas9 polypeptide cleaves both strands and results in a double stranded break. In some embodiments, an eaCas9 molecule cleaves only one strand, e.g., the strand to which the gRNA hybridizes to, or the strand complementary to the strand the gRNA hybridizes with. In some embodiments, an eaCas9 molecule or eaCas9 polypeptide comprises cleavage activity associated with an HNH-like domain. In some embodiments, an eaCas9 molecule or eaCas9 polypeptide comprises cleavage activity associated with an N-terminal RuvC-like domain. In some embodiments, an eaCas9 molecule or eaCas9 polypeptide comprises cleavage activity associated with an HNH-like domain and cleavage activity associated with an N-terminal RuvC-like domain. In some embodiments, an eaCas9 molecule or eaCas9 polypeptide comprises an active, or cleavage competent, HNH-like domain and an inactive, or cleavage incompetent, N-terminal RuvC-like domain. In some embodiments, an eaCas9 molecule or eaCas9 polypeptide comprises an inactive, or cleavage incompetent, HNH-like domain and an active, or cleavage competent, N-terminal RuvC-like domain.

Some Cas9 molecules or Cas9 polypeptides have the ability to interact with a gRNA molecule, and in conjunction with the gRNA molecule localize to a core target domain, but are incapable of cleaving the target nucleic acid, or incapable of cleaving at efficient rates. Cas9 molecules having no, or no substantial, cleavage activity are referred to herein as an eiCas9 molecule or eiCas9 polypeptide. For example, an eiCas9 molecule or eiCas9 polypeptide can lack cleavage activity or have substantially less, e.g., less than 20, 10, 5, 1 or 0.1% of the cleavage activity of a reference Cas9 molecule or eiCas9 polypeptide, as measured by an assay described herein.

G) Targeting and PAMs

A Cas9 molecule or Cas9 polypeptide, is a polypeptide that can interact with a guide RNA (gRNA) molecule and, in concert with the gRNA molecule, localizes to a site which comprises a target domain and a PAM sequence.

In some embodiments, the ability of an eaCas9 molecule or eaCas9 polypeptide to interact with and cleave a target nucleic acid is PAM sequence dependent. A PAM sequence is a sequence in the target nucleic acid. In some embodiments, cleavage of the target nucleic acid occurs upstream from the PAM sequence. EaCas9 molecules from different bacterial species can recognize different sequence motifs (e.g., PAM sequences). In some embodiments, an eaCas9 molecule of S. pyogenes recognizes the sequence motif NGG, NAG, NGA and directs cleavage of a target nucleic acid sequence 1 to 10, e.g., 3 to 5, base pairs upstream from that sequence. See, e.g., Mali et al., Science 2013; 339(6121): 823-826. In some embodiments, an eaCas9 molecule of S. thermophilus recognizes the sequence motif NGGNG and/or NNAGAAW (W=A or T) and directs cleavage of a target nucleic acid sequence 1 to 10, e.g., 3 to 5, base pairs upstream from these sequences. See, e.g., Horvath et al., Science 2010; 327(5962):167-170, and Deveau et al., J Bacteriol 2008; 190(4): 1390-1400. In some embodiments, an eaCas9 molecule of S. nutans recognizes the sequence motif NGG and/or NAAR (R=A or G)) and directs cleavage of a core target nucleic acid sequence 1 to 10, e.g., 3 to 5 base pairs, upstream from this sequence. See, e.g., Deveau et al., J Bacteriol 2008; 190(4): 1390-1400. In some embodiments, an eaCas9 molecule of S. aureus recognizes the sequence motif NNGRR (R=A or G) and directs cleavage of a target nucleic acid sequence 1 to 10, e.g., 3 to 5, base pairs upstream from that sequence. In some embodiments, an eaCas9 molecule of S. aureus recognizes the sequence motif NNGRRT (R=A or G) and directs cleavage of a target nucleic acid sequence 1 to 10, e.g., 3 to 5, base pairs upstream from that sequence. In some embodiments, an eaCas9 molecule of S. aureus recognizes the sequence motif NNGRRV (R=A or G) and directs cleavage of a target nucleic acid sequence 1 to 10, e.g., 3 to 5, base pairs upstream from that sequence. In some embodiments, an eaCas9 molecule of N. meningitidis recognizes the sequence motif NNNNGATT or NNNGCTT (R=A or G, V=A, G or C and directs cleavage of a target nucleic acid sequence 1 to 10, e.g., 3 to 5, base pairs upstream from that sequence. See, e.g., Hou et al., PNAS Early Edition 2013, 1-6. The ability of a Cas9 molecule to recognize a PAM sequence can be determined, e.g., using a transformation assay described in Jinek et al., Science 2012 337:816. In the aforementioned embodiments, N can be any nucleotide residue, e.g., any of A, G, C or T.

As is discussed herein, Cas9 molecules can be engineered to alter the PAM specificity of the Cas9 molecule.

Exemplary naturally occurring Cas9 molecules are described in Chylinski et al., RNA Biology 2013 10:5, 727-737. Such Cas9 molecules include Cas9 molecules of a cluster 1-78 bacterial family.

Exemplary naturally occurring Cas9 molecules include a Cas9 molecule of a cluster 1 bacterial family. Examples include a Cas9 molecule of: S. pyogenes (e.g., strain SF370, MGAS10270, MGAS10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA159, NN2025), S. macacae (e.g., strain NCTC11558), S. gallolyticus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F0211), S. agalactiae (e.g., strain NEM316, A909), Listeria monocytogenes (e.g., strain F6854), Listeria innocua (L. innocua, e.g., strain Clip11262), Enterococcus italicus (e.g., strain DSM 15952), or Enterococcus faecium (e.g., strain 1,231,408). Another exemplary Cas9 molecule is a Cas9 molecule of Neisseria meningitidis (Hou et al., PNAS Early Edition 2013, 1-6).

In some embodiments, a Cas9 molecule or Cas9 polypeptide, e.g., an eaCas9 molecule or eaCas9 polypeptide, comprises an amino acid sequence: having 60%, 65%, 70%, 75%, 80%, 85%, 90%, 95%, 96%, 97%, 98% or 99% homology with; differs at no more than, 2, 5, 10, 15, 20, 30, or 40% of the amino acid residues when compared with; differs by at least 1, 2, 5, 10 or 20 amino acids but by no more than 100, 80, 70, 60, 50, 40 or 30 amino acids from; or is identical to any Cas9 molecule sequence described herein, or a naturally occurring Cas9 molecule sequence, e.g., a Cas9 molecule from a species listed herein (e.g., SEQ ID NOS:157-162) or described in Chylinski et al., RNA Biology 2013 10:5, 727-737; Hou et al., PNAS Early Edition 2013, 1-6. In some embodiments, the Cas9 molecule or Cas9 polypeptide comprises one or more of the following activities: a nickase activity; a double stranded cleavage activity (e.g., an endonuclease and/or exonuclease activity); a helicase activity; or the ability, together with a gRNA molecule, to home to a target nucleic acid.

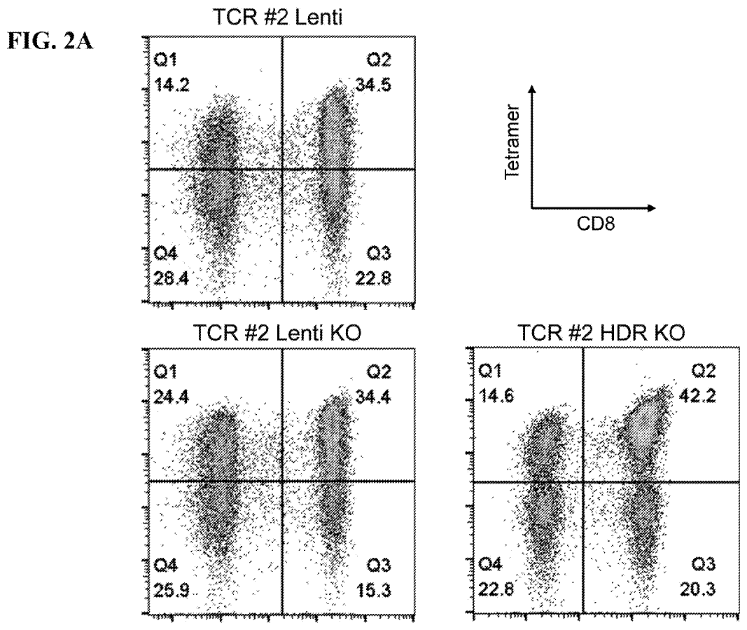

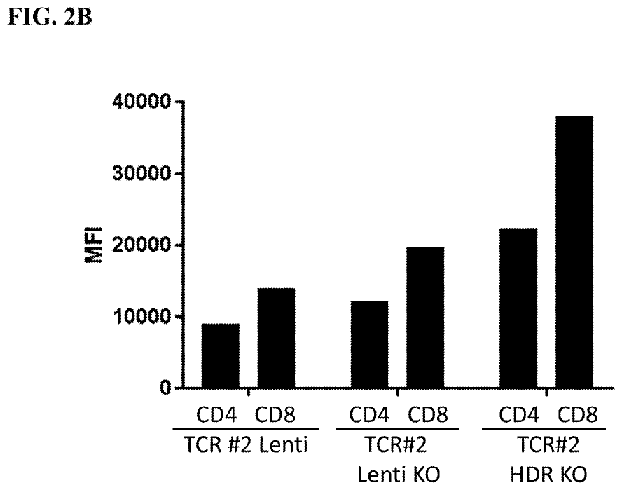

In some embodiments, a Cas9 molecule or Cas9 polypeptide comprises the amino acid sequence of the consensus sequence of WO2015/161276, e.g., in FIGS. 2A-2G therein, wherein “*” indicates any amino acid found in the corresponding position in the amino acid sequence of a Cas9 molecule of S. pyogenes, S. thermophilus, S. mutans and L. innocua, and “-” indicates any amino acid. In some embodiments, a Cas9 molecule or Cas9 polypeptide differs from the sequence of the consensus sequence of SEQ ID NOS: 157-162 or the consensus sequence disclosed in WO2015/161276, e.g., in FIGS. 2A-2G therein by at least 1, but no more than 2, 3, 4, 5, 6, 7, 8, 9, or 10 amino acid residues. In some embodiments, a Cas9 molecule or Cas9 polypeptide comprises the amino acid sequence of SEQ ID NO:162 or as described in WO2015/161276, e.g., in FIGS. 7A-7B therein, wherein “*” indicates any amino acid found in the corresponding position in the amino acid sequence of a Cas9 molecule of S. pyogenes, or N. meningitidis, “-” indicates any amino acid, and “-” indicates any amino acid or absent. In some embodiments, a Cas9 molecule or Cas9 polypeptide differs from the sequence of SEQ ID NO:161 or 162 or as described in WO2015/161276, e.g., in FIGS. 7A-7B thereinby at least 1, but no more than 2, 3, 4, 5, 6, 7, 8, 9, or 10 amino acid residues.

A comparison of the sequence of a number of Cas9 molecules indicate that certain regions are conserved. These are identified as: region 1 (residues 1 to 180, or in the case of region 1′residues 120 to 180); region 2 (residues 360 to 480); region 3 (residues 660 to 720); region 4 (residues 817 to 900); and region 5 (residues 900 to 960).

In some embodiments, a Cas9 molecule or Cas9 polypeptide comprises regions 1-5, together with sufficient additional Cas9 molecule sequence to provide a biologically active molecule, e.g., a Cas9 molecule having at least one activity described herein. In some embodiments, each of regions 1-6, independently, have, 50%, 60%, 70%, or 80% homology with the corresponding residues of a Cas9 molecule or Cas9 polypeptide described herein, e.g., set forth in SEQ ID NOS:157-162 or a sequence disclosed in WO2015/161276, e.g., from FIGS. 2A-2G or from FIGS. 7A-7B therein.

H) Engineered or Altered Cas9 Molecules and Cas9 Polypeptides

Cas9 molecules and Cas9 polypeptides described herein, e.g., naturally occurring Cas9 molecules, can possess any of a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In some embodiments, a Cas9 molecule or Cas9 polypeptide can include all or a subset of these properties. In typical embodiments, a Cas9 molecule or Cas9 polypeptide has the ability to interact with a gRNA molecule and, in concert with the gRNA molecule, localize to a site in a nucleic acid. Other activities, e.g., PAM specificity, cleavage activity, or helicase activity can vary more widely in Cas9 molecules and Cas9 polypeptides.

Cas9 molecules include engineered Cas9 molecules and engineered Cas9 polypeptides (“engineered,” as used in this context, means merely that the Cas9 molecule or Cas9 polypeptide differs from a reference sequences, and implies no process or origin limitation). An engineered Cas9 molecule or Cas9 polypeptide can comprise altered enzymatic properties, e.g., altered nuclease activity, (as compared with a naturally occurring or other reference Cas9 molecule) or altered helicase activity. As discussed herein, an engineered Cas9 molecule or Cas9 polypeptide can have nickase activity (as opposed to double strand nuclease activity). In some embodiments an engineered Cas9 molecule or Cas9 polypeptide can have an alteration that alters its size, e.g., a deletion of amino acid sequence that reduces its size, e.g., without significant effect on one or more, or any Cas9 activity. In some embodiments, an engineered Cas9 molecule or Cas9 polypeptide can comprise an alteration that affects PAM recognition. E.g., an engineered Cas9 molecule can be altered to recognize a PAM sequence other than that recognized by the endogenous wild-type PI domain. In some embodiments a Cas9 molecule or Cas9 polypeptide can differ in sequence from a naturally occurring Cas9 molecule but not have significant alteration in one or more Cas9 activities.

Cas9 molecules or Cas9 polypeptides with desired properties can be made in a number of ways, e.g., by alteration of a parental, e.g., naturally occurring, Cas9 molecules or Cas9 polypeptides, to provide an altered Cas9 molecule or Cas9 polypeptide having a desired property. For example, one or more mutations or differences relative to a parental Cas9 molecule, e.g., a naturally occurring or engineered Cas9 molecule, can be introduced. Such mutations and differences comprise: substitutions (e.g., conservative substitutions or substitutions of non-essential amino acids); insertions; or deletions. In some embodiments, a Cas9 molecule or Cas9 polypeptide can comprises one or more mutations or differences, e.g., at least 1, 2, 3, 4, 5, 10, 15, 20, 30, 40 or 50 mutations but less than 200, 100, or 80 mutations relative to a reference, e.g., a parental, Cas9 molecule.

In some embodiments, a mutation or mutations do not have a substantial effect on a Cas9 activity, e.g. a Cas9 activity described herein. In some embodiments, a mutation or mutations have a substantial effect on a Cas9 activity, e.g. a Cas9 activity described herein.

I) Non-Cleaving and Modified-Cleavage Cas9 Molecules and Cas9 Polypeptides

In some embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S. pyogenes, as follows: its ability to modulate, e.g., decreased or increased, cleavage of a double stranded nucleic acid (endonuclease and/or exonuclease activity), e.g., as compared to a naturally occurring Cas9 molecule (e.g., a Cas9 molecule of S. pyogenes); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. pyogenes); or the ability to cleave a nucleic acid molecule, e.g., a double stranded or single stranded nucleic acid molecule, can be eliminated.

J) Modified Cleavage eaCas9 Molecules and eaCas9 Polypeptides

In some embodiments, an eaCas9 molecule or eaCas9 polypeptide comprises one or more of the following activities: cleavage activity associated with an N-terminal RuvC-like domain; cleavage activity associated with an HNH-like domain; cleavage activity associated with an HNH-like domain and cleavage activity associated with an N-terminal RuvC-like domain.

In some embodiments, an eaCas9 molecule or eaCas9 polypeptide comprises an active, or cleavage competent, HNH-like domain and an inactive, or cleavage incompetent, N-terminal RuvC-like domain. An exemplary inactive, or cleavage incompetent N-terminal RuvC-like domain can have a mutation of an aspartic acid in an N-terminal RuvC-like domain, e.g., an aspartic acid at position 9 of the consensus sequence of SEQ ID NOS: 157-162 or the consensus sequence disclosed in WO2015/161276, e.g., in FIGS. 2A-2G therein or an aspartic acid at position 10 of SEQ ID NO:162, e.g., can be substituted with an alanine. In some embodiments, the eaCas9 molecule or eaCas9 polypeptide differs from wild type in the N-terminal RuvC-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

In some embodiments, an eaCas9 molecule or eaCas9 polypeptide comprises an inactive, or cleavage incompetent, HNH domain and an active, or cleavage competent, N-terminal RuvC-like domain. Exemplary inactive, or cleavage incompetent HNH-like domains can have a mutation at one or more of: a histidine in an HNH-like domain, e.g., a histidine shown at position 856 of the consensus sequence of SEQ ID NOS:157-162 or the consensus sequence disclosed in WO2015/161276, e.g., in FIGS. 2A-2G therein, e.g., can be substituted with an alanine; and one or more asparagines in an HNH-like domain, e.g., an asparagine shown at position 870 of the consensus sequence of SEQ ID NOS:157-162 or the consensus sequence disclosed in WO2015/161276, e.g., in FIGS. 2A-2G therein and/or at position 879 of the consensus sequence of SEQ ID NOS:157-162 or the consensus sequence disclosed in WO2015/161276, e.g., in FIGS. 2A-2G therein, e.g., can be substituted with an alanine. In some embodiments, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

In some embodiments, an eaCas9 molecule or eaCas9 polypeptide comprises an inactive, or cleavage incompetent, HNH domain and an active, or cleavage competent, N-terminal RuvC-like domain. Exemplary inactive, or cleavage incompetent HNH-like domains can have a mutation at one or more of: a histidine in an HNH-like domain, e.g., a histidine shown at position 856 of the consensus sequence of SEQ ID NOS:157-162 or the consensus sequence disclosed in WO2015/161276, e.g., in FIGS. 2A-2G therein, e.g., can be substituted with an alanine; and one or more asparagines in an HNH-like domain, e.g., an asparagine shown at position 870 of the consensus sequence of SEQ ID NOS:157-162 or the consensus sequence disclosed in WO2015/161276, e.g., in FIGS. 2A-2G therein and/or at position 879 of the consensus sequence of SEQ ID NOS:157-162 or the consensus sequence disclosed in WO2015/161276, e.g., in FIGS. 2A-2G therein, e.g., can be substituted with an alanine. In some embodiments, the eaCas9 differs from wild type in the HNH-like domain and does not cleave the target nucleic acid, or cleaves with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, or S. thermophilus. In some embodiments, the reference Cas9 molecule is the naturally occurring Cas9 molecule having the closest sequence identity or homology.

K) Alterations in the Ability to Cleave One or Both Strands of a Target Nucleic Acid

In some embodiments, exemplary Cas9 activities comprise one or more of PAM specificity, cleavage activity, and helicase activity. A mutation(s) can be present, e.g., in: one or more RuvC-like domain, e.g., an N-terminal RuvC-like domain; an HNH-like domain; a region outside the RuvC-like domains and the HNH-like domain. In some embodiments, a mutation(s) is present in a RuvC-like domain, e.g., an N-terminal RuvC-like. In some embodiments, a mutation(s) is present in an HNH-like domain. In some embodiments, mutations are present in both a RuvC-like domain, e.g., an N-terminal RuvC-like domain, and an HNH-like domain.

Exemplary mutations that may be made in the RuvC domain or HNH domain with reference to the S. pyogenes sequence include: D10A, E762A, H840A, N854A, N863A and/or D986A.

In some embodiments, a Cas9 molecule or Cas9 polypeptide is an eiCas9 molecule or eiCas9 polypeptide comprising one or more differences in a RuvC domain and/or in an HNH domain as compared to a reference Cas9 molecule, and the eiCas9 molecule or eiCas9 polypeptide does not cleave a nucleic acid, or cleaves with significantly less efficiency than does wild type, e.g., when compared with wild type in a cleavage assay, e.g., as described herein, cuts with less than 50, 25, 10, or 1% of a reference Cas9 molecule, as measured by an assay described herein.

Whether or not a particular sequence, e.g., a substitution, may affect one or more activity, such as targeting activity, cleavage activity, etc, can be evaluated or predicted, e.g., by evaluating whether the mutation is conservative. In some embodiments, a “non-essential” amino acid residue, as used in the context of a Cas9 molecule, is a residue that can be altered from the wild-type sequence of a Cas9 molecule, e.g., a naturally occurring Cas9 molecule, e.g., an eaCas9 molecule, without abolishing or more preferably, without substantially altering a Cas9 activity (e.g., cleavage activity), whereas changing an “essential” amino acid residue results in a substantial loss of activity (e.g., cleavage activity).

In some embodiments, a Cas9 molecule or Cas9 polypeptide comprises a cleavage property that differs from naturally occurring Cas9 molecules, e.g., that differs from the naturally occurring Cas9 molecule having the closest homology. For example, a Cas9 molecule or Cas9 polypeptide can differ from naturally occurring Cas9 molecules, e.g., a Cas9 molecule of S aureus, S. pyogenes, or C. jejuni as follows: its ability to modulate, e.g., decreased or increased, cleavage of a double stranded break (endonuclease and/or exonuclease activity), e.g., as compared to a naturally occurring Cas9 molecule (e.g., a Cas9 molecule of S aureus, S. pyogenes, or C. jejuni); its ability to modulate, e.g., decreased or increased, cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S aureus, S. pyogenes, or C. jejuni); or the ability to cleave a nucleic acid molecule, e.g., a double stranded or single stranded nucleic acid molecule, can be eliminated.

In some embodiments, the altered Cas9 molecule or Cas9 polypeptide is an eaCas9 molecule or eaCas9 polypeptide comprising one or more of the following activities: cleavage activity associated with a RuvC domain; cleavage activity associated with an HNH domain; cleavage activity associated with an HNH domain and cleavage activity associated with a RuvC domain.

In some embodiments, the altered Cas9 molecule or Cas9 polypeptide is an eiCas9 molecule or eaCas9 polypeptide which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can be a naturally occurring unmodified Cas9 molecule, e.g., a naturally occurring Cas9 molecule such as a Cas9 molecule of S. pyogenes, S. thermophilus, S. aureus, C. jejuni or N. meningitidis. In some embodiments, the reference Cas9 molecule is the naturally occurring Cas9 molecule having the closest sequence identity or homology. In some embodiments, the eiCas9 molecule or eiCas9 polypeptide lacks substantial cleavage activity associated with a RuvC domain and cleavage activity associated with an HNH domain.

In some embodiments, the altered Cas9 molecule or Cas9 polypeptide is an eaCas9 molecule or eaCas9 polypeptide comprising the fixed amino acid residues of S. pyogenes shown in the consensus sequence disclosed in WO2015/161276, e.g., in FIGS. 2A-2G therein, and has one or more amino acids that differ from the amino acid sequence of S. pyogenes (e.g., has a substitution) at one or more residue (e.g., 2, 3, 5, 10, 15, 20, 30, 50, 70, 80, 90, 100, 200 amino acid residues) in SEQ ID NO:162 or residue represented by an “-” in the consensus sequence disclosed in WO2015/161276, e.g., in FIGS. 2A-2G therein.

In some embodiments, the altered Cas9 molecule or Cas9 polypeptide, e.g., an eaCas9 molecule, can be a fusion, e.g., of two of more different Cas9 molecules or Cas9 polypeptides, e.g., of two or more naturally occurring Cas9 molecules of different species. For example, a fragment of a naturally occurring Cas9 molecule of one species can be fused to a fragment of a Cas9 molecule of a second species. As an example, a fragment of Cas9 molecule of S. pyogenes comprising an N-terminal RuvC-like domain can be fused to a fragment of Cas9 molecule of a species other than S. pyogenes (e.g., S. thermophilus) comprising an HNH-like domain.

L) Cas9 Molecules with Altered PAM Recognition or No PAM Recognition

Naturally occurring Cas9 molecules can recognize specific PAM sequences, for example the PAM recognition sequences described herein for, e.g., S. pyogenes, S. thermophilus, S. mutans, S. aureus and N. meningitidis.

In some embodiments, a Cas9 molecule or Cas9 polypeptide has the same PAM specificities as a naturally occurring Cas9 molecule. In other embodiments, a Cas9 molecule or Cas9 polypeptide has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule or Cas9 polypeptide recognizes to decrease off target sites and/or improve specificity; or eliminate a PAM recognition requirement. In some embodiments, a Cas9 molecule can be altered, e.g., to increase length of PAM recognition sequence and/or improve Cas9 specificity to high level of identity, e.g., to decrease off target sites and increase specificity. In some embodiments, the length of the PAM recognition sequence is at least 4, 5, 6, 7, 8, 9, 10 or 15 amino acids in length.

Cas9 molecules or Cas9 polypeptides that recognize different PAM sequences and/or have reduced off-target activity can be generated using directed evolution. Exemplary methods and systems that can be used for directed evolution of Cas9 molecules are described, e.g., in Esvelt et al. Nature 2011, 472(7344): 499-503. Candidate Cas9 molecules can be evaluated, e.g., by methods described herein.

Alterations of the PI domain, which mediates PAM recognition, are discussed herein.

M) Synthetic Cas9 Molecules and Cas9 Polypeptides with Altered PI Domains

Current genome-editing methods are limited in the diversity of target sequences that can be targeted by the PAM sequence that is recognized by the Cas9 molecule utilized. A synthetic Cas9 molecule (or Syn-Cas9 molecule), or synthetic Cas9 polypeptide (or Syn-Cas9 polypeptide), as that term is used herein, refers to a Cas9 molecule or Cas9 polypeptide that comprises a Cas9 core domain from one bacterial species and a functional altered PI domain, i.e., a PI domain other than that naturally associated with the Cas9 core domain, e.g., from a different bacterial species.

In some embodiments, the altered PI domain recognizes a PAM sequence that is different from the PAM sequence recognized by the naturally-occurring Cas9 from which the Cas9 core domain is derived. In some embodiments, the altered PI domain recognizes the same PAM sequence recognized by the naturally-occurring Cas9 from which the Cas9 core domain is derived, but with different affinity or specificity. A Syn-Cas9 molecule or Syn-Cas9 polypeptide can be, respectively, a Syn-eaCas9 molecule or Syn-eaCas9 polypeptide or a Syn-eiCas9 molecule Syn-eiCas9 polypeptide.

An exemplary Syn-Cas9 molecule or Syn-Cas9 polypeptide comprises: a) a Cas9 core domain, e.g., a Cas9 core domain, e.g., a S. aureus, S. pyogenes, or C. jejuni Cas9 core domain; and b) an altered PI domain from a species X Cas9 sequence.

In some embodiments, the RKR motif (the PAM binding motif) of said altered PI domain comprises: differences at 1, 2, or 3 amino acid residues; a difference in amino acid sequence at the first, second, or third position; differences in amino acid sequence at the first and second positions, the first and third positions, or the second and third positions; as compared with the sequence of the RKR motif of the native or endogenous PI domain associated with the Cas9 core domain.

In some embodiments, a Syn-Cas9 molecule or Syn-Cas9 polypeptide may also be size-optimized, e.g., the Syn-Cas9 molecule or Syn-Cas9 polypeptide comprises one or more deletions, and optionally one or more linkers disposed between the amino acid residues flanking the deletions. In some embodiments, a Syn-Cas9 molecule or Syn-Cas9 polypeptide comprises a REC deletion.

N) Size-Optimized Cas9 Molecules and Cas9 Polypeptides

Engineered Cas9 molecules and engineered Cas9 polypeptides described herein include a Cas9 molecule or Cas9 polypeptide comprising a deletion that reduces the size of the molecule while still retaining desired Cas9 properties, e.g., essentially native conformation, Cas9 nuclease activity, and/or target nucleic acid molecule recognition. Provided herein are Cas9 molecules or Cas9 polypeptides comprising one or more deletions and optionally one or more linkers, wherein a linker is disposed between the amino acid residues that flank the deletion. Methods for identifying suitable deletions in a reference Cas9 molecule, methods for generating Cas9 molecules with a deletion and a linker, and methods for using such Cas9 molecules will be apparent upon review of this document.

A Cas9 molecule, e.g., a S. aureus, S. pyogenes, or C. jejuni, Cas9 molecule, having a deletion is smaller, e.g., has reduced number of amino acids, than the corresponding naturally-occurring Cas9 molecule. The smaller size of the Cas9 molecules allows increased flexibility for delivery methods, and thereby increases utility for genome-editing. A Cas9 molecule or Cas9 polypeptide can comprise one or more deletions that do not substantially affect or decrease the activity of the resultant Cas9 molecules or Cas9 polypeptides described herein. Activities that are retained in the Cas9 molecules or Cas9 polypeptides comprising a deletion as described herein include one or more of the following: a nickase activity, i.e., the ability to cleave a single strand, e.g., the non-complementary strand or the complementary strand, of a nucleic acid molecule; a double stranded nuclease activity, i.e., the ability to cleave both strands of a double stranded nucleic acid and create a double stranded break, which in some embodiments is the presence of two nickase activities; an endonuclease activity; an exonuclease activity; a helicase activity, i.e., the ability to unwind the helical structure of a double stranded nucleic acid; and recognition activity of a nucleic acid molecule, e.g., a target nucleic acid or a gRNA.

Activity of the Cas9 molecules or Cas9 polypeptides described herein can be assessed using the activity assays described herein or are known.

O) Identifying Regions Suitable for Deletion

Suitable regions of Cas9 molecules for deletion can be identified by a variety of methods. Naturally-occurring orthologous Cas9 molecules from various bacterial species, can be modeled onto the crystal structure of S. pyogenes Cas9 (Nishimasu et al., Cell, 156:935-949, 2014) to examine the level of conservation across the selected Cas9 orthologs with respect to the three-dimensional conformation of the protein. Less conserved or unconserved regions that are spatially located distant from regions involved in Cas9 activity, e.g., interface with the target nucleic acid molecule and/or gRNA, represent regions or domains are candidates for deletion without substantially affecting or decreasing Cas9 activity.

P) REC-Optimized Cas9 Molecules and Cas9 Polypeptides

A REC-optimized Cas9 molecule, or a REC-optimized Cas9 polypeptide, as that term is used herein, refers to a Cas9 molecule or Cas9 polypeptide that comprises a deletion in one or both of the REC2 domain and the RE1_{CT} domain (collectively a REC deletion), wherein the deletion comprises at least 10% of the amino acid residues in the cognate domain. A REC-optimized Cas9 molecule or Cas9 polypeptide can be an eaCas9 molecule or eaCas9 polypeptide, or an eiCas9 molecule or eiCas9 polypeptide. An exemplary REC-optimized Cas9 molecule or REC-optimized Cas9 polypeptide comprises: a) a deletion selected from: i) a REC2 deletion; ii) a REC1_{CT} deletion; or iii) a REC1_{SUB} deletion.

Optionally, a linker is disposed between the amino acid residues that flank the deletion. In some embodiments a Cas9 molecule or Cas9 polypeptide includes only one deletion, or only two deletions. A Cas9 molecule or Cas9 polypeptide can comprise a REC2 deletion and a REC1_{CT} deletion. A Cas9 molecule or Cas9 polypeptide can comprise a REC2 deletion and a REC1_{SUB} deletion.

Generally, the deletion will contain at least 10% of the amino acids in the cognate domain, e.g., a REC2 deletion will include at least 10% of the amino acids in the REC2 domain. A deletion can comprise: at least 10, 20, 30, 40, 50, 60, 70, 80, or 90% of the amino acid residues of its cognate domain; all of the amino acid residues of its cognate domain; an amino acid residue outside its cognate domain; a plurality of amino acid residues outside its cognate domain; the amino acid residue immediately N terminal to its cognate domain; the amino acid residue immediately C terminal to its cognate domain; the amino acid residue immediately N terminal to its cognate and the amino acid residue immediately C terminal to its cognate domain; a plurality of, e.g., up to 5, 10, 15, or 20, amino acid residues N terminal to its cognate domain; a plurality of, e.g., up to 5, 10, 15, or 20, amino acid residues C terminal to its cognate domain; a plurality of, e.g., up to 5, 10, 15, or 20, amino acid residues N terminal to its cognate domain and a plurality of e.g., up to 5, 10, 15, or 20, amino acid residues C terminal to its cognate domain.

In some embodiments, a deletion does not extend beyond: its cognate domain; the N terminal amino acid residue of its cognate domain; the C terminal amino acid residue of its cognate domain.

A REC-optimized Cas9 molecule or REC-optimized Cas9 polypeptide can include a linker disposed between the amino acid residues that flank the deletion. Suitable linkers for use between the amino acid resides that flank a REC deletion in a REC-optimized Cas9 molecule is described herein.

In some embodiments, a REC-optimized Cas9 molecule or REC-optimized Cas9 polypeptide comprises an amino acid sequence that, other than any REC deletion and associated linker, has at least 50, 55, 60, 65, 70, 75, 80, 85, 90, 95, 99, or 100% homology with the amino acid sequence of a naturally occurring Cas9, e.g., a S. aureus Cas9 molecule, a S. pyogenes Cas9 molecule, or a C. jejuni Cas9 molecule.

In some embodiments, a REC-optimized Cas9 molecule or REC-optimized Cas9 polypeptide comprises an amino acid sequence that, other than any REC deletion and associated linker, differs by no more than 1, 2, 3, 4, 5, 6, 7, 8, 9, 10, 15, 20, or 25, amino acid residues from the amino acid sequence of a naturally occurring Cas9, e.g., a S. aureus Cas9 molecule, a S. pyogenes Cas9 molecule, or a C. jejuni Cas9 molecule.

In some embodiments, a REC-optimized Cas9 molecule or REC-optimized Cas9 polypeptide comprises an amino acid sequence that, other than any REC deletion and associate linker, differs by no more than 1, 2, 3, 4, 5, 6, 7, 8, 9, 10, 15, 20, or 25% of the, amino acid residues from the amino acid sequence of a naturally occurring Cas9, e.g., a S. aureus Cas9 molecule, a S. pyogenes Cas9 molecule, or a C. jejuni Cas9 molecule.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Sequence information for exemplary REC deletions are provided for 83 naturally-occurring Cas9 orthologs described in, e.g., International PCT Pub. Nos. WO2015/161276, WO2017/193107 and WO2017/093969.

Q) Nucleic Acids Encoding Cas9 Molecules

Nucleic acids encoding the Cas9 molecules or Cas9 polypeptides, e.g., an eaCas9 molecule or eaCas9 polypeptide, can be used in connection with any of the embodiments provided herein.

Exemplary nucleic acids encoding Cas9 molecules or Cas9 polypeptides are described in Cong et al., Science 2013, 399(6121):819-823; Wang et al., Cell 2013, 153(4):910-918; Mali et al., Science 2013, 399(6121):823-826; Jinek et al., Science 2012, 337(6096):816-821, and WO2015/161276, e.g., in FIG. 8 therein.

In some embodiments, a nucleic acid encoding a Cas9 molecule or Cas9 polypeptide can be a synthetic nucleic acid sequence. For example, the synthetic nucleic acid molecule can be chemically modified. In some embodiments, the Cas9 mRNA has one or more (e.g., all of the following properties: it is capped, polyadenylated, substituted with 5-methylcytidine and/or pseudouridine.

In addition, or alternatively, the synthetic nucleic acid sequence can be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein.

In addition, or alternatively, a nucleic acid encoding a Cas9 molecule or Cas9 polypeptide may comprise a nuclear localization sequence (NLS). Nuclear localization sequences are known.

If any of the Cas9 sequences are fused with a peptide or polypeptide at the C-terminus, it is understood that the stop codon will be removed.

R) Other Cas Molecules and Cas Polypeptides

Various types of Cas molecules or Cas polypeptides can be used to practice the inventions disclosed herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described, e.g., in Haft et al., PLoS Computational Biology 2005, 1(6): e60 and Makarova et al., Nature Review Microbiology 2011, 9:467-477, the contents of both references are incorporated herein by reference in their entirety. Exemplary Cas molecules (and Cas systems) are also shown in Table 6.

TABLE 6
Cas Systems

| | | | Structure of | Families (and | |
|---|---|---|---|---|---|
| | | | encoded | superfamily) of | |
| Gene | System type or | Name from | protein (PDB | encoded | |
| name^{‡} | subtype | Haft et al.^{§} | accessions)^{¶} | protein^{#}** | Representatives |
| cas1 | Type I | cas1 | 3GOD, 3LFX | COG1518 | SERP2463, |
| | Type II | | and 2YZS | | SPy1047 and ygbT |
| | Type III | | | | |
| cas2 | Type I | cas2 | 2IVY, 2I8E | COG1343 and | SERP2462, |
| | Type II | | and 3EXC | COG3512 | SPy1048, SPy1723 |
| | Type III | | | | (N-terminal |
| | | | | | domain) and ygbF |
| cas3′ | Type I^{‡‡} | cas3 | NA | COG1203 | APE1232 and ygcB |
| cas3″ | Subtype I-A | NA | NA | COG2254 | APE1231 and |
| | Subtype I-B | | | | BH0336 |
| cas4 | Subtype I-A | cas4 and | NA | COG1468 | APE1239 and |
| | Subtype I-B | csa1 | | | BH0340 |
| | Subtype I-C | | | | |
| | Subtype I-D | | | | |
| | Subtype II-B | | | | |
| cas5 | Subtype I-A | cas5a, | 3KG4 | COG1688 | APE1234, BH0337, |
| | Subtype I-B | cas5d, | | (RAMP) | devS and ygcI |
| | Subtype I-C | cas5e, | | | |
| | Subtype I-E | cas5h, | | | |
| | | cas5p, | | | |
| | | cas5t and | | | |
| | | cmx5 | | | |
| cas6 | Subtype I-A | cas6 and | 3I4H | COG1583 and | PF1131 and slr7014 |
| | Subtype I-B | cmx6 | | COG5551 | |
| | Subtype I-D | | | (RAMP) | |
| | Subtype III-A | | | | |
| | Subtype III-B | | | | |
| cas6e | Subtype I-E | cse3 | 1WJ9 | (RAMP) | ygcH |
| cas6f | Subtype I-F | csy4 | 2XLJ | (RAMP) | y1727 |
| cas7 | Subtype I-A | csa2, csd2, | NA | COG1857 and | devR and ygcJ |
| | Subtype I-B | cse4, csh2, | | COG3649 | |
| | Subtype I-C | csp1 and | | (RAMP) | |
| | Subtype I-E | cst2 | | | |
| cas8a1 | Subtype I-A^{‡‡} | cmx1, cst1, | NA | BH0338-like | LA3191^{§§} and |
| | | csx8, csx13 | | | PG2018^{§§} |
| | | and CXXC- | | | |
| | | CXXC | | | |
| cas8a2 | Subtype I-A^{‡‡} | csa4 and | NA | PH0918 | AF0070, AF1873, |
| | | csx9 | | | MJ0385, PF0637, |
| | | | | | PH0918 and |
| | | | | | SSO1401 |
| cas8b | Subtype I-B^{‡‡} | csh1 and | NA | BH0338-like | MTH1090 and |
| | | TM1802 | | | TM1802 |
| cas8c | Subtype I-C^{‡‡} | csd1 and | NA | BH0338-like | BH0338 |
| | | csp2 | | | |
| cas9 | Type II^{‡‡} | csn1 and | NA | COG3513 | FTN_0757 and |
| | | csx12 | | | SPy1046 |
| cas10 | Type III^{‡‡} | cmr2, csm1 | NA | COG1353 | MTH326, |
| | | and csx11 | | | Rv2823c^{§§} and |
| | | | | | TM1794^{§§} |
| cas10d | Subtype I-D^{‡‡} | csc3 | NA | COG1353 | slr7011 |
| csy1 | Subtype I-F^{‡‡} | csy1 | NA | y1724-like | y1724 |
| csy2 | Subtype I-F | csy2 | NA | (RAMP) | y1725 |
| csy3 | Subtype I-F | csy3 | NA | (RAMP) | y1726 |
| cse1 | Subtype I-E^{‡‡} | cse1 | NA | YgcL-like | ygcL |
| cse2 | Subtype I-E | cse2 | 2ZCA | YgcK-like | ygcK |
| csc1 | Subtype I-D | csc1 | NA | alr1563-like | alr1563 |
| | | | | (RAMP) | |
| csc2 | Subtype I-D | csc1 and | NA | COG1337 | slr7012 |
| | | csc2 | | (RAMP) | |
| csa5 | Subtype I-A | csa5 | NA | AF1870 | AF1870, MJ0380, |
| | | | | | PF0643 and |
| | | | | | SSO1398 |
| csn2 | Subtype II-A | csn2 | NA | SPy1049-like | SPy1049 |
| csm2 | Subtype III-A^{‡‡} | csm2 | NA | COG1421 | MTH1081 and |
| | | | | | SERP2460 |
| csm3 | Subtype III-A | csc2 and | NA | COG1337 | MTH1080 and |
| | | csm3 | | (RAMP) | SERP2459 |
| csm4 | Subtype III-A | csm4 | NA | COG1567 | MTH1079 and |
| | | | | (RAMP) | SERP2458 |
| csm5 | Subtype III-A | csm5 | NA | COG1332 | MTH1078 and |
| | | | | (RAMP) | SERP2457 |
| csm6 | Subtype III-A | APE2256 | 2WTE | COG1517 | APE2256 and |
| | | and csm6 | | | SSO1445 |
| cmr1 | Subtype III-B | cmr1 | NA | COG1367 | PF1130 |
| | | | | (RAMP) | |
| cmr3 | Subtype III-B | cmr3 | NA | COG1769 | PF1128 |
| | | | | (RAMP) | |
| cmr4 | Subtype III-B | cmr4 | NA | COG1336 | PF1126 |
| | | | | (RAMP) | |
| cmr5 | Subtype III-B^{‡‡} | cmr5 | 2ZOP and | COG3337 | MTH324 and |
| | | | 2OEB | | PF1125 |
| cmr6 | Subtype III-B | cmr6 | NA | COG1604 | PF1124 |
| | | | | (RAMP) | |
| csb1 | Subtype I-U | GSU0053 | NA | (RAMP) | Balac_1306 and |
| | | | | | GSU0053 |
| csb2 | Subtype I-U^{§§} | NA | NA | (RAMP) | Balac_1305 and |
| | | | | | GSU0054 |
| csb3 | Subtype I-U | NA | NA | (RAMP) | Balac_1303^{§§} |
| csx17 | Subtype I-U | NA | NA | NA | Btus_2683 |
| csx14 | Subtype I-U | NA | NA | NA | GSU0052 |
| csx10 | Subtype I-U | csx10 | NA | (RAMP) | Caur_2274 |
| csx16 | Subtype III-U | VVA1548 | NA | NA | VVA1548 |
| csaX | Subtype III-U | csaX | NA | NA | SSO1438 |
| csx3 | Subtype III-U | csx3 | NA | NA | AF1864 |
| csx1 | Subtype III-U | csa3, csx1, | 1XMX and | COG1517 and | MJ1666, NE0113, |
| | | csx2, | 2I71 | COG4006 | PF1127 and |
| | | DXTHG, | | | TM1812 |
| | | NE0113 | | | |
| | | and | | | |
| | | TIGR02710 | | | |
| csx15 | Unknown | NA | NA | TTE2665 | TTE2665 |
| csf1 | Type U | csf1 | NA | NA | AFE_1038 |
| csf2 | Type U | csf2 | NA | (RAMP) | AFE_1039 |
| csf3 | Type U | csf3 | NA | (RAMP) | AFE_1040 |
| csf4 | Type U | csf4 | NA | NA | AFE_1037 |

3) Cpf1

In some embodiments, the guide RNA or gRNA promotes the specific association targeting of an RNA-guided nuclease such as a Cas9 or a Cpf1 to a target sequence such as a genomic or episomal sequence in a cell. In general, gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature, for instance in Briner et al. (Molecular Cell 56(2), 333-339, Oct. 23, 2014 (Briner), which is incorporated by reference), and in Cotta-Ramusino.

Guide RNAs, whether unimolecular or modular, generally include a targeting domain that is fully or partially complementary to a target, and are typically 10-30 nucleotides in length, and in certain embodiments are 16-24 nucleotides in length (for instance, 16, 17, 18, 19, 20, 21, 22, 23 or 24 nucleotides in length). In some aspects, the targeting domains are at or near the 5′ terminus of the gRNA in the case of a Cas9 gRNA, and at or near the 3′ terminus in the case of a Cpf1 gRNA. While the foregoing description has focused on gRNAs for use with Cas9, it should be appreciated that other RNA-guided nucleases have been (or may in the future be) discovered or invented which utilize gRNAs that differ in some ways from those described to this point. For instance, Cpf1 (“CRISPR from Prevotella and Franciscella 1”) is a recently discovered RNA-guided nuclease that does not require a tracrRNA to function. (Zetsche et al., 2015, Cell 163, 759-771 Oct. 22, 2015 (Zetsche I), incorporated by reference herein). A gRNA for use in a Cpf1 genome editing system generally includes a targeting domain and a complementarity domain (alternately referred to as a “handle”). It should also be noted that, in gRNAs for use with Cpf1, the targeting domain is usually present at or near the 3′ end, rather than the 5′ end as described above in connection with Cas9 gRNAs (the handle is at or near the 5′ end of a Cpf1 gRNA).

Although structural differences may exist between gRNAs from different prokaryotic species, or between Cpf1 and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, in some aspects in this disclosure, gRNAs may be described solely in terms of their targeting domain sequences.

More generally, some aspects of the present disclosure relate to systems, methods and compositions that can be implemented using multiple RNA-guided nucleases. Unless otherwise specified, the term gRNA should be understood to encompass any suitable gRNA that can be used with any RNA-guided nuclease, and not only those gRNAs that are compatible with a particular species of Cas9 or Cpf1. By way of illustration, the term gRNA can, in certain embodiments, include a gRNA for use with any RNA-guided nuclease occurring in a Class 2 CRISPR system, such as a type II or type V or CRISPR system, or an RNA-guided nuclease derived or adapted therefrom.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

RNA-guided nucleases include, but are not limited to, naturally-occurring Class 2 CRISPR nucleases such as Cas9, and Cpf1, as well as other nucleases derived or obtained therefrom. In functional terms, RNA-guided nucleases are defined as those nucleases that: (a) interact with (e.g complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cpf1), species (e.g. S. pyogenes vs. S. aureus) or variation (e.g full-length vs. truncated or split; naturally-occurring PAM specificity vs. engineered PAM specificity, etc.) of RNA-guided nuclease.

In addition to recognizing specific sequential orientations of PAMs and protospacers, RNA-guided nucleases in some embodiments can also recognize specific PAM sequences. S. aureus Cas9, for instance, generally recognizes a PAM sequence of NNGRRT or NNGRRV, wherein the N residues are immediately 3′ of the region recognized by the gRNA targeting domain. S. pyogenes Cas9 generally recognizes NGG PAM sequences. And F. novicida Cpf1 generally recognizes a TTN PAM sequence.

The crystal structure of Acidaminococcus sp. Cpf1 in complex with crRNA and a double-stranded (ds) DNA target including a TTTN PAM sequence has been solved by Yamano et al. (Cell. 2016 May 5; 165(4): 949-962 (Yamano), incorporated by reference herein). Cpf1, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cpf1 REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cpf1 share similarities in structure and function, it should be appreciated that certain Cpf1 activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cpf1 gRNA (the handle) adopts a pseudoknot structure, rather than a stem loop structure formed by the repeat:antirepeat duplex in Cas9 gRNAs.

Nucleic acids encoding RNA-guided nucleases, e.g., Cas9, Cpf1 or functional fragments thereof, are provided herein. Exemplary nucleic acids encoding RNA-guided nucleases have been described previously (see, e.g., Cong 2013; Wang 2013; Mali 2013; Jinek 2012).

3. Delivery of Agents for Generic Disruption

In some embodiments, the targeted genetic disruption, e.g., DNA break, of the endogenous genes encoding TCR, such as TRAC and TRBC1 or TRBC2 in humans is carried out by delivering or introducing one or more agent(s) capable of inducing a genetic disruption, e.g., Cas9 and/or gRNA components, to a cell, using any of a number of known delivery method or vehicle for introduction or transfer to cells, for example, using lentiviral delivery vectors, or any of the known methods or vehicles for delivering Cas9 molecules and gRNAs. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequences encoding one or more components of one or more agent(s) capable of inducing a genetic disruption, e.g., DNA break, is introduced into the cells, e.g., by any methods for introducing nucleic acids into a cell described herein or known. In some embodiments, a vector encoding components of one or more agent(s) capable of inducing a genetic disruption such as a CRISPR guide RNA and/or a Cas9 enzyme can be delivered into the cell.

In some embodiments, the one or more agent(s) capable of inducing a genetic disruption, e.g., one or more agent(s) that is a Cas9/gRNA, is introduced into the cell as a ribonucleoprotein (RNP) complex. RNP complexes include a sequence of ribonucleotides, such as an RNA or a gRNA molecule, and a protein, such as a Cas9 protein or variant thereof. For example, the Cas9 protein is delivered as RNP complex that comprises a Cas9 protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more agent(s) capable of inducing genetic disruption, e.g., CRISPR/Cas9, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the integration of transgene (discussed further herein in Section I.B) can be inherited by the progeny cells, but without the agent itself, which may further introduce off-target genetic disruptions, being passed on to the progeny cells.

Agent(s) and components capable of inducing a genetic disruption, e.g., a Cas9 molecule and gRNA molecule, can be introduced into target cells in a variety of forms using a variety of delivery methods and formulations, as set forth in Tables 7 and 8, or methods described in, e.g., WO 2015/161276; US 2015/0056705, US 2016/0272999, US 2017/0211075; or US 2017/0016027. As described further herein, the delivery methods and formulations can be used to deliver template polynucleotides and/or other agents to the cell in prior or subsequent steps of the methods described herein.

TABLE 7
Exemplary Delivery Methods

| Elements | | |
|---|---|---|
| Cas9 | gRNA | |
| Molecule(s) | molecule(s) | Comments |
| DNA | DNA | In this embodiment, a Cas9 molecule and a gRNA are transcribed |
| | | from DNA. In this embodiment, they are encoded on separate |
| | | molecules. |
| DNA | | In this embodiment, a Cas9 molecule and a gRNA are transcribed |
| | | from DNA, here from a single molecule. |
| DNA | RNA | In this embodiment, a Cas9 molecule is transcribed from DNA, and |
| | | a gRNA is provided as in vitro transcribed or synthesized RNA |
| mRNA | RNA | In this embodiment, a Cas9 molecule is translated from in vitro |
| | | transcribed mRNA, and a gRNA is provided as in vitro transcribed |
| | | or synthesized RNA. |
| mRNA | DNA | In this embodiment, a Cas9 molecule is translated from in vitro |
| | | transcribed mRNA, and a gRNA is transcribed from DNA. |
| Protein | DNA | In this embodiment, a Cas9 molecule is provided as a protein, and a |
| | | gRNA is transcribed from DNA. |
| Protein | RNA | In this embodiment, a Cas9 molecule is provided as a protein, and a |
| | | gRNA is provided as transcribed or synthesized RNA. |

TABLE 8
Comparison of Exemplary Delivery Methods

| | | Delivery | | | |
|---|---|---|---|---|---|
| | | into Non- | | | |
| | | Dividing | Duration of | Genome | Molecule |
| Delivery Vector/Mode | | Cells | Expression | Integration | Delivered |
| Physical (e.g., electroporation, | | YES | Transient | NO | Nucleic |
| particle gun, Calcium Phosphate | | | | | Acids and |
| transfection, cell compression or | | | | | Proteins |
| squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex Virus | YES | Stable | NO | DNA |
| Non-Viral | Cationic Liposomes | YES | Transient | Depends on | Nucleic |
| | | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated Bacteria | YES | Transient | NO | Nucleic |
| Non-Viral | | | | | Acids |
| Delivery | Engineered | YES | Transient | NO | Nucleic |
| Vehicles | Bacteriophages | | | | Acids |
| | Mammalian Virus-like | YES | Transient | NO | Nucleic |
| | Particles | | | | Acids |
| | Biological liposomes: | YES | Transient | NO | Nucleic |
| | Erythrocyte Ghosts | | | | Acids |
| | and Exosomes | | | | |

In some embodiments, DNA encoding Cas9 molecules and/or gRNA molecules, or RNP complexes comprising a Cas9 molecule and/or gRNA molecules, can be delivered into cells by known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof. In some embodiments, the polynucleotide containing the agent(s) and/or components thereof is delivered by a vector (e.g., viral vector/virus or plasmid). The vector may be any described herein.

In some aspects, a CRISPR enzyme (e.g. Cas9 nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, a Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target gene (e.g. TRAC, TRBC1 and/or TRBC2 in humans) are introduced into cells. In some embodiments, gRNA sequences that is or comprises a targeting domain sequence targeting the target site in a particular gene, such as the TRAC, TRBC1 and/or TRBC2 genes, designed or identified. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the polynucleotide containing the agent(s) and/or components thereof or RNP complex is delivered by a non-vector based method (e.g., using naked DNA or DNA complexes). For example, the DNA or RNA or proteins or combination thereof, e.g., ribonucleoprotein (RNP) complexes, can be delivered, e.g., by organically modified silica or silicate (Ormosil), electroporation, transient cell compression or squeezing (e.g., as described in Lee, et al. (2012) Nano Lett 12: 6322-27, Kollmannsperger et al (2016) Nat Comm 7, 10372 doi:10.1038/ncomms10372).), gene gun, sonoporation, magnetofection, lipid-mediated transfection, dendrimers, inorganic nanoparticles, calcium phosphates, or a combination thereof.

In some embodiments, delivery via electroporation comprises mixing the cells with the Cas9- and/or gRNA-encoding DNA or RNP complex in a cartridge, chamber or cuvette and applying one or more electrical impulses of defined duration and amplitude. In some embodiments, delivery via electroporation is performed using a system in which cells are mixed with the Cas9- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the delivery vehicle is a non-viral vector. In some embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In some embodiments, the non-viral vector is an organic nanoparticle. Exemplary organic nanoparticles include, e.g., SNALP liposomes that contain cationic lipids together with neutral helper lipids which are coated with polyethylene glycol (PEG), and protamine-nucleic acid complexes coated with lipid. Exemplary lipids and/or polymers are known and can be used in the provided embodiments.

In some embodiments, the vehicle has targeting modifications to increase target cell update of nanoparticles and liposomes, e.g., cell specific antigens, monoclonal antibodies, single chain antibodies, aptamers, polymers, sugars, and cell penetrating peptides (e.g., described in US 2016/0272999). In some embodiments, the vehicle uses fusogenic and endosome-destabilizing peptides/polymers. In some embodiments, the vehicle undergoes acid-triggered conformational changes (e.g., to accelerate endosomal escape of the cargo). In some embodiments, a stimulus-cleavable polymer is used, e.g., for release in a cellular compartment. For example, disulfide-based cationic polymers that are cleaved in the reducing cellular environment can be used.

In some embodiments, the delivery vehicle is a biological non-viral delivery vehicle. In some embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific cells, bacteria having modified surface proteins to alter target cell specificity). In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue-specificity. In some embodiments, the vehicle is a biological liposome. For example, the biological liposome is a phospholipid-based particle derived from human cells, e.g., erythrocyte ghosts, which are red blood cells broken down into spherical structures derived from the subject (e.g., tissue targeting can be achieved by attachment of various tissue or cell-specific ligands), or secretory exosomes-subject-derived membrane-bound nanovescicles (30-100 nm) of endocytic origin (e.g., can be produced from various cell types and can therefore be taken up by cells without the need for targeting ligands).

In some embodiments, RNA encoding Cas9 molecules and/or gRNA molecules, can be delivered into cells, e.g., target cells described herein, by known methods or as described herein. For example, Cas9-encoding and/or gRNA-encoding RNA can be delivered, e.g., by microinjection, electroporation, transient cell compression or squeezing (e.g., as described in Lee, et al. (2012) Nano Lett 12: 6322-27), lipid-mediated transfection, peptide-mediated delivery, or a combination thereof.

In some embodiments, delivery via electroporation comprises mixing the cells with the RNA encoding Cas9 molecules and/or gRNA molecules in a cartridge, chamber or cuvette and applying one or more electrical impulses of defined duration and amplitude. In some embodiments, delivery via electroporation is performed using a system in which cells are mixed with the RNA encoding Cas9 molecules and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, Cas9 molecules can be delivered into cells by known methods or as described herein. For example, Cas9 protein molecules can be delivered, e.g., by microinjection, electroporation, transient cell compression or squeezing (e.g., as described in Lee, et al. (2012) Nano Lett 12: 6322-27), lipid-mediated transfection, peptide-mediated delivery, or a combination thereof. Delivery can be accompanied by DNA encoding a gRNA or by a gRNA.

In some embodiments, delivery via electroporation comprises mixing the cells with the Cas9 molecules with or without gRNA molecules in a cartridge, chamber or cuvette and applying one or more electrical impulses of defined duration and amplitude. In some embodiments, delivery via electroporation is performed using a system in which cells are mixed with the Cas9 molecules with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the polynucleotide containing the agent(s) and/or components thereof is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, more than one agent(s) or components thereof are delivered to the cell. For example, in some embodiments, agent(s) capable of inducing a genetic disruption of two or more locations in the genome, e.g., the TRAC, TRBC1 and/or TRBC2 loci, are delivered to the cell. In some embodiments, agent(s) and components thereof are delivered using one method. For example, in some embodiments, agent(s) for inducing a genetic disruption of TRAC, TRBC1 and/or TRBC2 loci are delivered as polynucleotides encoding the components for genetic disruption. In some embodiments, one polynucleotide can encode agents that target the TRAC, TRBC1 and/or TRBC2 loci. In some embodiments, two or more different polynucleotides can encode the agents that target TRAC, TRBC1 and/or TRBC2 loci. In some embodiments, the agents capable of inducing a genetic disruption can be delivered as ribonucleoprotein (RNP) complexes, and two or more different RNP complexes can be delivered together as a mixture, or separately.

In some embodiments, one or more nucleic acid molecules other than the one or more agent(s) capable of inducing a genetic disruption and/or component thereof, e.g., the Cas9 molecule component and/or the gRNA molecule component, such as a template polynucleotide for HDR-directed integration (e.g., described in Section I.B. herein), are delivered. In some embodiments, the nucleic acid molecule, e.g., template polynucleotide, is delivered at the same time as one or more of the components of the Cas system. In some embodiments, the nucleic acid molecule is delivered before or after (e.g., less than about 1 minute, 5 minutes, 10 minutes, 15 minutes, 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In some embodiments, the nucleic acid molecule, e.g., template polynucleotide, is delivered by a different means from one or more of the components of the Cas system, e.g., the Cas9 molecule component and/or the gRNA molecule component. The nucleic acid molecule, e.g., template polynucleotide, can be delivered by any of the delivery methods described herein. For example, the nucleic acid molecule, e.g., template polynucleotide, can be delivered by a viral vector, e.g., a retrovirus or a lentivirus, and the Cas9 molecule component and/or the gRNA molecule component can be delivered by electroporation. In some embodiments, the nucleic acid molecule, e.g., template polynucleotide, includes one or more transgenes, e.g., transgenes that encode a recombinant TCR, a recombinant CAR and/or other gene products.

B. Targeted Integration Via Homology Directed Repair (HDR)

In some of the embodiments provided herein, homology-directed repair (HDR) can be utilized for targeted integration of a specific portion of the template polynucleotide containing a transgene, e.g., nucleic acid sequence encoding a recombinant receptor, at a particular location in the genome, e.g., the TRAC, TRBC1 and/or TRBC2 locus. In some embodiments, the presence of a genetic disruption (e.g., a DNA break, such as described in Section I.A) and a template polynucleotide containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the 5′ and/or 3′ homology arms included in the template polynucleotide, cellular DNA repair machinery can use the template polynucleotide to repair the DNA break and resynthesize genetic information at the site of the genetic disruption, thereby effectively inserting or integrating the transgene sequences in the template polynucleotide at or near the site of the genetic disruption. In some embodiments, the genetic disruption, e.g., TRAC, TRBC1 and/or TRBC2 locus, can be generated by any of the methods for generating a targeted genetic disruption described herein.

Also provided are polynucleotides, e.g., template polynucleotides described herein. In some embodiments, the provided polynucleotides can be employed in the methods described herein, e.g., involving HDR, to target transgene sequences encoding a portion of a recombinant receptor, e.g., recombinant TCR, at the endogenous TRAC, TRBC1 and/or TRBC2 locus.

In some embodiments, the template polynucleotide is or comprises a polynucleotide containing a transgene (exogenous or heterologous nucleic acids sequences) encoding a recombinant receptor or a portion thereof (e.g., one or more chain(s), region(s) or domain(s) of the recombinant receptor), and homology sequences (e.g., homology arms) that are homologous to sequences at or near the endogenous genomic site, e.g., at the endogenous TRAC, TRBC1 and/or TRBC2 locus. In some aspects, the template polynucleotide is introduced as a linear DNA fragment or comprised in a vector. In some aspects, the step for inducing genetic disruption and the step for targeted integration (e.g., by introduction of the template polynucleotide) are performed simultaneously or sequentially.

1. Homology-Directed Repair (HDR)

In some embodiments, homology-directed repair (HDR) can be utilized for targeted integration or insertion of one or more nucleic acid sequences, e.g., transgene sequences, at one or more target site(s) in the genome, e.g., the TRAC, TRBC1 and/or TRBC2 locus. In some embodiments, the nuclease-induced HDR can be used to alter a target sequence, integrate a transgene at a particular target location, and/or to edit or repair a mutation in a particular target gene.

Alteration of nucleic acid sequences at the target site can occur by HDR with an exogenously provided template polynucleotide (also referred to as donor polynucleotide or template sequence). For example, the template polynucleotide provides for alteration of the target sequence, such as insertion of the transgene contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Template polynucleotide-effected alteration of a target sequence depends on cleavage by a nuclease, e.g., a targeted nuclease such as CRISPR/Cas9. Cleavage by the nuclease can comprise a double strand break or two single strand breaks.

In some embodiments, “recombination” refers to a process of exchange of genetic information between two polynucleotides. In some embodiments, “homologous recombination (HR)” refers to the specialized form of such exchange that takes place, for example, during repair of double-strand breaks in cells via homology-directed repair mechanisms. This process requires nucleotide sequence homology, uses a template polynucleotide to template repair of a target DNA (i.e., the one that experienced the double-strand break, e.g., target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide.

In some embodiments, a template polynucleotide, e.g., polynucleotide containing transgene, is integrated into the genome of a cell via homology-independent mechanisms. The methods comprise creating a double-stranded break (DSB) in the genome of a cell and cleaving the template polynucleotide molecule using a nuclease, such that the template polynucleotide is integrated at the site of the DSB. In some embodiments, the template polynucleotide is integrated via non-homology dependent methods (e.g., NHEJ). Upon in vivo cleavage the template polynucleotides can be integrated in a targeted manner into the genome of a cell at the location of a DSB. The template polynucleotide can include one or more of the same target sites for one or more of the nucleases used to create the DSB. Thus, the template polynucleotide may be cleaved by one or more of the same nucleases used to cleave the endogenous gene into which integration is desired. In some embodiments, the template polynucleotide includes different nuclease target sites from the nucleases used to induce the DSB. As described herein, the genetic disruption of the target site or target position can be created by any mechanisms, such as ZFNs, TALENs, CRISPR/Cas9 system, or TtAgo nucleases.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break. In some embodiments, a single-stranded template polynucleotide is used and the target site can be altered by alternative HDR.

Template polynucleotide-effected alteration of a target site depends on cleavage by a nuclease molecule. Cleavage by the nuclease can comprise a nick, a double strand break, or two single strand breaks, e.g., one on each strand of the DNA at the target site. After introduction of the breaks on the target site, resection occurs at the break ends resulting in single stranded overhanging DNA regions.

In canonical HDR, a double-stranded template polynucleotide is introduced, comprising homologous sequence to the target site that will either be directly incorporated into the target site or used as a template to insert the transgene or correct the sequence of the target site. After resection at the break, repair can progress by different pathways, e.g., by the double Holliday junction model (or double strand break repair, DSBR, pathway) or the synthesis-dependent strand annealing (SDSA) pathway.

In the double Holliday junction model, strand invasion by the two single stranded overhangs of the target site to the homologous sequences in the template polynucleotide occurs, resulting in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the target site, e.g., insertion of the transgene in template polynucleotide. Crossover with the template polynucleotide may occur upon resolution of the junctions.

In the SDSA pathway, only one single stranded overhang invades the template polynucleotide and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

In alternative HDR, a single strand template polynucleotide, e.g., template polynucleotide, is introduced. A nick, single strand break, or double strand break at the target site, for altering a desired target site, is mediated by a nuclease molecule, and resection at the break occurs to reveal single stranded overhangs. Incorporation of the sequence of the template polynucleotide to correct or alter the target site of the DNA typically occurs by the SDSA pathway, as described herein.

“Alternative HDR”, or alternative homology-directed repair, in some embodiments, refers to the process of repairing DNA damage using a homologous nucleic acid (e.g., an endogenous homologous sequence, e.g., a sister chromatid, or an exogenous nucleic acid, e.g., a template polynucleotide). Alternative HDR is distinct from canonical HDR in that the process utilizes different pathways from canonical HDR, and can be inhibited by the canonical HDR mediators, RAD51 and BRCA2. Also, alternative HDR uses a single-stranded or nicked homologous nucleic acid for repair of the break. “Canonical HDR”, or canonical homology-directed repair, in some embodiments, refers to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2 and the homologous nucleic acid is typically double-stranded. Unless indicated otherwise, the term “HDR” in some embodiments encompasses canonical HDR and alternative HDR.

In some embodiments, double strand cleavage is effected by a nuclease, e.g., a Cas9 molecule having cleavage activity associated with an HNH-like domain and cleavage activity associated with a RuvC-like domain, e.g., an N-terminal RuvC-like domain, e.g., a wild type Cas9. Such embodiments require only a single gRNA.

In some embodiments, one single strand break, or nick, is effected by a nuclease molecule having nickase activity, e.g., a Cas9 nickase. A nicked DNA at the target site can be a substrate for alternative HDR.

In some embodiments, two single strand breaks, or nicks, are effected by a nuclease, e.g., Cas9 molecule, having nickase activity, e.g., cleavage activity associated with an HNH-like domain or cleavage activity associated with an N-terminal RuvC-like domain. Such embodiments usually require two gRNAs, one for placement of each single strand break. In some embodiments, the Cas9 molecule having nickase activity cleaves the strand to which the gRNA hybridizes, but not the strand that is complementary to the strand to which the gRNA hybridizes. In some embodiments, the Cas9 molecule having nickase activity does not cleave the strand to which the gRNA hybridizes, but rather cleaves the strand that is complementary to the strand to which the gRNA hybridizes. In some embodiments, the nickase has HNH activity, e.g., a Cas9 molecule having the RuvC activity inactivated, e.g., a Cas9 molecule having a mutation at D10, e.g., the D10A mutation. D10A inactivates RuvC; therefore, the Cas9 nickase has (only) HNH activity and will cut on the strand to which the gRNA hybridizes (e.g., the complementary strand, which does not have the NGG PAM on it). In some embodiments, a Cas9 molecule having an H840, e.g., an H840A, mutation can be used as a nickase. H840A inactivates HNH; therefore, the Cas9 nickase has (only) RuvC activity and cuts on the non-complementary strand (e.g., the strand that has the NGG PAM and whose sequence is identical to the gRNA). In some embodiments, the Cas9 molecule is an N-terminal RuvC-like domain nickase, e.g., the Cas9 molecule comprises a mutation at N863, e.g., N863A.

In some embodiments, in which a nickase and two gRNAs are used to position two single strand nicks, one nick is on the + strand and one nick is on the—strand of the target DNA. The PAMs are outwardly facing. The gRNAs can be selected such that the gRNAs are separated by, from about 0-50, 0-100, or 0-200 nucleotides. In some embodiments, there is no overlap between the target sequences that are complementary to the targeting domains of the two gRNAs. In some embodiments, the gRNAs do not overlap and are separated by as much as 50, 100, or 200 nucleotides. In some embodiments, the use of two gRNAs can increase specificity, e.g., by decreasing off-target binding (Ran et al., Cell 2013).

In some embodiments, a single nick can be used to induce HDR, e.g., alternative HDR. It is contemplated herein that a single nick can be used to increase the ratio of HR to NHEJ at a given cleavage site, e.g., target site. In some embodiments, a single strand break is formed in the strand of the DNA at the target site to which the targeting domain of said gRNA is complementary. In another embodiment, a single strand break is formed in the strand of the DNA at the target site other than the strand to which the targeting domain of said gRNA is complementary.

In some embodiments, other DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

2. Placement of the Genetic Disruption (e.g., DNA Strand Breaks)

Targeted integration results in the transgene being integrated into a specific gene or locus in the genome. The transgene may be integrated anywhere at or near one of the at least one target site(s) or site in the genome. In some embodiments, the transgene is integrated at or near one of the at least one target site(s), for example, within 300, 250, 200, 150, 100, 50, 10, 5, 4, 3, 2, 1 or fewer base pairs upstream or downstream of the site of cleavage, such as within 100, 50, 10, 5, 4, 3, 2, 1 base pairs of either side of the target site, such as within 50, 10, 5, 4, 3, 2, 1 base pairs of either side of the target site. In some embodiments, the integrated sequence comprising the transgene does not include any vector sequences (e.g., viral vector sequences). In some embodiments, the integrated sequence includes a portion of the vector sequences (e.g., viral vector sequences).

The double strand break or single strand break in one of the strands should be sufficiently close to the site for targeted integration such that an alteration is produced in the desired region, e.g., insertion of transgene or correction of a mutation occurs. In some embodiments, the distance is not more than 10, 25, 50, 100, 200, 300, 350, 400 or 500 nucleotides. In some embodiments, it is believed that the break should be sufficiently close to the site for targeted integration such that the break is within the region that is subject to exonuclease-mediated removal during end resection. In some embodiments, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150, 200, 300, 350, 400 or 500 nucleotides of the region desired to be altered, e.g., site for targeted insertion. The break, e.g., a double strand or single strand break, can be positioned upstream or downstream of the region desired to be altered, e.g., site for targeted insertion. In some embodiments, a break is positioned within the region desired to be altered, e.g., within a region defined by at least two mutant nucleotides. In some embodiments, a break is positioned immediately adjacent to the region desired to be altered, e.g., immediately upstream or downstream of site for targeted integration.

In some embodiments, a single strand break is accompanied by an additional single strand break, positioned by a second gRNA molecule. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150, 200, 300, 350, 400 or 500 nucleotides of a site for targeted integration. In some embodiments, the first and second gRNA molecules are configured such, that when guiding a Cas9 nickase, a single strand break will be accompanied by an additional single strand break, positioned by a second gRNA, sufficiently close to one another to result in alteration of the desired region. In some embodiments, the first and second gRNA molecules are configured such that a single strand break positioned by said second gRNA is within 10, 20, 30, 40, or 50 nucleotides of the break positioned by said first gRNA molecule, e.g., when the Cas9 is a nickase. In some embodiments, the two gRNA molecules are configured to position cuts at the same position, or within a few nucleotides of one another, on different strands, e.g., essentially mimicking a double strand break.

In some embodiments, in which a gRNA (unimolecular (or chimeric) or modular gRNA) and Cas9 nuclease induce a double strand break for the purpose of inducing HDR-mediated insertion of transgene or correction, the cleavage site is between 0-200 bp (e.g., 0-175, 0 to 150, 0 to 125, 0 to 100, 0 to 75, 0 to 50, 0 to 25, 25 to 200, 25 to 175, 25 to 150, 25 to 125, 25 to 100, 25 to 75, 25 to 50, 50 to 200, 50 to 175, 50 to 150, 50 to 125, 50 to 100, 50 to 75, 75 to 200, 75 to 175, 75 to 150, 75 to 125, 75 to 100 bp) away from the site for targeted integration. In some embodiments, the cleavage site is between 0-100 bp (e.g., 0 to 75, 0 to 50, 0 to 25, 25 to 100, 25 to 75, 25 to 50, 50 to 100, 50 to 75 or 75 to 100 bp) away from the site for targeted integration.

In some embodiments, one can promote HDR by using nickases to generate a break with overhangs. In some embodiments, the single stranded nature of the overhangs can enhance the cell's likelihood of repairing the break by HDR as opposed to, e.g., NHEJ.

Specifically, in some embodiments, HDR is promoted by selecting a first gRNA that targets a first nickase to a first target site, and a second gRNA that targets a second nickase to a second target site which is on the opposite DNA strand from the first target site and offset from the first nick. In some embodiments, the targeting domain of a gRNA molecule is configured to position a cleavage event sufficiently far from a preselected nucleotide, e.g., the nucleotide of a coding region, such that the nucleotide is not altered. In some embodiments, the targeting domain of a gRNA molecule is configured to position an intronic cleavage event sufficiently far from an intron/exon border, or naturally occurring splice signal, to avoid alteration of the exonic sequence or unwanted splicing events. In some embodiments, the targeting domain of a gRNA molecule is configured to position in an early exon, to allow deletion or knock-out of the endogenous gene, and/or allow in-frame integration of the transgene at or near one of the at least one target site(s).

In some embodiments, a double strand break can be accompanied by an additional double strand break, positioned by a second gRNA molecule. In some embodiments, a double strand break can be accompanied by two additional single strand breaks, positioned by a second gRNA molecule and a third gRNA molecule.

In some embodiments, two gRNAs, e.g., independently, unimolecular (or chimeric) or modular gRNA, are configured to position a double-strand break on both sides of a site for targeted integration.

3. Template Polynucleotides

A template polynucleotide having homology with sequences at or near one or more target site(s) in the endogenous DNA can be used to alter the structure of a target DNA, e.g., targeted insertion of the transgene. In some embodiments, the template polynucleotide contains homology sequences (e.g., homology arms) flanking the transgene, e.g., nucleic acid sequences encoding a recombinant receptor, for targeted insertion. In some embodiments, the homology sequences target the transgene at one or more of the TRAC, TRBC1 and/or TRBC2 loci. In some embodiments, the template polynucleotide includes additional sequences (coding or non-coding sequences) between the homology arms, such as a regulatory sequences, such as promoters and/or enhancers, splice donor and/or acceptor sites, internal ribosome entry site (IRES), sequences encoding ribosome skipping elements (e.g., 2A peptides), markers and/or SA sites, and/or one or more additional transgenes.

The sequence of interest in the template polynucleotide may comprise one or more sequences encoding a functional polypeptide (e.g., a cDNA), with or without a promoter.

In some embodiments, the transgene contained in the template polynucleotide comprises a sequence encoding a cell surface receptor (e.g., a recombinant receptor) or a chain thereof, an antibody, an antigen, an enzyme, a growth factor, a nuclear receptor, a hormone, a lymphokine, a cytokine, a reporter, functional fragments or functional variants of any of the herein and combinations of the herein. The transgene may encode a one or more proteins useful in cancer therapies, for example one or more chimeric antigen receptors (CARs) and/or a recombinant T cell receptor (TCR). In some embodiments, the transgene can encode any of the recombinant receptors described in Section IV herein or any chains, regions and/or domains thereof. In some embodiments, the transgene encodes a recombinant T cell receptor (TCR) or any chains, regions and/or domains thereof.

In certain embodiments, the polynucleotide, e.g., template polynucleotide contains and/or includes a transgene encoding all or a portion of a recombinant receptor, e.g., a recombinant TCR or a chain thereof. In particular embodiments, the transgene is targeted at a target site(s) that is within a gene, locus, or open reading frame that encodes an endogenous receptor, e.g., an endogenous gene encoding one or more regions, chains or portions of a TCR.

In certain embodiments, the template polynucleotide includes or contains a transgene, a portion of a transgene, and/or a nucleic acid encodes recombinant receptor is a recombinant TCR or chain thereof that contains one or more variable domains and one or more constant domains. In certain embodiments, the recombinant TCR or chain thereof contains one or more constant domains that shares complete, e.g., at or about 100% identity, to all or a portion and/or fragment of an endogenous TCR constant domain. In some embodiments, the transgene encodes all or a portion of a constant domain, e.g., a portion or fragment of the constant domain that is completely or partially identical to an endogenous TCR constant domain. In some embodiments, the transgene contains nucleotides of a sequence having at or at least 70%, 75%, 80%, 85%, 90%, 95%, 97%, 98%, 99%, 99.5%, or 99.9% sequence identity to all or a portion of the nucleic acid sequence set forth in SEQ ID NOS: 1, 2, or 3.

In some of embodiments, the transgene contains a sequence encoding a TCRα and/or TCRβ chain or a portion thereof that has been codon-optimized. In some embodiments, the transgene encodes a portion of a TCRα and/or TCRβ chain with less than 100% amino acid sequence identity to a corresponding portion of a native or endogenous TCRα and/or TCRβ chain. In some embodiments, the encoded TCRα and/or TCRβ chain contains an amino acid sequence with, with about, or with at least 70%, 75%, 80%, 85%, 90%, 95%, 98%, 99%, or greater than 99% identity but less than 100% identity to a corresponding native or endogenous TCRα and/or TCRβ chain. In particular embodiments, the transgene encodes a TCRα and/or TCRβ constant domain or portion thereof with less than 100% amino acid sequence identity to a corresponding native or endogenous TCRα and/or TCRβ constant domain. In some embodiments, the TCRα and/or TCRβ constant domain contains an amino acid sequence with, with about, or with at least 70%, 75%, 80%, 85%, 90%, 95%, 98%, 99%, or greater than 99% identity but less than 100% identity to a corresponding native or endogenous TCRα and/or TCRβ chain.

In certain embodiments, the transgene contains one or more modifications(s) to introduce one or more cysteine residues that are capable of forming one or more non-native disulfide bridges between the TCRα chain and TCRβ chain. In some embodiments, the transgene encodes a TCRα chain or a portion thereof containing a TCRα constant domain containing a cysteine at a position corresponding to position 48 with numbering as set forth in SEQ ID NO: 24. In some embodiments, the TCRα constant domain has an amino acid sequence set forth in any of SEQ ID NOS: 19 or 24, or a sequence of amino acids that has, has about, or has at least 70%, 75%, 80%, 85% 90%, 95%, 97%, 98%, 99% sequence identity thereto containing one or more cysteine residues capable of forming a non-native disulfide bond with a TCRβ chain. In some embodiments, the transgene encodes a TCRβ chain or a portion thereof containing a TCRβ constant domain containing a cysteine at a position corresponding to position 57 with numbering as set forth in SEQ ID NO: 20. In some embodiments, the TCRβ constant domain has an amino acid sequence set forth in any of SEQ ID NOS: 20, 21 or 25, or a sequence of amino acids that has, has about, or has at least 70%, 75%, 80%, 85% 90%, 95%, 97%, 98%, 99% sequence identity thereto containing one or more cysteine residues capable of forming a non-native disulfide bond with a TCRα chain.

In particular embodiments, the transgene encodes a TCRα and/or TCRβ chain and/or a TCRα and/or TCRβ chain constant domains containing one or more modifications to introduce one or more disulfide bonds. In some embodiments, the transgene encodes a TCRα and/or TCRβ chain and/or a TCRα and/or TCRβ with one or more modifications to remove or prevent a native disulfide bond, e.g., between the TCRα encoded by the transgene and the endogenous TCRβ chain, or between the TCR β encoded by the transgene and the endogenous TCR α chain. In some embodiments, one or more native cysteines that form and/or are capable of forming a native inter-chain disulfide bond are substituted to another residue, e.g., serine or alanine. In some embodiments, the TCRα and/or TCRβ chain and/or a TCRα and/or TCRβ chain constant domains are modified to replace one or more non-cysteine residues to a cysteine. In some embodiments, the one or more non-native cysteine residues are capable of forming non-native disulfide bonds, e.g., between the recombinant TCRα and TCRβ chain encoded by the transgene. In some embodiments, the cysteine is introduced at one or more of residue Thr48, Thr45, Tyr10, Thr45, and Ser15 with reference to numbering of a TCRα constant domain set forth in SEQ ID NO: 24. In certain embodiments, cysteines can be introduced at residue Ser57, Ser77, Ser17, Asp59, of Glu15 of the TCR β chain with reference to numbering of TCRβ chain set forth in SEQ ID NO: 20. Exemplary non-native disulfide bonds of a TCR are described in published International PCT No. WO2006/000830, WO 2006/037960 and Kuball et al. (2007) Blood, 109:2331-2338. In some embodiments, the transgene encodes a portion of a TCRα chain and/or a TCRα constant domain containing one or more modifications to introduce one or more disulfide bonds.

In some embodiments, the transgene encodes all or a portion of a TCRα chain and/or a TCRα constant domain with one or more modifications to remove or prevent a native disulfide bond, e.g., between the TCRα chain encoded by the transgene and the endogenous TCRβ chain. In some embodiments, one or more native cysteines that form and/or are capable of forming a native interchain disulfide bond are substituted to another residue, e.g., serine or alanine. In some embodiments, the portion of the TCRα chain and/or TCRα constant domain is modified to replace one or more non-cysteine residues to a cysteine. In some embodiments, the one or more non-native cysteine residues are capable of forming non-native disulfide bonds, e.g., with a TCRβ chain encoded by the transgene. In some embodiments, the transgene encodes all or a portion of a TCRβ chain and/or a TCRβ constant domain with one or more modifications to remove or prevent a native disulfide bond, e.g., between the TCRβ chain encoded by the transgene and the endogenous TCRα chain. In some embodiments, one or more native cysteines that form and/or are capable of forming a native interchain disulfide bond are substituted to another residue, e.g., serine or alanine. In some embodiments, the portion of the TCRβ chain and/or TCRβ constant domain is modified to replace one or more non-cysteine residues to a cysteine.

In some embodiments, the one or more non-native cysteine residues are capable of forming non-native disulfide bonds, e.g., with a TCRα chain encoded by the transgene. In some embodiments, one or more different template polynucleotides are used for targeting integration of the transgene at one or more different target sites. For targeting integration at different target sites, one or more genetic disruptions (e.g., DNA break) are generated at one or more of the target sites; and one or more different homology sequences are used for targeting integration of the transgene into the respective target site. In some embodiments, the transgene inserted at each site is the same or substantially the same. In some embodiments, transgene inserted at each site are different. In some embodiments, two or more different transgenes, encoding two or more different domains or chains of a protein, is inserted at one or more target sites. In some embodiments, the transgene encoding the recombinant TCR or antigen-binding fragment or chain thereof encodes one chain of a recombinant TCR and the second transgene encodes a different chain of the recombinant TCR. In some embodiments, the transgene encoding the recombinant TCR or antigen-binding fragment or chain thereof encodes the alpha (TCRα) chain of the recombinant TCR and the second transgene encodes the beta (TCRβ) chain of the recombinant TCR. In some embodiments, two or more transgene encoding different domains of the recombinant receptors are targeted for integration at two or more target sites. For example, in some embodiments, transgene encoding a recombinant TCR alpha chain is targeted for integration at the TRAC locus, and transgene encoding a recombinant TCR beta chain is targeted for integration at the TRBC1 and/or TRBC2 loci.

In some embodiments, two or more different template polynucleotides are used to target two or more transgene for integration at two or more different endogenous gene loci. In some embodiments, the first template polynucleotide includes transgene encoding a recombinant receptor. In some embodiments, the second template polynucleotide includes one or more second transgene(s), e.g., one or more second transgenes encoding one or more different molecules, polypeptides and/or factors. Any of the description or characterization of the template polynucleotide provided herein, can also apply to the one or more second template polynucleotide(s).

In some embodiments, the one or more second transgene is targeted for integration at or near one of the at least one target site(s) in the TRAC gene. In some embodiments, the one or more second transgene is targeted for integration at or near one of the at least one target site(s) in the TRBC1 or the TRBC2 gene. In some embodiments, the transgene encoding the recombinant TCR or antigen-binding fragment or chain thereof is targeted for integration at or near one of the at least one target site(s) in the TRAC gene, the TRBC1 gene or the TRBC2 gene, and the one or more second transgene is targeted for integration at or near one or more of the target site that is not targeted by the transgene encoding the recombinant TCR or antigen-binding fragment or chain thereof.

In some embodiments, the molecule, polypeptide or factor encoded by the one or more second transgene is a molecule, polypeptide, factor or agent that can provide co-stimulatory signal to the immune cell, e.g. T cell. In some embodiments, the molecule, polypeptide, factor or agent encoded by the second transgene is an additional receptor, e.g., an additional recombinant receptor. In some embodiments, the additional receptor can provide co-stimulatory signal and/or counters or reverses an inhibitory signal.

In some embodiments, the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor.

In some embodiments, the molecule, polypeptide or factor encoded by the one or more second transgene is a co-stimulatory ligand. Exemplary co-stimulatory ligands include tumor necrosis factor (TNF) ligand or an immunoglobulin (Ig) superfamily ligand. In some embodiments, exemplary TNF ligands include 4-1BBL, OX40L, CD70, LIGHT, and CD30L. In some embodiments, exemplary Ig superfamily ligands include CD80 and CD86. In some embodiments, the co-stimulatory ligand includes CD3, CD27, CD28, CD83, CD127, 4-1BB, PD-1 or PDIL. In some embodiments, the molecule, polypeptide or factor encoded by the one or more second transgene is a cytokine, such as IL-2, IL-3, IL-6, IL-11, IL-12, IL7, IL-15, IL-21, granulocyte macrophage colony stimulating factor (GM-CSF), interferon alpha (IFN-α), interferon beta (IFN-β) or interferon gamma (IFN-γ) and erythropoietin. Exemplary co-stimulatory ligands and cytokines that can be encoded by the one or more second transgene include those described in, e.g., WO 2008121420.

In some embodiments, the molecule, polypeptide or factor encoded by the one or more second transgene is a soluble single-chain variable fragment (scFv), such as an scFv that binds a polypeptide that has immunosuppressive activity or immunostimulatory activity such as CD47, PD-1, CTLA-4 and ligands thereof or CD28, OX-40, 4-1BB and ligands thereof. Exemplary scFvs that can be encoded by the one or more second transgene include those described in, e.g., WO 2014134165.

In some embodiments, the molecule, polypeptide or factor encoded by the one or more second transgene is an immunomodulatory fusion protein or a chimeric switch receptor (CSR). In some embodiments, the encoded immunomodulatory fusion protein comprises (a) an extracellular component comprised of a binding domain that specifically binds a target, (b) an intracellular component comprised of an intracellular signaling domain, and (c) a hydrophobic component connecting the extracellular and intracellular components. In some embodiments, the encoded immunomodulatory fusion protein comprises (a) an extracellular binding domain that specifically binds an antigen derived from CD200R, SIRPα, CD279 (PD-1), CD2, CD95 (Fas), CD152 (CTLA4), CD223 (LAG3), CD272 (BTLA), A2aR, KIR, TIM3, CD300 or LPA5; (b) an intracellular signaling domain derived from CD3ε, CD3δ, CD3ζ, CD25, CD27, CD28, CD40, CD47, CD79A, CD79B, CD134 (OX40), CD137 (4-1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRFM, Zap70, PTCH2, or any combination thereof; and (c) a hydrophobic transmembrane domain derived from CD2, CD3ε, CD3δ, CD3ζ, CD25, CD27, CD28, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5 or Zap70. In some embodiments, the molecule, polypeptide or factor encoded by the one or more second transgene is a chimeric switch receptor (CSR), such as a CSR comprising a truncated extracellular domain of PD1 and the transmembrane and cytoplasmic signaling domains of CD28. Exemplary immunomodulatory fusion protein or CSR that can be encoded by the one or more second transgene include those described in, e.g., WO 2014134165, US 2014/0219975, WO 2013/019615 and Liu et al., Cancer Res. (2016) 76(6):1578-90.

In some embodiments, the molecule, polypeptide or factor encoded by the one or more second transgene is a co-receptor. In some embodiments, exemplary co-receptors include CD4 or CD8.

In some embodiments, the one or more target sites are at or near one or more of the TRAC, TRBC1 and/or TRBC2 loci. In some embodiments, the first target site is at or near the coding sequence of the TRAC gene locus, and the second target site is at or near the coding sequence of the TRBC1 gene locus. In some embodiments, the first target site is at or near the coding sequence of the TRAC gene locus, and the second target site is at or near the coding sequence of the TRBC2 gene locus. In some embodiments, the first target site is at or near the coding sequence of the TRAC gene locus, and the second target site both the TRBC1 and TRBC2 loci, e.g., at a sequence that is conserved between TRBC1 and TRBC2.

In some embodiments, one or more different DNA sites, e.g., TRAC, TRBC1 and/or TRBC2 loci, are targeted, and one or more transgene are inserted at each site. In some embodiments, the transgene inserted at each site is the same or substantially the same. In some embodiments, transgene inserted at each site are different. In some embodiments, a transgene is only inserted at one of the target sites (e.g., TRAC locus), and another target site is targeted for gene editing (e.g., knock-out).

In some embodiments, any of the lengths and positions of the homology arms and relative position to the target site(s), such as any described herein, can also apply to the one or more second template polynucleotide(s).

In some embodiments, nuclease-induced HDR results in an insertion of a transgene (also called “exogenous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

In some aspects, nucleic acid sequences of interest, including coding and/or non-coding sequences and/or partial coding sequences, that are inserted or integrated at the target location in the genome can also be referred to as “transgene,” “transgene sequences,” “exogenous nucleic acids sequences,” “heterologous sequences” or “donor sequences.” In some aspects, the transgene is a nucleic acid sequence that is exogenous or heterologous to an endogenous genomic sequences, such as the endogenous genomic sequences at a specific target locus or target location in the genome, of a T cell, e.g., a human T cell. In some aspects, the transgene is a sequence that is modified or different compared to an endogenous genomic sequence at a target locus or target location of a T cell, e.g., a human T cell. In some aspects, the transgene is a nucleic acid sequence that originates from or is modified compared to nucleic acid sequences from different genes, species and/or origins. In some aspects, the transgene is a sequence that is derived from a sequence from a different locus, e.g., a different genomic region or a different gene, of the same species.

Polynucleotides for insertion can also be referred to as “transgene” or “exogenous sequences” or “donor” polynucleotides or molecules. The template polynucleotide can be DNA, single-stranded and/or double-stranded and can be introduced into a cell in linear or circular form. The template polynucleotide can be RNA single-stranded and/or double-stranded and can be introduced as a RNA molecule (e.g., part of an RNA virus). See also, U.S. Patent Publication Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by known methods. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Publication No. 20130326645.

In some embodiments, the double-stranded template polynucleotide includes sequences (also referred to as transgene) greater than 1 kb in length, for example between 2 and 200 kb, between 2 and 10 kb (or any value therebetween). The double-stranded template polynucleotide also includes at least one nuclease target site, for example. In some embodiments, the template polynucleotide includes at least 2 target sites, for example for a pair of ZFNs or TALENs. Typically, the nuclease target sites are outside the transgene sequences, for example, 5′ and/or 3′ to the transgene sequences, for cleavage of the transgene. The nuclease cleavage site(s) may be for any nuclease(s). In some embodiments, the nuclease target site(s) contained in the double-stranded template polynucleotide are for the same nuclease(s) used to cleave the endogenous target into which the cleaved template polynucleotide is integrated via homology-independent methods.

In some embodiments, the nucleic acid template system is double stranded. In some embodiments, the nucleic acid template system is single stranded. In some embodiments, the nucleic acid template system comprises a single stranded portion and a double stranded portion.

In some embodiments, the template polynucleotide contains the transgene, e.g., recombinant receptor-encoding nucleic acid sequences, flanked by homology sequences (also called “homology arms”) on the 5′ and 3′ ends, to allow the DNA repair machinery, e.g., homologous recombination machinery, to use the template polynucleotide as a template for repair, effectively inserting the transgene into the target site of integration in the genome. The homology arm should extend at least as far as the region in which end resection may occur, e.g., in order to allow the resected single stranded overhang to find a complementary region within the template polynucleotide. The overall length could be limited by parameters such as plasmid size or viral packaging limits. In some embodiments, a homology arm does not extend into repeated elements, e.g., ALU repeats or LINE repeats.

Exemplary homology arm lengths include at least or at least about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides. Exemplary homology arm lengths include less than or less than about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides.

Target site (also known as “target position,” “target DNA sequence” or “target location”), in some embodiments, refers to a site on a target DNA (e.g., the chromosome) that is modified by the one or more agent(s) capable of inducing a genetic disruption, e.g., a Cas9 molecule. For example, the target site can be a modified Cas9 molecule cleavage of the DNA at the target site and template polynucleotide directed modification, e.g., targeted insertion of the transgene, at the target site. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence (e.g., the sequence to which the gRNA binds). In some aspects, a pair of single stranded breaks (e.g., nicks) on each side of the target site can be generated.

In some embodiments, the template polynucleotide comprises about 500 to 1000, e.g., 600 to 900 or 700 to 800, base pairs of homology on either side of the target site at the endogenous gene. In some embodiments, the template polynucleotide comprises at least or less than or about 200, 300, 400, 500, 600, 700, 800, 900 or 1000 base pairs homology 5′ of the target site, 3′ of the target site, or both 5′ and 3′ of the target site, e.g., within the TRAC, TRBC1, and/or TRBC2 gene, locus, or open reading frame (e.g., described in Tables 1-3 herein).

In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 3′ of the target site. In some embodiments, the template polynucleotide comprises about 100 to 500, 200 to 400 or 250 to 350, base pairs homology 3′ of the transgene and/or target site. In some embodiments, the template polynucleotide comprises less than about 100, 90, 80, 70, 60, 50, 40, 30, 20, 15, or 10 base pairs homology 5′ of the target site, e.g., within the TRAC, TRBC1, and/or TRBC2 gene, locus, or open reading frame (e.g., described in Tables 1-3 herein).

In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 5′ of the target site. In some embodiments, the template polynucleotide comprises about 100 to 500, 200 to 400 or 250 to 350, base pairs homology 5′ of the transgene and/or target site. In some embodiments, the template polynucleotide comprises less than about 100, 90, 80, 70, 60, 50, 40, 30, 20, 15, or 10 base pairs homology 3′ of the target site, e.g., within the TRAC, TRBC1, and/or TRBC2 gene, locus, or open reading frame (e.g., described in Tables 1-3 herein).

In some embodiments, a template polynucleotide is to a nucleic acid sequence which can be used in conjunction with one or more agent(s) capable of introducing a genetic disruption to alter the structure of a target site. In some embodiments, the target site is modified to have the some or all of the sequence of the template polynucleotide, typically at or near cleavage site(s). In some embodiments, the template polynucleotide is single stranded. In some embodiments, the template polynucleotide is double stranded. In some embodiments, the template polynucleotide is DNA, e.g., double stranded DNA In some embodiments, the template polynucleotide is single stranded DNA. In some embodiments, the template polynucleotide is encoded on the same vector backbone, e.g. AAV genome, plasmid DNA, as the Cas9 and gRNA. In some embodiments, the template polynucleotide is excised from a vector backbone in vivo, e.g., it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, e.g., in a vector.

In some embodiments, the template polynucleotide alters the structure of the target site, e.g., insertion of transgene, by participating in a homology directed repair event. In some embodiments, the template polynucleotide alters the sequence of the target site.

In some embodiments, the template polynucleotide includes sequence that corresponds to a site on the target sequence that is cleaved by one or more agent(s) capable of introducing a genetic disruption. In some embodiments, the template polynucleotide includes sequence that corresponds to both, a first site on the target sequence that is cleaved in a first agent capable of introducing a genetic disruption, and a second site on the target sequence that is cleaved in a second agent capable of introducing a genetic disruption.

In some embodiments, a template polynucleotide comprises the following components: [5′ homology arm]-[transgene]-[3′ homology arm]. The homology arms provide for recombination into the chromosome, thus insertion of the transgene into the DNA at or near the cleavage site, e.g., target site(s). In some embodiments, the homology arms flank the most distal target site(s).

In some embodiments, the 3′ end of the 5′ homology arm is the position next to the 5′ end of the transgene. In some embodiments, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the transgene.

In some embodiments, the 5′ end of the 3′ homology arm is the position next to the 3′ end of the transgene. In some embodiments, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ homology arms, may each comprise about 1000 base pairs (bp) of sequence flanking the most distal gRNAs (e.g., 1000 bp of sequence on either side of the mutation).

In some embodiments, one or more second template polynucleotide comprising one or more second transgene can be introduced. In some embodiments, the one or more second transgene is targeted for integration at or near one of the at least one target site via homology directed repair (HDR).

In some embodiments, the one or more second template polynucleotide comprises the structure [second 5′ homology arm]-[one or more second transgene]-[second 3′ homology arm]. The homology arms provide for recombination into the chromosome, thus insertion of the transgene into the DNA at or near the cleavage site e.g., target site(s). In some embodiments, the homology arms flank the most distal cleavage sites. In some embodiments, the second 5′ homology arm and second 3′ homology arm comprises nucleic acid sequences homologous to nucleic acid sequences surrounding the at least one target site. In some embodiments, the second 5′ homology arm comprises nucleic acid sequences that are homologous to nucleic acid sequences second 5′ of the target site. In some embodiments, the second 3′ homology arm comprises nucleic acid sequences that are homologous to nucleic acid sequences second 3′ of the target site. In some embodiments, the second 5′ homology arm and second 3′ homology arm independently are at least or at least about or is or is about 10, 20, 30, 40, 50, 100, 200, 300, 400, 500, 600, 700, 800, 900, 1000, 1500, or 2000 base pairs, or less than or less than about 10, 20, 30, 40, 50, 100, 200, 300, 400, 500, 600, 700, 800, 900, 1000, 1500, or 2000 base pairs. In some embodiments, the second 5′ homology arm and second 3′ homology arm independently are between about 50 and 100, 100 and 250, 250 and 500, 500 and 750, 750 and 1000, 1000 and 2000 base pairs. In some embodiments, the second 5′ homology arm and second 3′ homology arm independently are about 10, 20, 30, 40, 50, 100, 200, 300, 400, 500, 600, 700, 800, 900, 1000, 1500, or 2000 base pairs.

In some embodiments, the one or more second transgene is targeted for integration at or near the target site in the TRAC gene (e.g., described in Table 1 herein). In some embodiments, the one or more second transgene is targeted for integration at or near the target site in the TRBC1 or the TRBC2 gene (e.g., described in Tables 2-3 herein).

It is contemplated herein that one or both homology arms may be shortened to avoid including certain sequence repeat elements, e.g., Alu repeats or LINE elements. For example, a 5′ homology arm may be shortened to avoid a sequence repeat element. In some embodiments, a 3′ homology arm may be shortened to avoid a sequence repeat element. In some embodiments, both the 5′ and the 3′ homology arms may be shortened to avoid including certain sequence repeat elements. It is contemplated herein that template polynucleotides for targeted insertion may be designed for use as a single-stranded oligonucleotide, e.g., a single-stranded oligodeoxynucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made. In some embodiments, a longer homology arm is made by a method other than chemical synthesis, e.g., by denaturing a long double stranded nucleic acid and purifying one of the strands, e.g., by affinity for a strand-specific sequence anchored to a solid substrate.

In some embodiments, alternative HDR proceeds more efficiently when the template polynucleotide has extended homology 5′ to the target site (i.e., in the 5′ direction of the target site strand). Accordingly, in some embodiments, the template polynucleotide has a longer homology arm and a shorter homology arm, wherein the longer homology arm can anneal 5′ of the target site. In some embodiments, the arm that can anneal 5′ to the target site is at least 25, 50, 75, 100, 125, 150, 175, or 200, 300, 400, 500, 600, 700, 800, 900, 1000, 1500, 2000, 3000, 4000, or 5000 nucleotides from the target site or the 5′ or 3′ end of the transgene. In some embodiments, the arm that can anneal 5′ to the target site is at least 10%, 20%, 30%, 40%, or 50% longer than the arm that can anneal 3′ to the target site. In some embodiments, the arm that can anneal 5′ to the target site is at least 2×, 3×, 4×, or 5× longer than the arm that can anneal 3′ to the target site. Depending on whether a ssDNA template can anneal to the intact strand or the target site strand, the homology arm that anneals 5′ to the target site may be at the 5′ end of the ssDNA template or the 3′ end of the ssDNA template, respectively.

Similarly, in some embodiments, the template polynucleotide has a 5′ homology arm, a transgene, and a 3′ homology arm, such that the template polynucleotide contains extended homology to the 5′ of the target site. For example, the 5′ homology arm and 3′ homology arm may be substantially the same length, but the transgene may extend farther 5′ of the target site than 3′ of the target site. In some embodiments, the homology arm extends at least 10%, 20%, 30%, 40%, 50%, 2×, 3×, 4×, or 5× further to the 5′ end of the target site than the 3′ end of the target site.

In some embodiments alternative HDR proceeds more efficiently when the template polynucleotide is centered on the target site. Accordingly, in some embodiments, the template polynucleotide has two homology arms that are essentially the same size.

For instance, the first homology arm of a template polynucleotide may have a length that is within 10%, 9%, 8%, 7%, 6%, 5%, 4%, 3%, 2%, or 1% of the second homology arm of the template polynucleotide.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

In some embodiments, the template polynucleotide is a single stranded nucleic acid. In another embodiment, the template polynucleotide is a double stranded nucleic acid. In some embodiments, the template polynucleotide comprises a nucleotide sequence, e.g., of one or more nucleotides, that will be added to or will template a change in the target DNA. In some embodiments, the template polynucleotide comprises a nucleotide sequence that may be used to modify the target site. In some embodiments, the template polynucleotide comprises a nucleotide sequence, e.g., of one or more nucleotides, that corresponds to wild type sequence of the target DNA, e.g., of the target site.

The template polynucleotide may comprise a transgene. In some embodiments, the template polynucleotide comprises a 5′ homology arm. In some embodiments, the template nucleic acid comprises a 3′ homology arm.

In some embodiments, the template polynucleotide is linear double stranded DNA. The length may be, e.g., about 200-5000 base pairs, e.g., about 200, 300, 400, 500, 600, 700, 800, 900, 1000, 1200, 1400, 1600, 1800, 2000, 2500, 3000, 4000 or 5000 base pairs. The length may be, e.g., at least 200, 300, 400, 500, 600, 700, 800, 900, 1000, 1200, 1400, 1600, 1800, 2000, 2500, 3000, 4000 or 5000 base pairs. In some embodiments, the length is no greater than 200, 300, 400, 500, 600, 700, 800, 900, 1000, 1200, 1400, 1600, 1800, 2000, 2500, 3000, 4000 or 5000 base pairs. In some embodiments, a double stranded template polynucleotide has a length of about 160 base pairs, e.g., about 200-4000, 300-3500, 400-3000, 500-2500, 600-2000, 700-1900, 800-1800, 900-1700, 1000-1600, 1100-1500 or 1200-1400 base pairs.

The template polynucleotide can be linear single stranded DNA In some embodiments, the template polynucleotide is (i) linear single stranded DNA that can anneal to the nicked strand of the target DNA, (ii) linear single stranded DNA that can anneal to the intact strand of the target DNA, (iii) linear single stranded DNA that can anneal to the transcribed strand of the target DNA, (iv) linear single stranded DNA that can anneal to the non-transcribed strand of the target DNA, or more than one of the preceding.

The length may be, e.g., about 200-5000 base pairs, e.g., about 200, 300, 400, 500, 600, 700, 800, 900, 1000, 1200, 1400, 1600, 1800, 2000, 2500, 3000, 4000 or 5000 nucleotides. The length may be, e.g., at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a single stranded template polynucleotide has a length of about 160 nucleotides, e.g., about 200-4000, 300-3500, 400-3000, 500-2500, 600-2000, 700-1900, 800-1800, 900-1700, 1000-1600, 1100-1500 or 1200-1400 nucleotides.

In some embodiments, the template polynucleotide is circular double stranded DNA, e.g., a plasmid. In some embodiments, the template polynucleotide comprises about 500 to 1000 base pairs of homology on either side of the transgene and/or the target site. In some embodiments, the template polynucleotide comprises about 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 10, 20, 30, 40, 50, 100, 200, 300, 400, 500, 600, 700, 800, 900, 1000, 1500, or 2000 base pairs of homology 5′ of the target site or transgene, 3′ of the target site or transgene, or both 5′ and 3′ of the target site or transgene.

In some embodiments, the length of any of the polynucleotides, e.g., template polynucleotides, may be, e.g., at or about 200-10000 nucleotides, e.g., at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides, or a value between any of the foregoing. In some embodiments, the length may be, e.g., at least at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides, or a value between any of the foregoing. In some embodiments, the length is no greater than at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides. In some embodiments, the length is at or about 200-4000, 300-3500, 400-3000, 500-2500, 600-2000, 700-1900, 800-1800, 900-1700, 1000-1600, 1100-1500 or 1200-1400 nucleotides. In some embodiments, the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some embodiments, the polynucleotide is between at or about 2500 and at or about 5000 nucleotides, at or about 3500 and at or about 4500 nucleotides, or at or about 3750 nucleotides and at or about 4250 nucleotides in length. In some embodiments, the polynucleotide is at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length.

In some embodiments, the template polynucleotide contains homology arms for targeting the endogenous TRAC locus (exemplary nucleotide sequence of the human TRAC gene locus set forth in SEQ ID NO:1; NCBI Reference Sequence: NG_001332.3, TRAC or described in Table 1 herein). In some embodiments, the genetic disruption of the TRAC locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described in Section I.A herein. In some embodiments, the template polynucleotide comprises about 500 to 1000, e.g., 600 to 900 or 700 to 800, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequences, which is homologous to 500, 600, 700, 800, 900 or 1000 base pairs of sequences 5′ of the genetic disruption (e.g., at TRAC locus), the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption (e.g., at TRAC locus). In some embodiments, exemplary 5′ and 3′ homology arms for targeted integration at the TRAC locus are set forth in SEQ ID NO: 124 and 125, respectively. In some embodiments, exemplary 5′ and 3′ homology arms for targeted integration at the TRAC locus are set forth in SEQ ID NOS: 227-233 and 234-240, respectively.

In some embodiments, the template polynucleotide contains homology arms for targeting the endogenous TRBC1 or TRBC2 locus (exemplary nucleotide sequence of the human TRBC1 gene locus set forth in SEQ ID NO:2; NCBI Reference Sequence: NG_001333.2, TRBC1, described in Table 2 herein; exemplary nucleotide sequence of the human TRBC2 gene locus set forth in SEQ ID NO:3; NCBI Reference Sequence: NG_001333.2, TRBC2, described in Table 3 herein). In some embodiments, the genetic disruption of the TRBC1 or TRBC2 locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described in Section I.A herein. In some embodiments, the template polynucleotide comprises about 500 to 1000, e.g., 600 to 900 or 700 to 800, base pairs of homology on either side of the genetic disruption introduced by the targeted nucleases and/or gRNAs. In some embodiments, the template polynucleotide comprises about 500, 600, 700, 800, 900 or 1000 base pairs of 5′ homology arm sequences, which is homologous to 500, 600, 700, 800, 900 or 1000 base pairs of sequences 5′ of the genetic disruption (e.g., at TRBC1 or TRBC2 locus), the transgene, and about 500, 600, 700, 800, 900 or 1000 base pairs of 3′ homology arm sequences, which is homologous to 500, 600, 700, 800, 900 or 1000 base pairs of sequences 3′ of the genetic disruption (e.g., at TRBC1 or TRBC2 locus).

In some embodiments, any of the lengths and positions of the homology arms and relative position to the target site(s), such as any described herein, can also apply to the one or more second template polynucleotide(s).

In some instances, the template polynucleotide comprises a promoter, e.g., a promoter that is exogenous and/or not present at or near the target locus. In some embodiments, the promoter drives expression only in a specific cell type (e.g., a T cell or B cell or NK cell specific promoter). In some embodiments in which the functional polypeptide encoding sequences are promoterless, expression of the integrated transgene is then ensured by transcription driven by an endogenous promoter or other control element in the region of interest.

The transgene, including the transgene encoding the recombinant receptor or antigen-binding portion thereof or a chain thereof and/or the one or more second transgene, can be inserted so that its expression is driven by the endogenous promoter at the integration site, namely the promoter that drives expression of the endogenous gene into which the transgene is inserted (e.g., TRAC, TRBC1 and/or TRBC2). For example, the coding sequences in the transgene can be inserted without a promoter, but in-frame with the coding sequence of the endogenous target gene, such that expression of the integrated transgene is controlled by the transcription of the endogenous promoter at the integration site. In some embodiments, the transgene encoding the recombinant TCR or antigen-binding fragment or chain thereof and/or the one or more second transgene independently is operably linked to the endogenous promoter of the gene at the target site. In some embodiments, a ribosome skipping element/self-cleavage element, such as a 2A element, is placed upstream of the transgene coding sequence, such that the ribosome skipping element/self-cleavage element is placed in-frame with the endogenous gene, such that the expression of the transgene encoding the recombinant or antigen-binding fragment or chain thereof and/or the one or more second transgene is operably linked to the endogenous TCRα promoter.

In some embodiments, the transgene encoding the recombinant TCR or antigen-binding fragment or chain thereof and/or the one or more second transgene independently comprises one or more multicistronic element(s). In some embodiments, the one or more multicistronic element(s) are upstream of the transgene encoding the recombinant TCR or antigen-binding fragment or chain thereof and/or the one or more second transgene. In some embodiments, the multicistronic element(s) is positioned between the transgene encoding the recombinant TCR or antigen-binding fragment or chain thereof and the one or more second transgene. In some embodiments, the multicistronic element(s) is positioned between the nucleic acid sequence encoding the TCRα or a portion thereof and the nucleic acid sequence encoding the TCRβ or a portion thereof. In some embodiments, the ribosome skip element comprises a sequence encoding a ribosome skip element selected from among a T2A, a P2A, a E2A or a F2A or an internal ribosome entry site (IRES).

In some embodiments, the encoded TCRα chain and TCRβ chain are separated by a linker or a spacer region. In some embodiments, a linker sequence is included that links the TCRα and TCRβ chains to form the single polypeptide strand. In some embodiments, the linker is of sufficient length to span the distance between the C terminus of the α chain and the N terminus of the β chain, or vice versa, while also ensuring that the linker length is not so long so that it blocks or reduces bonding to a target peptide-MHC complex. In some embodiments, the linker may be any linker capable of forming a single polypeptide strand, while retaining TCR binding specificity. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)n-P-, wherein n is 5 or 6 and P is proline, G is glycine and S is serine (SEQ ID NO: 22). In some embodiments, the linker has the sequence GSADDAKKDAAKKDGKS (SEQ ID NO: 23). In some embodiments, the linker or spacer between the TCRα chain or portion thereof and the TCRβ chain or portion thereof that is recognized by and/or is capable of being cleaved by a protease. In certain embodiments, the linker or spacer between the TCRα chain or portion thereof and the TCRβ chain or portion thereof contains a ribosome skipping element or a self-cleaving element.

In some embodiments, the transgene is or include a sequence of nucleotides that is or includes the structure [TCRβ chain]-[linker]-[TCRα chain]. In particular embodiments, the transgene is or include a sequence of nucleotides that is or includes the structure [TCRβ chain]-[self-cleaving element]-[TCRα chain]. In certain embodiments, the transgene is or include a sequence of nucleotides that is or includes the structure [TCRβ chain]-[ribosome skipping sequence]-[TCRα chain]. In some embodiments, the transgene is or include a sequence of nucleotides that is or includes the structure [TCRα chain]-[linker]-[TCRβ chain]. In particular embodiments, the transgene is or include a sequence of nucleotides that is or includes the structure [TCRα chain]-[self-cleaving element]-[TCRβ chain]. In certain embodiments, the transgene is or include a sequence of nucleotides that is or includes the structure [TCRα chain]-[ribosome skipping sequence]-[TCRβ chain]. In some embodiments, the structures are encoded by a polynucleotide strand of a single or double stranded polynucleotide, in a 5′ to 3′ orientation.

In some cases, the ribosome skipping element/self-cleavage element, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). This allows the inserted transgene to be controlled by the transcription of the endogenous promoter at the integration site, e.g., TRAC, TRBC1 and/or TRBC2 promoter. Exemplary ribosome skipping element/self-cleavage element include 2A sequences from the foot-and-mouth disease virus (F2A, e.g., SEQ ID NO: 11), equine rhinitis A virus (E2A, e.g., SEQ ID NO: 10), Thosea asigna virus (T2A, e.g., SEQ ID NO: 6 or 7), and porcine teschovirus-1 (P2A, e.g., SEQ ID NO: 8 or 9) as described in U.S. Patent Publication No. 20070116690. In some embodiments, the template polynucleotide includes a P2A ribosome skipping element (sequence set forth in SEQ ID NO: 8 or 9) upstream of the transgene, e.g., recombinant receptor encoding nucleic acids.

In some embodiments, transgene may comprise a promoter and/or enhancer, for example a constitutive promoter or an inducible or tissue-specific promoter. In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (sequence set forth in SEQ ID NO:18 or 126; see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some cases, the promoter is selected from among human elongation factor 1 alpha (EF1α) promoter (sequence set forth in SEQ ID NO:4 or 5) or a modified form thereof (EF1α promoter with HTLV1 enhancer; sequence set forth in SEQ ID NO: 127) or the MND promoter (sequence set forth in SEQ ID NO:18 or 126). In some embodiments, the transgene does not include a regulatory element, e.g. promoter.

In some embodiments, a “tandem” cassette is integrated into the selected site. In some embodiments, one or more of the “tandem” cassettes encode one or more polypeptide or factors, each independently controlled by a regulatory element or all controlled as a multi-cistronic expression system. In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two or three polypeptides separated from one another by sequences encoding a self-cleavage peptide (e.g., 2A sequences) or a protease recognition site (e.g., furin), as described herein. The ORF thus encodes a single polypeptide, which, either during (in the case of 2A) or after translation, is processed into the individual proteins. In some embodiments, the “tandem cassette” includes the first component of the cassette comprising a promoterless sequence, followed by a transcription termination sequence, and a second sequence, encoding an autonomous expression cassette or a multi-cistronic expression sequence. In some embodiments, the tandem cassette encodes two or more different polypeptides or factors, e.g., two or more chains or domains of a recombinant receptor. In some embodiments, nucleic acid sequences encoding two or more chains or domains of the recombinant receptor are introduced as tandem expression cassettes or bi- or multi-cistronic cassettes, into one target DNA integration site.

The transgene may be inserted into an endogenous gene such that all, some or none of the endogenous gene is expressed. In some embodiments, the transgene (e.g., with or without peptide-encoding sequences) is integrated into any endogenous locus. In some embodiments, the transgene is integrated into the TRAC, TRBC1 and/or TRBC2 gene loci.

In some embodiments, exogenous sequences may also include transcriptional or translational regulatory sequences, for example, promoters, enhancers, insulators, internal ribosome entry sites, sequences encoding 2A peptides and/or polyadenylation signals. Further, the control elements of the genes of interest can be operably linked to reporter genes to create chimeric genes (e.g., reporter expression cassettes). Additionally, splice acceptor sequences may be included. Exemplary known splice acceptor site sequences include, e.g., CTGACCTCTTCTCTTCCTCCCACAG, (SEQ ID NO:119) (from the human HBB gene) and TTTCTCTCCACAG (SEQ ID NO:120) (from the human Immunoglobulin-gamma gene).

In an exemplary embodiment, the template polynucleotide includes homology arms for targeting at the TRAC locus, regulatory sequences, e.g., promoter, and nucleic acid sequences encoding a recombinant receptor, e.g., TCR. In an exemplary embodiment, an additional template polynucleotide is employed, that includes homology arms for targeting at TRBC1 and/or TRBC2 loci, regulatory sequences, e.g., promoter, and nucleic acid sequences encoding another factor.

In some embodiments, exemplary template polynucleotides contain transgene encoding a recombinant T cell receptor under the operable control of the human elongation factor 1 alpha (EF1α) promoter with HTLV1 enhancer (sequence set forth in SEQ ID NO:127) or the MND promoter (sequence set forth in SEQ ID NO:126) or linked to nucleic acid sequences encoding a P2A ribosome skipping element (sequence set forth in SEQ ID NO:8) to drive expression of the recombinant TCR from the endogenous target gene locus (e.g., TRAC), 5′ homology arm sequence of approximately 600 bp (e.g., set forth in SEQ ID NO:124), 3′ homology arm sequence of approximately 600 bp (e.g., set forth in SEQ ID NO:125) that are homologous to sequences surrounding the target integration site in exon 1 of the human TCR α constant region (TRAC) gene. In some embodiments, the template polynucleotide further contains other nucleic acid sequences, e.g., nucleic acid sequences encoding a marker, e.g., a surface marker or a selection marker. In some embodiments, the template polynucleotide further contains viral vector sequences, e.g., adeno-associated virus (AAV) vector sequences.

The transgene contained on the template polynucleotide described herein may be isolated from plasmids, cells or other sources using known standard techniques such as PCR. Template polynucleotide for use can include varying types of topology, including circular supercoiled, circular relaxed, linear and the like. Alternatively, they may be chemically synthesized using standard oligonucleotide synthesis techniques. In addition, template polynucleotides may be methylated or lack methylation. Template polynucleotides may be in the form of bacterial or yeast artificial chromosomes (BACs or YACs).

A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In other aspects, the template polynucleotide is delivered by viral and/or non-viral gene transfer methods. In some embodiments, the template polynucleotide is delivered to the cell via an adeno associated virus (AAV). Any AAV vector can be used, including, but not limited to, AAV1, AAV2, AAV3, AAV4, AAV5, AAV6, AAV7, AAV8 and combinations thereof. In some instances, the AAV comprises LTRs that are of a heterologous serotype in comparison with the capsid serotype (e.g., AAV2 ITRs with AAV5, AAV6, or AAV8 capsids). The template polynucleotide may be delivered using the same gene transfer system as used to deliver the nuclease (including on the same vector) or may be delivered using a different delivery system that is used for the nuclease. In some embodiments, the template polynucleotide is delivered using a viral vector (e.g., AAV) and the nuclease(s) is(are) delivered in mRNA form. The cell may also be treated with one or more molecules that inhibit binding of the viral vector to a cell surface receptor as described herein prior to, simultaneously and/or after delivery of the viral vector (e.g., carrying the nuclease(s) and/or template polynucleotide).

In some embodiments, the template polynucleotide is comprised in a viral vector, and is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some embodiments, the polynucleotide is comprised in a viral vector, and is between at or about 2500 and at or about 5000 nucleotides, at or about 3500 and at or about 4500 nucleotides, or at or about 3750 nucleotides and at or about 4250 nucleotides in length. In some embodiments, the polynucleotide is comprised in a viral vector, and is at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length.

In some embodiments, the template polynucleotide is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In some embodiments, the template polynucleotide comprises about 150 to 1000 nucleotides of homology on either side of the transgene and/or the target site. In some embodiments, the template polynucleotide comprises about 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at least 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at most 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene.

In some embodiments, the template polynucleotide is a lentiviral vector, e.g., an IDLV (integration deficiency lentivirus). In some embodiments, the template polynucleotide comprises about 500 to 1000 base pairs of homology on either side of the transgene and/or the target site. In some embodiments, the template polynucleotide comprises about 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises at least 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises no more than 300, 400, 500, 600, 700, 800, 900, 1000, 1500, or 2000 base pairs of homology 5′ of the target site or transgene, 3′ of the target site or transgene, or both 5′ and 3′ of the target site or transgene. In some embodiments, the template polynucleotide comprises one or more mutations, e.g., silent mutations, that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered. In some embodiments, the cDNA comprises one or more mutations, e.g., silent mutations that prevent Cas9 from recognizing and cleaving the template polynucleotide. The template polynucleotide may comprise, e.g., at least 1, 2, 3, 4, 5, 10, 20, or 30 silent mutations relative to the corresponding sequence in the genome of the cell to be altered. In some embodiments, the template polynucleotide comprises at most 2, 3, 4, 5, 10, 20, 30, or 50 silent mutations relative to the corresponding sequence in the genome of the cell to be altered.

The double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

The template polynucleotide may comprise any transgene of interest (exogenous sequence). Exemplary exogenous sequences include, but are not limited to any polypeptide coding sequence (e.g., cDNAs or fragments thereof), promoter sequences, enhancer sequences, epitope tags, marker genes, cleavage enzyme recognition sites and various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence.

In some embodiments, the transgene comprises a polynucleotide encoding any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the foregoing. In some embodiments, the exogenous sequence (transgene) comprises a polynucleotide encoding one or more recombinant receptor(s), e.g., functional non-TCR antigen receptors, chimeric antigen receptors (CARs), and T cell receptors (TCRs), such as transgenic TCRs, engineered TCRs or recombinant TCRs, and components of any of the foregoing.

In some embodiments, the coding sequences may be, for example, cDNAs. The exogenous sequences may also be a fragment of a transgene for linking with an endogenous gene sequence of interest. For example, a fragment of a transgene comprising sequence at the 3′ end of a gene of interest may be utilized to correct, via insertion or replacement, of a sequence encoding a mutation in the 3′ end of an endogenous gene sequence. Similarly, the fragment may comprise sequences similar to the 5′ end of the endogenous gene for insertion/replacement of the endogenous sequences to correct or modify such endogenous sequence. Additionally the fragment may encode a functional domain of interest (catalytic, secretory or the like) for linking in situ to an endogenous gene sequence to produce a fusion protein.

In some embodiments, the transgene further encodes one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. TCR or CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides includes truncated forms of growth factors or other receptors such as a truncated human epidermal growth factor receptor 2 (tHER2), a truncated epidermal growth factor receptor (tEGFR, exemplary tEGFR sequence set forth in SEQ ID NO:12 or 13) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 12 or 13 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12 or 13.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, such transgene further includes a T2A ribosomal skip element and/or a sequence encoding a marker such as a tEGFR sequence, e.g., downstream of the TCR or a CAR, such as set forth in SEQ ID NO: 12 or 13, respectively, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12 or 13.

In some embodiments, the template polynucleotide encodes a recombinant receptor that serves to direct the function of a T cell. Chimeric Antigen Receptors (CARs) are molecules designed to target immune cells to specific molecular targets expressed on cell surfaces. In their most basic form, they are receptors introduced to a cell that couple a specificity domain expressed on the outside of the cell to signaling pathways on the inside of the cell such that when the specificity domain interacts with its target, the cell becomes activated. Often CARs are made from variants of T-cell receptors (TCRs) where a specificity domain such as an scFv or some type of receptor is fused to the signaling domain of a TCR. These constructs are then introduced into a T cell allowing the T cell to become activated in the presence of a cell expressing the target antigen, resulting in the attack on the targeted cell by the activated T cell in a non-MHC dependent manner (see Chicaybam et at (2011) Int Rev Immunol 30:294-311). Alternatively, CAR expression cassettes can be introduced into an immune cell for later engraftment such that the CAR cassette is under the control of a T cell specific promoter (e.g., the FOXP3 promoter, see Mantel et. al (2006) J. Immunol 176: 3593-3602).

In an exemplary embodiment, the template polynucleotide is included as an adeno-associated virus (AAV) vector construct, containing a nucleic acid sequence encoding a recombinant TCR α and TCR β chains under the control of a constitutive promoter, flanked by homology arms of about 600 base pairs each on the 5′ and 3′ side of the nucleic acid sequence encoding the recombinant TCR for targeting at exon 1 of the endogenous TRAC gene. Exemplary 5′ homology arm for targeting at TRAC include the sequence set forth in SEQ ID NO:124. Exemplary 3′ homology arm for targeting at TRAC include the sequence set forth in SEQ ID NO:125.

Construction of such expression cassettes, following the teachings of the present specification, utilizes methodologies well known in molecular biology (see, for example, Ausubel or Maniatis). Before use of the expression cassette to generate a transgenic animal, the responsiveness of the expression cassette to the stress-inducer associated with selected control elements can be tested by introducing the expression cassette into a suitable cell line (e.g., primary cells, transformed cells, or immortalized cell lines).

Targeted insertion of non-coding nucleic acid sequence may also be achieved. Sequences encoding antisense RNAs, RNAi, shRNAs and micro RNAs (miRNAs) may also be used for targeted insertions. In additional embodiments, the template polynucleotide may comprise non-coding sequences that are specific target sites for additional nuclease designs. Subsequently, additional nucleases may be expressed in cells such that the original template polynucleotide is cleaved and modified by insertion of another template polynucleotide of interest. In this way, reiterative integrations of template polynucleotides may be generated allowing for trait stacking at a particular locus of interest, e.g., TRAC, TRBC1 and/or TRBC2 gene loci.

In some embodiments, the polynucleotide contains the structure: [5′ homology arm]-[transgene sequence]-[3′ homology arm]. In some embodiments, the polynucleotide contains the structure: [5′ homology arm]-[multicistronic element]-[transgene sequence]-[3′ homology arm]. In some embodiments, the polynucleotide contains the structure: [5′ homology arm]-[promoter]-[transgene sequence]-[3′ homology arm].

4. Delivery of Template Polynucleotides

In some embodiments, the polynucleotide, e.g., a polynucleotide such as a template polynucleotide encoding the chimeric receptor, are introduced into the cells in nucleotide form, e.g., as a polynucleotide or a vector. In particular embodiments, the polynucleotide contains a transgene that encodes the chimeric receptor or a portion thereof.

In some embodiments, the template polynucleotide is introduced into the cell for engineering, in addition to the agent(s) capable of inducing a targeted genetic disruption, e.g., nuclease and/or gRNAs. In some embodiments, the template polynucleotide(s) may be delivered prior to, simultaneously or after the agent(s) capable of inducing a targeted genetic disruption is introduced into a cell. In some embodiments, the template polynucleotide(s) are delivered simultaneously with the agents. In some embodiments, the template polynucleotides are delivered prior to the agents, for example, seconds to hours to days before the agents, including, but not limited to, 1 to 60 minutes (or any time therebetween) before the agents, 1 to 24 hours (or any time therebetween) before the agents or more than 24 hours before the agents. In some embodiments, the template polynucleotides are delivered after the agents, seconds to hours to days after the agents, including immediately after delivery of the agent, e.g., between or between about between 30 seconds to 4 hours, such as about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after delivery of the agents and/or preferably within 4 hours of delivery of the agents. In some embodiments, the template polynucleotide is delivered more than 4 hours after delivery of the agents. In some embodiments, the template polynucleotides are delivered after the agents, for example, including, but not limited to, within 1 second to 60 minutes (or any time therebetween) after the agents, 1 to 4 hours (or any time therebetween) after the agents or more than 4 hours after the agents.

In some embodiments, the template polynucleotides may be delivered using the same delivery systems as the agent(s) capable of inducing a targeted genetic disruption, e.g., nuclease and/or gRNAs. In some embodiments, the template polynucleotides may be delivered using different same delivery systems as the agent(s) capable of inducing a targeted genetic disruption, e.g., nuclease and/or gRNAs. In some embodiments, the template polynucleotide is delivered simultaneously with the agent(s). In other embodiments, the template polynucleotide is delivered at a different time, before or after delivery of the agent(s). Any of the delivery method described herein in Section I.A.3 (e.g., in Tables 7 and 8) for delivery of nucleic acids in the agent(s) capable of inducing a targeted genetic disruption, e.g., nuclease and/or gRNAs, can be used to deliver the template polynucleotide.

In some embodiments, the one or more agent(s) and the template polynucleotide are delivered in the same format or method. For example, in some embodiments, the one or more agent(s) and the template polynucleotide are both comprised in a vector, e.g., viral vector. In some embodiments, the template polynucleotide is encoded on the same vector backbone, e.g. AAV genome, plasmid DNA, as the Cas9 and gRNA. In some aspects, the one or more agent(s) and the template polynucleotide are in different formats, e.g., ribonucleic acid-protein complex (RNP) for the Cas9-gRNA agent and a linear DNA for the template polynucleotide, but they are delivered using the same method. In some aspects, the one or more agent(s) and the template polynucleotide are in different formats, e.g., ribonucleic acid-protein complex (RNP) for the Cas9-gRNA agent and the template polynucleotide is in contained in an AAV vector, and the RNP is delivered using a physical delivery method (e.g., electroporation) and the template polynucleotide is delivered via transduction of AAV viral preparations. In some aspects, the template polynucleotide is delivered immediately after, e.g., within about 1, 2, 3, 4, 5, 10, 20, 30, 40, 50 or 60 minutes after, the delivery of the one or more agent(s).

In some embodiments, the template polynucleotide is a linear or circular nucleic acid molecule, such as a linear or circular DNA or linear RNA, and can be delivered using any of the methods described in Section I.A.3 herein (e.g., Tables 7 and 8) for delivering nucleic acid molecules into the cell.

In particular embodiments, the polynucleotide, e.g., the template polynucleotide, are introduced into the cells in nucleotide form, e.g., as or within a non-viral vector. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (e.g., as described in Lee, et al. (2012) Nano Lett 12: 6322-27), lipid-mediated transfection, peptide-mediated delivery, e.g., cell-penetrating peptides, or a combination thereof. In some embodiments, the non-viral polynucleotide is delivered into the cell by a non-viral method described herein, such as a non-viral method listed in Table 8 herein.

In some embodiments, the template polynucleotide sequence can be comprised in a vector molecule containing sequences that are not homologous to the region of interest in the genomic DNA. In some embodiments, the virus is a DNA virus (e.g., dsDNA or ssDNA virus). In some embodiments, the virus is an RNA virus (e.g., ssRNA or dsRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein.

In some embodiments, the template polynucleotide can be transferred into cells using recombinant infectious virus particles, such as, e.g., vectors derived from simian virus 40 (SV40), adenoviruses, adeno-associated virus (AAV). In some embodiments, the template polynucleotide are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557) or HIV-1 derived lentiviral vectors.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

In some embodiments, the template polynucleotides and nucleases may be on the same vector, for example an AAV vector (e.g., AAV6). In some embodiments, the template polynucleotides are delivered using an AAV vector and the agent(s) capable of inducing a targeted genetic disruption, e.g., nuclease and/or gRNAs are delivered as a different form, e.g., as mRNAs encoding the nucleases and/or gRNAs. In some embodiments, the template polynucleotides and nucleases are delivered using the same type of method, e.g., a viral vector, but on separate vectors. In some embodiments, the template polynucleotides are delivered in a different delivery system as the agents capable of inducing a genetic disruption, e.g., nucleases and/or gRNAs. In some embodiments, the template polynucleotide is excised from a vector backbone in vivo, e.g., it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, e.g., in a vector or a linear nucleic acid molecule, e.g., linear DNA. Types or nucleic acids and vectors for delivery include any of those described in Section III herein.

C. Assessment of Engineered T Cells and Compositions

In some of the embodiments, the methods include assessing the T cells or T cell compositions engineered to express the recombinant TCRs for particular properties. For example, the methods include assessing the T cells or T cell compositions for cell surface expression of the recombinant TCR and/or for recognition of a peptide in the context of an MHC molecule. For example, in any of the embodiments provided herein, functional assays can be performed on the T cells or T cell compositions expressing the exogenous recombinant TCR, generated or produced using any of the methods provided herein. In some embodiments, assays to detect functionality of the TCRs and activity of TCR signaling can also be performed.

In some embodiments, the T cells or T cell compositions are assessed for cell surface expression of the recombinant TCR, e.g., for the ability or capability to express a functional TCR, such as TCRαβ, on the surface of the cell. In some embodiments, the T cells or T cell compositions are assessed for the ability or capability of the expressed TCRs for recognition of a peptide in the context of an MHC molecule, e.g., binding antigens or epitopes in the context of an MHC molecule. In some embodiments, the methods include assessing the T cells or T cell compositions for T cell activity and/or functionality. In some embodiments, the T cells or T cell compositions are assessed for is expression of the marker for transduction or introduction of the transgene.

In some embodiments, the T cells or T cell compositions are assessed for cell surface expression of the recombinant TCR, e.g., for the ability or capability to express a functional TCR, such as TCRαβ, on the surface of the cell. In some embodiments, assessing surface expression of the TCR comprises contacting cells of each T cell composition with a binding reagent specific for the TCRα chain or the TCRβ chain and assessing binding of the reagent to the cells. In some embodiments, the binding reagent is an antibody. In some embodiments, the binding reagent is detectably labeled, optionally fluorescently labeled, directly or indirectly. In some embodiments, the binding reagent is a fluorescently labeled antibody, such as an antibody labeled directly or indirectly. In some embodiments, the binding reagent is an anti-pan-TCR Vβ antibody or is an anti-pan-TCR Vα antibody. In some embodiments, the binding reagent recognizes a specific family of chains. In some embodiments, the binding reagent is an anti-TCR Vβ or anti-TCR Vα antibody that recognizes or binds a specific family, such as an anti-TCR Vβ22 antibody or an anti-TCR Vβ2 antibody. In some embodiments, the expression is detected using antibodies against one or more common portions, e.g., extracellular portions, of the TCR. For example, expression of TCR on the surface of the cell can be detected using pan-reactive anti-TCR antibodies, such as a pan-reactive TCR Vβ antibody, or a pan-reactive TCR Vα antibody. Pan-reactive antibodies can detect the TCR regions regardless of its antigen or epitope binding specificity. In some embodiments, the cells are stained using a binding reagent, e.g., a labeled antibody that recognizes TCR cell surface expression, such as a fluorescently labeled pan-reactive TCR Vα antibody or antigen-binding fragment thereof, and detecting using fluorescence microscopy, flow cytometry or fluorescence activated cell sorting (FACS). In some embodiments, T cells or T cell compositions that express the TCR on the surface of the cell, e.g., stain positive using pan-reactive anti-TCR antibodies, such as a pan-reactive TCR Vβ antibody, or a pan-reactive TCR Vα antibody, are identified and/or selected.

In some embodiments, the T cells or T cell compositions are assessed for the ability or capability of the expressed TCRs for recognition of a peptide in the context of an MHC molecule, e.g., binding antigens or epitopes in the context of an MHC molecule. For example, in some embodiments, assessing the T cells or T cell compositions for recognition of a peptide in the context of an MHC molecule comprises: (1) contacting the cells or the cells of the T cell composition with a target antigen comprising a peptide-MHC complex and (2) determining the presence or absence of binding of the peptide-MHC complex to the cells and/or determining the presence or absence of T cell activation of the TCR-expressing cells upon engagement with the peptide-MHC complex.

In some embodiments, the T cells or T cell compositions to which nucleic acid sequences encoding recombinant TCRs are introduced, are tested by confirming that the recombinant TCRs bind to the desired or known antigen, such as a TCR ligand (MHC-peptide complex). In some embodiments, the binding of the cells to an antigen or an epitope can be detected by a number of methods. In some methods, a particular antigen, e.g. MHC-peptide complex, can be detectably labeled so that binding to the receptor, e.g. TCR, can be visualized. In some embodiments, the antigen can be soluble or expressed in a soluble form. In some embodiments, the TCR ligand can be a peptide-MHC tetramer, and in some cases the peptide-MHC tetramer can be detectably labeled, such as labeled with a fluorescent label. The peptide-MHC tetramer can be labeled directly or indirectly. In some embodiments, the fluorescent label can be detected using flow cytometry or fluorescence activated cell sorting (FACS) or fluorescence microscopy. In some embodiments, the methods include identifying one or more T cells or T cell compositions that recognize the peptide in the context of the MHC molecule, i.e. peptide-MHC complex.

In some cases, the binding of TCR, such as a recombinant TCR, to a peptide epitope, e.g. in complex with an MHC, results in or effects a functional property of the interaction. For example, a T cell expressing a TCR, such as a recombinant TCR, when specifically bound to an MHC-peptide complex, can induces a signal transduction pathway in the cell, induce cellular expression or secretion of an effector molecule (e.g. cytokine), reporter or other detectable readout of the interaction, or induce T cell activation or a T cell response, such as T cell proliferation, cytokine production, a cytotoxic T cell response or other response. In some embodiments, the TCR, such as a recombinant TCR, can specifically bind to and immunologically recognize a peptide epitope, such that binding to the peptide epitope elicits an immune response.

Methods of testing a TCR for the ability to recognize a peptide epitope of a target polypeptide and for antigen specificity are known. In some embodiments, T cells or T cell compositions produced in accord with the provided method are contacted with a peptide-MHC complex, either in soluble form or via co-culture with peptide pulsed antigen presenting cells (e.g. T2 cells or other known antigen presenting cell that matches the MHC allele of the recombinant TCR). Exemplary antigens and MHC alleles of recombinant TCRs are described in Section III. In some embodiments, the methods include assessment of properties such as functional properties, of the exogenous recombinant TCR. In some embodiments, the method includes assessing T cell activation via the exogenous recombinant TCR, for example, determining the presence or absence of T cell activation of the TCR-expressing cells upon engagement with the peptide-MHC complex. In some embodiments, a readout of T cell activation by such methods includes release of cytokines (e.g., interferon-γ, granulocyte/monocyte colony stimulating factor (GM-CSF), tumor necrosis factor a (TNF-α) or interleukin 2 (IL-2)). In addition, TCR function can be evaluated by measurement of cellular cytotoxicity, as described in Zhao et al., J. Immunol., 174:4415-4423 (2005).

In some embodiments, assessing T cell activation includes assessing activity or expression of a nucleic acid molecule encoding a reporter, e.g. a T cell activation reporter, assessing release of cytokines, and/or assessing functional activity of the T cell.

In some embodiments, the one or more assays involve one or more instrumentation, type of result or analysis, and/or read-outs. In some embodiments, the one or more assays are performed using fluorescently labeled reagents, such as antibodies directly or indirectly labeled with fluorophores, and are detected using a flow cytometry or fluorescence activated cell sorting (FACS) instrument. For example, for flow cytometry or FACS, multiple different fluorophores that have different peak excitation and emission wavelength can be detected. Thus, multiple fluorophore labels can be used to assess multiple properties, for example, expression of the TCR, recognition of the peptide in the context of an MHC molecule and/or T cell activation reporter expression, in one experimental reaction. In some embodiments, the one or more assays are performed in a high-throughput, multiplexed and/or large-scale manner.

In some embodiments, the methods further include assessing aspects of T cell activation, such as assessing release of cytokines and/or assessing functional activity of the T cell, e.g., cytolytic activity and/or helper T cell activity. In some embodiments, the assessments can be performed in T cells or T cell compositions generated using the embodiments described herein.

In some embodiments, the functional assays are performed in primary T cells, such as those isolated directly from a subject and/or isolated from a subject and frozen, such as primary CD4+ and/or CD8+ T cells, that have been engineered employing the embodiments provided herein.

In some embodiments, the methods include performing functional assays or detecting function of the TCR or the T cell. For example, functional assays for determining TCR activity or T cell activity include detection of cytokine secretion, cytolytic activity and/or helper T cell activity. For example, assessment of T cell activation includes assessing release of cytokines, and/or assessing functional activity of the T cell. In some embodiments, upon binding of the TCR to an antigen or an epitope, the cytoplasmic domain or intracellular signaling domain of the TCR activates at least one of the normal effector functions or responses of an immune cell, e.g., T cell engineered to express the TCR. For example, in some contexts, the TCR induces a function of a T cell such as cytolytic activity and/or helper T cell activity, such as secretion of cytokines or other factors. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In some embodiments, T cells or T cell compositions containing the exogenous recombinant TCRs are assessed for an immunological readout, such as using a T cell assay. In some embodiments, the TCR-expressing cells can activate a CD8+ T cell response. In some embodiments, CD8+ T cell responses can be assessed by monitoring CTL reactivity using assays that include, but are not limited to, target cell lysis via ⁵¹Cr release, target cell lysis assays using real-time imaging reagents, target cell lysis assays using apoptosis detection reagent (e.g., Caspase 3/7 reagent), or detection of interferon gamma release, such as by enzyme-linked immunosorbent spot assay (ELISA), intracellular cytokine staining or ELISPOT. In some embodiments, the TCR-expressing cells can activate a CD4+ T cell response. In some aspects, CD4+ T cell responses can be assessed by assays that measure proliferation, such as by incorporation of [³H]-thymidine into cellular DNA and/or by the production of cytokines, such as by ELISA, intracellular cytokine staining or ELISPOT. In some cases, the cytokine can include, for example, interleukin-2 (IL-2), interferon-gamma (IFN-gamma), interleukin-4 (IL-4), TNF-α, interleukin-6 (IL-6), interleukin-10 (IL-10), interleukin-12 (IL-12) or TGF β. In some embodiments, recognition or binding of the peptide epitope, such as a MHC class I or class II epitope, by the TCR can elicit or activate a CD8+ T cell response and/or a CD4+ T cell response.

### II. CELLS FOR GENETIC ENGINEERING

In some of the provided embodiments, the cells for engineering are immune cells, such as T cells. Provided are genetically engineered cells or cell populations wherein one or more of the cells contain a knock-out of one or more endogenous TCR genes and recombinant receptor-encoding nucleic acids and/or other transgene that are integrated into one or more of the endogenous TCR genes. Also provided are populations or compositions of such cells, compositions containing such cells and/or enriched for cells that are engineered using the provided methods.

In some embodiments, the cells for engineering include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{Cm}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as T_{H}1 cells, T_{H}2 cells, T_{H}3 cells, T_{H}17 cells, T_{H}9 cells, T_{H}22 cells, follicular helper T cells, alpha/beta T cells, and delta/gamma T cells. In some embodiments, the cell is a regulatory T cell (Treg). In some embodiments, the cell further comprises a recombinant FOXP3 or variant thereof.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for engineering may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished in a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻ CD8⁺ and/or CD62L⁺ CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or ROR1, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher © Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain aspects, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International PCT Publication No. WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10:1567-1573; and Godin et al. (2008) J Biophoton. 1(5):355-376). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of nucleic acids encoding a recombinant receptor, e.g., a recombinant TCR.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling region of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

Various methods for the introduction of genetically engineered components, e.g., agents for inducing a genetic disruption and/or nucleic acids encoding recombinant receptors, e.g., CARs or TCRs, are known and may be used with the provided methods and compositions. Exemplary methods include those for transfer of nucleic acids encoding the polypeptides or receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell, or any delivery methods described in Section I.A herein. Other approaches and vectors for transfer of the nucleic acids encoding the recombinant products are those described, e.g., in WO2014055668 and U.S. Pat. No. 7,446,190.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, it may be desired to safeguard against the potential that overexpression of a stimulatory factor (for example, a lymphokine or a cytokine) could potentially result in an unwanted outcome or lower efficacy in a subject, such as a factor associated with toxicity in a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 11:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, the cells, e.g., T cells, may be engineered either during or after expansion. This engineering for the introduction of the gene of the desired polypeptide or receptor can be carried out with any suitable retroviral vector, for example. The genetically modified cell population can then be liberated from the initial stimulus (the CD3/CD28 stimulus, for example) and subsequently be stimulated with a second type of stimulus (e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

As described herein, in some embodiments, the cells are incubated and/or cultured prior to or in connection with genetic engineering. The incubation steps can include culture, cultivation, stimulation, activation, propagation and/or freezing for preservation, e.g. cryopreservation.

### III. NUCLEIC ACIDS, VECTORS AND DELIVERY

In some embodiments, the one or more agent for genetic disruption and/or template polynucleotides, e.g., template polynucleotides containing transgene encoding the recombinant receptor or antigen-binding fragment or chain thereof or the one or more second template polynucleotides, are introduced into the cells in nucleic acid form, e.g., as polynucleotides and/or vectors. As described in Section I herein, the components for engineering can be delivered in various forms using various delivery methods, including as polynucleotides encoding the components. Also provided are one or more polynucleotides (e.g., nucleic acid molecules) encoding one or more components of the one or more agent(s) capable of inducing a genetic disruption, and/or one or more template polynucleotides containing transgene, and vectors for genetically engineering cells for targeted integration of the transgene.

In some embodiments, provided are template polynucleotides, e.g., template polynucleotides for targeting transgene at a specific genomic target location, e.g., at the TRAC, TRBC1 and/or TRBC2 locus. In some embodiments, provided are any template polynucleotides described in Section I.B herein. In some embodiments, the template polynucleotide contains transgene that include nucleic acid sequences that encode a recombinant receptor or other polypeptides and/or factors, and homology arms for targeted integration. In some embodiments, the template polynucleotide can be contained in a vector.

In some embodiments, agents capable of inducing a genetic disruption can be encoded in one or more polynucleotides. In some embodiments, the component of the agents, e.g., Cas9 molecule and/or a gRNA molecule, can be encoded in one or more polynucleotides, and introduced into the cells. In some embodiments, the polynucleotide encoding one or more component of the agents can be included in a vector.

In some embodiments, a vector may comprise a sequence that encodes a Cas9 molecule and/or a gRNA molecule and/or template polynucleotides. A vector may also comprise a sequence encoding a signal peptide (e.g., for nuclear localization, nucleolar localization, mitochondrial localization), fused, e.g., to a Cas9 molecule sequence. For example, a vector may comprise a nuclear localization sequence (e.g., from SV40) fused to the sequence encoding the Cas9 molecule.

One or more regulatory/control elements, e.g., a promoter, an enhancer, an intron, a polyadenylation signal, a Kozak consensus sequence, internal ribosome entry sites (IRES), a 2A sequence, and splice acceptor or donor can be included in the vectors. In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter).

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (sequence set forth in SEQ ID NO:18 or 126; see Challita et al. (1995) J. Virol. 69(2):748-755). In some embodiments, the promoter is a tissue-specific promoter. In another embodiment, the promoter is a viral promoter. In another embodiment, the promoter is a non-viral promoter. In some embodiments, exemplary promoters can include, but are not limited to, human elongation factor 1 alpha (EF1α) promoter (sequence set forth in SEQ ID NO:4 or 5) or a modified form thereof (EF1α promoter with HTLV1 enhancer; sequence set forth in SEQ ID NO: 127) or the MND promoter (sequence set forth in SEQ ID NO:18 or 126). In some embodiments, the polynucleotide and/or vector does not include a regulatory element, e.g. promoter.

In some embodiments, the vector or delivery vehicle is a viral vector (e.g., for generation of recombinant viruses). In some embodiments, the virus is a DNA virus (e.g., dsDNA or ssDNA virus). In some embodiments, the virus is an RNA virus (e.g., ssRNA virus). Exemplary viral vectors/viruses include, e.g., retroviruses, lentiviruses, adenovirus, adeno-associated virus (AAV), vaccinia viruses, poxviruses, and herpes simplex viruses, or any of the viruses described elsewhere herein.

In some embodiments, the virus infects dividing cells. In another embodiment, the virus infects non-dividing cells. In another embodiment, the virus infects both dividing and non-dividing cells. In another embodiment, the virus can integrate into the host genome. In another embodiment, the virus is engineered to have reduced immunity, e.g., in human. In another embodiment, the virus is replication-competent. In another embodiment, the virus is replication-defective, e.g., having one or more coding regions for the genes necessary for additional rounds of virion replication and/or packaging replaced with other genes or deleted. In another embodiment, the virus causes transient expression of the Cas9 molecule and/or the gRNA molecule for the purposes of transient induction of genetic disruption. In another embodiment, the virus causes long-lasting, e.g., at least 1 week, 2 weeks, 1 month, 2 months, 3 months, 6 months, 9 months, 1 year, 2 years, or permanent expression, of the Cas9 molecule and/or the gRNA molecule. The packaging capacity of the viruses may vary, e.g., from at least about 4 kb to at least about 30 kb, e.g., at least about 5 kb, 10 kb, 15 kb, 20 kb, 25 kb, 30 kb, 35 kb, 40 kb, 45 kb, or 50 kb.

In some embodiments, the polynucleotide containing the agent(s) and/or template polynucleotide is delivered by a recombinant retrovirus. In another embodiment, the retrovirus (e.g., Moloney murine leukemia virus) comprises a reverse transcriptase, e.g., that allows integration into the host genome. In some embodiments, the retrovirus is replication-competent. In another embodiment, the retrovirus is replication-defective, e.g., having one of more coding regions for the genes necessary for additional rounds of virion replication and packaging replaced with other genes, or deleted.

In some embodiments, the polynucleotide containing the agent(s) and/or template polynucleotide is delivered by a recombinant lentivirus. For example, the lentivirus is replication-defective, e.g., does not comprise one or more genes required for viral replication. In some embodiments, the lentivirus is an HIV-derived lentivirus.

In some embodiments, the polynucleotide containing the agent(s) and/or template polynucleotide is delivered by a recombinant adenovirus. In another embodiment, the adenovirus is engineered to have reduced immunity in humans.

In some embodiments, the polynucleotide containing the agent(s) and/or template polynucleotide is delivered by a recombinant AAV. In some embodiments, the AAV can incorporate its genome into that of a host cell, e.g., a target cell as described herein. In another embodiment, the AAV is a self-complementary adeno-associated virus (scAAV), e.g., a scAAV that packages both strands which anneal together to form double stranded DNA. AAV serotypes that may be used in the disclosed methods, include AAV1, AAV2, modified AAV2 (e.g., modifications at Y444F, Y500F, Y730F and/or S662V), AAV3, modified AAV3 (e.g., modifications at Y705F, Y731F and/or T492V), AAV4, AAV5, AAV6, modified AAV6 (e.g., modifications at S663V and/or T492V), AAV7, AAV8, AAV 8.2, AAV9, AAV.rh10, modified AAV.rh10, AAV.rh32/33, modified AAV.rh32/33, AAV.rh43, modified AAV.rh43, AAV.rh64R1, modified AAV.rh64R1, and pseudotyped AAV, such as AAV2/8, AAV2/5 and AAV2/6 can also be used in the disclosed methods.

In some embodiments, the polynucleotide containing the agent(s) and/or template polynucleotide is delivered by a hybrid virus, e.g., a hybrid of one or more of the viruses described herein.

A packaging cell is used to form a virus particle that is capable of infecting a target cell. Such a cell includes a 293 cell, which can package adenovirus, and a ψ2 cell or a PA317 cell, which can package retrovirus. A viral vector used in gene therapy is usually generated by a producer cell line that packages a nucleic acid vector into a viral particle. The vector typically contains the minimal viral sequences required for packaging and subsequent integration into a host or target cell (if applicable), with other viral sequences being replaced by an expression cassette encoding the protein to be expressed, e.g., Cas9. For example, an AAV vector used in gene therapy typically only possesses inverted terminal repeat (ITR) sequences from the AAV genome which are required for packaging and gene expression in the host or target cell. The missing viral functions are supplied in trans by the packaging cell line. Henceforth, the viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, the viral vector has the ability of cell type recognition. For example, the viral vector can be pseudotyped with a different/alternative viral envelope glycoprotein; engineered with a cell type-specific receptor (e.g., genetic modification of the viral envelope glycoproteins to incorporate targeting ligands such as a peptide ligand, a single chain antibody, a growth factor); and/or engineered to have a molecular bridge with dual specificities with one end recognizing a viral glycoprotein and the other end recognizing a moiety of the target cell surface (e.g., ligand-receptor, monoclonal antibody, avidin-biotin and chemical conjugation).

In some embodiments, the viral vector achieves cell type specific expression. For example, a tissue-specific promoter can be constructed to restrict expression of the transgene (Cas9 and gRNA) in only a specific target cell. The specificity of the vector can also be mediated by microRNA-dependent control of transgene expression. In some embodiments, the viral vector has increased efficiency of fusion of the viral vector and a target cell membrane. For example, a fusion protein such as fusion-competent hemagglutinin (HA) can be incorporated to increase viral uptake into cells. In some embodiments, the viral vector has the ability of nuclear localization. For example, a virus that requires the breakdown of the nuclear membrane (during cell division) and therefore will not infect a non-diving cell can be altered to incorporate a nuclear localization peptide in the matrix protein of the virus thereby enabling the transduction of non-proliferating cells.

### IV. RECOMBINANT RECEPTORS

In some embodiments, the transgene for targeted integration encodes a recombinant receptor or an antigen-binding fragment thereof or a chain thereof. In some embodiments, the recombinant receptor is a recombinant antigen receptor, or a recombinant receptor that binds to an antigen. In some embodiments, the recombinant receptor is a recombinant or engineered T cell receptor (TCR), that is different from the endogenous TCR encoded by the T cell. In some embodiments, the recombinant receptor is a chimeric antigen receptor (CAR) or a TCR-like CAR. In some embodiments, the transgene can encode a domain, region or chain of a recombinant receptor, and one or more second transgenes can encode other domains, regions or chains of the recombinant receptor. In some embodiments, the provided polynucleotides, vectors, compositions, methods, articles of manufacture, and/or kits are useful for engineering cells that express a recombinant TCR or an antigen-binding fragment thereof.

In some embodiments, the provided recombinant receptors, e.g., TCRs or CARs, are capable of binding to or recognizing, such as specifically binding to or recognizing, an antigen that is associated with, specific to, and/or expressed on a cell or tissue of a disease, disorder or condition, such as a cancer or a tumor. In some aspects, the antigen is in a form of a peptide, e.g., is a peptide antigen or a peptide epitope. In some embodiments, the provided TCRs bind to, such as specifically bind to, an antigen that is a peptide, in the context of a major histocompatibility (MHC) molecule.

The observation that recombinant receptor binds to an antigen, e.g., peptide antigen, or specifically binds to an antigen, e.g., peptide antigen, does not necessarily mean that it binds to an antigen of every species. For example, in some embodiments, features of binding to the antigen, e.g., peptide antigen in the context of an MHC, such as the ability to specifically bind thereto and/or to compete for binding thereto with a reference binding molecule or a receptor, and/or to bind with a particular affinity or compete to a particular degree, in some embodiments, refers to the ability with respect to a human antigen and the recombinant receptor may not have this feature with respect to the antigen from another species, such as mouse. In some aspects, the extent of binding of the recombinant receptor or an antigen-binding fragment thereof to an unrelated antigen or protein, such as an unrelated peptide antigen, is less than at or about 10% of the binding of the recombinant receptor or an antigen-binding fragment thereof to the antigen, e.g., cognate antigen as measured, e.g., by a radioimmunoassay (RIA), a peptide titration assay or a reporter assay.

A. T Cell Receptors (TCRs)

In some embodiments, the recombinant receptor that is introduced into the cell is a T cell receptor (TCR) or an antigen-binding fragment thereof.

In some embodiments, a “T cell receptor” or “TCR” is a molecule that contains α and β chains (also known as TCRα and TCRβ, respectively) or γ and δ chains (also known as TCRγ and TCRδ, respectively), or antigen-binding portions thereof, and which is capable of specifically binding to an antigen, e.g., a peptide antigen or peptide epitope, bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Typically, specific binding of recombinant receptor, e.g. TCR, to a peptide epitope, e.g. in complex with an MHC, is governed by the presence of an antigen-binding site containing one or more complementarity determining regions (CDRs). In general, it is understood that specifically binds does not mean that the particular peptide epitope, e.g. in complex with an MHC, is the only thing to which the MHC-peptide molecule may bind, since non-specific binding interactions with other molecules may also occur. In some embodiments, binding of recombinant receptor to a peptide in the context of an MHC molecule is with a higher affinity than binding to such other molecules, e.g. another peptide in the context of an MHC molecule or an irrelevant (control) peptide in the context of an MHC molecule, such as at least about 2-fold, at least about 10-fold, at least about 20-fold, at least about 50-fold, or at least about 100-fold higher than binding affinity to such other molecules.

In some embodiments, the recombinant receptor, e.g., TCR, can be assessed for safety or off-target binding activity using any of a number of known screening assays. In some embodiments, generation of an immune response to a particular recombinant receptor, e.g., TCR, can be measured in the presence of cells that are known not to express the target peptide epitope, such as cells derived from normal tissue(s), allogenic cell lines that express one or more different MHC types or other tissue or cell sources. In some embodiments, the cells or tissues include normal cells or tissues. In some embodiments, the binding to cells can be tested in 2 dimensional cultures. In some embodiments, the binding to cells can be tested in 3 dimensional cultures. In some embodiments, as a control, the tissues or cells can be ones that are known to express the target epitope. The immune response can be assessed directly or indirectly, such as by assessing activation of immune cells such as T cells (e.g. cytotoxic activity), production of cytokine (e.g. interferon gamma), or activation of a signaling cascade, such as by reporter assays.

Unless otherwise stated, the term “TCR” should be understood to encompass full TCRs as well as antigen-binding portions or antigen-binding fragments thereof. In some embodiments, the TCR is an intact or full-length TCR, such as a TCR containing the alpha (TCRα) chain and beta (TCRβ) chain. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α (V_{α}) chain and variable β (V_{β}) chain of a TCR, or antigen-binding fragments thereof sufficient to form a binding site for binding to a specific MHC-peptide complex.

In some embodiments, the variable domains of the TCR contain complementarity determining regions (CDRs), which generally are the primary contributors to antigen recognition and binding capabilities and specificity of the peptide, MHC and/or MHC-peptide complex. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the α chain can interact with the N-terminal part of certain antigenic peptides. In some contexts, CDR1 of the β chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the β-chain can contain a further hypervariable region (CDR4 or HVR4), which generally is involved in superantigen binding and not antigen recognition (Kotb (1995) Clinical Microbiology Reviews, 8:411-426).

In some embodiments, the α-chain and/or β-chain of a TCR also can contain a constant domain, a transmembrane domain and/or a short cytoplasmic tail (see, e.g., Janeway et al., Immunobiology: The Immune System in Health and Disease, 3^{rd} Ed., Current Biology Publications, p. 4:33, 1997). In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM and generally are involved in the signaling capacity of the TCR complex.

In some embodiments, the various domains or regions of a TCR can be determined. In some cases, the exact locus of a domain or region can vary depending on the particular structural or homology modeling or other features used to describe a particular domain. It is understood that reference to amino acids, including to a specific sequence set forth as a SEQ ID NO used to describe domain organization of a recombinant receptor, e.g., TCR, are for illustrative purposes and are not meant to limit the scope of the embodiments provided. In some cases, the specific domain (e.g. variable or constant) can be several amino acids (such as one, two, three or four) longer or shorter. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). Using this system, the CDR1 sequences within a TCR Vα chains and/or Vβ chain correspond to the amino acids present between residue numbers 27-38, inclusive, the CDR2 sequences within a TCR Vα chain and/or Vβ chain correspond to the amino acids present between residue numbers 56-65, inclusive, and the CDR3 sequences within a TCR Vα chain and/or Vβ chain correspond to the amino acids present between residue numbers 105-117, inclusive.

In some embodiments, the α chain and β chain of a TCR each further contain a constant domain. In some embodiments, the α chain constant domain (Ca) and β chain constant domain (Cβ) individually are mammalian, such as is a human or murine constant domain. In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

In some embodiments, each of the Cα and Cβ domains is human. In some embodiments, the Cα is encoded by the TRAC gene (IMGT nomenclature) or is a variant thereof. In some embodiments, the Cα has or comprises the sequence of amino acids set forth in SEQ ID NO: 19 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 19. In some embodiments, the Cα has or comprises the sequence of amino acids set forth in any of SEQ ID NO:19. In some embodiments, the Cα has or comprises the sequence of amino acids, e.g., mature polypeptide, encoded by the nucleic acid sequence set forth in SEQ ID NO:1 or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids, e.g., mature polypeptide, encoded by the nucleic acid sequence set forth in SEQ ID NO:1. In some embodiments, the Cβ is encoded by TRBC1 or TRBC2 genes (IMGT nomenclature) or is a variant thereof. In some embodiments, the Cβ has or comprises the sequence of amino acids set forth in SEQ ID NO:20 or 21 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 20 or 21. In some embodiments, the Cβ has or comprises the sequence of amino acids set forth in SEQ ID NO: 20 or 21. In some embodiments, the Cβ has or comprises the sequence of amino acids, e.g., mature polypeptide, encoded by the nucleic acid sequence set forth in SEQ ID NO:2 or 3 or a sequence of amino acids that exhibits at least 85%, 86%, 87%, 88%, 89%, 90%, 91%, 92%, 93%, 94%, 95%, 96%, 97%, 98%, 99% or more sequence identity to the sequence of amino acids, e.g., mature polypeptide, encoded by the nucleic acid sequence set forth in SEQ ID NO:2 or 3.

In some embodiments, any of the provided TCRs or antigen-binding fragments thereof can be a human/mouse chimeric TCR. In some cases, the TCR or antigen-binding fragment thereof have α chain and/or a β chain comprising a mouse constant region. In some aspects, the Cα and/or Cβ regions are mouse constant regions. In some embodiments, the Cα is a mouse constant region that is or comprises the sequence of amino acids set forth in SEQ ID NO: 14, 15, 121 or 122 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 14, 15, 121 or 122. In some embodiments, the Cα is or comprises the sequence of amino acids set forth in SEQ ID NO: 14, 15, 121 or 122. In some embodiments, the Cβ is a mouse constant region that is or comprises the sequence of amino acids set forth in SEQ ID NO: 16, 17 or 123 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 16, 17 or 123. In some embodiments, the Cβ is or comprises the sequence of amino acids set forth in SEQ ID NO: 16, 17 or 123.

In some of any such embodiments, the TCR or antigen-binding fragment thereof containing one or more modifications in the α chain and/or β chain such that when the TCR or antigen-binding fragment thereof is expressed in a cell, the frequency of mispairing between the TCR α chain and β chain and an endogenous TCR α chain and β chain is reduced, the expression of the TCR α chain and β chain is increased and/or the stability of the TCR α chain and β chain is increased. In some embodiments, the one or more modifications is a replacement, deletion, or insertion of one or more amino acids in the Cα region and/or the Cβ region. In some aspects, the one or more modifications contain replacement(s) to introduce one or more cysteine residues that are capable of forming one or more non-native disulfide bridges between the α chain and β chain.

In some of any such embodiments, the TCR or antigen-binding fragment thereof containing a Cα region containing a cysteine at a position corresponding to position 48 with numbering as set forth in SEQ ID NO: 24 and/or a Cβ region containing a cysteine at a position corresponding to position 57 with numbering as set forth in SEQ ID NO: 20. In some embodiments, said Cα region contains the amino acid sequence set forth in any of SEQ ID NOS: 19 or 24, or a sequence of amino acids that has at least 90% sequence identity thereto containing one or more cysteine residues capable of forming a non-native disulfide bond with the β chain; and/or said Cβ region contains the amino acid sequence set forth in any of SEQ ID NOS: 20, 21 or 25, or a sequence of amino acids that has at least 90% sequence identity thereto that contains one or more cysteine residues capable of forming a non-native disulfide bond with the α chain.

In some of any such embodiments, the TCR or antigen-binding fragment thereof is encoded by a nucleotide sequence that has been codon-optimized.

In some of any such embodiments, the binding molecule or TCR or antigen-binding fragment thereof is isolated or purified or is recombinant. In some of any such embodiments, the binding molecule or TCR or antigen-binding fragment thereof is human.

In some embodiments, the TCR may be a heterodimer of two chains α and β that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains. In some embodiments, each of the constant and variable domains contains disulfide bonds formed by cysteine residues.

In some embodiments, the TCR can contain an introduced disulfide bond or bonds. In some embodiments, the native disulfide bonds are not present. In some embodiments, the one or more of the native cysteines (e.g. in the constant domain of the α chain and β chain) that form a native interchain disulfide bond are substituted to another residue, such as to a serine or alanine. In some embodiments, an introduced disulfide bond can be formed by mutating non-cysteine residues on the alpha and β chains, such as in the constant domain of the α chain and β chain, to cysteine. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

Exemplary non-native disulfide bonds of a TCR are described in published International PCT No. WO2006/000830, WO2006037960, and Kuball et al. (2007) Blood, 109:2331-2338. In some embodiments, cysteines can be introduced at residue Thr48 of the Cα chain and Ser57 of the Cβ chain, at residue Thr45 of the Cα chain and Ser77 of the Cβ chain, at residue Tyr10 of the Cα chain and Ser17 of the Cβ chain, at residue Thr45 of the Cα chain and Asp59 of the Cβ chain and/or at residue Ser15 of the Cα chain and Glu15 of the Cβ chain with reference to numbering of a Cα set forth in SEQ ID NO: 24 or Cβ set forth in SEQ ID NO:20. In some embodiments, any of the provided cysteine mutations can be made at a corresponding position in another sequence, for example, in the mouse Cα and Cβ sequences described herein. The term “corresponding” with reference to positions of a protein, such as recitation that amino acid positions “correspond to” amino acid positions in a disclosed sequence, such as set forth in the Sequence listing, refers to amino acid positions identified upon alignment with the disclosed sequence based on structural sequence alignment or using a standard alignment algorithm, such as the GAP algorithm. For example, corresponding residues can be determined by alignment of a reference sequence with the Cα sequence set forth in any of SEQ ID NO: 24 or the Cβ sequence set forth in SEQ ID NO: 20 by structural alignment methods as described herein. By aligning the sequences, corresponding residues can be identified, for example, using conserved and identical amino acid residues as guides.

Exemplary sequences (e.g. CDRs, V_{α} and/or V_{β} and constant region sequences) of provided TCRs are described herein.

In some embodiments, the recombinant TCR or antigen-binding portion thereof (or other MHC-peptide binding molecule, such as TCR-like antibody) is known to or likely or may recognize a peptide epitope or T cell epitope of a target polypeptide when presented by cells in the context of an MHC molecule, i.e. MHC-peptide complex of the target polypeptide. In some embodiments, the recombinant TCR (or other MHC-peptide binding molecule or TCR-like antibody) is known to or likely to exhibit specific binding for the T cell epitope of the target polypeptide, for example when displayed as an MHC-peptide complex. Methods of assessing binding or interaction of an MHC-peptide binding molecule (e.g. TCR or TCR-like antibody) are known, including any of the exemplary methods described herein.

In some embodiments, the MHC molecule is an MHC class I or an MHC class II molecule. In some embodiments, the MHC contains a polymorphic peptide binding site or binding groove that can, in some cases, complex with peptide epitopes of polypeptides, including peptide epitopes processed by the cell machinery. In some cases, MHC molecules can be displayed or expressed on the cell surface, including as a complex with peptide, i.e. MHC-peptide complex, for presentation of an antigen in a conformation recognizable by TCRs on T cells, or other MHC-peptide binding molecules. Generally, MHC class I molecules are heterodimers having a membrane spanning α chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate binding molecule, such as TCR. In some embodiments, MHC class I molecules deliver peptides originating in the cytosol to the cell surface, where a peptide:MHC complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. In some aspects, human MHC can also be referred to as human leukocyte antigen (HLA).

In some embodiments, the peptide epitope or T cell epitope is a peptide that may be derived from or based on a fragment of a longer biological molecule, such as a polypeptide or protein, and which is capable of associating with or forming a complex with an MHC molecule. In some embodiments, the peptide is about 8 to about 24 amino acids in length. In some embodiments, the peptide has a length of from or from about 9 to 22 amino acids for recognition in the MHC Class II complex. In some embodiments, the peptide has a length of from or from about 8 to 13 amino acids for recognition in the MHC Class I complex. In some embodiments, the MHC molecule and peptide epitope or T cell epitope are complexed or associated via non-covalent interactions of the peptide in the binding groove or cleft of the MHC molecule.

In some embodiments, the MHC-peptide complex is present or displayed on the surface of cells. In some embodiments, the MHC-peptide complex can be specifically recognized by a TCR or antigen-binding portion thereof, or other MHC-peptide binding molecule. In some embodiments, the T cell epitope or peptide epitope is capable of inducing an immune response in an animal by its binding characteristics to MHC molecules. In some embodiments, upon recognition of the T cell epitope, such as MHC-peptide complex, the TCR (or other MHC-peptide binding molecule) produces or triggers an activation signal to the T cell that induces a T cell response, such as T cell proliferation, cytokine production, a cytotoxic T cell response or other response.

In some embodiments, the TCR, or other MHC-peptide binding molecule, recognizes or potentially recognizes the T cell epitope in the context of an MHC class I molecule. MHC class I proteins are expressed in all nucleated cells of higher vertebrates. The MHC class I molecule is a heterodimer composed of a 46-kDa heavy chain which is non-covalently associated with the 12-kDa light chain β-2 microglobulin. In humans, there are several MHC alleles, such as, for example, HLA-A2, HLA-A1, HLA-A3, HLA-A24, HLA-A28, HLA-A31, HLA-A33, HLA-A34, HLA-B7, HLA-B45 and HLA-Cw8. The sequences of MHC alleles are known and can be found, for example, at the IMGT/HLA database available at www.ebi.ac.uk/ipd/imgt/hla. In some embodiments, the MHC class I allele is an HLA-A2 allele, which in some populations is expressed by approximately 50% of the population. In some embodiments, the HLA-A2 allele can be an HLA-A*0201, *0202, *0203, *0206, or *0207 gene product. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206 and 23% HLA-A*0203.

In some embodiments, MHC-class I restricted peptides are 8 to 15 amino acids in length, such as 8 to 10 amino acids in length. In some embodiments, MHC class I molecules bind peptides derived from endogenous antigens, such as tumor, viral or bacterial proteins produced within a diseased or infected cell, which have been processed within the cytoplasm of the cell via the cytosolic pathway. In some embodiments, MHC class I-peptide complexes displayed on the surface of the cell are typically recognized by TCRs expressed on CD8+ T cells, such as cytotoxic T cells. In some embodiments, MHC class I-peptide complexes can be recognized by TCRs expressed on CD4+ T cells, such as by TCRs exhibiting CD8- or partial CD8-independent binding.

In some embodiments, the TCR, or other MHC-peptide binding molecule, recognizes or potentially recognizes the T cell epitope in the context of an MHC class II molecule. MHC class II proteins are expressed in a subset of nucleated vertebrate cells, generally called antigen presenting cells (APCs). In humans, there are several MHC class II alleles, such as, for example, DR1, DR3, DR4, DR7, DR52, DQ1, DQ2, DQ4, DQ8 and DP1. In some embodiments, the MHC class II allele that is HLA-DRB1*0101, an HLA-DRB*0301, HLA-DRB*0701, HLA-DRB*0401 an HLA-DQB1*0201. The sequences of MHC alleles are known and can be found, for example, at the IMGT/HLA database available at www.ebi.ac.uk/ipd/imgt/hla.

In some embodiments, MHC-class II restricted peptides are generally between about 9 and 25 residues in length, such as between 15 and 25 residues or 13 and 18 residues in length, and, in some cases, contains a binding core region of about 9 amino acids or about 12 amino acids. In some embodiments, MHC class II molecules bind peptides derived from exogenous antigens, which are internalized by phagocytosis or endocytosis and processed within the endosomal/lysosomal pathway. In some embodiments, MHC class II-peptide complexes displayed on the surface of cells are typically recognized by CD4+ cells, such as helper T cells. In some embodiments, MHC class II-peptide complexes displayed can be recognized by TCRs expressed on CD8+ T cells.

Typically, the peptide epitope or T cell epitope is a peptide portion of an antigen. In some embodiments, the antigen is known, and in some cases the peptide epitope recognized by the TCR or antigen-binding portion thereof (or other MHC-peptide binding molecules) also may be known, such a known prior to performing the provided method.

In some embodiments, the antigen is a tumor-associated antigen, an antigen expressed in a particular cell type associated with an autoimmune or inflammatory disease, or an antigen derived from a viral pathogen or a bacterial pathogen. In some embodiments, the antigen is an antigen involved in a disease. In some embodiments, the disease can be caused by malignancy or transformation of cells, such as a cancer. In some embodiments, the antigen can be an intracellular protein antigen from a tumor or cancer cell, such as a tumor-associated antigen. In some cases, because the majority of cancer antigens are derived from intracellular proteins that can only be targeted at the cell surface in the context of an MHC molecule, TCRs make the ideal candidate for therapeutics as they have evolved to recognize this class of antigen. In some embodiments, the disease can be caused by infection, such as by bacterial or viral infection. In some embodiments, the antigen is a viral-associated cancer antigen. In some cases, a recombinant TCR or antigen-binding portions thereof (and other MHC-peptide binding molecules) recognize or potentially recognize peptides derived from viral proteins that have been naturally processed in infected cells and displayed by an MHC molecule on the cell surface. In some embodiments, the disease can be an autoimmune disease. Other targets include those listed in The HLA Factsbook (Marsh et al. (2000)) and others known.

In some embodiments, the antigen is one that is associated with a tumor or cancer. In some embodiments, a tumor or cancer antigen is one that can be found on a malignant cell, found inside a malignant cell or is a mediator of tumor cell growth. In some embodiments, a tumor or cancer antigen is one that is predominantly expressed or over-expressed by a tumor cell or cancer cell. A number of tumor antigens have been identified and are known, including MHC-restricted, T cell-defined tumor antigens (see e.g. cancerimmunity.org/peptide/; Boon and Old (1997) Curr Opin Immunol, 9:681-3; Cheever et al. (2009) Clin Cancer Res, 15:5323-37). In some embodiments, tumor antigens include, but are not limited to, mutated peptides, differentiation antigens, and overexpressed antigens, all of which could serve as targets for therapies.

In some embodiments, the tumor or cancer antigen is a lymphoma antigen, (e.g., non-Hodgkin's lymphoma or Hodgkin's lymphoma), a B-cell lymphoma cancer antigen, a leukemia antigen, a myeloma (i.e., multiple myeloma or plasma cell myeloma) antigen, an acute lymphoblastic leukemia antigen, a chronic myeloid leukemia antigen, or an acute myelogenous leukemia antigen. In some embodiments, the cancer antigen is an antigen that is overexpressed in or associated with a cancer that is an adenocarcinomas, such as pancreas, colon, breast, ovarian, lung, prostate, head and neck, including multiple myelomas and some B cell lymphomas. In some embodiments, the antigen is associated with a cancer, such as prostate cancer, lung cancer, breast cancer, ovarian cancer, pancreatic cancer, skin cancer, liver cancer (e.g., hepatocellular adenocarcinoma), intestinal cancer, or bladder cancer.

In some embodiments, the antigen is a tumor antigen that can be a glioma-associated antigen, β-human chorionic gonadotropin, alphafetoprotein (AFP), B-cell maturation antigen (BCMA, BCM), B-cell activating factor receptor (BAFFR, BR3), and/or transmembrane activator and CAML interactor (TACT). Fc Receptor-like 5 (FCRL5, FcRH5), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, and melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, beta-catenin, Bcr-abl, E2A-PRL, H4-RET, IGH-IGK, MYL-RAR, Caspase 8 or a B-Raf antigen. Other tumor antigens can include any derived from FRa, CD24, CD44, CD133, CD 166, epCAM, CA-125, HE4, Oval, estrogen receptor, progesterone receptor, uPA, PAI-1, CD19, CD20, CD22, ROR1, mesothelin, CD33/IL3Ra, c-Met, PSMA, Glycolipid F77, GD-2, insulin growth factor (IGF)-I, IGF-II and IGF-I receptor. Specific tumor-associated antigens or T cell epitopes are known (see e.g. van der Bruggen et al. (2013) Cancer Immun, available at www.cancerimmunity.org/peptide/; Cheever et al. (2009) Clin Cancer Res, 15, 5323-37).

In some embodiments, the antigen is a viral antigen. Many viral antigen targets have been identified and are known, including peptides derived from viral genomes in HIV, HTLV and other viruses (see e.g., Addo et al. (2007) PLoS ONE, 2, e321; Tsomides et al. (1994) J Exp Med, 180, 1283-93; Utz et al. (1996) J Virol, 70, 843-51). Exemplary viral antigens include, but are not limited to, an antigen from hepatitis A, hepatitis B (e.g., HBV core and surface antigens (HBVc, HBVs)), hepatitis C (HCV), Epstein-Barr virus (e.g. EBVA), human papillomavirus (HPV; e.g. E6 and E7), human immunodeficiency type-1 virus (HIV1), Kaposi's sarcoma herpes virus (KSHV), human papilloma virus (HPV), influenza virus, Lassa virus, HTLN-1, HIN-1, HIN-II, CMN, EBN or HPN. In some embodiments, the target protein is a bacterial antigen or other pathogenic antigen, such as Mycobacterium tuberculosis (MT) antigens, trypanosome, e.g., Tiypansoma cruzi (T. cruzi), antigens such as surface antigen (TSA), or malaria antigens. Specific viral antigen or epitopes or other pathogenic antigens or T cell epitopes are known (see e.g., Addo et al. (2007) PLoS ONE, 2:e321; Anikeeva et al. (2009) Clin Immunol, 130:98-109).

In some embodiments, the antigen is an antigen derived from a virus associated with cancer, such as an oncogenic virus. For example, an oncogenic virus is one in which infection from certain viruses are known to lead to the development of different types of cancers, for example, hepatitis A, hepatitis B (e.g., HBV core and surface antigens (HBVc, HBVs)), hepatitis C (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV) antigen.

In some embodiments, the viral antigen is an HPV antigen, which, in some cases, can lead to a greater risk of developing cervical cancer. In some embodiments, the antigen can be a HPV-16 antigen, and HPV-18 antigen, and HPV-31 antigen, an HPV-33 antigen or an HPV-35 antigen. In some embodiments, the viral antigen is an HPV-16 antigen (e.g., seroreactive regions of the E1, E2, E6 and/or E7 proteins of HPV-16, see e.g., U.S. Pat. No. 6,531,127) or an HPV-18 antigen (e.g., seroreactive regions of the L1 and/or L2 proteins of HPV-18, such as described in U.S. Pat. No. 5,840,306). In some embodiments, the viral antigen is an HPV-16 antigen that is from the E6 and/or E7 proteins of HPV-16. In some embodiments, the TCR is a TCR directed against an HPV-16 E6 or HPV-16 E7. In some embodiments, the TCR is a TCR described in, e.g., WO 2015/184228, WO 2015/009604 and WO 2015/009606.

In some embodiments, the viral antigen is a HBV or HCV antigen, which, in some cases, can lead to a greater risk of developing liver cancer than HBV or HCV negative subjects. For example, in some embodiments, the heterologous antigen is an HBV antigen, such as a hepatitis B core antigen or a hepatitis B envelope antigen (US2012/0308580).

In some embodiments, the viral antigen is an EBV antigen, which, in some cases, can lead to a greater risk for developing Burkitt's lymphoma, nasopharyngeal carcinoma and Hodgkin's disease than EBV negative subjects. For example, EBV is a human herpes virus that, in some cases, is found associated with numerous human tumors of diverse tissue origin. While primarily found as an asymptomatic infection, EBV-positive tumors can be characterized by active expression of viral gene products, such as EBNA-1, LMP-1 and LMP-2A. In some embodiments, the heterologous antigen is an EBV antigen that can include Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA or EBV-VCA.

In some embodiments, the viral antigen is an HTLV-1 or HTLV-2 antigen, which, in some cases, can lead to a greater risk for developing T-cell leukemia than HTLV-1 or HTLV-2 negative subjects. For example, in some embodiments, the heterologous antigen is an HTLV-antigen, such as TAX.

In some embodiments, the viral antigen is a HHV-8 antigen, which, in some cases, can lead to a greater risk for developing Kaposi's sarcoma than HHV-8 negative subjects. In some embodiments, the heterologous antigen is a CMV antigen, such as pp65 or pp64 (see U.S. Pat. No. 8,361,473).

In some embodiments, the antigen is an autoantigen, such as an antigen of a polypeptide associated with an autoimmune disease or disorder. In some embodiments, the autoimmune disease or disorder can be multiple sclerosis (MS), rheumatoid arthritis (RA), Sjogren syndrome, scleroderma, polymyositis, dermatomyositis, systemic lupus erythematosus, juvenile rheumatoid arthritis, ankylosing spondylitis, myasthenia gravis (MG), bullous pemphigoid (antibodies to basement membrane at dermal-epidermal junction), pemphigus (antibodies to mucopolysaccharide protein complex or intracellular cement substance), glomerulonephritis (antibodies to glomerular basement membrane), Goodpasture's syndrome, autoimmune hemolytic anemia (antibodies to erythrocytes), Hashimoto's disease (antibodies to thyroid), pernicious anemia (antibodies to intrinsic factor), idiopathic thrombocytopenic purpura (antibodies to platelets), Grave's disease, or Addison's disease (antibodies to thyroglobulin). In some embodiments, the autoantigen, such as an autoantigen associated with one of the foregoing autoimmune disease, can be collagen, such as type II collagen, mycobacterial heat shock protein, thyroglobulin, acetyl choline receptor (AcHR), myelin basic protein (MBP) or proteolipid protein (PLP). Specific autoimmune associated epitopes or antigens are known (see e.g., Bulek et al. (2012) Nat Immunol, 13:283-9; Harkiolaki et al. (2009) Immunity, 30:348-57; Skowera et al. (2008) J Clin Invest, 1(18): 3390-402).

In some embodiments, the identity of the peptide epitope of the target antigen is known, which, in some cases, can be used in producing or generating a TCR of interest or in assessing a functional activity or property, including in connection with the provided methods. In some embodiments, peptide epitopes can be determined or identified based on the presence of an HLA-restricted motif in a target antigen of interest. In some embodiments, peptides are identified using known computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule. In some aspects, HLA-A*0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007). Provided are methods of screening and cells employed in the methods of screening, such as T cells, that recognize an antigen or an epitope, in the context of a major histocompatibility complex (MHC) molecule.

In some embodiments, the MHC contains a polymorphic peptide binding site or binding groove that can, in some cases, complex with peptide epitopes of polypeptides, including peptide epitopes processed by the cell machinery. In some cases, MHC molecules can be displayed or expressed on the cell surface, including as a complex with peptide, i.e. MHC-peptide complex, for presentation of an antigen in a conformation recognizable by TCRs on T cells, or other MHC-peptide binding molecules. Generally, MHC class I molecules are heterodimers having a membrane spanning α chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an epitope binding site or sites for binding a peptide and the sequences necessary for recognition by the appropriate binding molecule, such as TCR. In some embodiments, MHC class I molecules deliver peptides originating in the cytosol to the cell surface, where a peptide:MHC complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. In some aspects, human MHC can also be referred to as human leukocyte antigen (HLA).

In some embodiments, the peptide epitope or T cell epitope is a peptide that may be derived from or based on a fragment of a longer biological molecule, such as a polypeptide or protein, and which is capable of associating with or forming a complex with an MHC molecule. In some embodiments, the peptide is about 8 to about 24 amino acids in length. In some embodiments, the peptide has a length of from or from about 9 to 22 amino acids for recognition in the MHC Class II complex. In some embodiments, the peptide has a length of from or from about 8 to 13 amino acids for recognition in the MHC Class I complex. In some embodiments, the MHC molecule and peptide epitope or T cell epitope are complexed or associated via non-covalent interactions of the peptide in the binding groove or cleft of the MHC molecule.

In some embodiments, the MHC-peptide complex is present or displayed on the surface of cells. In some embodiments, the MHC-peptide complex can be specifically recognized by a TCR or antigen-binding portion thereof, or other MHC-peptide binding molecule. In some embodiments, the T cell epitope or peptide epitope is capable of inducing an immune response in an animal by its binding characteristics to MHC molecules. In some embodiments, upon recognition of the T cell epitope, such as MHC-peptide complex, the TCR (or other MHC-peptide binding molecule) produces or triggers an activation signal to the T cell that induces a T cell response, such as T cell proliferation, cytokine production, a cytotoxic T cell response or other response.

In some embodiments, the MHC-peptide binding molecule is a TCR or epitope binding fragment thereof. In some embodiments, the MHC-peptide binding molecule is a TCR-like CAR that contains an antibody or epitope binding fragment thereof, such as a TCR-like antibody, such as one that has been engineered to bind to MHC-peptide complexes. In some embodiments, such binding molecules bind to a binding sequence, such as a T cell epitope, containing an amino acid sequence or antigen of a target polypeptide. In some embodiments, the binding sequence of the target peptide or target polypeptide is known. In some embodiments, the MHC-peptide binding molecule can be derived from natural sources, or it may be partly or wholly synthetically or recombinantly produced.

In some embodiments, the MHC-peptide binding molecule is a molecule or portion thereof that possesses the ability to bind, e.g. specifically bind, to a peptide epitope that is presented or displayed in the context of an MHC molecule, i.e. an MHC-peptide complex, such as on the surface of a cell. In some embodiments, a binding molecule may include any naturally occurring, synthetic, semi-synthetic, or recombinantly produced molecule that can bind, e.g. specifically bind, to an MHC-peptide complex. Exemplary MHC-peptide binding molecules include T cell receptors or antibodies, or antigen-binding portions thereof, including single chain immunoglobulin variable regions (e.g., scTCR, scFv) thereof, that exhibit specific ability to bind to an MHC-peptide complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, the TCR is in cell-bound form expressed on the surface of a cell.

In some embodiments a dTCR contains a first polypeptide wherein a sequence corresponding to a provided TCR α chain variable region sequence is fused to the N terminus of a sequence corresponding to a TCR α chain constant region extracellular sequence, and a second polypeptide wherein a sequence corresponding to a provided TCR β chain variable region sequence is fused to the N terminus a sequence corresponding to a TCR β chain constant region extracellular sequence, the first and second polypeptides being linked by a disulfide bond. In some embodiments, the bond can correspond to the native interchain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a provided TCR α chain containing a variable α domain, a constant α domain and a first dimerization motif attached to the C-terminus of the constant α domain, and a provided TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR, which is a single amino acid strand containing an α chain and a β chain that is able to bind to MHC-peptide complexes. Typically, a scTCR can be generated using known methods, See e.g., International published PCT Nos. WO 96/13593, WO 96/18105, WO99/18129, WO 04/033685, WO2006/037960, WO2011/044186; U.S. Pat. No. 7,569,664; and Schlueter, C. J. et al. J. Mol. Biol. 256, 859 (1996).

In some embodiments, a scTCR contains a first segment constituted by an amino acid sequence corresponding to a sequence of a provided TCR α chain variable region, a second segment constituted by an amino acid sequence corresponding to a provided TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an amino acid sequence corresponding to a provided TCR β chain variable region, a second segment constituted by an amino acid sequence corresponding to a provided TCR α chain variable region sequence fused to the N terminus of an amino acid sequence corresponding to a TCR α chain constant domain extracellular sequence, and a linker sequence linking the C terminus of the first segment to the N terminus of the second segment.

In some embodiments, a scTCR contains a first segment constituted by a provided α chain variable region sequence fused to the N terminus of an α chain extracellular constant domain sequence, and a second segment constituted by a provided β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a provided TCR β chain variable region sequence fused to the N terminus of a β chain extracellular constant domain sequence, and a second segment constituted by a provided α chain variable region sequence fused to the N terminus of a sequence α chain extracellular constant and transmembrane sequence, and, optionally, a linker sequence linking the C terminus of the first segment to the N terminus of the second segment.

In some embodiments, for the scTCR to bind an MHC-peptide complex, the α and β chains must be paired so that the variable region sequences thereof are orientated for such binding. Various methods of promoting pairing of an α and β in a scTCR are known. In some embodiments, a linker sequence is included that links the α and β chains to form the single polypeptide strand. In some embodiments, the linker should have sufficient length to span the distance between the C terminus of the α chain and the N terminus of the β chain, or vice versa, while also ensuring that the linker length is not so long so that it blocks or reduces bonding of the scTCR to the target peptide-MHC complex.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P-, wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)ₙ-P-, wherein n is 5 or 6 and P is proline, G is glycine and S is serine (SEQ ID NO: 22). In some embodiments, the linker has the sequence GSADDAKKDAAKKDGKS (SEQ ID NO: 23).

In some embodiments, a scTCR contains a disulfide bond between residues of the single amino acid strand, which, in some cases, can promote stability of the pairing between the α and β regions of the single chain molecule (see e.g. U.S. Pat. No. 7,569,664). In some embodiments, the scTCR contains a covalent disulfide bond linking a residue of the immunoglobulin region of the constant domain of the α chain to a residue of the immunoglobulin region of the constant domain of the β chain of the single chain molecule. In some embodiments, the disulfide bond corresponds to the native disulfide bond present in a native dTCR. In some embodiments, the disulfide bond in a native TCR is not present. In some embodiments, the disulfide bond is an introduced non-native disulfide bond, for example, by incorporating one or more cysteines into the constant region extracellular sequences of the first and second chain regions of the scTCR polypeptide. Exemplary cysteine mutations include any as described herein. In some cases, both a native and a non-native disulfide bond may be present.

In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, any of the provided TCRs, including a dTCR or scTCR, can be linked to signaling domains that yield an active TCR on the surface of a T cell. In some embodiments, the TCR is expressed on the surface of cells. In some embodiments, the TCR does contain a sequence corresponding to a transmembrane sequence. In some embodiments, the transmembrane domain is positively charged. In some embodiments, the transmembrane domain can be a Cα or Cβ transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3.

In some embodiments, the TCR is a soluble TCR. In some embodiments, the soluble TCR has a structure as described in WO99/60120 or WO 03/020763. In some embodiments, the TCR does not contain a sequence corresponding to the transmembrane sequence, for example, to permit membrane anchoring into the cell in which it is expressed. In some embodiments, the TCR does not contain a sequence corresponding to cytoplasmic sequences.

In some embodiments, the recombinant receptor, e.g., TCR or antigen-binding fragment thereof, is or has been modified compared to a known recombinant receptor. In certain embodiments, the recombinant receptors, e.g., TCRs or antigen-binding fragments thereof, include one or more amino acid variations, e.g., substitutions, deletions, insertions, and/or mutations, compared to the sequence of a recombinant receptor, e.g., TCR, described herein or known. Exemplary variants include those designed to improve the binding affinity and/or other biological properties of the binding molecule. Amino acid sequence variants of a binding molecule may be prepared by introducing appropriate modifications into the nucleotide sequence encoding the binding molecule, or by peptide synthesis. Such modifications include, for example, deletions from, and/or insertions into and/or substitutions of residues within the amino acid sequences of the binding molecule. Any combination of deletion, insertion, and substitution can be made to arrive at the final construct, provided that the final construct possesses the desired characteristics, e.g., antigen-binding.

In some embodiments, directed evolution methods are used to generate TCRs with altered properties, such as with higher affinity for a specific peptide in the context of an MHC molecule. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a reference TCR, such as any provided herein, can be used as a template for producing mutagenized TCRs in which in one or more residues of the CDRs are mutated, and mutants with a desired altered property, such as higher affinity for peptide epitope in the context of an MHC molecule, are selected.

In certain embodiments, the recombinant receptors, e.g., TCRs or antigen-binding fragments thereof, include one or more amino acid substitutions, e.g., as compared to a recombinant receptor, e.g., TCR, sequence compared to a sequence of a natural repertoire, e.g., human repertoire. Sites of interest for substitutional mutagenesis include the CDRs, FRs and/or constant regions. Amino acid substitutions may be introduced into a binding molecule of interest and the products screened for a desired activity, e.g., retained/improved antigen affinity or avidity, decreased immunogenicity, improved half-life, CD8-independent binding or activity, surface expression, promotion of TCR chain pairing and/or other improved properties or functions.

In some embodiments, one or more residues within a CDR of a recombinant receptor, e.g., TCR, is/are substituted. In some embodiments, the substitution is made to revert a sequence or position in the sequence to a germline sequence, such as a binding molecule sequence found in the germline (e.g., human germline), for example, to reduce the likelihood of immunogenicity, e.g., upon administration to a human subject.

In certain embodiments, substitutions, insertions, or deletions may occur within one or more CDRs so long as such alterations do not substantially reduce the ability of the recombinant receptor, e.g., TCR or antigen-binding fragment thereof, to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variable sequences provided herein, each CDR either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues.

In some aspects, the TCR or antigen-binding fragment thereof may contain one or more modifications in the α chain and/or β chain such that when the TCR or antigen-binding fragment thereof is expressed in a cell, the frequency of mis-pairing between the TCR α chain and β chain and an endogenous TCR α chain and β chain is reduced, the expression of the TCR α chain and β chain is increased, and/or the stability of the TCR α chain and β chain is increased.

In some embodiments, the TCR contains one or more non-native cysteine residues to introduce a covalent disulfide bond linking a residue of the immunoglobulin region of the constant domain of the α chain to a residue of the immunoglobulin region of the constant domain of the β chain. In some embodiments, one or more cysteines can be incorporated into the constant region extracellular sequences of the first and second segments of the TCR polypeptide. Exemplary non-limiting modifications in a TCR to introduce a non-native cysteine residues are described herein (see also, International PCT No. WO2006/000830 and WO2006037960). In some cases, both a native and a non-native disulfide bond may be desirable. In some embodiments, the TCR or antigen-binding fragment is modified such that the interchain disulfide bond in a native TCR is not present.

In some embodiments, the transmembrane domain of the constant region of the TCR can be modified to contain a greater number of hydrophobic residues (see e.g. Haga-Friedman et al. (2012) Journal of Immunology, 188:5538-5546). In some embodiments, the tranmembrane region of TCR α chain contains one or more mutations corresponding to S116L, G119V or F120L, with reference to numbering of a Cα set forth in SEQ ID NO:24.

In some embodiments, the TCR or antigen-binding fragment thereof is encoded by a nucleotide sequence that is or has been codon-optimized. Exemplary codon-optimized variants are described elsewhere herein.

B. Chimeric Antigen Receptors (CARs)

In some embodiments, the recombinant receptor that is introduced into the cell is a chimeric antigen receptor (CAR) or an antigen-binding fragment thereof. In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain (also interchangeably called an intracellular signaling domain), such as a cytoplasmic (intracellular) region capable of inducing a primary activation signal in a T cell, for example, a cytoplasmic signaling domain of a T cell receptor (TCR) component (e.g. a cytoplasmic signaling domain of a zeta chain of a CD3-zeta (CD3ζ) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g. antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g. CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the CAR contains an antibody or an antigen-binding fragment (e.g. scFv) that specifically recognizes an antigen, such as an intact antigen, expressed on the surface of a cell.

In some embodiments, the antigen (or a ligand) is a tumor antigen or cancer marker. In some embodiments, the antigen (or a ligand) is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, 0-acetylated GD2 (OGD2), ganglioside GD3, glycoprotein 100 (gp100), glypican-3 (GPC3), G Protein Coupled Receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Ra), IL-13 receptor alpha 2 (IL-13Ra2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the recombinant receptor such as the CAR, such as the antibody portion thereof, further includes a spacer, which may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer.

In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is less than 250 amino acids in length, less than 200 amino acids in length, less than 150 amino acids in length, less than 100 amino acids in length, less than 75 amino acids in length, less than 50 amino acids in length, less than 25 amino acids in length, less than 20 amino acids in length, less than 15 amino acids in length, less than 12 amino acids in length, or less than 10 amino acids in length. In some embodiments, the spacer is from or from about 10 to 250 amino acids in length, 10 to 150 amino acids in length, 10 to 100 amino acids in length, 10 to 50 amino acids in length, 10 to 25 amino acids in length, 10 to 15 amino acids in length, 15 to 250 amino acids in length, 15 to 150 amino acids in length, 15 to 100 amino acids in length, 15 to 50 amino acids in length, 15 to 25 amino acids in length, 25 to 250 amino acids in length, 25 to 100 amino acids in length, 25 to 50 amino acids in length, 50 to 250 amino acids in length, 50 to 150 amino acids in length, 50 to 100 amino acids in length, 100 to 250 amino acids in length, 100 to 150 amino acids in length, or 150 to 250 amino acids in length.

Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the spacer has the sequence set forth in SEQ ID NO: 131, and is encoded by the sequence set forth in SEQ ID NO: 132. In some embodiments, the spacer has the sequence set forth in SEQ ID NO: 133. In some embodiments, the spacer has the sequence set forth in SEQ ID NO: 134.

In some embodiments, the constant region or portion is of IgD. In some embodiments, the spacer has the sequence set forth in SEQ ID NO: 135. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 131, 133, 134 and 135.

The antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen binding component (e.g., antibody) is linked to one or more transmembrane and intracellular signaling regions. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the ROR1-binding antibody is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR includes a chimeric molecule between CD3-zeta (CD3-ζ) or Fc receptor γ and CD8, CD4, CD25 or CD16.

In some embodiments, upon ligation of the CAR, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, CD3 gamma, CD3 delta, CD3 epsilon, FcR gamma or FcR beta. In some embodiments, cytoplasmic signaling molecule(s) in the CAR contain(s) a cytoplasmic signaling domain, portion thereof, or sequence derived from CD3 zeta.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion containing the antibody or fragment described herein. In some aspects, the chimeric antigen receptor includes an extracellular portion containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain disposed between the extracellular domain and the intracellular signaling region.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 136 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:136; in some embodiments, the transmembrane-domain containing portion of the recombinant receptor comprises the sequence of amino acids set forth in SEQ ID NO: 137 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular signaling region comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 138 or 139 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 138 or 139. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 140 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 140.

In some embodiments, the intracellular signaling region comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or 8,911,993. In some embodiments, the intracellular signaling region comprises the sequence of amino acids set forth in SEQ ID NO: 129, 130 or 141 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 129, 130 or 141.

In some aspects, the spacer contains only a hinge region of an IgG, such as only a hinge of IgG4 or IgG1, such as the hinge only spacer set forth in SEQ ID NO:131. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:133. In some embodiments, the spacer is an Ig hinge, e.g., an IgG4 hinge, linked to a C_{H}3 domain only, such as set forth in SEQ ID NO:134. In some embodiments, the spacer is or comprises a glycine-serine rich sequence or other flexible linker such as known flexible linkers.

In some embodiments, the CAR includes an anti-HPV 16 E6 or E7 antibody or fragment, including sdAbs (e.g. containing only the V_{H} region) and scFvs, a spacer such as any of the Ig-hinge containing spacers, a CD28 transmembrane domain, a CD28 intracellular signaling domain, and a CD3 zeta signaling domain. In some embodiments, the CAR includes the HPV 16 antibody or fragment, including sdAbs and scFvs, a spacer such as any of the Ig-hinge containing spacers, a CD28 transmembrane domain, a CD28 intracellular signaling domain, and a CD3 zeta signaling domain. In some embodiments, such CAR constructs further includes a T2A ribosomal skip element and/or a tEGFR sequence, e.g., downstream of the CAR.

In some embodiments, the CAR or antigen-binding fragment thereof is encoded by a nucleotide sequence that is or has been codon-optimized. Exemplary codon-optimized variants are described elsewhere herein.

C. TCR-Like CARs

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against a peptide in the context of an MHC molecule also may be referred to as a TCR-like CAR.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning α chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods known (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described herein. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen C J. et al. (2003) J Mol. Recogn. 16:324-332.

Among the provided embodiments are recombinant receptors, such as those that include antibodies, e.g., TCR-like antibodies. In some embodiments, the antigen receptors and other chimeric receptors specifically bind to a region or epitope of an antigen, e.g. TCR-like antibodies. Among the antigen receptors are functional non-TCR antigen receptors, such as chimeric antigen receptors (CARs). Also provided are cells expressing the CARs and uses thereof in adoptive cell therapy, such as treatment of diseases and disorders associated with the expression of the antigen and/or epitope.

Thus, provided herein are TCR-like CARs that contain a non-TCR molecule that exhibits T cell receptor specificity, such as for a T cell epitope or peptide epitope when displayed or presented in the context of an MHC molecule. In some embodiments, a TCR-like CAR can contain an antibody or antigen-binding portion thereof, e.g., TCR-like antibody, such as described herein. In some embodiments, the antibody or antibody-binding portion thereof is reactive against specific peptide epitope in the context of an MHC molecule, wherein the antibody or antibody fragment can differentiate the specific peptide in the context of the MHC molecule from the MHC molecule alone, the specific peptide alone, and, in some cases, an irrelevant peptide in the context of an MHC molecule. In some embodiments, an antibody or antigen-binding portion thereof can exhibit a higher binding affinity than a T cell receptor.

In some aspects, the transgene can include nucleic acids encoding one or more CARs, e.g., a first CAR which contains signaling domains to induce the primary signal and a second CAR which binds to a second antigen and contains the component for generating a costimulatory signal. For example, a first CAR can be an activating CAR and the second CAR can be a costimulatory CAR. In some aspects, both CARs must be ligated in order to induce a particular effector function in the cell, which can provide specificity and selectivity for the cell type being targeted.

In some embodiments, the activating domain is included within one CAR, whereas the costimulatory component is provided by another chimeric receptor recognizing another antigen. In some embodiments, the CARs include activating or stimulatory CARs, and costimulatory receptors, both expressed on the same cell (see WO2014/055668). In some aspects, the HPV 16 E6 or E7 antibody-containing receptor is the stimulatory or activating CAR; in other aspects, it is the costimulatory receptor. In some embodiments, the transgene further encodes inhibitory CARs (iCARs, see Fedorov et al., Sci. Transl. Medicine, 5(215) (December, 2013), such as an inhibitory receptor recognizing a peptide epitope other than HPV 16 E6 or HPV16 E7, whereby an activating signal delivered through the HPV 16-targeting CAR is diminished or inhibited by binding of the inhibitory CAR to its ligand, e.g., to reduce off-target effects.

In some embodiments, transgene can include nucleic acids encoding a recombinant receptor can further encode an additional receptor, such as a receptor capable of delivering a costimulatory or survival-promoting signal, such as a costimulatory receptor (see WO2014/055668) and/or to block or change the outcome of an inhibitory signal, such as one typically delivered via an immune checkpoint or other immunoinhibitory molecule, such as one expressed in the tumor microenvironment, e.g., in order to promote increased efficacy of such engineered cells. See, e.g., Tang et al., Am J Transl Res. 2015; 7(3): 460-473. In some embodiments, the cell may further include one or more other exogenous or recombinant or engineered components, such as one or more exogenous factors and/or costimulatory ligands, which are expressed on or in or secreted by the cells and can promote function, e.g., in the microenviroment. Exemplary of such ligands and components include, e.g., TNFR and/or Ig family receptors or ligands, e.g., 4-1BBL, CD40, CD40L, CD80, CD86, cytokines, chemokines, and/or antibodies or other molecules, such as scFvs. See, e.g., patent application publication Nos WO2008121420 A1, WO2014134165 A1, US20140219975 A1.

D. Chimeric Auto-Antibody Receptor (CAAR)

In some embodiments, the chimeric receptor is a chimeric autoantibody receptor (CAAR). In some embodiments, the CAAR binds, e.g., specifically binds, or recognizes, an autoantibody. In some embodiments, a cell expressing the CAAR, such as a T cell engineered to express a CAAR, can be used to bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the chimeric receptor is a CAAR, such as any described in U.S. Patent Application Pub. No. US 2017/0051035.

In some embodiments, the CAAR comprises an autoantibody binding domain, a transmembrane domain, and one or more intracellular signaling region or domain (also interchangeably called a cytoplasmic signaling domain or region). In some embodiments, the intracellular signaling region comprises an intracellular signaling domain. In some embodiments, the intracellular signaling domain is or comprises a primary signaling region, a signaling domain that is capable of stimulating and/or inducing a primary activation signal in a T cell, a signaling domain of a T cell receptor (TCR) component (e.g. an intracellular signaling domain or region of a CD3-zeta (CD3ζ) chain or a functional variant or signaling portion thereof), and/or a signaling domain comprising an immunoreceptor tyrosine-based activation motif (ITAM).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

### V. COMPOSITIONS AND FORMULATIONS

Also provided are populations of engineered cells, compositions containing such cells and/or enriched for such cells. Among the compositions are pharmaceutical compositions and formulations for administration, such as for adoptive cell therapy. Also provided are therapeutic methods for administering the cells and compositions to subjects, e.g., patients.

In some embodiments, the provided cell population and/or compositions containing engineered cells include a cell population that exhibits more improved, uniform, homogeneous and/or stable expression and/or antigen binding by the recombinant receptor, e.g., exhibit reduced coefficient of variation, compared to the expression and/or antigen binding of cell populations and/or compositions generated using conventional methods. In some embodiments, the cell population and/or compositions exhibit at least 100%, 95%, 90%, 80%, 70%, 60%, 50%, 40%, 30%, 20% or 10% lower coefficient of variation of expression of the transgene and/or antigen binding by the recombinant receptor compared to a respective population generated using conventional methods, e.g., random integration of transgene. The coefficient of variation is defined as standard deviation of expression of the nucleic acid of interest (e.g., transgene encoding a recombinant receptor) within a population of cells, for example CD4+ and/or CD8+ T cells, divided by the mean of expression of the respective nucleic acid of interest in the respective population of cells. In some embodiments, the cell population and/or compositions exhibit a coefficient of variation that is lower than 0.70, 0.65, 0.60, 0.55, 0.50, 0.45, 0.40, 0.35 or 0.30 or less, when measured among CD4+ and/or CD8+ T cell populations that have been engineered using the methods provided herein.

In some embodiments, provided are cell population and/or compositions that include cells that have a targeted knock-in of the recombinant receptor-encoding transgene into one or more of the endogenous TCR gene loci, thereby having a knock-out of the one or more of the endogenous TCR gene loci, e.g., knock out of the target gene for integration, such as TRAC, TRBC1 and/or TRBC2. In some embodiments, all or substantially all of the cells in the cell population that have integration of the recombinant receptor-encoding transgene also have a knock-out of the one or more of the endogenous TCR gene loci. In some embodiments, at least 80%, 85%, 90%, 91%, 92%, 93%, 94%, 95%, 96%, 97%, 98%, 99%, or more of the cells in the cell population and/or composition that express the recombinant receptor, contain a knock-out of the one or more of the endogenous TCR gene loci, e.g., TRAC, TRBC1 and/or TRBC2. Thus, in the provided cell population and/or compositions, all or substantially all of the engineered cells that express the recombinant receptor, also contain a knock-out of the endogenous TCR, by virtue of targeted knock-in of the transgene into the endogenous TCR gene loci.

In some embodiments, provided are cell population and/or compositions that include a plurality of engineered immune cells comprising a recombinant receptor or an antigen-binding fragment thereof encoded by a transgene and a genetic disruption of at least one target site within a T cell receptor alpha constant (TRAC) gene and/or a T cell receptor beta constant (TRBC) gene, wherein at least or greater than 35%, 40%, 45%, 50%, 55%, 60%, 65%, 70%, 75%, 80%, or 90% of the cells in the composition comprise a genetic disruption at a target position within a T cell receptor alpha constant (TRAC) gene and/or a T cell receptor beta constant (TRBC) gene; and the transgene encoding the recombinant TCR or antigen-binding fragment thereof or a chain thereof is targeted at or near the target position via homology directed repair (HDR).

In some embodiments, expression and/or antigen binding by the recombinant receptor can be assessed using any reagents and/or assays described herein, e.g., in Section I.C. In some embodiments, expression is measured using a binding molecule that recognizes and/or specifically binds to the recombinant receptor or a portion thereof. For example, in some embodiments, expression of the recombinant receptor encoded by the transgene is assessed using an anti-TCR Vβ 22 antibody, e.g., by flow cytometry. In some embodiments, antigen binding of a recombinant receptor that is a TCR, can be assessed using antigen that is isolated or purified or recombinant, cells expressing particular antigen, and/or using a TCR ligand (MHC-peptide complex).

In some embodiments, the provided compositions containing cells such as in which cells expressing the recombinant receptor and/or contain a knock-out of one or more of the endogenous TCR-encoding genes make up at least 30%, 40%, 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. In some aspects, the cells are isolated from a subject, engineered, and administered to the same subject. In other aspects, they are isolated from one subject, engineered, and administered to another subject. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### VI. METHODS OF ADMINISTRATION AND USES IN ADOPTIVE CELL THERAPY

Provided are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. A sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means. Dosing and administration may depend in part on whether the administration is brief or chronic. Various dosing schedules include but are not limited to single or multiple administrations over various time-points, bolus administration, and pulse infusion.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse.

In some aspects, the subject has not received prior treatment with another therapeutic agent.

The disease or condition that is treated in some aspects can be any in which expression of an antigen is associated with, specific to, and/or expressed on a cell or tissue of a disease, disorder or condition and/or involved in the etiology of a disease, condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described herein. In particular embodiments, the immunomodulatory polypeptide and/or recombinant receptor, e.g., the chimeric antigen receptor or TCR, specifically binds to an antigen associated with the disease or condition. In some embodiments, the subject has a disease, disorder or condition, optionally a cancer, a tumor, an autoimmune disease, disorder or condition, or an infectious disease.

In some embodiments, the disease, disorder or condition includes tumors associated with various cancers. The cancer can in some embodiments be any cancer located in the body of a subject, such as, but not limited to, cancers located at the head and neck, breast, liver, colon, ovary, prostate, pancreas, brain, cervix, bone, skin, eye, bladder, stomach, esophagus, peritoneum, or lung. For example, the anti-cancer agent can be used for the treatment of colon cancer, cervical cancer, cancer of the central nervous system, breast cancer, bladder cancer, anal carcinoma, head and neck cancer, ovarian cancer, endometrial cancer, small cell lung cancer, non-small cell lung carcinoma, neuroendocrine cancer, soft tissue carcinoma, penile cancer, prostate cancer, pancreatic cancer, gastric cancer, gall bladder cancer or espohageal cancer. In some cases, the cancer can be a cancer of the blood. In some embodiments, the disease, disorder or condition is a tumor, such as a solid tumor, lymphoma, leukemia, blood tumor, metastatic tumor, or other cancer or tumor type. In some embodiments, the disease, disorder or condition is selected from among cancers of the colon, lung, liver, breast, prostate, ovarian, skin, melanoma, bone, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, and/or mesothelioma.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM), a B cell malignancy is selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is a tumor antigen that can be a glioma-associated antigen, β-human chorionic gonadotropin, alphafetoprotein (AFP), B-cell maturation antigen (BCMA, BCM), B-cell activating factor receptor (BAFFR, BR3), and/or transmembrane activator and CAML, interactor (TACI), Fc Receptor-like 5 (FCRL5, FcRH5), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase (e.g. tyrosinase-related protein 1 (TRP-1) or tyrosinase-related protein 2 (TRP-2)), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, and melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, beta-catenin, Bcr-abl, E2A-PRL, H4-RET, IGH-IGK, MYL-RAR, Caspase 8 or a B-Raf antigen. Other tumor antigens can include any derived from FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Ra, c-Met, PSMA, Glycolipid F77, GD-2, insulin growth factor (IGF)-I, IGF-II, IGF-I receptor and mesothelin. Specific tumor-associated antigens or T cell epitopes are known (see e.g. van der Bruggen et al. (2013) Cancer Immun, available at www.cancerimmunity.org/peptide/; Cheever et al. (2009) Clin Cancer Res, 15, 5323-37).

In some embodiments, the antigen associated with the disease or disorder is a viral antigen. Many viral antigen targets have been identified and are known, including peptides derived from viral genomes in HIV, HTLV and other viruses (see e.g., Addo et al. (2007) PLoS ONE, 2, e321; Tsomides et al. (1994) J Exp Med, 180, 1283-93; Utz et al. (1996) J Virol, 70, 843-51). Exemplary viral antigens include, but are not limited to, an antigen from hepatitis A, hepatitis B (e.g., HBV core and surface antigens (HBVc, HBVs)), hepatitis C (HCV), Epstein-Barr virus (e.g. EBVA), human papillomavirus (HPV; e.g. E6 and E7), human immunodeficiency type-1 virus (HIV1), Kaposi's sarcoma herpes virus (KSHV), human papilloma virus (HPV), influenza virus, Lassa virus, HTLN-1, HIN-1, HIN-II, CMN, EBN or HPN. In some embodiments, the target protein is a bacterial antigen or other pathogenic antigen, such as Mycobacterium tuberculosis (MT) antigens, trypanosome, e.g., Tiypansoma cruzi (T. cruzi), antigens such as surface antigen (TSA), or malaria antigens. Specific viral antigen or epitopes or other pathogenic antigens or T cell epitopes are known (see e.g., Addo et al. (2007) PLoS ONE, 2:e321; Anikeeva et al. (2009) Clin Immunol, 130:98-109).

In some embodiments, the antigen associated with the disease or disorder is an antigen derived from a virus associated with cancer, such as an oncogenic virus. For example, an oncogenic virus is one in which infection from certain viruses are known to lead to the development of different types of cancers, for example, hepatitis A, hepatitis B (e.g., HBV core and surface antigens (HBVc, HBVs)), hepatitis C (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV) antigen, or any antigens targeted by the recombinant receptors described herein, e.g., in Section IV.

In some embodiments, antigen associated with the disease, disorder or condition is selected from αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, 0-acetylated GD2 (OGD2), ganglioside GD3, glycoprotein 100 (gp100), glypican-3(GPC3), G Protein Coupled Receptor Class C Group 5 Member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the dose of genetically engineered cells comprises from or from about 1×10⁵ to 5×10⁸ total CAR-expressing T cells, 1×10⁵ to 2.5×10⁸ total CAR-expressing T cells, 1×10⁵ to 1×10⁸ total CAR-expressing T cells, 1×10⁵ to 5×10⁷ total CAR-expressing T cells, 1×10⁵ to 2.5×10⁷ total CAR-expressing T cells, 1×10⁵ to 1×10⁷ total CAR-expressing T cells, 1×10⁵ to 5×10⁶ total CAR-expressing T cells, 1×10⁵ to 2.5×10⁶ total CAR-expressing T cells, 1×10⁵ to 1×10⁶ total CAR-expressing T cells, 1×10⁶ to 5×10⁸ total CAR-expressing T cells, 1×10⁶ to 2.5×10⁸ total CAR-expressing T cells, 1×10⁶ to 1×10⁸ total CAR-expressing T cells, 1×10⁶ to 5×10⁷ total CAR-expressing T cells, 1×10⁶ to 2.5×10⁷ total CAR-expressing T cells, 1×10⁶ to 1×10⁷ total CAR-expressing T cells, 1×10⁶ to 5×10⁶ total CAR-expressing T cells, 1×10⁶ to 2.5×10⁶ total CAR-expressing T cells, 2.5×10⁶ to 5×10⁸ total CAR-expressing T cells, 2.5×10⁶ to 2.5×10⁸ total CAR-expressing T cells, 2.5×10⁶ to 1×10⁸ total CAR-expressing T cells, 2.5×10⁶ to 5×10⁷ total CAR-expressing T cells, 2.5×10⁶ to 2.5×10⁷ total CAR-expressing T cells, 2.5×10⁶ to 1×10⁷ total CAR-expressing T cells, 2.5×10⁶ to 5×10⁶ total CAR-expressing T cells, 5×10⁶ to 5×10⁸ total CAR-expressing T cells, 5×10⁶ to 2.5×10⁸ total CAR-expressing T cells, 5×10⁶ to 1×10⁸ total CAR-expressing T cells, 5×10⁶ to 5×10⁷ total CAR-expressing T cells, 5×10⁶ to 2.5×10⁷ total CAR-expressing T cells, 5×10⁶ to 1×10⁷ total CAR-expressing T cells, 1×10⁷ to 5×10⁸ total CAR-expressing T cells, 1×10⁷ to 2.5×10⁸ total CAR-expressing T cells, 1×10⁷ to 1×10⁸ total CAR-expressing T cells, 1×10⁷ to 5×10⁷ total CAR-expressing T cells, 1×10⁷ to 2.5×10⁷ total CAR-expressing T cells, 2.5×10⁷ to 5×10⁸ total CAR-expressing T cells, 2.5×10⁷ to 2.5×10⁸ total CAR-expressing T cells, 2.5×10⁷ to 1×10⁸ total CAR-expressing T cells, 2.5×10⁷ to 5×10⁷ total CAR-expressing T cells, 5×10⁷ to 5×10⁸ total CAR-expressing T cells, 5×10⁷ to 2.5×10⁸ total CAR-expressing T cells, 5×10⁷ to 1×10⁸ total CAR-expressing T cells, 1×10⁸ to 5×10⁸ total CAR-expressing T cells, 1×10⁸ to 2.5×10⁸ total CAR-expressing T cells, or 2.5×10⁸ to 5×10⁸ total CAR-expressing T cells.

In some embodiments, the dose of genetically engineered cells comprises at least or at least about or is or is about 1×10⁵ CAR-expressing cells, at least or at least about or is or is about 2.5×10⁵ CAR-expressing cells, at least or at least about or is or is about 5×10⁵ CAR-expressing cells, at least or at least about or is or is about 1×10⁶ CAR-expressing cells, at least or at least about or is or is about 2.5×10⁶ CAR-expressing cells, at least or at least about or is or is about 5×10⁶ CAR-expressing cells, at least or at least about or is or is about 1×10⁷ CAR-expressing cells, at least or at least about or is or is about 2.5×10⁷ CAR-expressing cells, at least or at least about or is or is about 5×10⁷ CAR-expressing cells, at least or at least about or is or is about 1×10⁸ CAR-expressing cells, at least or at least about or is or is about 2.5×10⁸ CAR-expressing cells, or at least or at least about or is or is about 5×10⁸ CAR-expressing cells.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such as 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In some embodiments, for example, where the subject is a human, the dose includes fewer than about 1×10⁸ total recombinant receptor (e.g., recombinant TCR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 1:5 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the size of the dose is determined by the burden of the disease or condition in the subject. For example, in some aspects, the number of cells administered in the dose is determined based on the tumor burden that is present in the subject immediately prior to administration of the initiation of the dose of cells. In some embodiments, the size of the first and/or subsequent dose is inversely correlated with disease burden. In some aspects, as in the context of a large disease burden, the subject is administered a low number of cells. In other embodiments, as in the context of a lower disease burden, the subject is administered a larger number of cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known. See, for instance, Wadwa et al., J. Drug Targeting 3: 1 1 1 (1995), and U.S. Pat. No. 5,087,616.

### VII. KITS AND ARTICLES OF MANUFACTURE

Also provided are articles of manufacture, systems, apparatuses, and kits useful in performing the provided embodiments. In some embodiments, the provided articles of manufacture or kits contain one or more components of the one or more agent(s) capable of inducing genetic disruption and/or template polynucleotide(s), e.g., template polynucleotides containing transgene encoding the recombinant receptor or antigen-binding fragment or chain thereof or the one or more second template polynucleotides. In some embodiments, the articles of manufacture or kits can be used in methods for engineering T cells to express a recombinant receptor and/or other polypeptides and assessing the produced cells and/or cell populations in accord with the provided methods. In some embodiments, the articles of manufacture or kits provided herein contain T cells and/or T cell compositions, such as any T cells and/or T cell compositions described herein.

In some embodiments, the articles of manufacture or kits include polypeptides, nucleic acids, vectors and/or polynucleotides useful in performing the provided methods. In some embodiments, the articles of manufacture or kits include one or more nucleic acid molecules, e.g., a plasmid or a DNA fragment, that comprises one or more components of the one or more agent(s) capable of inducing genetic disruption and/or template polynucleotide(s), e.g., template polynucleotides containing transgene encoding the recombinant receptor or antigen-binding fragment or chain thereof or the one or more second template polynucleotides. In some embodiments, the articles of manufacture or kits provided herein contain control vectors.

In some embodiments, the articles of manufacture or kits provided herein contain one or more agent, wherein each of the one or more agent is independently capable of inducing a genetic disruption of a target site within a T cell receptor alpha constant (TRAC) gene and/or a T cell receptor beta constant (TRBC) gene; and a template polynucleotide comprising a transgene encoding a recombinant TCR or an antigen-binding fragment or α chain thereof, wherein the transgene encoding the recombinant TCR or antigen-binding fragment or chain thereof is targeted for integration at or near the target site via homology directed repair (HDR).

In some embodiments, the articles of manufacture or kits provided herein contain T cells, and/or T cell compositions, such as any T cells, and/or T cell compositions described herein. In some embodiments, the T cells, and/or T cell compositions any of the modified T cells used the screening methods described herein. In some embodiments, the articles of manufacture or kits provided herein contain control T cells, and/or T cell compositions.

In some embodiments, the articles of manufacture or kits include one or more components used to assess the properties of the population and/or composition of engineered cells expressing a recombinant receptor. For example, the articles of manufacture or kits can include binding reagents, e.g., antibodies, MHC-peptide tetramers and/or probes, used to assess particular properties of the introduced recombinant TCRs, e.g., cell surface expression of the recombinant TCR, recognition of a peptide in the context of an MHC molecule and/or detectable signal produced by the reporter, e.g., a T cell activation reporter. In some embodiments, the articles of manufacture or kits can include components that are used for detection of particular properties, such as labeled components, e.g., fluorescently labeled components and/or components that can produce a detectable signal, e.g., substrates that can produce fluorescence or luminescence.

In some embodiments, the articles of manufacture or kits include one or more containers, typically a plurality of containers, packaging material, and a label or package insert on or associated with the container or containers and/or packaging, generally including instructions for use, e.g., instructions for introducing the components into the cells for engineering and/or for assessing the engineered cell populations and/or compositions. In some embodiments, the article of manufacture or kits include one or more instructions for administration of the engineered cells and/or cell compositions for therapy.

The articles of manufacture provided herein contain packaging materials. Packaging materials for use in packaging the provided materials are well known. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the p agent capable of inducing genetic disruption and/or template polynucleotide(s) are packaged separately. In some embodiments, each container can have a single compartment. In some embodiments, other components of the articles of manufacture or kits are packaged separately, or together in a single compartment.

### VIII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a TCR or an antibody” refers to one or more nucleic acid molecules encoding TCR alpha or β chains (or fragments thereof) or antibody heavy and light chains (or fragments thereof), including such nucleic acid molecule(s) in a single vector or separate vectors, and such nucleic acid molecule(s) present at one or more locations in a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known, for instance, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In some embodiments, “about” may refer to ±25%, ±20%, ±15%, ±10%, ±5%, or ±1%.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### IX. EXEMPLARY EMBODIMENTS

Among the provided embodiments are:

1. A method of producing a genetically engineered immune cell, comprising:

(a) introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and

(b) introducing into the immune cell a template polynucleotide comprising a transgene encoding a recombinant T cell receptor (TCR) or an antigen-binding fragment thereof or α chain thereof, wherein the transgene encoding the recombinant TCR or antigen-binding fragment or chain thereof is targeted for integration at or near one of the at least one target site via homology directed repair (HDR).

2. A method of producing a genetically engineered immune cell, comprising:

introducing into an immune cell having a genetic disruption of at least one target site within a T cell receptor alpha constant (TRAC) gene and/or a T cell receptor beta constant (TRBC) gene a template polynucleotide comprising a transgene encoding a recombinant T cell receptor (TCR) or an antigen-binding fragment thereof or α chain thereof, wherein the genetic disruption has been induced by one or more agent, wherein each of the one or more agent is independently capable of inducing a genetic disruption, and the transgene encoding the recombinant TCR or antigen-binding fragment or chain thereof is targeted for integration at or near one of the at least one target position via homology directed repair (HDR).

3. The method of embodiment 1 or embodiment 2, wherein at least one of the one or more agent is capable of inducing a genetic disruption of a target site in a TRAC gene.

4. The method of any of embodiments 1-3, wherein at least one of the one or more agent is capable of inducing a genetic disruption of a target site in a TRBC gene.

5. The method of any of embodiments 1-4, wherein the one or more agents comprises at least one agent that capable of inducing a genetic disruption of a target site in a TRAC gene and at least one agent that is capable of inducing a genetic disruption of a target site in a TRBC gene.

6. The method of embodiment 4 or embodiment 5, wherein the TRBC gene is one or both of a T cell receptor beta constant 1 (TRBC1) or T cell receptor beta constant 2 (TRBC2) gene.

7. A method of producing a genetically engineered immune cell, comprising:

(a) introducing into an immune cell one or more agent, wherein each of the one or more agent is independently capable of inducing a genetic disruption of a target site within a T cell receptor alpha constant (TRAC) gene and a T cell receptor beta constant (TRBC) gene, thereby inducing a genetic disruption of the target sites; and

(b) introducing into the immune cell a template polynucleotide comprising a transgene encoding a recombinant T cell receptor (TCR) or an antigen-binding fragment thereof or α chain thereof, wherein the transgene encoding the recombinant TCR or antigen-binding fragment or chain thereof is targeted for integration at or near the target site via homology directed repair (HDR).

8. A method of producing a genetically engineered immune cell, comprising:

introducing into an immune cell having a genetic disruption of at least one target site within a T cell receptor alpha constant (TRAC) gene and/or a T cell receptor beta constant (TRBC) gene a template polynucleotide comprising a transgene encoding a recombinant T cell receptor (TCR) or an antigen-binding fragment thereof or α chain thereof, wherein the genetic disruption has been induced by one or more agent wherein each of the one or more agent is independently capable of inducing a genetic disruption, and the transgene encoding the recombinant TCR or antigen-binding fragment or chain thereof is targeted for integration at or near one of the at least one target site via homology directed repair (HDR).

9. The method of embodiment 7 or embodiment 8, wherein the TRBC gene is one or both of a T cell receptor beta constant 1 (TRBC1) or T cell receptor beta constant 2 (TRBC2) gene.

10. The method of any of embodiments 1-9, wherein the one or more agent capable of inducing a genetic disruption comprises a DNA binding protein or DNA-binding nucleic acid that specifically binds to or hybridizes to the target site.

11. The method of embodiment 10, wherein the one or more agent capable of inducing a genetic disruption comprises (a) a fusion protein comprising a DNA-targeting protein and a nuclease or (b) an RNA-guided nuclease.

12. The method of embodiment 11, wherein the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site.

13. The method of any of embodiments 10-12, wherein the one or more agent comprises a zinc finger nuclease (ZFN), a TAL-effector nuclease (TALEN), or and a CRISPR-Cas9 combination that specifically binds to, recognizes, or hybridizes to the target site.

14. The method of any of embodiments 1-13, wherein the each of the one or more agent comprises a guide RNA (gRNA) having a targeting domain that is complementary to the at least one target site.

15. The method of embodiment 14, wherein the one or more agent is introduced as a ribonucleoprotein (RNP) complex comprising the gRNA and a Cas9 protein.

16. The method of embodiment 15, wherein the RNP is introduced via electroporation, particle gun, calcium phosphate transfection, cell compression or squeezing.

17. The method of embodiment 15 or embodiment 16, wherein the RNP is introduced via electroporation.

18. The method of any of embodiments 1-13, wherein the one or more agent is introduced as one or more polynucleotide encoding the gRNA and/or a Cas9 protein.

19. The method of any of embodiments 1-18, wherein the at least one target site is within an exon of the TRAC, TRBC1 and/or TRBC2 gene.

20. The method of any of embodiments 14-19, wherein the gRNA has a targeting domain that is complementary to a target site in a TRAC gene and comprises a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

21. The method of embodiment 20, wherein the gRNA has a targeting domain comprising the sequence GAGAAUCAAAAUCGGUGAAU (SEQ ID NO:31).

22. The method of any of embodiments 14-21, wherein the gRNA has a targeting domain that is complementary to a target site in one or both of a TRBC1 and a TRBC2 gene and comprises a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

23. The method of embodiment 22, wherein the gRNA has a targeting domain comprising the sequence GGCCUCGGCGCUGACGAUCU (SEQ ID NO:63).

24. The method of any of embodiments 1-23, wherein the template polynucleotide comprises the structure [5′ homology arm]-[transgene]-[3′ homology arm].

25. The method of embodiment 24, wherein the 5′ homology arm and 3′ homology arm comprises nucleic acid sequences homologous to nucleic acid sequences surrounding the at least one target site.

26. The method of embodiment 24 or embodiment 25, wherein the 5′ homology arm comprises nucleic acid sequences that are homologous to nucleic acid sequences 5′ of the target site.

27. The method of embodiment 24 or embodiment 25, wherein the 3′ homology arm comprises nucleic acid sequences that are homologous to nucleic acid sequences 3′ of the target site.

28. The method of any of embodiments 24-27, wherein the 5′ homology arm and 3′ homology arm independently are at least or at least about 10, 20, 30, 40, 50, 100, 200, 300, 400, 500, 600, 700, 800, 900, 1000, 1500, or 2000 base pairs, or less than or less than about 10, 20, 30, 40, 50, 100, 200, 300, 400, 500, 600, 700, 800, 900, 1000, 1500, or 2000 base pairs.

29. The method of embodiment 28, wherein the 5′ homology arm and 3′ homology arm independently are between about 50 and 100, 100 and 250, 250 and 500, 500 and 750, 750 and 1000, 1000 and 2000 base pairs.

30. The method of embodiment 29, wherein the 5′ homology arm and 3′ homology arm independently are about 10, 20, 30, 40, 50, 100, 200, 300, 400, 500, 600, 700, 800, 900, 1000, 1500, or 2000 base pairs.

31. The method of any of embodiments 1-30, wherein the transgene encoding the recombinant TCR or antigen-binding fragment or chain thereof is targeted for integration at or near the target site in the TRAC gene.

32. The method of any of embodiments 1-32, wherein the transgene encoding the recombinant TCR or antigen-binding fragment or chain thereof is targeted for integration at or near the target site in one or both of the TRBC1 and the TRBC2 gene.

33. The method of any of embodiments 1-32, further comprising introducing into the immune cell one or more second template polynucleotide comprising one or more second transgene, wherein the second transgene is targeted for integration at or near one of the at least one target site via homology directed repair (HDR).

34. The method of embodiment 33, wherein the second template polynucleotide comprises the structure [second 5′ homology arm]-[one or more second transgene]-[second 3′ homology arm].

35. The method of embodiment 34, wherein the second 5′ homology arm and second 3′ homology arm comprise nucleic acid sequences homologous to nucleic acid sequences surrounding the at least one target site.

36. The method of embodiment 34 or embodiment 35, wherein the second 5′ homology arm comprises nucleic acid sequences that are homologous to nucleic acid sequences second 5′ of the target site.

37. The method of embodiment 34 or embodiment 35, wherein the second 3′ homology arm comprises nucleic acid sequences that are homologous to nucleic acid sequences second 3′ of the target site.

38. The method of any of embodiments 34-37, wherein the second 5′ homology arm and second 3′ homology arm independently are at least or at least about 10, 20, 30, 40, 50, 100, 200, 300, 400, 500, 600, 700, 800, 900, 1000, 1500, or 2000 base pairs, or less than or less than about 10, 20, 30, 40, 50, 100, 200, 300, 400, 500, 600, 700, 800, 900, 1000, 1500, or 2000 base pairs.

39. The method of embodiment 38, wherein the second 5′ homology arm and second 3′ homology arm independently are between about 50 and 100, 100 and 250, 250 and 500, 500 and 750, 750 and 1000, 1000 and 2000 base pairs.

40. The method of embodiment 39, wherein the second 5′ homology arm and second 3′ homology arm independently are about 10, 20, 30, 40, 50, 100, 200, 300, 400, 500, 600, 700, 800, 900, 1000, 1500, or 2000 base pairs.

41. The method of any of embodiments 33-40, wherein the one or more second transgene is targeted for integration at or near the target site in the TRAC gene.

42. The method of any of embodiments 33-41, wherein the one or more second transgene is targeted for integration at or near the target site in the TRBC1 or the TRBC2 gene.

43. The method of any of embodiments 33-42, wherein transgene encoding the recombinant TCR or antigen-binding fragment or chain thereof is targeted for integration at or near the target site in the TRAC gene, the TRBC1 gene or the TRBC2 gene, and the one or more second transgene is targeted for integration at or near one or more of the target site that is not targeted by the transgene encoding the recombinant TCR or antigen-binding fragment or chain thereof.

44. The method of any of embodiments 33-43, wherein the transgene encoding the recombinant TCR or antigen-binding fragment or chain thereof is targeted for integration at or near the target site in the TRAC gene, and the one or more second transgene is targeted for integration at or near one or more of the target site in the TRBC1 gene and/or the TRBC2 gene.

45. The method of any of embodiments 33-44, wherein the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor.

46. The method of embodiment 45, wherein the encoded molecule is a co-stimulatory ligand optionally selected from among a tumor necrosis factor (TNF) ligand selected from 4-1BBL, OX40L, CD70, LIGHT and CD30L, or an immunoglobulin (Ig) superfamily ligand selected from CD80 and CD86.

47. The method of embodiment 45, wherein the encoded molecule is a cytokine optionally selected from among IL-2, IL-3, IL-6, IL-11, IL-12, IL-7, IL-15, IL-21, granulocyte macrophage colony stimulating factor (GM-CSF), interferon alpha (IFN-α), interferon beta (IFN-β) or interferon gamma (IFN-γ) and erythropoietin.

48. The method of embodiment 45, wherein the encoded molecule is a soluble single-chain variable fragment (scFv) that optionally binds a polypeptide that has immunosuppressive activity or immunostimulatory activity selected from CD47, PD-1, CTLA-4 and ligands thereof or CD28, OX-40, 4-1BB and ligands thereof.

49. The method of embodiment 45, wherein the encoded molecule is an immunomodulatory fusion protein, optionally comprising (a) an extracellular binding domain that specifically binds an antigen derived from CD200R, SIRPα, CD279 (PD-1), CD2, CD95 (Fas), CD152 (CTLA4) CD223 (LAG3), CD272 (BTLA). A2aR, KIR, TIM3, CD300 or LPA5: (b) an intracellular signaling domain derived from CD3ε, CD3δ, CD3ζ, CD25, CD27, CD28, CD40, CD47, CD79A, CD79B, CD134 (OX40), CD137 (4-1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRFM, Zap70, PTCH2, or any combination thereof; and (c) a hydrophobic transmembrane domain derived from CD2, CD3ε, CD3δ, CD3ζ, CD25, CD27, CD28, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5 or Zap70.

50. The method of embodiment 45, wherein the encoded molecule is a chimeric switch receptor (CSR) that optionally comprises a truncated extracellular domain of PD1 and the transmembrane and cytoplasmic signaling domains of CD28.

51. The method of embodiment 45, wherein the encoded molecule is a co-receptor optionally selected from CD4 or CD8.

52. The method of any of embodiments 33-44, wherein transgene encoding the recombinant TCR or antigen-binding fragment or chain thereof encodes one chain of a recombinant TCR and the second transgene encodes a different chain of the recombinant TCR.

53. The method of embodiment 52, wherein transgene encoding the recombinant TCR or antigen-binding fragment or chain thereof encodes the alpha (TCRα) chain of the recombinant TCR and the second transgene encodes the beta (TCRβ) chain of the recombinant TCR.

54. The method of any of embodiments 1-53, wherein the transgene encoding the recombinant TCR or antigen-binding fragment or chain thereof and/or the one or more second transgene independently further comprises a regulatory or control element.

55. The method of embodiment 54, wherein the regulatory or control element comprises a promoter, an enhancer, an intron, a polyadenylation signal, a Kozak consensus sequence, an internal ribosome entry sites (IRES), a sequence encoding a ribosome skip sequence, a splice acceptor sequence or a splice donor sequence.

56. The method of embodiment 55, wherein the regulatory or control element comprises a promoter.

57. The method of embodiment 56, wherein the promoter is selected from among a constitutive promoter, an inducible promoter, a repressible promoter and/or a tissue-specific promoter.

58. The method of embodiment 56 or embodiment 57, wherein the promoter is selected from among an RNA pol I, pol II or pol III promoter.

59. The method of embodiment 58, wherein the promoter is selected from:

a pol III promoter that is a U6 or H1 promoter; or

a pol II promoter that is a CMV, SV40 early region or adenovirus major late promoter.

60. The method of any of embodiments 56-58, wherein the promoter is or comprises a human elongation factor 1 alpha (EF1α) promoter or an MND promoter or a variant thereof.

61. The method of any of embodiments 56-58, wherein the promoter is an inducible promoter or a repressible promoter.

62. The method of embodiment 61, wherein the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

63. The method of any of embodiments 1-62, wherein the transgene encoding the recombinant TCR or antigen-binding fragment or chain thereof and/or the one or more second transgene independently comprises one or more multicistronic element(s).

64. The method of embodiment 63, wherein the one or more multicistronic element(s) are upstream of the transgene encoding the recombinant TCR or antigen-binding fragment or chain thereof and/or the one or more second transgene.

65. The method of embodiment 63 or embodiment 64, wherein the multicistronic element(s) is positioned between the transgene encoding the recombinant TCR or antigen-binding fragment or chain thereof and the one or more second transgene.

66. The method of any of embodiments 63-65, wherein the multicistronic element(s) is positioned between the nucleic acid sequence encoding the TCRα or a portion thereof and the nucleic acid sequence encoding the TCRβ or a portion thereof.

67. The method of embodiment 66, wherein the multicistronic element(s) comprises a sequence encoding a ribosome skip element selected from among a T2A, a P2A, a E2A or a F2A or an internal ribosome entry site (IRES).

68. The method of embodiment 67, wherein the sequence encoding a ribosome skip element is targeted to be in-frame with the gene at the target site.

69. The method of any of embodiments 1-53, wherein the transgene encoding the recombinant TCR or antigen-binding fragment or chain thereof and/or the one or more second transgene independently is operably linked to the endogenous promoter of the gene at the target site.

70. The method of any of embodiments 1-69, wherein the recombinant TCR is capable of binding to an antigen that is associated with, specific to, and/or expressed on a cell or tissue of a disease, disorder or condition.

71. The method of embodiment 70, wherein the disease, disorder or condition is an infectious disease or disorder, an autoimmune disease, an inflammatory disease, or a tumor or a cancer.

72. The method of embodiment 70 or embodiment 71, wherein the antigen is a tumor antigen or a pathogenic antigen.

73. The method of embodiment 72, wherein the pathogenic antigen is a bacterial antigen or viral antigen.

74. The method of embodiment 73, wherein the antigen is a viral antigen and the viral antigen is from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV).

75. The method of embodiment 74, wherein the antigen is an antigen from an HPV selected from among HPV-16, HPV-18, HPV-31, HPV-33 and HPV-35.

76. The method of embodiment 75, wherein the antigen is an HPV-16 antigen that is an HPV-16 E6 or HPV-16 E7 antigen.

77. The method of embodiment 73, wherein the viral antigen is an EBV antigen selected from among Epstein-Ban nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA.

78. The method of embodiment 73, wherein the viral antigen is an HTLV-antigen that is TAX.

79. The method of embodiment 73, wherein the viral antigen is an HBV antigen that is a hepatitis B core antigen or a hepatitis B envelope antigen.

80. The method of any of embodiments 70-72, wherein the antigen is a tumor antigen.

81. The method of embodiment 80, wherein the antigen is selected from among glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Rα, c-Met, PSMA, Glycolipid F77, GD-2, insulin growth factor (IGF)-I, IGF-II, IGF-I receptor and mesothelin.

82. The method of any of embodiments 1-81, wherein the immune cell is a T cell.

83. The method of embodiment 82, wherein the T cell is a CD8+ T cell or subtypes thereof.

84. The method of embodiment 82, wherein the T cell is a CD4+ T cell or subtypes thereof.

85. The method of any of embodiments 1-81, wherein the immune cell is derived from a multipotent or pluripotent cell, which optionally is an iPSC.

86. The method of any of embodiments 1-85, wherein the immune cell comprises a T cell that is autologous to the subject.

87. The method of any of embodiments 1-85, wherein the immune cell comprises a T cell that is allogeneic to the subject.

88. The method of any of embodiments 1-87, wherein the first template polynucleotide, the one or more second template polynucleotide and/or the one or more polynucleotide encoding the gRNA and/or a Cas9 protein is comprised in one or more vector(s), which optionally are viral vector(s).

89. The method of embodiment 88, wherein the vector is an AAV vector.

90. The method of embodiment 89, wherein the AAV vector is selected from among AAV1, AAV2, AAV3, AAV4, AAV5, AAV6, AAV7 or AAV8 vector.

91. The method of embodiment 90, wherein the AAV vector is an AAV2 or AAV6 vector.

92. The method of embodiment 88, wherein the viral vector is a retroviral vector.

93. The method of embodiment 92, wherein the viral vector is a lentiviral vector.

94. The method of any of embodiments 1-93, wherein the introduction of the one or more agent capable of inducing a genetic disruption and the introduction of the template polynucleotide are performed simultaneously or sequentially, in any order.

95. The method of any of embodiments 1-94, wherein the introduction of the template polynucleotide is performed after the introduction of the one or more agent capable of inducing a genetic disruption.

96. The method of embodiment 95, wherein the template polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption.

97. The method of any of embodiments 33-96, wherein the introduction of the template polynucleotide and the introduction of the one or more second template polynucleotide are performed simultaneously or sequentially, in any order.

98. The method of any of embodiments 1-97, wherein introduction of the one or more agent capable of inducing a genetic disruption and the introduction of the template polynucleotide are performed in one experimental reaction.

99. The method of any of embodiments 33-98, wherein introduction of the one or more agent capable of inducing a genetic disruption and the introduction of the template polynucleotide and the second template polynucleotide(s) are performed in one experimental reaction.

100. The method of any of embodiments 1-99, wherein at least or greater than 35%, 40%, 45%, 50%, 55%, 60%, 65%, 70%, 75%, 80%, or 90% of the cells in a plurality of engineered cells comprise a genetic disruption of at least one target site within a gene encoding a domain or region of T cell receptor alpha constant (TRAC) gene and/or a T cell receptor beta constant (TRBC) gene.

101. The method of any of embodiments 1-99, wherein at least or greater than 35%, 40%, 45%, 50%, 55%, 60%, 65%, 70%, 75%, 80%, or 90% of the cells in a plurality of engineered cells express the recombinant receptor or antigen-binding fragment thereof and/or exhibit antigen binding.

102. The method of any of embodiments 1-99, wherein the coefficient of variation of expression and/or antigen binding of the recombinant receptor or antigen-binding fragment thereof among a plurality of engineered cells is lower than 0.70, 0.65, 0.60, 0.55, 0.50, 0.45, 0.40, 0.35 or 0.30 or less.

103. The method of any of embodiments 1-99, wherein the coefficient of variation of expression and/or antigen binding of the recombinant receptor or antigen-binding fragment thereof among a plurality of engineered cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

104. The method of any of embodiments 100-103, wherein expression and/or antigen-binding of the recombinant receptor or antigen-binding fragment thereof is assessed by contacting the cells in the composition with a binding reagent specific for the TCRα chain or the TCRβ chain and assessing binding of the reagent to the cells.

105. The method of embodiment 104, wherein the binding reagent is an anti-TCR Vβ antibody or is an anti-TCR Vα antibody that specifically recognizes a specific family of Vβ or Vα chains.

106. The method of embodiment 104, wherein the binding agent is a peptide antigen-MHC complex, which optionally is a tetramer.

107. An engineered cell or a plurality of engineered cells, generated using the method of any of embodiments 1-106.

108. A composition, comprising the engineered cell or plurality of engineered cells of embodiment 107.

109. The composition of embodiment 108, wherein at least or greater than 35%, 40%, 45%, 50%, 55%, 60%, 65%, 70%, 75%, 80%, or 90% of the cells in the composition comprise a genetic disruption of at least one target site within a gene encoding a domain or region of T cell receptor alpha constant (TRAC) gene and/or a T cell receptor beta constant (TRBC) gene.

110. The composition of embodiment 108, wherein at least or greater than 35%, 40%, 45%, 50%, 55%, 60%, 65%, 70%, 75%, 80%, or 90% of the cells in the composition express the recombinant receptor or antigen-binding fragment thereof and/or exhibit antigen binding.

111. The composition of embodiment 108, wherein the coefficient of variation of expression and/or antigen binding of the recombinant receptor or antigen-binding fragment thereof among the plurality of cells is lower than 0.70, 0.65, 0.60, 0.55, 0.50, 0.45, 0.40, 0.35 or 0.30 or less.

112. The composition of embodiment 108, wherein the coefficient of variation of expression and/or antigen binding of the recombinant receptor or antigen-binding fragment thereof among the plurality of cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

113. The composition of any of embodiments 109-112, wherein expression and/or antigen-binding of the recombinant receptor or antigen-binding fragment thereof is assessed by contacting the cells in the composition with a binding reagent specific for the TCRα chain or the TCRβ chain and assessing binding of the reagent to the cells.

114. The composition of embodiment 113, wherein the binding reagent is an anti-TCR Vβ antibody or is an anti-TCR Vα antibody that specifically recognizes a specific family of Vβ or Vα chains.

115. The composition of embodiment 113, wherein the binding agent is a peptide antigen-MHC complex, which optionally is a tetramer.

116. The composition of any of embodiments 108-115, further comprising a pharmaceutically acceptable carrier.

117. A composition, comprising a plurality of engineered T cells comprising a recombinant receptor or an antigen-binding fragment or chain thereof encoded by a transgene and a genetic disruption of at least one target site within a T cell receptor alpha constant (TRAC) gene and/or a T cell receptor beta constant (TRBC) gene, wherein at least or greater than 35%, 40%, 45%, 50%, 55%, 60%, 65%, 70%, 75%, 80%, or 90% of the cells in the composition comprise a genetic disruption of at least one target site within a TRAC gene and/or a TRBC gene; and

the transgene encoding the recombinant TCR or antigen-binding fragment or chain thereof is targeted for integration at or near one of the at least one target site via homology directed repair (HDR).

118. A composition, comprising a plurality of engineered T cells comprising a recombinant receptor or an antigen-binding fragment or chain thereof encoded by a transgene and a genetic disruption of at least one target site within a T cell receptor alpha constant (TRAC) gene and/or a T cell receptor beta constant (TRBC) gene, wherein at least or greater than 35%, 40%, 45%, 50%, 55%, 60%, 65%, 70%, 75%, 80%, or 90% of the cells in the composition express the recombinant receptor or antigen-binding fragment thereof and/or exhibit antigen binding; and

the transgene encoding the recombinant TCR or antigen-binding fragment or chain thereof is targeted for integration at or near one of the at least one target site via homology directed repair (HDR).

119. A composition, comprising a plurality of engineered T cells comprising a recombinant receptor or an antigen-binding fragment thereof encoded by a transgene and a genetic disruption of at least one target site within a T cell receptor alpha constant (TRAC) gene and/or a T cell receptor beta constant (TRBC) gene, wherein the coefficient of variation of expression and/or antigen binding of the recombinant receptor among the plurality of cells is lower than 0.70, 0.65, 0.60, 0.55, 0.50, 0.45, 0.40, 0.35 or 0.30 or less.

120. A composition, comprising a plurality of engineered T cells comprising a recombinant receptor or an antigen-binding fragment thereof encoded by a transgene and a genetic disruption of at least one target site within a T cell receptor alpha constant (TRAC) gene and/or a T cell receptor beta constant (TRBC) gene, wherein the coefficient of variation of expression and/or antigen binding of the recombinant receptor among the plurality of cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

121. The composition of any of embodiments 117-120, wherein the composition is generated by:

(a) introducing into a plurality of T cells one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and

(b) introducing into the plurality of T cells a template polynucleotide comprising a transgene encoding a recombinant T cell receptor (TCR) or an antigen-binding fragment or a chain thereof, wherein the transgene encoding the recombinant TCR or antigen-binding fragment or chain thereof is targeted for integration at or near one of the at least one target site via homology directed repair (HDR).

122. The composition of any of embodiments 117-121, wherein expression and/or antigen-binding of the recombinant receptor or antigen-binding fragment thereof is assessed by contacting the cells in the composition with a binding reagent specific for the TCRα chain or the TCRβ chain and assessing binding of the reagent to the cells.

123. The composition of embodiment 122, wherein the binding reagent is an anti-TCR Vβ antibody or is an anti-TCR Vα antibody that specifically recognizes a specific family of Vβ or Vα chains.

124. The composition of embodiment 122, wherein the binding agent is a peptide antigen-MHC complex, which optionally is a tetramer.

125. The composition of any of embodiments 121-124, wherein at least one of the one or more agent is capable of inducing a genetic disruption of a target site in a TRAC gene.

126. The composition of any of embodiments 121-125, wherein at least one of the one or more agent is capable of inducing a genetic disruption of a target site in a TRBC gene.

127. The composition of any of embodiments 121-126, wherein the one or more agents comprises at least one agent that capable of inducing a genetic disruption of a target site in a TRAC gene and at least one agent that is capable of inducing a genetic disruption of a target site in a TRBC gene.

128. The composition of any of embodiments 117-127, wherein the TRBC gene is one or both of a T cell receptor beta constant 1 (TRBC1) or T cell receptor beta constant 2 (TRBC2) gene.

129. The composition of any of embodiments 121-128, wherein the one or more agent capable of inducing a genetic disruption comprises a DNA binding protein or DNA-binding nucleic acid that specifically binds to or hybridizes to the target site.

130. The composition of embodiment 129, wherein the one or more agent capable of inducing a genetic disruption comprises (a) a fusion protein comprising a DNA-targeting protein and a nuclease or (b) an RNA-guided nuclease.

131. The composition of embodiment 130, wherein the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site.

132. The composition of any of embodiments 129-131, wherein the one or more agent comprises a zinc finger nuclease (ZFN), a TAL-effector nuclease (TALEN), or and a CRISPR-Cas9 combination that specifically binds to, recognizes, or hybridizes to the target site.

133. The composition of any of embodiments 121-132, wherein the each of the one or more agent comprises a guide RNA (gRNA) having a targeting domain that is complementary to the at least one target site.

134. The composition of embodiment 133, wherein the one or more agent is introduced as a ribonucleoprotein (RNP) complex comprising the gRNA and a Cas9 protein.

135. The composition of embodiment 134, wherein the RNP is introduced via electroporation, particle gun, calcium phosphate transfection, cell compression or squeezing.

136. The composition of embodiment 134 or embodiment 135, wherein the RNP is introduced via electroporation.

137. The composition of any of embodiments 121-132, wherein the one or more agent is introduced as one or more polynucleotide encoding the gRNA and/or a Cas9 protein.

138. The composition of any of embodiments 121-137, wherein the at least one target site is within an exon of the TRAC, TRBC1 and/or TRBC2 gene.

139. The composition of any of embodiments 133-138, wherein the gRNA has a targeting domain that is complementary to a target site in a TRAC gene and comprises a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

140. The composition of embodiment 139, wherein the gRNA has a targeting domain comprising the sequence GAGAAUCAAAAUCGGUGAAU (SEQ ID NO:31).

141. The composition of any of embodiments 133-140, wherein the gRNA has a targeting domain that is complementary to a target site in one or both of a TRBC1 and a TRBC2 gene and comprises a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

142. The composition of embodiment 141, wherein the gRNA has a targeting domain comprising the sequence GGCCUCGGCGCUGACGAUCU (SEQ ID NO:63).

143. The composition of any of embodiments 121-142, wherein the template polynucleotide comprises the structure [5′ homology arm]-[transgene]-[3′ homology arm].

144. The composition of embodiment 143, wherein the 5′ homology arm and 3′ homology arm comprises nucleic acid sequences homologous to nucleic acid sequences surrounding the at least one target site.

145. The composition of embodiment 143 or embodiment 144, wherein the 5′ homology arm comprises nucleic acid sequences that are homologous to nucleic acid sequences 5′ of the target site.

146. The composition of embodiment 143 or embodiment 144, wherein the 3′ homology arm comprises nucleic acid sequences that are homologous to nucleic acid sequences 3′ of the target site.

147. The composition of any of embodiments 143-146, wherein the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides.

148. The composition of embodiment 147, wherein the 5′ homology arm and 3′ homology arm independently are between about 50 and 100, 100 and 250, 250 and 500, 500 and 750, 750 and 1000, 1000 and 2000 nucleotides.

149. The composition of embodiment 148, wherein the 5′ homology arm and 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides.

150. The composition of any of embodiments 117-149, wherein the transgene encoding the recombinant TCR or antigen-binding fragment or chain thereof is targeted for integration at or near the target site in the TRAC gene.

151. The composition of any of embodiments 117-150, wherein the transgene encoding the recombinant TCR or antigen-binding fragment or chain thereof is targeted for integration at or near the target site in one or both of the TRBC1 and the TRBC2 gene.

152. The composition of any of embodiments 117-151, wherein the composition is generated by further introducing into the immune cell one or more second template polynucleotide comprising one or more second transgene, wherein the second transgene is targeted for integration at or near one of the at least one target site via homology directed repair (HDR).

153. The composition of embodiment 152, wherein the second template polynucleotide comprises the structure [second 5′ homology arm]-[one or more second transgene]-[second 3′ homology arm].

154. The composition of embodiment 153, wherein the second 5′ homology arm and second 3′ homology arm comprises nucleic acid sequences homologous to nucleic acid sequences surrounding the at least one target site.

55. The composition of embodiment 153 or embodiment 154, wherein the second 5′ homology arm comprises nucleic acid sequences that are homologous to nucleic acid sequences second 5′ of the target site.

156. The composition of embodiment 153 or embodiment 154, wherein the second 3′ homology arm comprises nucleic acid sequences that are homologous to nucleic acid sequences second 3′ of the target site.

157. The composition of any of embodiments 153-156, wherein the second 5′ homology arm and second 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides.

158. The composition of embodiment 157, wherein the second 5′ homology arm and second 3′ homology arm independently are between about 50 and 100, 100 and 250, 250 and 500, 500 and 750, 750 and 1000, 1000 and 2000 nucleotides.

159. The composition of embodiment 158, wherein the second 5′ homology arm and second 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides.

160. The composition of any of embodiments 152-159, wherein the one or more second transgene is targeted for integration at or near the target site in the TRAC gene.

161. The composition of any of embodiments 152-160, wherein the one or more second transgene is targeted for integration at or near the target site in the TRBC1 or the TRBC2 gene.

162. The composition of any of embodiments 152-161, wherein transgene encoding the recombinant TCR or antigen-binding fragment or chain thereof is targeted for integration at or near the target site in the TRAC gene, the TRBC1 gene or the TRBC2 gene, and the one or more second transgene is targeted for integration at or near one or more of the target site that is not targeted by the transgene encoding the recombinant TCR or antigen-binding fragment or chain thereof.

163. The composition of any of embodiments 152-162, wherein the transgene encoding the recombinant TCR or antigen-binding fragment or chain thereof is targeted for integration at or near the target site in the TRAC gene, and the one or more second transgene is targeted for integration at or near one or more of the target site in the TRBC1 gene and/or the TRBC2 gene.

164. The composition of any of embodiments 152-163, wherein the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor.

165. The composition of embodiment 164, wherein the encoded molecule is a co-stimulatory ligand optionally selected from among a tumor necrosis factor (TNF) ligand selected from 4-1BBL, OX40L, CD70, LIGHT and CD30L, or an immunoglobulin (Ig) superfamily ligand selected from CD80 and CD86.

166. The composition of embodiment 164, wherein the encoded molecule is a cytokine optionally selected from among IL-2, IL-3, IL-6, IL-11, IL-12, IL-7, IL-15, IL-21, granulocyte macrophage colony stimulating factor (GM-CSF), interferon alpha (IFN-α), interferon beta (IFN-β) or interferon gamma (IFN-γ) and erythropoietin.

167. The composition of embodiment 164, wherein the encoded molecule is a soluble single-chain variable fragment (scFv) that optionally binds a polypeptide that has immunosuppressive activity or immunostimulatory activity selected from CD47, PD-1, CTLA-4 and ligands thereof or CD28, OX-40, 4-1BB and ligands thereof.

168. The composition of embodiment 164, wherein the encoded molecule is an immunomodulatory fusion protein, optionally comprising:

(a) an extracellular binding domain that specifically binds an antigen derived from CD200R. SIRPα, CD279 (PD-1), CD2, CD95 (Fas). CD152 (CTLA4), CD223 (LAG3), CD272 A2aR, KIR, TIM3, CD300 or LPA5;

(b) an intracellular signaling domain derived from CD3ε, CD3δ, CD3ζ, CD25, CD27, CD28, CD40, CD47, CD79A, CD79B, CD134 (OX40), CD137 (4-1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRFM, Zap70, PTCH2, or any combination thereof; and

(c) a hydrophobic transmembrane domain derived from CD2, CD3ε, CD3δ, CD3ζ, CD25, CD27, CD28, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5 or Zap70.

169. The composition of embodiment 164, wherein the encoded molecule is a chimeric switch receptor (CSR) that optionally comprises a truncated extracellular domain of PD1 and the transmembrane and cytoplasmic signaling domains of CD28.

170. The composition of embodiment 164, wherein the encoded molecule is a co-receptor optionally selected from CD4 or CD8.

171. The composition of any of embodiments 152-163, wherein transgene encoding the recombinant TCR or antigen-binding fragment or chain thereof encodes one chain of a recombinant TCR and the second transgene encodes a different chain of the recombinant TCR.

172. The composition of embodiment 171, wherein transgene encoding the recombinant TCR or antigen-binding fragment or chain thereof encodes the alpha (TCRα) chain of the recombinant TCR and the second transgene encodes the beta (TCRβ) chain of the recombinant TCR.

173. The composition of any of embodiments 117-172, wherein the transgene encoding the recombinant TCR or antigen-binding fragment or chain thereof and/or the one or more second transgene independently further comprises a regulatory or control element.

174. The composition of embodiment 173, wherein the regulatory or control element comprises a promoter, an enhancer, an intron, a polyadenylation signal, a Kozak consensus sequence, a splice acceptor sequence or a splice donor sequence.

175. The composition of embodiment 174, wherein the regulatory or control element comprises a promoter.

176. The composition of embodiment 175, wherein the promoter is selected from among a constitutive promoter, an inducible promoter, a repressible promoter and/or a tissue-specific promoter.

177. The composition of embodiment 175 or embodiment 176, wherein the promoter is selected from among an RNA pol I, pol II or pol III promoter.

178. The composition of embodiment 177, wherein the promoter is selected from:

a pol III promoter that is a U6 or H1 promoter; or

a pol II promoter that is a CMV, SV40 early region or adenovirus major late promoter.

179. The composition of any of embodiments 175-177, wherein the promoter is or comprises a human elongation factor 1 alpha (EF1α) promoter or an MND promoter or a variant thereof.

180. The composition of any of embodiments 175-177, wherein the promoter is an inducible promoter or a repressible promoter.

181. The composition of embodiment 180, wherein the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

182. The composition of any of embodiments 108-181, wherein the transgene encoding the recombinant TCR or antigen-binding fragment or chain thereof and/or the one or more second transgene independently comprises one or more multicistronic element(s).

183. The composition of embodiment 182, wherein the one or more multicistronic element(s) are upstream of the transgene encoding the recombinant TCR or antigen-binding fragment or chain thereof and/or the one or more second transgene.

184. The composition of embodiment 182 or embodiment 183, wherein the multicistronic element(s) is positioned between the transgene encoding the recombinant TCR or antigen-binding fragment or chain thereof and the one or more second transgene.

185. The composition of any of embodiments 182-184, wherein the multicistronic element(s) is positioned between the nucleic acid sequence encoding the TCRα or a portion thereof and the nucleic acid sequence encoding the TCRβ or a portion thereof.

186. The composition of any of embodiments 182-185, wherein the multicistronic element(s) comprises a sequence encoding a ribosome skip element selected from among a T2A, a P2A, a E2A or a F2A or an internal ribosome entry site (IRES).

187. The composition of embodiment 186, wherein the sequence encoding a ribosome skip element is targeted to be in-frame with the gene at the target site.

188. The composition of any of embodiments 117-172, wherein upon HDR, the transgene encoding the recombinant TCR or antigen-binding fragment or chain thereof and/or the one or more second transgene independently is operably linked to the endogenous promoter of the gene at the target site.

189. The composition of any of embodiments 117-188, wherein the recombinant TCR is capable of binding to an antigen that is associated with, specific to, and/or expressed on a cell or tissue of a disease, disorder or condition.

190. The composition of embodiment 189, wherein the disease, disorder or condition is an infectious disease or disorder, an autoimmune disease, an inflammatory disease, or a tumor or a cancer.

191. The composition of embodiment 189 or embodiment 190, wherein the antigen is a tumor antigen or a pathogenic antigen.

192. The composition of embodiment 191, wherein the pathogenic antigen is a bacterial antigen or viral antigen.

193. The composition of embodiment 192, wherein the antigen is a viral antigen and the viral antigen is from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV).

194. The composition of embodiment 193, wherein the antigen is an antigen from an HPV selected from among HPV-16, HPV-18, HPV-31, HPV-33 and HPV-35.

195. The composition of embodiment 194, wherein the antigen is an HPV-16 antigen that is an HPV-16 E6 or HPV-16 E7 antigen.

196. The composition of embodiment 192, wherein the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA.

197. The composition of embodiment 192, wherein the viral antigen is an HTLV-antigen that is TAX.

198. The composition of embodiment 192, wherein the viral antigen is an HBV antigen that is a hepatitis B core antigen or a hepatitis B envelope antigen.

199. The composition of any of embodiments 189-191, wherein the antigen is a tumor antigen.

200. The composition of embodiment 199, wherein the antigen is selected from among glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD133, CD 166, epCAM, CA-125, HE4, Oval, estrogen receptor, progesterone receptor, uPA, PAI-1, CD19, CD20, CD22, ROR1, CD33/IL3Rα, c-Met, PSMA, Glycolipid F77, GD-2, insulin growth factor (IGF)-I, IGF-II, IGF-I receptor and mesothelin.

201. The composition of any of embodiments 1173-200, wherein the T cell is a CD8+ T cell or subtypes thereof.

202. The composition of any of embodiments 117-200, wherein the T cell is a CD4+ T cell or subtypes thereof.

203. The composition of any of embodiments 117-202, wherein the T cell is autologous to the subject.

204. The composition of any of embodiments 117-202, wherein the T cell is allogeneic to the subject.

205. The composition of any of embodiments 117-204, wherein the first template polynucleotide, the one or more second template polynucleotide and/or the one or more polynucleotide encoding the gRNA and/or a Cas9 protein is comprised in one or more vector(s), which optionally are viral vector(s).

206. The composition of embodiment 205, wherein the vector is an AAV vector.

207. The composition of embodiment 206, wherein the AAV vector is selected from among AAV1, AAV2, AAV3, AAV4, AAV5, AAV6, AAV7 or AAV8 vector.

208. The composition of embodiment 207, wherein the AAV vector is an AAV2 or AAV6 vector.

209. The composition of embodiment 205, wherein the viral vector is a retroviral vector.

210. The composition of embodiment 209, wherein the viral vector is a lentiviral vector.

211. The composition of any of embodiments 117-210, wherein the introduction of the one or more agent capable of inducing a genetic disruption and the introduction of the template polynucleotide are performed simultaneously or sequentially, in any order.

212. The composition of any of embodiments 117-211, wherein the introduction of the template polynucleotide is performed after the introduction of the one or more agent capable of inducing a genetic disruption.

213. The composition of embodiment 212, wherein the template polynucleotide is introduced immediately after, or within about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption.

214. The composition of any of embodiments 152-213, wherein the introduction of the template polynucleotide and the introduction of the one or more second template polynucleotide are performed simultaneously or sequentially, in any order.

215. The composition of any of embodiments 117-214, wherein introduction of the one or more agent capable of inducing a genetic disruption and the introduction of the template polynucleotide are performed in one experimental reaction.

216. The composition of any of embodiments 152-215, wherein introduction of the one or more agent capable of inducing a genetic disruption and the introduction of the template polynucleotide and the second template polynucleotide(s) are performed in one experimental reaction.

217. The composition of any of embodiments 108-216, further comprising a pharmaceutically acceptable carrier.

218. A method of treatment comprising administering the engineered cell, plurality of engineered cells or composition of any of embodiments 107-216 to a subject.

219. Use of the engineered cell, plurality of engineered cells or composition of any of embodiments 107-216 for treating cancer.

220. Use of the engineered cell, plurality of engineered cells or composition of any of embodiments 107-216 in the manufacture of a medicament for treating cancer.

221. The engineered cell, plurality of engineered cells or composition of any of embodiments 107-216 for use in treating cancer.

222. A kit, comprising:

one or more agent, wherein each of the one or more agent is independently capable of inducing a genetic disruption of a target site within a T cell receptor alpha constant (TRAC) gene and/or a T cell receptor beta constant (TRBC) gene; and

a template polynucleotide comprising a transgene encoding a recombinant TCR or an antigen-binding fragment or α chain thereof, wherein the transgene encoding the recombinant TCR or antigen-binding fragment or chain thereof is targeted for integration at or near the target site via homology directed repair (HDR).

223. The kit of embodiment 222, wherein the one or more agent capable of inducing a genetic disruption comprises a DNA binding protein or DNA-binding nucleic acid that specifically binds to or hybridizes to the target site.

224. The kit of embodiment 223, wherein the one or more agent capable of inducing a genetic disruption comprises (a) a fusion protein comprising a DNA-targeting protein and a nuclease or (b) an RNA-guided nuclease.

225. The kit of embodiment 224, wherein the DNA-targeting protein or RNA-guided nuclease comprises a zinc finger protein (ZFP), a TAL protein, or a clustered regularly interspaced short palindromic nucleic acid (CRISPR)-associated nuclease (Cas) specific for the target site.

226. The kit of any of embodiments 222-225, wherein the one or more agent comprises a zinc finger nuclease (ZFN), a TAL-effector nuclease (TALEN), or and a CRISPR-Cas9 combination that specifically binds to, recognizes, or hybridizes to the target site.

227. The kit of any of embodiments 222-226, wherein the each of the one or more agent comprises a guide RNA (gRNA) having a targeting domain that is complementary to the at least one target site.

228. The kit of embodiment 227, wherein the one or more agent is introduced as a ribonucleoprotein (RNP) complex comprising the gRNA and a Cas9 protein.

229. The kit of embodiment 228, wherein the RNP is introduced via electroporation, particle gun, calcium phosphate transfection, cell compression or squeezing.

230. The kit of embodiment 228 or embodiment 229, wherein the RNP is introduced via electroporation.

231. The kit of any of embodiments 222-230, wherein the one or more agent is introduced as one or more polynucleotide encoding the gRNA and/or a Cas9 protein.

232. The kit of any of embodiments 222-231, wherein the at least one target site is within an exon of the TRAC, TRBC1 and/or TRBC2 gene.

233. The kit of any of embodiments 227-232, wherein the gRNA has a targeting domain that is complementary to a target site in a TRAC gene and comprises a sequence selected from UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

234. The kit of embodiment 233, wherein the gRNA has a targeting domain comprising the sequence GAGAAUCAAAAUCGGUGAAU (SEQ ID NO:31).

235. The kit of any of embodiments 227-234, wherein the gRNA has a targeting domain that is complementary to a target site in one or both of a TRBC1 and a TRBC2 gene and comprises a sequence selected from CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

236. The kit of embodiment 235, wherein the gRNA has a targeting domain comprising the sequence GGCCUCGGCGCUGACGAUCU (SEQ ID NO:63).

237. The kit of any of embodiments 222-236, wherein the template polynucleotide comprises the structure [5′ homology arm]-[transgene]-[3′ homology arm].

238. The kit of embodiment 237, wherein the 5′ homology arm and 3′ homology arm comprises nucleic acid sequences homologous to nucleic acid sequences surrounding the at least one target site.

239. The kit of embodiment 237 or embodiment 238, wherein the 5′ homology arm comprises nucleic acid sequences that are homologous to nucleic acid sequences 5′ of the target site.

240. The kit of embodiment 237 or embodiment 238, wherein the 3′ homology arm comprises nucleic acid sequences that are homologous to nucleic acid sequences 3′ of the target site.

241. The kit of any of embodiments 237-240, wherein the 5′ homology arm and 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides.

242. The kit of embodiment 241, wherein the 5′ homology arm and 3′ homology arm independently are between about 50 and 100, 100 and 250, 250 and 500, 500 and 750, 750 and 1000, 1000 and 2000 nucleotides.

243. The kit of embodiment 242, wherein the 5′ homology arm and 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides.

244. The kit of any of embodiments 222-243, wherein the transgene encoding the recombinant TCR or antigen-binding fragment or chain thereof is targeted for integration at or near the target site in the TRAC gene.

245. The kit of any of embodiments 222-244, wherein the transgene encoding the recombinant TCR or antigen-binding fragment or chain thereof is targeted for integration at or near the target site in one or both of the TRBC1 and the TRBC2 gene.

246. The kit of any of embodiments 222-245, further comprising one or more second template polynucleotide comprising one or more second transgene, wherein the second transgene is targeted for integration at or near one of the at least one target site via homology directed repair (HDR).

247. The kit of embodiment 246, wherein the second template polynucleotide comprises the structure [second 5′ homology arm]-[one or more second transgene]-[second 3′ homology arm].

248. The kit of embodiment 247, wherein the second 5′ homology arm and second 3′ homology arm comprises nucleic acid sequences homologous to nucleic acid sequences surrounding the at least one target site.

249. The kit of embodiment 247 or embodiment 248, wherein the second 5′ homology arm comprises nucleic acid sequences that are homologous to nucleic acid sequences second 5′ of the target site.

250. The kit of embodiment 247 or embodiment 248, wherein the second 3′ homology arm comprises nucleic acid sequences that are homologous to nucleic acid sequences second 3′ of the target site.

251. The kit of any of embodiments 247-250, wherein the second 5′ homology arm and second 3′ homology arm independently are at least or at least about 10, 20, 30, 40, 50, 100, 200, 300, 400, 500, 600, 700, 800, 900, 1000, 1500, or 2000 nucleotides, or less than or less than about 10, 20, 30, 40, 50, 100, 200, 300, 400, 500, 600, 700, 800, 900, 1000, 1500, or 2000 nucleotides.

252. The kit of embodiment 251, wherein the second 5′ homology arm and second 3′ homology arm independently are between about 50 and 100, 100 and 250, 250 and 500, 500 and 750, 750 and 1000, 1000 and 2000 nucleotides.

253. The kit of embodiment 252, wherein the second 5′ homology arm and second 3′ homology arm independently are from or from about 100 to 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides.

254. The kit of any of embodiments 246-253, wherein the one or more second transgene is targeted for integration at or near the target site in the TRAC gene.

255. The kit of any of embodiments 246-253, wherein the one or more second transgene is targeted for integration at or near the target site in the TRBC1 or the TRBC2 gene.

256. The kit of any of embodiments 246-255, wherein transgene encoding the recombinant TCR or antigen-binding fragment or chain thereof is targeted for integration at or near the target site in the TRAC gene, the TRBC1 gene or the TRBC2 gene, and the one or more second transgene is targeted for integration at or near one or more of the target site that is not targeted by the transgene encoding the recombinant TCR or antigen-binding fragment or chain thereof.

257. The kit of any of embodiments 246-256, wherein the transgene encoding the recombinant TCR or antigen-binding fragment or chain thereof is targeted for integration at or near the target site in the TRAC gene, and the one or more second transgene is targeted for integration at or near one or more of the target site in the TRBC1 gene and/or the TRBC2 gene.

258. The kit of any of embodiments 246-257, wherein the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor.

259. The kit of embodiment 258, wherein the encoded molecule is a co-stimulatory ligand optionally selected from among a tumor necrosis factor (TNF) ligand selected from 4-1BBL, OX40L, CD70, LIGHT and CD30L, or an immunoglobulin (Ig) superfamily ligand selected from CD80 and CD86.

260. The kit of embodiment 258, wherein the encoded molecule is a cytokine optionally selected from among IL-2, IL-3, IL-6, IL-11, IL-30, IL-7, IL-24, IL-30, granulocyte macrophage colony stimulating factor (GM-CSF), interferon alpha (IFN-α), interferon beta (IFN-β) or interferon gamma (IFN-γ) and erythropoietin.

261. The kit of embodiment 258, wherein the encoded molecule is a soluble single-chain variable fragment (scFv) that optionally binds a polypeptide that has immunosuppressive activity or immunostimulatory activity selected from CD47, PD-1, CTLA-4 and ligands thereof or CD28, OX-40, 4-1BB and ligands thereof.

262. The kit of embodiment 258, wherein the encoded molecule is an immunomodulatory fusion protein, optionally comprising:

(a) an extracellular binding domain that specifically binds an antigen derived from CD290R, SIRPα, CD279 (PD-1). CD2, CD95 (Fas), CD242 (CTLA4), CD223 (LAG3), CD272 (BTLA), A2aR, KIR, TIM3, CD300 or LPA5;

(b) an intracellular signaling domain derived from CD3ε, CD3δ, CD3ζ, CD25, CD27, CD28, CD40, CD47, CD79A, CD79B, CD224 (OX40), CD227 (4-1BB), CD240 (SLAMF1), CD278 (ICOS), CD357 (GITR), CARD11, DAP10, DAP30, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRFM, Zap70, PTCH2, or any combination thereof; and

(c) a hydrophobic transmembrane domain derived from CD2, CD3ε, CD3δ, CD3ζ, CD25, CD27, CD28, CD40, CD79A, CD79B, CD80, CD86, CD95 (Fas), CD224 (OX40), CD227 (4-1BB), CD240 (SLAMF1), CD242 (CTLA4), CD290R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5 or Zap70.

263. The kit of embodiment 258, wherein the encoded molecule is a chimeric switch receptor (CSR) that optionally comprises a truncated extracellular domain of PD1 and the transmembrane and cytoplasmic signaling domains of CD28.

264. The kit of embodiment 258, wherein the encoded molecule is a co-receptor optionally selected from CD4 or CD8.

265. The kit of any of embodiments 246-257, wherein transgene encoding the recombinant TCR or antigen-binding fragment or chain thereof encodes one chain of a recombinant TCR and the second transgene encodes a different chain of the recombinant TCR.

266. The kit of embodiment 265, wherein transgene encoding the recombinant TCR or antigen-binding fragment or chain thereof encodes the alpha (TCRα) chain of the recombinant TCR and the second transgene encodes the beta (TCRβ) chain of the recombinant TCR.

267. The kit of any of embodiments 222-266, wherein the transgene encoding the recombinant TCR or antigen-binding fragment or chain thereof and/or the one or more second transgene independently further comprises a regulatory or control element.

268. The kit of embodiment 267, wherein the regulatory or control element comprises a promoter, an enhancer, an intron, a polyadenylation signal, a Kozak consensus sequence, a splice acceptor sequence or a splice donor sequence.

269. The kit of embodiment 268, wherein the regulatory or control element comprises a promoter.

270. The kit of embodiment 269, wherein the promoter is selected from among a constitutive promoter, an inducible promoter, a repressible promoter and/or a tissue-specific promoter.

271. The kit of embodiment 269 or embodiment 270, wherein the promoter is selected from among an RNA pol I, pol II or pol III promoter.

272. The kit of embodiment 271, wherein the promoter is selected from:

a pol III promoter that is a U6 or H1 promoter; or

a pol II promoter that is a CMV, SV40 early region or adenovirus major late promoter.

273. The kit of any of embodiments 269-271, wherein the promoter is or comprises a human elongation factor 1 alpha (EF1α) promoter or an MND promoter or a variant thereof.

274. The kit of any of embodiments 269-271, wherein the promoter is an inducible promoter or a repressible promoter.

275. The kit of embodiment 274, wherein the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof.

276. The kit of any of embodiments 222-275, wherein the transgene encoding the recombinant TCR or antigen-binding fragment or chain thereof and/or the one or more second transgene independently comprises one or more multicistronic element(s).

277. The kit of embodiment 276, wherein the one or more multicistronic element(s) are upstream of the transgene encoding the recombinant TCR or antigen-binding fragment or chain thereof and/or the one or more second transgene.

278. The kit of embodiment 276 or embodiment 277, wherein the multicistronic element(s) is positioned between the transgene encoding the recombinant TCR or antigen-binding fragment or chain thereof and the one or more second transgene.

279. The kit of any of embodiments 276-278, wherein the multicistronic element(s) is positioned between the nucleic acid sequence encoding the TCRα or a portion thereof and the nucleic acid sequence encoding the TCRβ or a portion thereof.

280. The kit of any of embodiments 276-279, wherein the multicistronic element(s) comprises a sequence encoding a ribosome skip element selected from among a T2A, a P2A, a E2A or a F2A or an internal ribosome entry site (IRES).

281. The kit of embodiment 280, wherein the sequence encoding a ribosome skip element is targeted to be in-frame with the gene at the target site.

282. The kit of any of embodiments 222-266, wherein upon HDR, the transgene encoding the recombinant TCR or antigen-binding fragment or chain thereof and/or the one or more second transgene independently is operably linked to the endogenous promoter of the gene at the target site.

283. The kit of any of embodiments 222-282, wherein the recombinant TCR is capable of binding to an antigen that is associated with, specific to, and/or expressed on a cell or tissue of a disease, disorder or condition.

284. The kit of embodiment 283, wherein the disease, disorder or condition is an infectious disease or disorder, an autoimmune disease, an inflammatory disease, or a tumor or a cancer.

285. The kit of embodiment 283 or embodiment 284, wherein the antigen is a tumor antigen or a pathogenic antigen.

286. The kit of embodiment 285, wherein the pathogenic antigen is a bacterial antigen or viral antigen.

287. The kit of embodiment 286, wherein the antigen is a viral antigen and the viral antigen is from hepatitis A, hepatitis B, hepatitis C virus (HCV), human papilloma virus (HPV), hepatitis viral infections, Epstein-Barr virus (EBV), human herpes virus 8 (HHV-8), human T-cell leukemia virus-1 (HTLV-1), human T-cell leukemia virus-2 (HTLV-2), or a cytomegalovirus (CMV).

288. The kit of embodiment 287, wherein the antigen is an antigen from an HPV selected from among HPV-25, HPV-27, HPV-31, HPV-33 and HPV-35.

289. The kit of embodiment 288, wherein the antigen is an HPV-25 antigen that is an HPV-25 E6 or HPV-25 E7 antigen.

290. The kit of embodiment 286, wherein the viral antigen is an EBV antigen selected from among Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA and EBV-VCA.

291. The kit of embodiment 286, wherein the viral antigen is an HTLV-antigen that is TAX.

292. The kit of embodiment 286, wherein the viral antigen is an HBV antigen that is a hepatitis B core antigen or a hepatitis B envelope antigen.

293. The kit of any of embodiments 283-285, wherein the antigen is a tumor antigen.

294. The kit of embodiment 293, wherein the antigen is selected from among glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, Melanin-A/MART-1, WT-1, S-100, MBP, CD63, MUC1 (e.g. MUC1-8), p53, Ras, cyclin B1, HER-2/neu, carcinoembryonic antigen (CEA), gp100, MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A11, MAGE-B1, MAGE-B2, MAGE-B3, MAGE-B4, MAGE-C1, BAGE, GAGE-1, GAGE-2, p15, tyrosinase, tyrosinase-related protein 1 (TRP-1), tyrosinase-related protein 2 (TRP-2), β-catenin, NY-ESO-1, LAGE-1a, PP1, MDM2, MDM4, EGVFvIII, Tax, SSX2, telomerase, TARP, pp65, CDK4, vimentin, S100, eIF-4A1, IFN-inducible p78, melanotransferrin (p97), Uroplakin II, prostate specific antigen (PSA), human kallikrein (huK2), prostate specific membrane antigen (PSM), and prostatic acid phosphatase (PAP), neutrophil elastase, ephrin B2, BA-46, Bcr-abl, E2A-PRL, H4-RET, IGH-IGK, MYL-RAR, Caspase 8, FRa, CD24, CD44, CD223, CD 256, epCAM, CA-224, HE4, Oval, estrogen receptor, progesterone receptor, uPA, PAI-1, CD28, CD29, CD22, ROR1, CD33/IL3Rα, c-Met, PSMA, Glycolipid F77, GD-2, insulin growth factor (IGF)-I, IGF-II, IGF-I receptor and mesothelin.

295. The kit of any of embodiments 222-294, wherein the first template polynucleotide, the one or more second template polynucleotide and/or the one or more polynucleotide encoding the gRNA and/or a Cas9 protein is comprised in one or more vector(s), which optionally are viral vector(s).

296. The kit of embodiment 295, wherein the vector is an AAV vector.

297. The kit of embodiment 296, wherein the AAV vector is selected from among AAV1, AAV2, AAV3, AAV4, AAV5, AAV6, AAV7 or AAV8 vector.

298. The kit of embodiment 297, wherein the AAV vector is an AAV2 or AAV6 vector.

299. The kit of embodiment 295, wherein the viral vector is a retroviral vector. 300. The kit of embodiment 299, wherein the viral vector is a lentiviral vector.

### X. EXAMPLES

The following examples are included for illustrative purposes only and are not intended to limit the scope of the invention.

### Example 1: Generation and Assessment of Engineered T Cells Expressing a Recombinant T Cell Receptor (TCR) by Targeted Knock-in or Random Integration of Sequences Encoding the TCR

Polynucleotides encoding exemplary recombinant T cell receptors (TCRs) were introduced into T cells with a genetic disruption at the endogenous gene locus that encode the T cell receptor alpha (TCRα) chain, by CRISPR/Cas9 mediated gene editing and targeted integration at the site of genetic disruption via homology-dependent repair (HDR), or by random integration via lentiviral transduction.

A. Recombinant TCR Transgene Constructs

Exemplary template polynucleotides were generated for targeted integration by HDR of a transgene containing nucleic acid sequences encoding one of two exemplary recombinant TCRs. The general structure of the exemplary template polynucleotides were as follows: [5′ homology arm]-[transgene sequences]-[3′ homology arm]. The homology arms included approximately 600 bp of nucleic acid sequences homologous to sequences surrounding the target integration site in exon 1 of the human TCR α constant region (TRAC) gene (5′ homology arm sequence set forth in SEQ ID NO:124; 3′ homology arm sequence set forth in SEQ ID NO:125).

The transgene included nucleic acid sequences encoding the α and β chains of one of two exemplary recombinant TCRs that recognizes an epitope of the human papilloma virus (HPV) 16 oncoprotein E7 (TCR #1 and TCR #2), in which the sequences encoding the TCRα and TCRβ chains were separated by a 2A ribosome skip element. The nucleotide sequences encoding TCR #1 and the constant region for TCR #2 also was modified by codon optimization and/or by mutation(s) to promote the formation of a non-native disulfide bond in the interface between the TCR constant domains to increase pairing and stability of the TCR. The non-native disulfide bond was promoted by modifying the TCR chains at residue 48 in the TCR alpha chain constant region (Ca) region from Thr to Cys and residue 57 of the TCR beta chain constant region (Cβ) region from Ser to Cys (see Kuball et al. (2007) Blood, 109:2331-2338).

The transgene also included either a) the human elongation factor 1 alpha (EF1α) promoter to drive the expression of the recombinant TCR-encoding sequences (sequence set forth in SEQ ID NO:127); or b) sequences encoding a P2A ribosome skip element (sequence set forth in SEQ ID NO:128) upstream of the recombinant TCR-encoding sequences, to drive expression of the recombinant TCR from the endogenous TCRα locus upon HDR-mediated targeted integration in-frame into the human TCR α constant region (TRAC) gene.

For targeted integration by HDR, adeno-associated virus (AAV) vector constructs containing the template polynucleotides described above were generated. AAV stocks were produced by triple transfection of an AAV vector that included the template polynucleotide, serotype helper plasmid and adenoviral helper plasmid into a 293T cell line. Transfected cells were collected, lysed and AAV stock was collected for transduction of cells.

As control, for random integration, nucleic acid sequences encoding the exemplary recombinant TCR transgene constructs described above, or sequences encoding a reference TCR capable of binding to HPV 16 E7 but containing mouse Cα and the Cβ regions, under the control of the EF1α promoter, were incorporated into an exemplary HIV-1 derived lentiviral vector. Pseudotyped lentiviral vector particles were produced by standard procedures by transiently transfecting HEK-293T cells with the resulting vectors, helper plasmids (containing gagpol plasmids and rev plasmid), and a pseudotyping plasmid and used to transduce cells.

B. Generation of Engineered T Cells

Primary human CD4+ and CD8+ T cells from 2 different human donors were isolated by immunoaffinity-based selection from human peripheral blood mononuclear cells (PBMCs) obtained from healthy donors. CD8+ cells (for TCR #1) or CD4+ and CD8+ cells combined at a 1:1 ratio (for TCR #2) were stimulated for 72 hours at 37° C. by culturing with an anti-CD3/anti-CD28 reagent at a 1:1 bead:cell ratio in media containing human serum, IL-2, IL-7 and IL-15. For introducing a genetic disruption at the endogenous TRAC locus by CRISPR/Cas9-mediated gene editing, the anti-CD3/anti-CD28 reagent was removed, and the cells were electroporated with 2 μM ribonucleoprotein (RNP) complexes containing Streptococcus pyogenes Cas9 and a guide RNA (gRNA) with the targeting domain sequence GAGAAUCAAAAUCGGUGAAU (SEQ ID NO:31), which targets a genetic disruption within exon 1 of the endogenous TCR α constant region (TRAC) gene. Following electroporation, cells were mixed with media containing AAV preparation containing the HDR template polynucleotide encoding the exemplary recombinant TCR under the control of the EF1α promoter for transduction (HDR KO). As controls, cells were treated under the same conditions used for electroporation but without addition of an RNP (mock KO), transduced with a lentiviral vector encoding the recombinant TCR (Lenti), or a reference TCR capable of binding to HPV 16 E7 but containing mouse Cα and the Cβ regions (Lenti Ref), or transduced with a lentiviral vector encoding the recombinant TCR and also electroporated with RNP complexes targeting a genetic disruption at the TRAC locus (Lenti KO). Following transduction, the cells were cultured for approximately 7 days in media containing human serum and IL-2, IL-7 and IL-15.

C. Expression of TCRs

On day 7 after electroporation, the cells were assessed by flow cytometry for staining with an anti-CD3 antibody, an anti-CD4 antibody, an anti-CD8 antibody, an anti-Vbeta antibody specific for each of the recombinant TCR, and with a peptide-MHC tetramer complexed with the HPV16 E7 peptide.

The results for TCR #1-expressing CD8+ cells are shown in FIGS. 1A-1C. As shown, the cells expressing TCR #1 by HDR-mediated targeted integration at the TRAC locus exhibited the highest proportion of cells bound by the tetramer (FIG. 1A) and highest mean fluorescence intensity of tetramer staining in CD8+ cells (FIG. 1B). Among the CD8+ cells bound by the tetramer, the extent of binding by the tetramer was generally more uniform in cells that were subjected to HDR-mediated integration compared to random integration by lentiviral transduction, as shown by lower coefficient of variation (the standard deviation of signal within a population of cells divided by the mean of the signal in the respective population; see FIG. 1C). The results for TCR #2-expressing cells are shown in FIGS. 2A-2B. As shown, CD4+ and CD8+ cells expressing TCR #2 by HDR-mediated integration at the TRAC locus exhibited the highest proportion of cells bound by the tetramer (FIG. 2A) and highest mean fluorescence intensity of tetramer staining (FIG. 2B).

D. Cytolytic Activity and Cytokine Production

Cytolytic activity and cytokine production of cells engineered to express recombinant TCR #1 or recombinant TCR #2 by HDR or by random integration as described above were assessed after incubating with target cells expressing HPV 16 E7 in vitro.

Cytolytic activity was assessed by culturing recombinant TCR-expressing effector cells with target cells expressing HPV 16 E7 labeled with NucLight Red (NLR) at an effector to target (E:T) ratio of 10:1, 5:1 and 2.5:1. The ability of the T cells to antigen-specifically lyse the target cells was assessed by measuring the loss of labeled target cells every 2 hours up to 44 hours post co-culture. Cytolytic activity was determined from the average from 2 donors in each group, of the area under the curve (AUC) of % killing, normalized to Vbeta expression for each of the recombinant TCR, and compared to mock transduction control. Cytokine production was measured following incubation of recombinant TCR-expressing effector cells with target cells. Interferon-gamma (IFNγ) and interleukin-2 (IL-2) secretion in the supernatant was determined by ELISA, normalized to Vbeta expression and averaged for the 2 donors in each group.

The results for TCR #1 are shown in FIGS. 3A-3B. The degree of killing and IFNγ secretion was higher in cells in which the recombinant TCR #1 driven by the EF1α promoter was introduced by HDR-mediated targeted integration at the TRAC locus (EF1α-TCR #1 HDR KO), compared to by random integration, with (TCR #1 Lenti) or without knockout of the TRAC locus (TCR #1 Lenti KO).

Exemplary results for TCR #2 are shown in FIGS. 4A-4G. The target cell AUC and IFNγ and IL-2 secretion after 24 hours was similar or higher in cells in which the recombinant TCR #2 driven by the EF1α promoter was introduced by HDR-mediated targeted integration (EF1α-TCR #2 HDR KO), compared to by random integration, with (TCR #2 Lenti) or without knockout of the TRAC locus (TCR #2 Lenti KO), and the reference TCR introduced by random integration (Lenti Ref) (FIGS. 4A-4C). CD8+ and CD4+ cells expressing TCR #2 introduced by HDR at the TRAC locus exhibited higher killing of target cells compared to by random integration, with or without knockout of the TRAC locus, and the reference TCR introduced by random integration (FIGS. 4D-4E). IFNγ production by CD8+ cells at an E:T ratio of 2.5:1 or by CD4+ cells at a 10:1 E:T ratio was observed to be similar for cells expressing TCR #2 introduced by HDR at the TRAC locus or by random integration (FIGS. 4F-4G).

E. Viability of Cells Expressing Recombinant TCRs

Viability of the CD4+ and CD8+ cells engineered to express TCR #2 introduced by the various methods were assessed at cryopreservation and after thawing preserved cells, by acridine orange (AO) and propidium iodide (PI) staining. As shown in FIGS. 5A-5B, viability of the cells was not substantially affected by introduction of the TCR #2-encoding sequences by HDR, compared to mock treated cells or cells expressing the reference TCR.

F. Conclusion

In general, the results were consistent with the observation that targeted integration by HDR of nucleic acid sequences encoding exemplary recombinant TCRs resulted in higher recombinant TCR expression of the human TCR in human T cells compared to introduction of the TCR by random integration, thereby leading to higher functional activity of cells expressing the recombinant TCR.

### Example 2: Generation and Assessment of Expression of Recombinant T Cell Receptors (TCR) by Targeted Knock-in or Random Integration of Sequences Encoding the TCR in T Cells with Knock-Out of Endogenous TCRα and TCRβ Chains

A polynucleotide encoding one of the exemplary recombinant TCR was targeted for integration at one of the sites of genetic disruption via homology-dependent repair (HDR), or was introduced by random integration via lentiviral transduction, in cells engineered to genetically disrupt the endogenous gene loci that encode the T cell receptor alpha (TCRα) and beta (TCRβ) chains or both, by CRISPR/Cas9 mediated gene editing.

For targeted integration by HDR, AAV preparations containing template polynucleotide constructs encoding the exemplary recombinant TCR #1 were generated substantially as described in Examples 1, except with the following differences: an additional AAV construct was generated containing the MND promoter (sequence set forth in SEQ ID NO:126), which is a synthetic promoter that contains the U3 region of a modified MoMuLV LTR with myeloproliferative sarcoma virus enhancer, to control expression of the recombinant TCR-encoding sequences.

For random integration, lentiviral preparations containing nucleic acid sequences encoding the exemplary recombinant TCR #1 were generated generally as described in Example 1. For these studies, the lentiviral transduction construct further contained a polynucleotide encoding a truncated receptor separated from the recombinant TCR transgene by a sequence encoding a T2A ribosome skip sequence for expression of both the recombinant TCR and the truncated receptor from the same construct; the truncated receptor was for use as a surrogate marker for transduction. As a control, a polynucleotide encoding a chimeric TCR was generated where the Cα and the Cβ of the recombinant TCR were replaced by constant regions from a mouse TCR (mouse Cα sequence set forth in SEQ ID NO:122; mouse Cβ sequence set forth in SEQ ID NO:123) or the cells were subject to mock transduction.

Primary human CD4+ and CD8+ T cells were isolated and engineered to introduce a genetic disruption at the endogenous TRAC and TRBC loci by CRISPR/Cas9-mediated gene editing and transduced with the AAV preparations containing template polynucleotides for HDR or lentiviral preparations for random integration, generally as described in Example 1B above. Cells were electroporated with ribonucleoprotein (RNP) complexes containing the TRAC-targeting guide RNA (gRNA) described in Example 1B and an RNP containing a gRNA targeting a consensus target site sequence common to exon 1 of both TCR β constant regions 1 and 2 (with the targeting domain sequence GGCCUCGGCGCUGACGAUCU (SEQ ID NO:63)) (TCRαβ KO). As a control, cells were treated under the same conditions used for electroporation but without addition of an RNP (Mock KO; also designated as TCRαβ WT).

The cells were subsequently cultured for four (4) days, then assessed by flow cytometry for staining with an anti-CD3 antibody, an anti-Vbeta antibody that recognizes recombinant TCR #1, and with a peptide-MHC tetramer complexed with the antigen recognized by the recombinant TCR (HPV16 E7 peptide). The cells also were co-stained for CD8 or CD4.

The results are shown in FIGS. 6A-6E. As shown in FIGS. 6A and 6B, CRISPR/Cas9 mediated knockout (KO) of TRAC and TRBC (panel labeled “TCRαβ KO” in figures) resulted in almost complete disruption of TCR expression in CD8+ cells as observed by the absence of CD3 staining in cells subjected to KO and mock transduction (panel labeled TCRαβ KO/mock transd). Expression of the recombinant TCR (as indicated by cells stained by the specific Vbeta antibody or cells positive for tetramer staining among CD8+ cells) was slightly improved following lentiviral transduction in cells that were KO for the endogenous TCR (TCRαβ KO/lenti human) compared to cells that retained expression of the endogenous TCR (TCRαβ WT/lenti human). In cells retaining the endogenous TCR, recombinant TCR expression was improved by lentiviral transduction of a recombinant TCR containing a mouse constant domain compared to lentiviral transduction of a fully human recombinant TCR (compare TCRαβ WT/lenti human and TCRαβ WT/lenti mouse).

HDR-mediated targeted knock-in of the recombinant TCR and KO of the endogenous TCR resulted in a substantially greater proportion of cells expressing the recombinant TCR than observed following lentiviral transduction (first two left panels designated “HDR” compared to TCRαβ KO/lenti human in FIGS. 6A and 6B). The geometric mean fluorescence (gMFI) of recombinant TCR expression, as assessed by Vbeta or tetramer staining in CD8+ cells (FIG. 6C) or Vbeta staining in CD4+ cells (FIG. 6D), also was substantially higher in cells subjected to HDR compared to lentiviral transduction. The degree of recombinant TCR expression by HDR was similar whether the recombinant TCR was under the control of the EF1α or MND promoter.

Among the cells that were positive for expression of the recombinant TCR, the extent of expression was generally more uniform or tighter in cells that were subjected to HDR-mediated targeted integration compared to random integration lentiviral transduction (see FIGS. 6A and 6B). As shown in FIG. 6E and FIG. 6F, a lower coefficient of variation (the standard deviation of signal within a population of cells divided by the mean of the signal in the respective population) of recombinant TCR expression as determined by peptide-MHC tetramer binding and Vbeta expression, respectively, was observed in CD8+ cells that were subjected to HDR-mediated integration compared to random integration. The results are consistent with a finding that targeted knock-in of a recombinant TCR into the endogenous TCRα locus, in combination with knock out of the endogenous TCRαβ chains, results in a higher and a more uniform level of expression in population of cells engineered to express the recombinant TCR compared to other methods.

### Example 3: Assessment of HDR-Mediated Knock-in of Sequences Encoding a Recombinant T Cell Receptor (TCR) in T Cells with Knock-Out of Endogenous TCRα or TCRβ Chains or Both

To further assess recombinant TCR expression by HDR, nucleic acid sequences encoding an exemplary recombinant TCR into the TRAC locus was targeted for integration in cells containing a dual knock-out of the TRAC and TRBC loci, knock-out of only the TRAC locus or knock-out of only the TRBC locus.

A. Recombinant TCR Transgene Constructs and Generation of Engineered Cells

These studies were carried out using AAV (for HDR) and lentiviral constructs (for random integration) encoding the exemplary TCR #1 substantially as described in Examples 1 and 2, except with the following differences: lentiviral constructs were generated containing a polynucleotide encoding the recombinant TCR under the operable control of the EF1α or the MND promoter. A lentiviral construct containing a polynucleotide encoding the recombinant TCR #1 under the operable control of the EF1α promoter, and truncated receptor separated from the recombinant TCR transgene by a sequence encoding a T2A ribosome skip sequence, also was generated for comparison.

For targeted integration by HDR, primary human CD4+ and CD8+ T cells were stimulated, cultured and subject to electroporation with ribonucleoprotein (RNP) complexes containing only TRAC-targeting gRNA, only TRBC-targeting gRNA, or both TRAC- and TRBC-targeting gRNA generally as described in Examples 1 and 2. After electroporation, the cells were transduced with AAV preparations containing polynucleotides that encoded the recombinant TCR for targeting to the endogenous TRAC locus, generally as described above.

For random integration, the primary human CD4+ and CD8+ T cells were thawed, stimulated and cultured substantially as described in Examples 1 and 2, followed by transduction with a lentiviral preparation that encoded the recombinant TCR. In this study, the lentivirus preparations were transduced into primary T cells that retained the endogenous TCR. As a control, cells were treated under the same conditions used for lentiviral transduction but without addition of lentivirus (mock transduction).

B. Expression of TCRs

The cells were subsequently cultured for 4-10 additional days, and assessed by flow cytometry after staining with an anti-CD3 antibody, an anti-Vbeta antibody specific for the recombinant TCR #1, and with a peptide-MHC tetramer complexed with the antigen recognized by the TCR (HPV16 E7 peptide). The cells also were co-stained for CD8 or CD4.

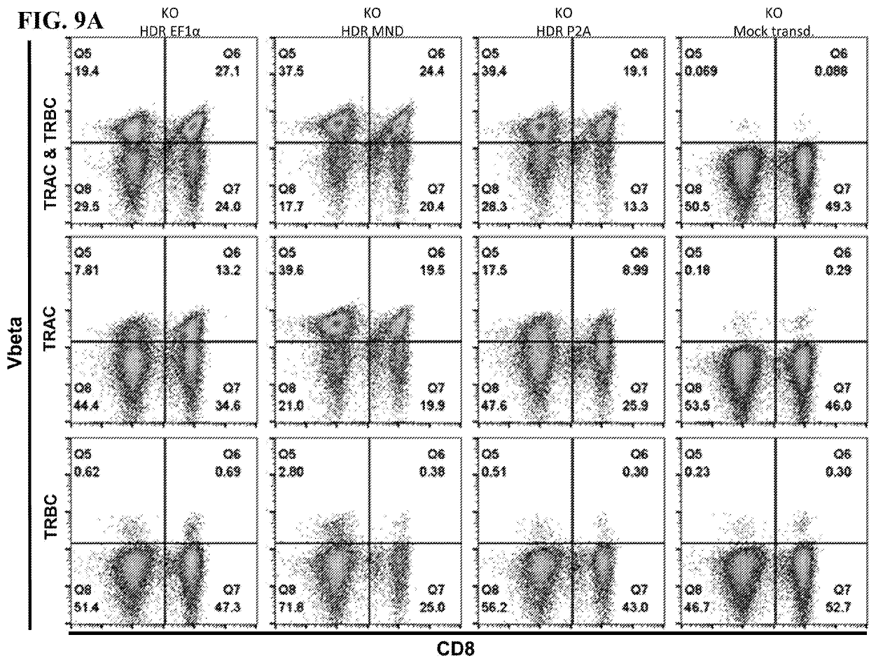

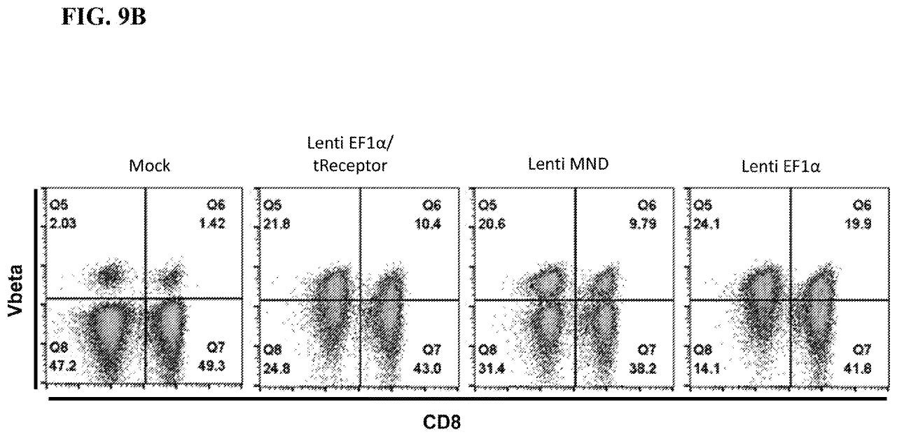

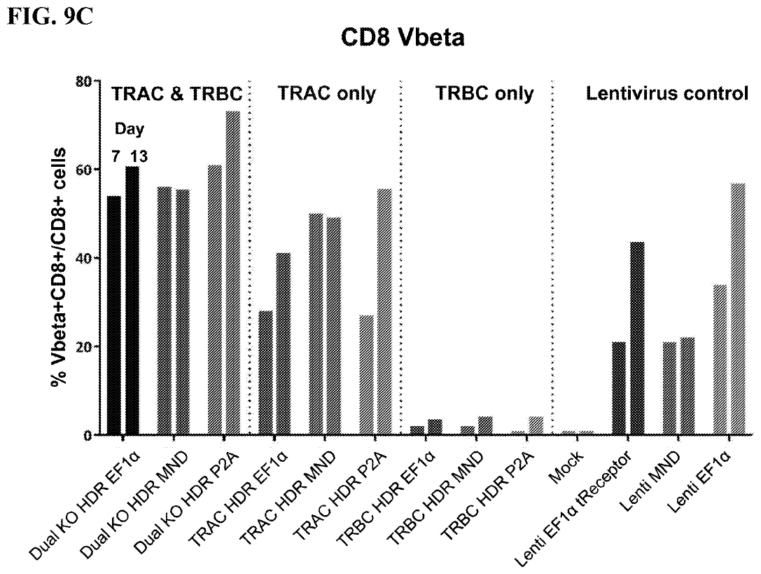

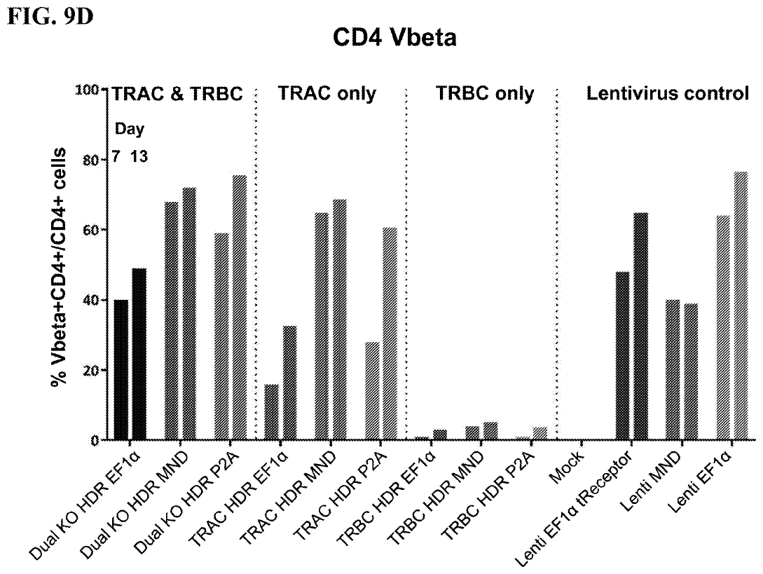

The results for CD3 staining are shown in FIGS. 7A-7C, for tetramer staining is shown in FIGS. 8A-8C, and for Vbeta staining is shown in FIGS. 9A-9D.

As shown in FIGS. 7A and 7C, electroporation with RNPs complexed with gRNAs targeting TRAC and TRBC resulted in efficient knock-out of the endogenous TCR as evidenced by the absence of CD3 surface expression (see panel labeled KO/mock transd. in FIG. 7A; see also group labeled KO mock FIG. 7C, which shows the percentage of CD3+CD8+ cells among CD8+ cells). The degree of KO with gRNAs targeting both TRAC and TRBC was greater than for cells electroporated with RNP complexed with gRNA targeting only TRAC or only TRBC, which is consistent with an observation that dual-targeting of both the constant domains of TCR chains α and β improves the efficiency of disrupting endogenous TCR expression. In cells transduced with lentiviral vectors in which no disruption of the endogenous TCR was carried out, CD3 expression was similar in all tested conditions (FIGS. 7B and 7C). As shown in FIGS. 7A and 7C, CD3 expression also was similar among cells in which the recombinant TCR was introduced by HDR, which is consistent with TCR/CD3 surface expression in cells introduced with the recombinant TCR.

As shown in FIGS. 8A and 8C, the proportion of CD8+ cells that bound the peptide-MHC tetramer, indicated recombinant TCR expression, were higher under conditions in which HDR was carried out in cells knocked out for both TRAC and TRBC as compared to TRAC only (compare top and middle rows in FIG. 8A; compare TRAC & TRBC with TRAC only in FIG. 8C). As shown in FIG. 8C, similar results were observed on days 7 and 13. Similar expression of the recombinant TCR was observed in cells whether HDR was carried out with a construct for integration under the control of an exogenous EF1α or MND promoter or under the control of the endogenous TCR promoter (P2A-containing construct). As shown in FIG. 8B and FIG. 8C, fewer cells expressed the recombinant TCR, as assessed by tetramer staining, following lentiviral-mediated transduction, regardless of the presence of a truncated receptor in lentiviral constructs.

As shown in FIGS. 9A-9C, similar to the results above, expression of the recombinant TCR on CD8+ T cells was observed when directly staining for the recombinant TCR with an antibody that specifically recognizes the Vbeta chain of the recombinant TCR. Staining with anti-Vbeta, which also is capable of detecting the recombinant TCR on CD4+T cells (CD8 negative population), also showed that expression of the recombinant TCR was observed in CD4+ cells (FIG. 9A and FIG. 9D).

For all methods of assessing recombinant TCR expression shown above (anti-CD3, tetramer and anti-Vbeta), the results above also showed that targeted integration of the recombinant TCR to the TRAC via HDR was specific for nuclease-induced DNA break at the TRAC locus, as the cells electroporated with TRBC-targeted RNP did not express the recombinant TCR (see, e.g., “TRBC only” condition in FIGS. 8A, 8C, 9A, 9C and 9D).

C. Cytolytic Activity and Cytokine Production

Cytolytic activity and cytokine production of CD8+ cells engineered to express recombinant TCR #1 by HDR or by random integration as described above were assessed after incubating with target cells expressing HPV 16 E7 in vitro. In addition to cells described above, primary human CD8+ cells transduced with a lentivirus encoding a reference TCR capable of binding to HPV 16 E7 but containing mouse Cα and the Cβ regions, also was assessed.

Cytolytic activity was assessed by incubating recombinant TCR-expressing effector cells with target cells expressing HPV 16 E7 at an effector to target (E:T) ratio of 10:1, 5:1 and 2.5:1. The ability of the T cells to antigen-specifically lyse the target cells was assessed 4 hours post co-culture. Cytolytic activity was determined from the area under the curve (AUC) of % killing, normalized to Vbeta expression for each of the recombinant TCR and compared to mock transduction control. The results are shown in FIG. 10. The degree of killing was higher in cells in which the recombinant TCR was introduced by HDR-mediated targeted integration compared to by random integration, which is consistent with a finding that higher expression of the recombinant TCR in cells results in higher functional activity.

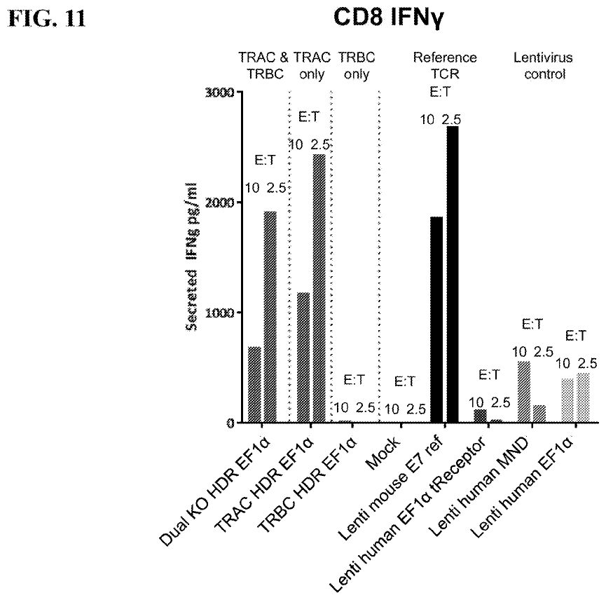

Cytokine production was also monitored following incubation of recombinant TCR-expressing CD8+ effector cells with target cells expressing HPV 16 E7 at an E:T ratio of 10:1 and 2.5:1 for 48 hours. IFNγ secretion in the supernatant was determined by ELISA and was normalized to Vbeta expression for each group. The results are shown in FIG. 11. Similar to the results above for cytolytic activity, a greater production of IFNγ was observed by cells subjected to HDR-mediated integration compared to random integration. In the cytolytic activity assay and assessment of IFNγ secretion, the functional activity of cells expressing the recombinant TCR, by HDR-mediated integration, was similar to the activity of cells expressing, via lentiviral transduction, a reference TCR containing mouse constant domains.

Proliferation of the recombinant TCR-expressing cells was assessed following incubation with SCC152 target cells or T2 target cells pulsed the antigen peptide was assessed. The cells were labeled with CellTrace™ violet (ThermoFisher) dye. Division of live T cells was indicated by CellTrace™ violet dye dilution, as assessed by flow cytometry.

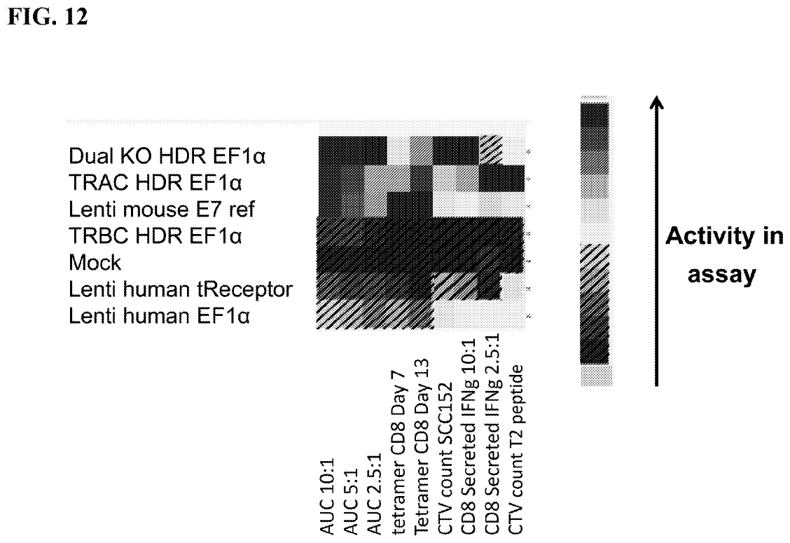

The results of various functional assays are depicted in FIG. 12. As shown in the heat map depicting the relative activity of recombinant TCR-expressing cell populations in various functional activities (AUC of % killing at E:T ratios of 10:1, 5:1 and 2.5:1 (designated “AUC”), tetramer binding in CD8+ cells on days 7 and 13 (designated “tetramer CD8”), proliferation assay (designated “CTV count”) using SCC152 cells or T2 target cells pulsed the antigen peptide and secretion of IFNγ from CD8+ cells (designated “CD8 secreted IFNg”)), functional activity of cells with recombinant TCR targeted for knock-in at the endogenous TCRα chain constant domain locus and knockout of the endogenous TCRαβ genes or TCRα gene was generally observed to be higher compared to cells where the polynucleotide encoding the recombinant TCR was randomly integrated.

In general, the results were consistent with the observation that targeted integration by HDR results in higher recombinant TCR expression of the human TCR in human T cells compared to introduction of the TCR by random integration, thereby leading to higher functional activity of cells expressing the recombinant TCR.

### Example 4: Assessment of In Vivo Anti-Tumor Effects in Mice of T Cells Engineered to Express a Recombinant T Cell Receptor (TCR) Generated by HDR-Mediated Knock-in

Anti-tumor activity and pharmacokinetics of CD4+ and CD8+ cells expressing an exemplary TCR generated by HDR-mediated knock-in or by random integration via lentiviral transduction of the recombinant TCR-encoding sequences, was assessed by administration of the engineered cells in a tumor mouse model.

A. Tumor Burden and Survival

A mouse tumor model was generated by subcutaneous injection 4×10⁶ of squamous cell carcinoma cell line UPCI:SCC152 (ATCC® CRL-3240™) cells in female NOD/SCID/IL-2Rγ^{null} (NSG) mice. The tumor was established for approximately 25 days, and staged based on tumor volume (P>0.95) prior to administration of engineered cells.

Primary CD4+ cells and CD8+ cells engineered to express the exemplary recombinant TCR #2 by targeted knock-in or by random integration of the sequence encoding the TCR, generated substantially as described above in Example 1, were administered 26 days after injection of the tumor cells. Two different total doses of recombinant TCR-expressing cells (3×10⁶ or 6×10⁶ TCR-expressing cells) were administered, the dose based on the percentage of cells positive for staining with an anti-Vbeta antibody that recognizes the recombinant TCR #2. The following groups were compared: TCR #2 controlled by the human elongation factor 1 alpha (EF1α) promoter, targeted for integration at the TRAC locus by HDR (TCR #2 HDR KO EF1α); TCR #2 controlled by the endogenous TRAC promoter (by upstream in-frame P2A ribosome skip element), targeted for integration at the TRAC locus by HDR (TCR #2 HDR KO P2A); TCR #2 randomly integrated using lentiviral construct (TCR #2 Lenti); TCR #2 randomly integrated using lentiviral construct in cells containing a knock-out of the endogenous TRAC (TCR #2 Lenti KO); and reference TCR capable of binding to HPV 16 E7 but containing mouse Cα and the Cβ regions randomly integrated using lentiviral construct (Lenti Ref). As controls, mice that received no engineered cells (tumor alone) or that were administered cells treated under the same conditions used for electroporation but without addition of an RNP (mock KO) were used. Table E1 also sets forth the number of mice and the number of total T cells administered for each dose in each group.

TABLE E1
Study Design for assessment of in vivo anti-tumor effect in mouse tumor model.

| | | | | TCR per | | Total | |
|---|---|---|---|---|---|---|---|
| | | | | mouse | Total T | No. of | |
| Group | | CD4 | CD8 | (1:1TCR + | cells/ | mouse/ | SCC152 |
| No. | Group Description | (Vbeta) | (Vbeta) | CD4:CD8) | mouse | group | per mouse |
| 1 | Tumor alone | | | 0 | 0 | 7 | 4.00E+06 |
| 2 | Mock KO | | | 0.00E+00 | 1.98E+07 | 7 | 4.00E+06 |
| 3 | Lenti Ref | 62.6% | 59.7% | 6.00E+06 | 9.82E+06 | 7 | 4.00E+06 |
| 4 | Lenti Ref | | | 3.00E+06 | 4.91E+06 | 7 | 4.00E+06 |
| 5 | TCR #2 Lenti | 79.2% | 88.7% | 6.00E+06 | 7.17E+06 | 7 | 4.00E+06 |
| 6 | TCR #2 Lenti | | | 3.00E+06 | 3.59E+06 | 7 | 4.00E+06 |
| 7 | TCR #2 Leni KO EF1α | 91.0% | 88.4% | 6.00E+06 | 6.69E+06 | 7 | 4.00E+06 |
| 8 | TCR #2 Leni KO EF1α | | | 3.00E+06 | 3.35E+06 | 7 | 4.00E+06 |
| 9 | TCR #2 HDR KO EF1α | 33.5% | 30.3% | 6.00E+06 | 1.89E+07 | 7 | 4.00E+06 |
| 10 | TCR #2 HDR KO EF1α | | | 3.00E+06 | 9.43E+06 | 7 | 4.00E+06 |
| 11 | TCR #2 HDR KO P2A | 39.3% | 41.1% | 6.00E+06 | 1.49E+07 | 7 | 4.00E+06 |
| 12 | TCR #2 HDR KO P2A | | | 3.00E+06 | 7.47E+06 | 7 | 4.00E+06 |

The mean tumor volume was assessed twice a week for up to 58 days after administration of the engineered cells. Changes in body weight, survival of the mice and the number of mice free of tumor on day 58 was also monitored.

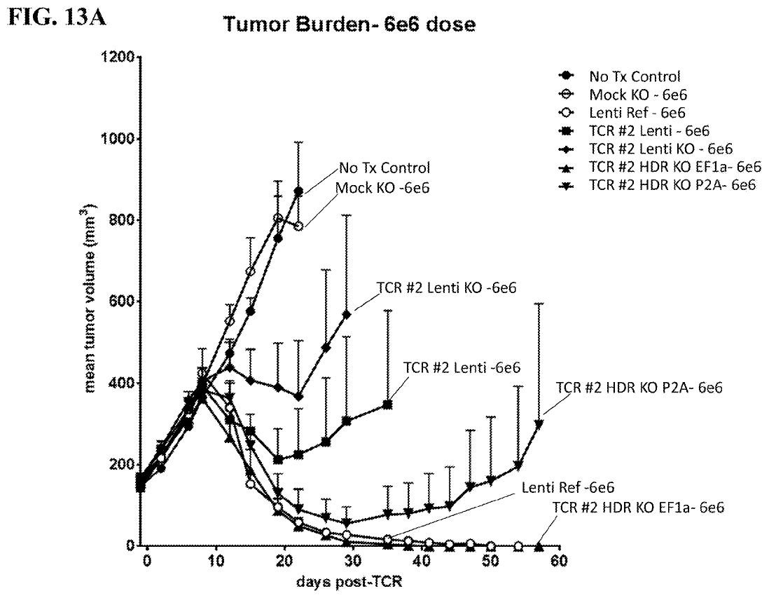

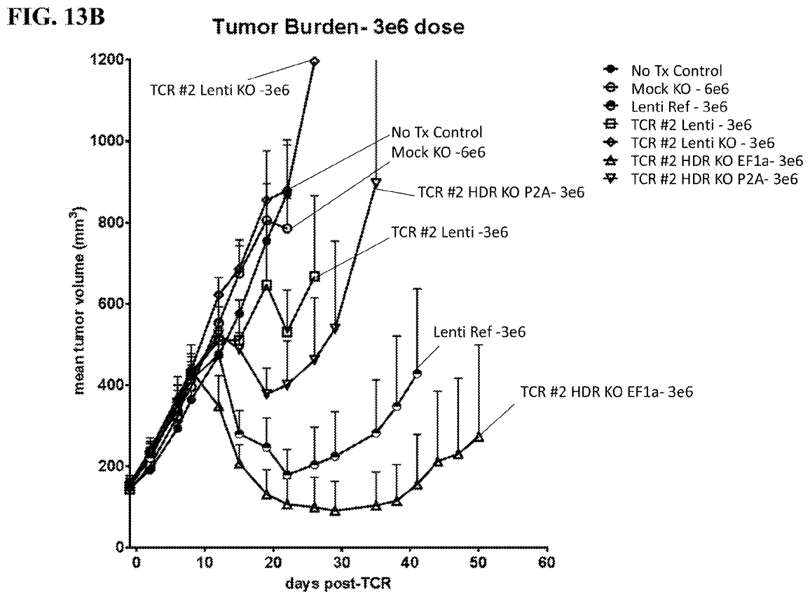

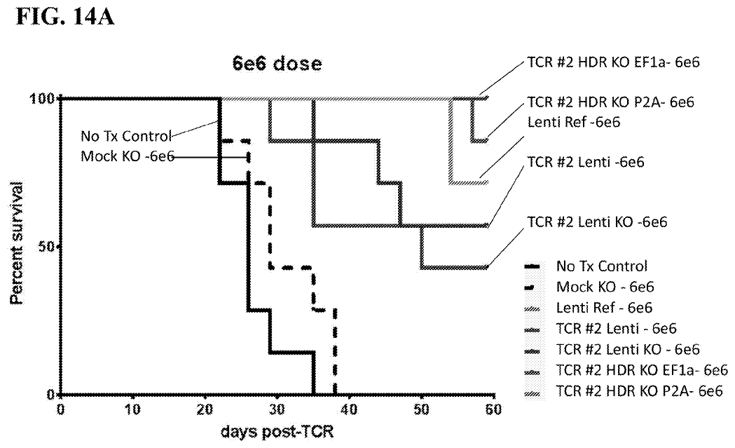

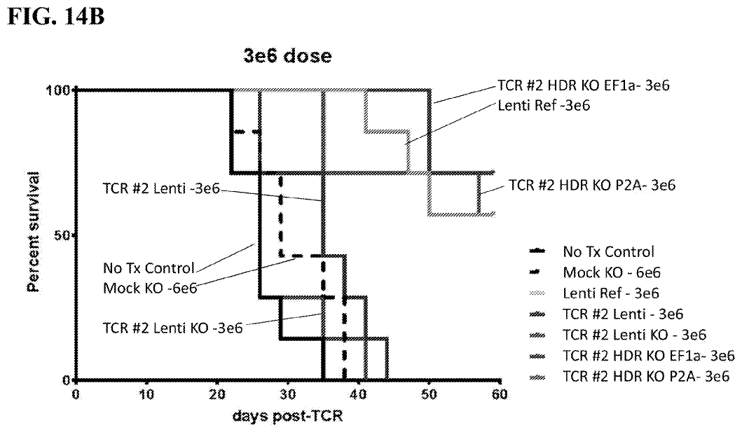

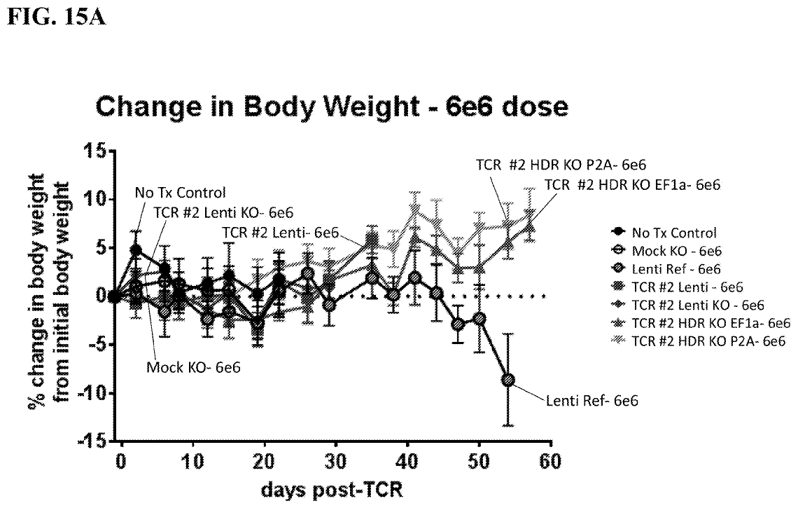

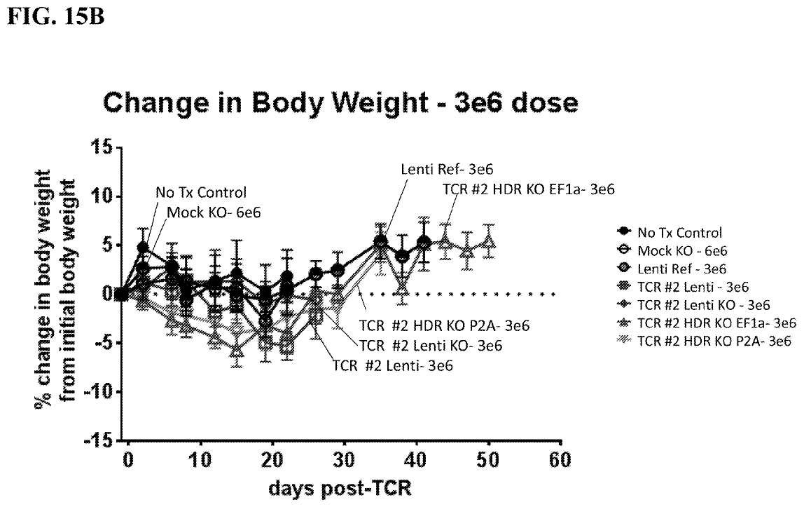

The results of anti-tumor activity (reduction in tumor burden) and survival for mice administered TCR #2-expressing cells at two different doses are shown in FIGS. 13A-13B and FIGS. 14A-14B. As shown in FIGS. 13A-13B, reduction in tumor volume in mice administered cells expressing TCR #2 by HDR-mediated targeted integration, under control of either the EF1α (TCR #2 HDR KO EF1α) or the endogenous TRAC promoter (TCR #2 HDR KO P2A), was greater compared to in mice administered cells expressing TCR #2 by random integration by lentiviral transduction (TCR #2 Lenti or TCR #2 Lenti KO). At both doses, reduction in tumor volume in mice administered cells expressing TCR #2 by HDR-mediated integration of the sequences encoding the TCR under control of the EF1α promoter (TCR #2 HDR KO EF1α) was comparable or greater compared to mice administered cells expressing the reference TCR (Lenti Ref). As shown in FIGS. 14A-14B, the % survival of mice administered cells expressing TCR #2 by HDR-mediated integration was higher than of mice administered cells expressing a TCR generated by random integration (TCR #2 Lenti or TCR #2 Lenti KO). As shown in Table E2, the number of mice that did not have a tumor at day 58 after administration of the cells was also greater in groups administered cells expressing TCR #2 by HDR-mediated integration, under control of either the EF1α (TCR #2 HDR KO EF1α) or the endogenous TRAC promoter (TCR #2 HDR KO P2A), compared to the other groups. Change in body weight over the course of the study was also monitored as shown in FIGS. 15A-15B.

TABLE E2
Number of tumor-free mice at day 58 after administration of engineered cells.

| | No. of tumor free mice at day 58 | No. of tumor free mice at day 58 |
|---|---|---|
| Group | (out of 7), 6 × 10⁶ dose | (out of 7), 3 × 10⁶ dose |
| Tumor alone | 0 | 0 |
| Mock KO | 0 | 0 |
| Lenti Ref | 5 | 0 |
| TCR #2 Lenti | 4 | 0 |
| TCR #2 Leni KO EF1α | 1 | 0 |
| TCR #2 HDR KO EF1α | 7 | 4 |
| TCR #2 HDR KO P2A | 6 | 2 |

These results were consistent with an observation that administration of cells generated by targeted integration of sequences encoding exemplary recombinant TCRs via HDR resulted in greater in vivo anti-tumor activity compared to cells generated by introduction of the TCR-encoding sequences by random integration.

B. Assessment of Pharmacokinetics (PK)

The persistence and expansion of cells was assessed in the mouse model described above in Example 4A. Mice in each group listed in Table E1 were bled alternately every 2 weeks (first cohort with 4 mice on days 7 and 21; second cohort with 3 mice on days 14 and 28), and each treatment group was assessed once per week (on days 7, 14, 21, and 28). For assessing pharmacokinetics of the administered cells, cells were counted and expression of various markers (CD3, CD4, CD8, CD45, CD45RA, CCR7, PD-1) and the recombinant TCR (using anti-Vbeta specific for the recombinant TCR), and binding of peptide-MHC tetramer were determined by flow cytometry at each time point.

Cells expressing TCR #2 by HDR-mediated integration under control of either the EF1α or P2A promoter showed an early initial increase followed by a rapid decrease in blood counts of TCR-expressing cells between day 14 and day 21. The cells expressing the reference TCR did showed different expansion and persistence kinetics in vivo, of slower decline in circulating TCR-expressing cells. From an analysis of the percentage of CD4 or CD8 cells in the total TCR+ cells over time, cells retaining expression of the endogenous TCR were observed to exhibit a at a higher CD4 percentage in the early time points compared to cells that contained a knockout of the endogenous TRAC locus. In general, an increase in CD4 percentage was observed over time for most groups.

Staining of cell phenotype markers such as CD45RA and CCR7 showed that the phenotype of the majority of circulating recombinant TCR-expressing cells changed from CCR7+CD45RA+(associated with more naïve phenotype) to CCR7-CD45RA- (associated with more mature phenotype) between day 7 and day 14. The percentage of PD-1-expressingTCR+ cells also increased over time, from near 0% staining to about 60% of the cells exhibiting PD-1 staining, in most groups.

In general, pharmacokinetic analysis demonstrated an increased in peripheral expansion and increased counts of cells expressing recombinant TCR #2 by HDR-mediated integration under control of the EF1α promoter, consistent with the observation of high anti-tumor activity at low dose. Further, cells expressing recombinant TCR #2 generally exhibited a more mature phenotype over time, and the overall expansion and persistence kinetics were observed to be different compared to cells expressing a reference TCR.

### Example 5: Assessment of Homology Arm Lengths for Efficient HDR-Mediated Integration of Transgene Sequences

Polynucleotides containing an exemplary transgene, flanked by varying length of homology arm were introduced into T cells for targeted integration of the transgene via homology-dependent repair (HDR), and the efficiency of integration was assessed.

A. Recombinant Transgene Constructs and Generation of Engineered T Cells

Exemplary HDR template polynucleotides containing a transgene sequence encoding a green fluorescent protein (GFP), flanked by varying length of homology arms for targeted integration into the human TCRα constant region (TRAC) locus were introduced into T cells. Specifically, for integration by HDR, AAV preparations containing template nucleotide constructs encoding GFP were generated substantially as described in Examples 1-3, except with the following differences: AAV constructs were generated containing a polynucleotide encoding GFP under the operable control of the MND promoter and linked to an SV40 poly(A) sequence, flanked by 50, 100, 200, 300, 400, 500, or 600 base pair of 5′ and 3′ homology arms (SEQ ID NOS: 227-233 and 234-240, respectively) homologous to sequences surrounding the target integration site in the human TCR α constant region (TRAC) gene. The total length of the AAV construct was made constant by using filler DNA sequences between the SV40 poly(A) sequence and the 3′ homology arm.

For targeted integration by HDR, primary human CD4+ and CD8+ T cells from four human donors (Donors 1-4) were combined at a 1:1 ratio, stimulated and subject to electroporation with ribonucleoprotein (RNP) complexes containing TRAC-targeting gRNA, generally as described in Examples 1-3. After electroporation, the cells were transduced with AAV preparations containing the HDR template polynucleotides containing various homology arm lengths described above. GFP expression was measured by flow cytometry at 24, 48, 72, 96 hours and 7 days after transduction with AAV, to determine an integration ratio (representing the percentage of total AAV inside of the cells that have integrated into the genome) based on the following formula:

$$Integration ratio = \frac{( \% high MFI GFP )}{( \% high MFI GFP + \% low MFI GFP )} .$$

The % high MFI and % low MFI indicate the percentage of cells that were above or below a threshold MFI by flow cytometry, representing cells containing integrated GFP transgene or non-integrated AAV construct, respectively. The changes in integration ratio with an increase of the length of the homology arm was assessed by subtracting the integration ratio of the next shortest arm length from the integration ratio of a particular arm length.

A. Assessment of Integration Ratio

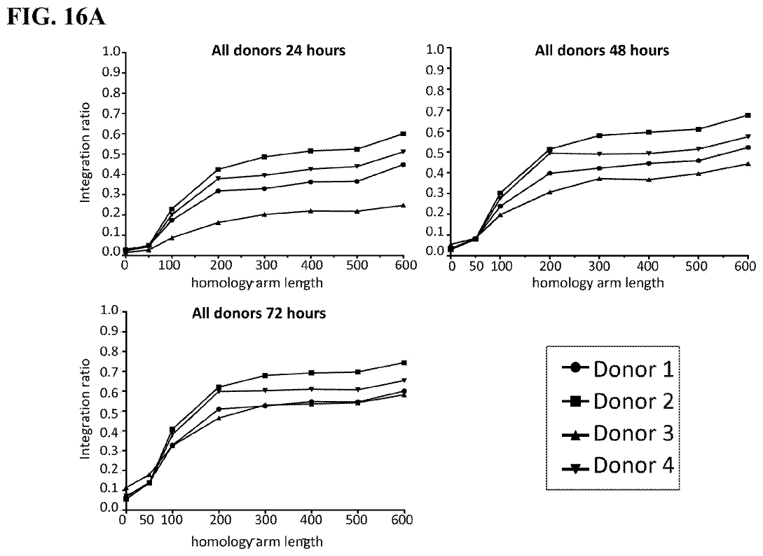

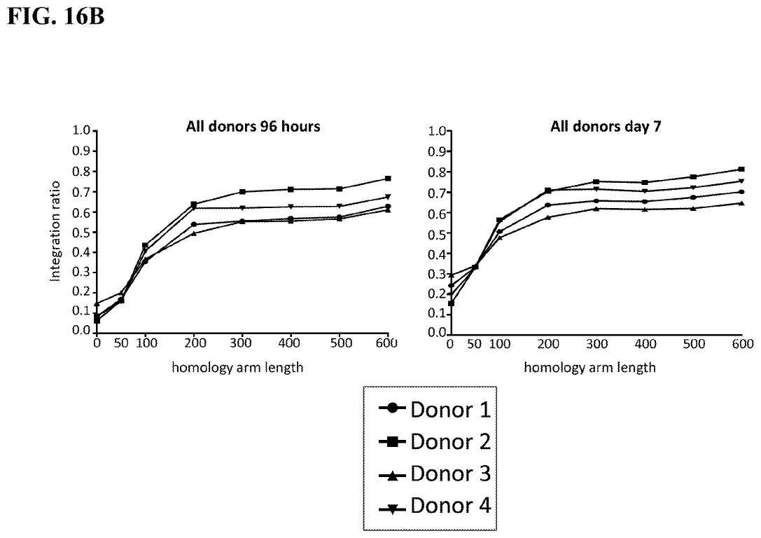

As shown in FIGS. 16A-16B, a constant or increased integration ratio was observed at various time points assessed for each homology arm length, consistent with non-integrated AAV constructs diluting out over time. Assessing the changes in GFP expression patterns over time showed that the percentage of cells with integrated GFP transgene (high MFI) generally remained constant after 72 hours, but the percentage of cells containing non-integrated AAV only (low MFI) continued to decrease.

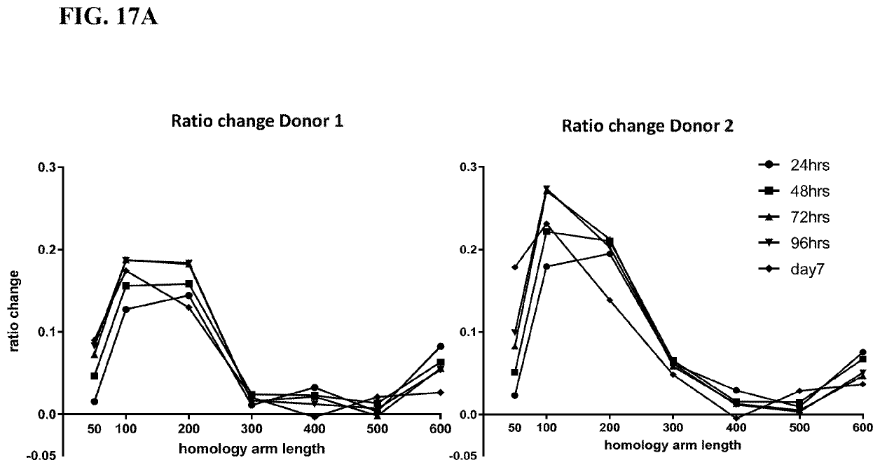

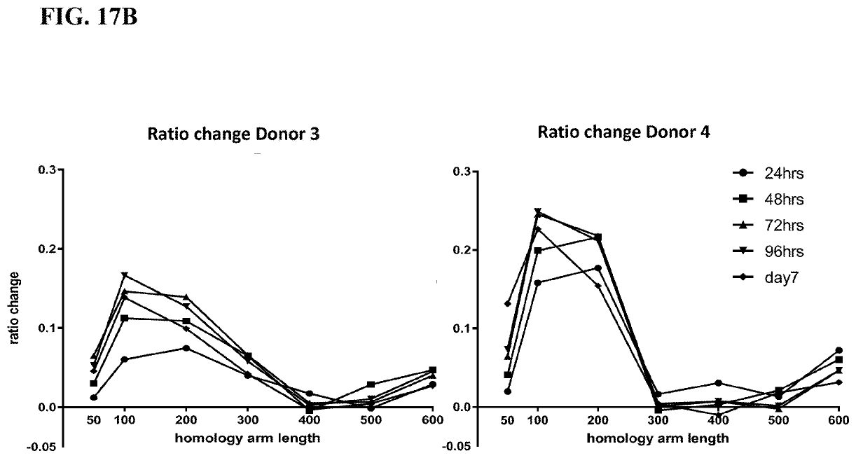

As shown in FIGS. 17A-17B, the most substantial gains in integration ratio appeared to occur at 200 bp of homology arm, with minor gains at 300 bp. No substantial gain was observed between 300 bp and 500 bp, and an increase in integration ratio was observed between 500 bp and 600 bp, in all donors. The results support using a minimum of 300 bp homology arms for high integration efficiency, with 600 bp homology arms providing even higher integration efficiency.

### Example 6: Generation and Assessment of Engineered T Cells Expressing a Chimeric Antigen Receptor (CAR) by Targeted Knock-in or Random Integration of Sequences Encoding the TCR

Polynucleotides encoding exemplary chimeric antigen receptors (CARs) were introduced into T cells with a genetic disruption at the endogenous gene locus that encodes the T cell receptor alpha (TCRα) chain, by CRISPR/Cas9 mediated gene editing and targeted integration at the site of genetic disruption via homology-dependent repair (HDR), or by random integration.

A. Exemplary Anti-CD19 CAR

a Expression of an Exemplary anti-CD19 CAR

Nucleic acid sequences encoding an exemplary CAR specific for the antigen cluster of differentiation 19 (an anti-CD19 CAR) was targeted for integration at one of the sites of genetic disruption via homology-dependent repair (HDR), or was introduced by random integration via retroviral transduction, in cells engineered to genetically disrupt the endogenous gene locus that encodes the T cell receptor alpha (TCRα) chain, by CRISPR/Cas9 mediated gene editing, generally as described in Examples 1 and 2.

These studies were carried out using AAV (for HDR) and retroviral constructs (for random integration) substantially as described in Examples 1 and 2, except with the following differences: the transgene sequences included nucleic acid sequences encoding an exemplary anti-CD19 CAR and an EF1α promoter to drive the expression of the exemplary anti-CD19 CAR sequences (TRAC HDR EF1α Promoter), or sequences encoding a P2A ribosome skip element upstream of the exemplary anti-CD19 CAR sequences (TRAC HDR P2A), to drive expression of the exemplary anti-CD19 CAR from the endogenous TCRα locus upon HDR-mediated targeted integration in-frame into the human TCR α constant region (TRAC) gene.

For targeted integration by HDR, primary human CD4+ and CD8+ T cells were stimulated, cultured and subject to electroporation with ribonucleoprotein (RNP) complexes containing TRAC-targeting gRNA generally as described in Examples 1 and 2. After electroporation, the cells were transduced with AAV preparations containing polynucleotides that encoded the exemplary anti-CD19 CAR for targeting to the endogenous TRAC locus, generally as described above. For random integration, the primary human CD4+ and CD8+ T cells were transduced with a retroviral preparation that encoded the exemplary anti-CD19 CAR, with (Retrovirus TRAC RNP or Retrovirus TCR KO) or without (Retrovirus only) electroporation of RNP complexes containing a TRAC-targeting gRNA. As controls, cells were subject to HDR targeting at the TRAC locus after electroporation with RNP complexes containing a TRBC-targeting gRNA, or were mock treated (Mock). On day 9 after thawing the cells, the engineered cells were assessed by flow cytometry for staining with an anti-CD3 antibody, or with an anti-idiotypic antibody (anti-ID) that specifically recognizes the anti-CD19 CAR.

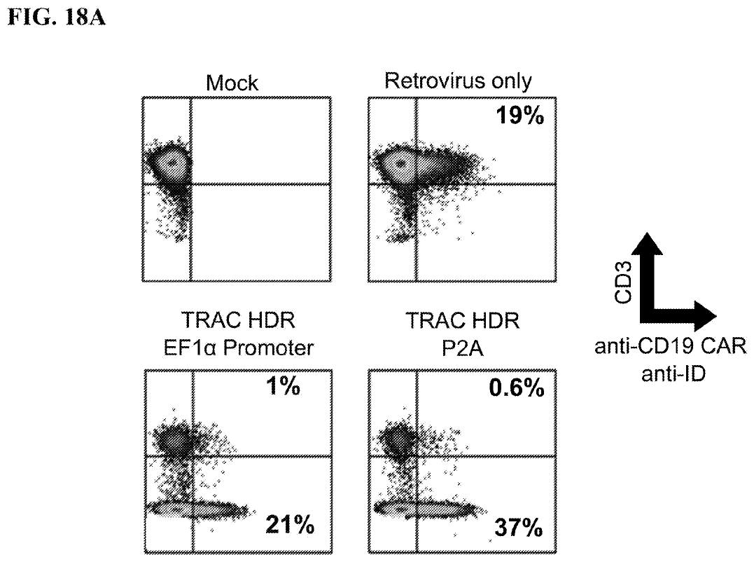

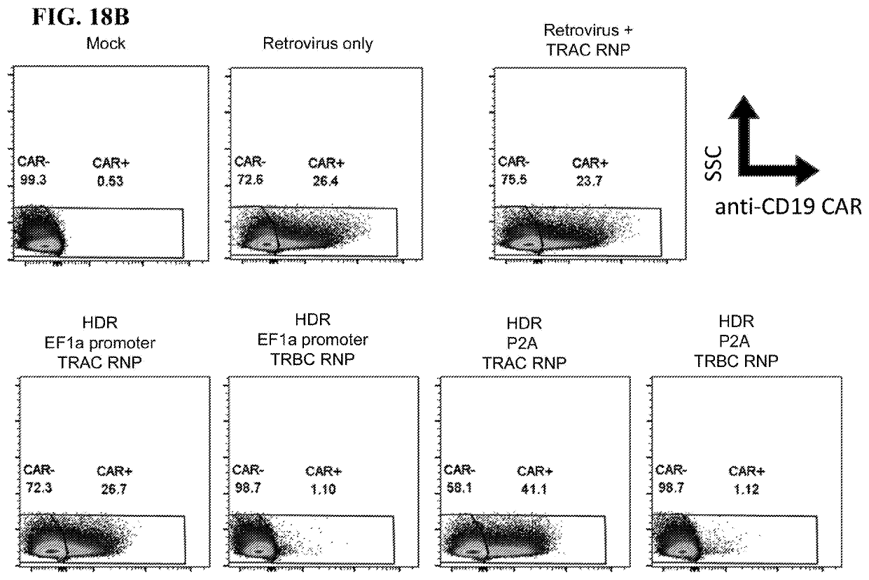

As shown in FIG. 18A, anti-CD19 CAR-expressing T cells generated by HDR-mediated targeted integration at the TRAC locus exhibited the highest proportion of cells bound by the anti-ID antibody. As shown in FIG. 18B, the efficiency of integration and expression of the exemplary anti-CD19 CAR, generated by HDR-mediated targeted integration at the TRAC locus, was comparable to or higher than the efficiency observed in cells engineered using random integration, with or without gene editing of the endogenous TRAC locus. The results were consistent with an observation of similar or improved expression of an exemplary anti-CD19 CAR in cells engineered by HDR-mediated targeted integration at the TRAC locus, compared to in cells engineered by random integration.

b. Expression of an Exemplary Anti-CD19 CAR after Serial Restimulation

Cell surface expression of the CAR was assessed after multiple rounds of exposure to the target antigen. The ability of CAR T cells to expand and exhibit antigen-specific function ex vivo following repeated rounds of antigen stimulation can correlate with in vivo function and/or capacity of the cells to persist in vivo (e.g., following administration and initial activation in response to encounter with antigen) (Zhao et al. (2015) Cancer Cell, 28:415-28).

Primary human T cells expressing the exemplary anti-CD19 CAR, generated by HDR-mediated targeted integration or random integration as described above, were incubated with K562 human chronic myelogenous leukemia (CML) cells engineered to express CD19 (K562-CD19). Irradiated K562-CD19 target cells were added to at an effector-to-target (E:T) ratio of 2.5:1. Every 4 days (start of each new round), CAR T cells were counted, harvested and re-plated at the initial seeding density with fresh media and newly-thawed, newly-irradiated target cells for a total of 3 rounds. At each round, the percentage of CAR-expressing cells, the mean fluorescence intensity (MFI), the coefficient of variation (CV) of CAR expression were assessed by flow cytometry.

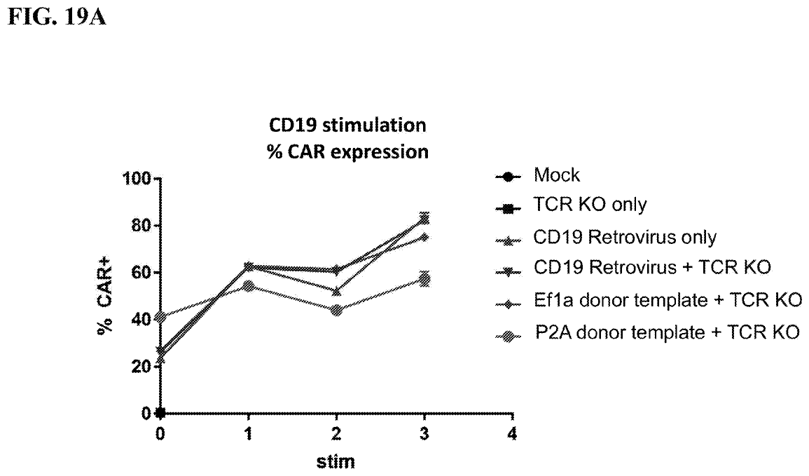

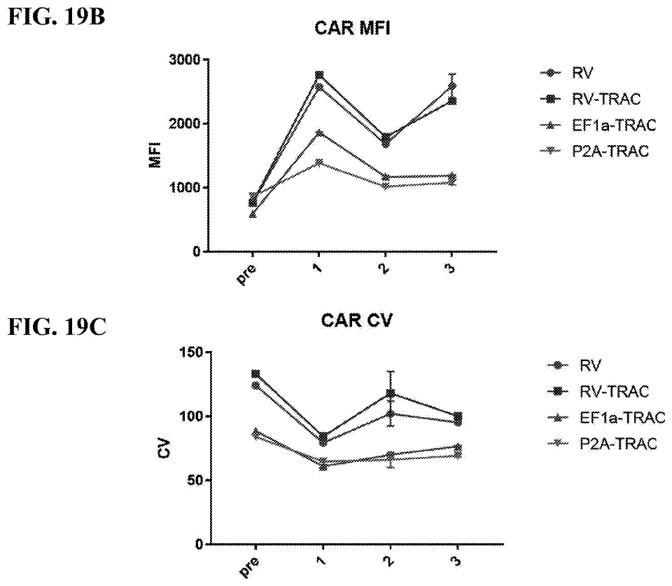

As shown in FIG. 19A, the percentage of cells expressing the anti-CD19 CAR generally increased or was stable in all tested groups. As shown in FIGS. 19B and 19C, over the course of repeated stimulation, exemplary anti-CD19 CAR-expressing cells, engineered by HDR-mediated targeted integration of the nucleic acid sequences at the endogenous TRAC locus, under the control of the EF1α promoter or P2A (endogenous TRAC promoter), exhibited more uniform and less variable expression of the CAR, compared to cells engineered by random integration using a retroviral vector. The MFI was generally higher in cells engineered by random integration (FIG. 19B). Anti-CD19 CAR-expressing cells engineered using HDR-mediated targeting at the TRAC locus exhibited a lower coefficient of variation (the standard deviation of signal within a population of cells divided by the mean of the signal in the respective population; FIG. 19C), indicating less fluctuation in expression over the repeated stimulation.

c. Cytolytic Activity and Cytokine Production

Cytokine production (interferon-gamma; IFNγ) and cytolytic activity were monitored following incubation of anti-CD19 CAR+T effector cells with K562 target cells engineered to express CD19 (K562-CD19) or non-engineered control (K562 parental), at an E:T ratio of 2:1 for 48 hours, generally as described in Example 1.D above.

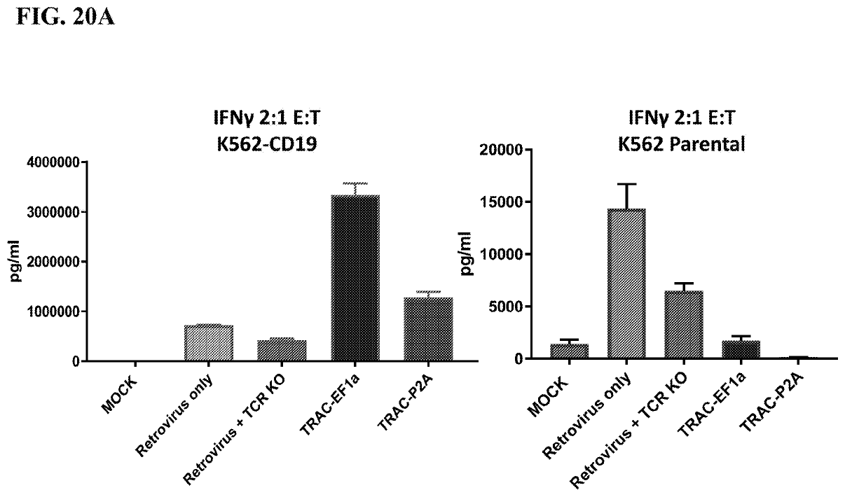

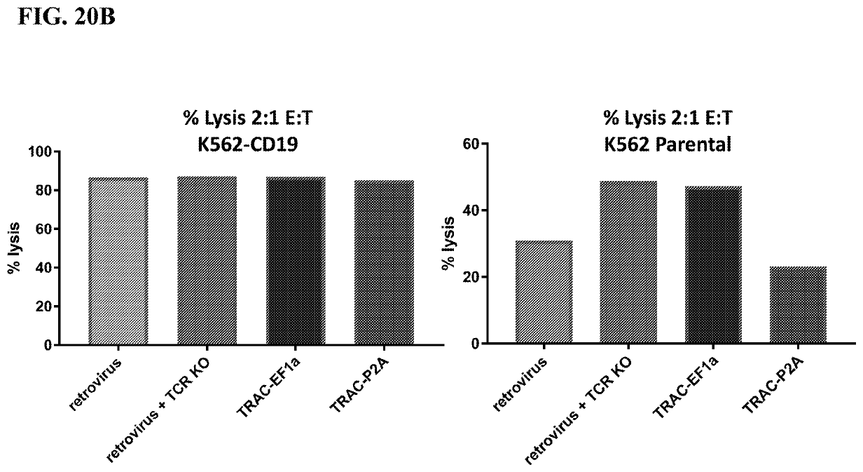

As shown in FIG. 20A, anti-CD19 CAR-expressing cells engineered using HDR-mediated targeting at the TRAC locus exhibited higher antigen-specific IFNγ production, with cells engineered by HDR with an EF1α promoter exhibiting the highest antigen-specific IFNγ production (left panel), and lower non-specific or off-target cytokine production (right panel). As shown in FIG. 20B, the degree of target cell killing was similar for all groups of anti-CD19 CAR-expressing cells (left panel). Non-specific cell killing varied, with cells engineered by HDR with P2A (endogenous TRAC promoter) exhibiting the lowest non-specific cell killing activity (right panel).

B. Exemplary Anti-BCMA CAR

a Expression of an Exemplary Anti-BCMA CAR

Nucleic acid sequences encoding a different exemplary CAR specific for B cell maturation antigen (an anti-BCMA CAR) was introduced by targeted integration mediated by HDR at the TRAC locus under the control of an EF1α or the endogenous TRAC promoter (by integrating a P2A sequence), or by random integration using a lentiviral vector, with (LV-TRAC or LV-KO) or without (LV only) introduction of RNP complexes containing a TRAC-targeting gRNA, generally as described in Examples 1, 2 and 6A above. As controls, cells were subject to HDR targeting at the TRAC locus after electroporation with RNP complexes containing a TRBC-targeting gRNA, or were mock treated (mock). On day 9 after thawing the cells, the engineered cells were assessed by flow cytometry, to detect anti-BCMA CAR expression by staining with a BCMA-Fc fusion polypeptide, containing recombinant soluble human BCMA fused at its C-terminus to an Fc region of IgG, that specifically binds to the anti-BCMA CAR, and CD3 expression.

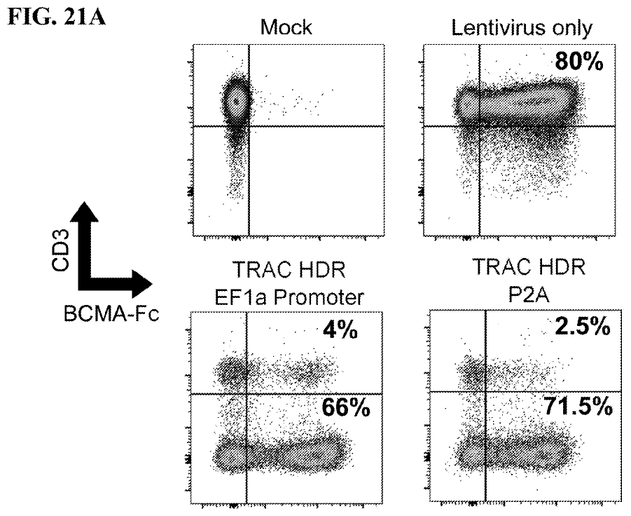

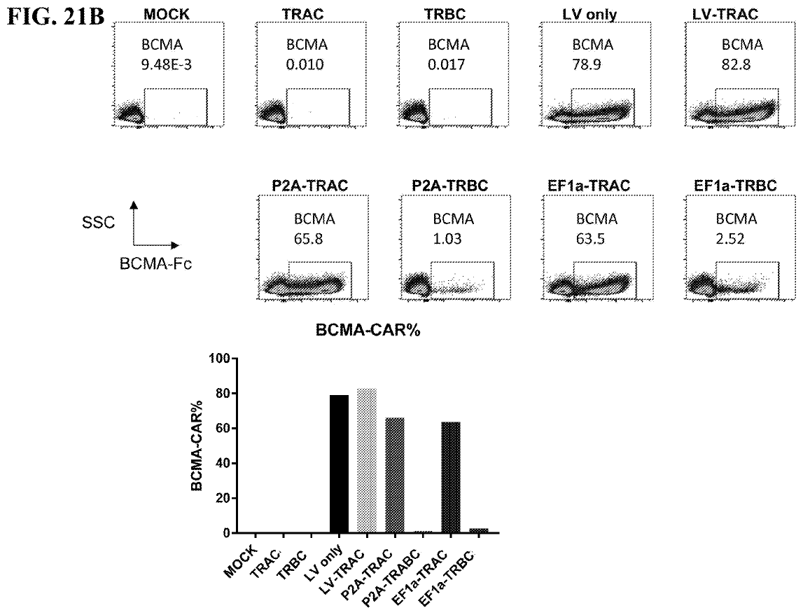

As shown in FIG. 21A, anti-BCMA CAR-expressing T cells generated by HDR-mediated targeted integration at the TRAC locus exhibited comparable proportion of cells bound by BCMA-Fc, as cells engineered using random integration. As shown in FIG. 21B, the percentage of cells expressing the exemplary anti-BCMA CAR, generated by HDR-mediated targeted integration at the TRAC locus, was comparable to the percentage observed in cells engineered using random integration, with or without gene editing of the endogenous TRAC locus. The results were consistent with an observation of similar or improved expression of an exemplary anti-BCMACAR in cells engineered by HDR-mediated targeted integration at the TRAC locus, compared to cells engineered using random integration.

h. Expression and Activity of an Exemplary anti-BCMA CAR After Serial Restimulation

Cell surface expression of the CAR and antigen-specific activity of the engineered cells were assessed after multiple rounds of exposure to the target antigen. Anti-BCMA CAR-expressing cells generated as described above were incubated with irradiated BCMA-expressing RPMI-8226 (a BCMA+ multiple myeloma cell line) target cells, at an effector-to-target (E:T) ratio of 1:1. Every 3-7 days after the start of each new round, CAR T cells were counted, harvested and re-plated at the initial seeding density with fresh media and newly-thawed, newly-irradiated target cells for a total of 4 rounds. The percentage of cells expressing the anti-BCMA CAR was determined by flow cytometry. For assessing cytokine production, cells at the first, second or third round of serial stimulation were collected 24 hours after stimulation, and the production of interferon gamma (IFNγ) and interleukin-2 (IL-2) was assessed.

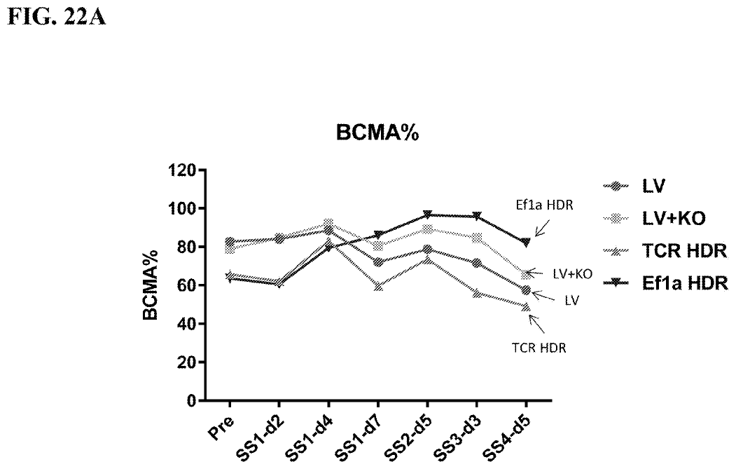

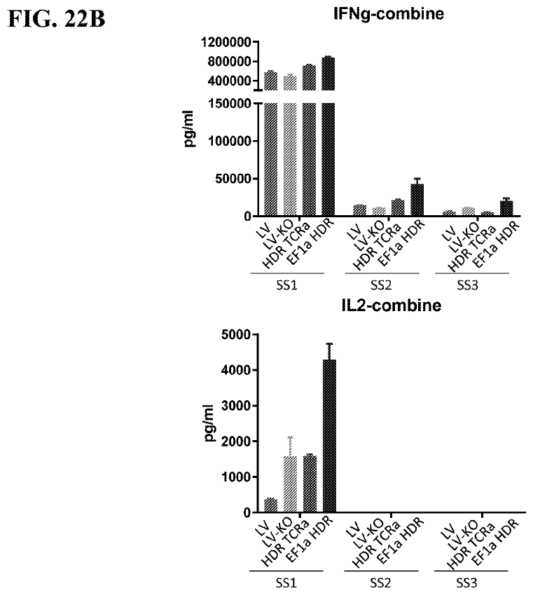

As shown in FIG. 22A, the percentage of anti-BCMA CAR-expressing cells generally were observed to be similar over the course of multiple rounds of stimulation, in cells engineered by the various methods. As shown in FIG. 22B, IFNγ production (top panel) was observed to be similar in cells engineered using the various methods. Cells engineered by targeted integration at the TRAC locus by HDR, under operable control of the EF1α promoter, exhibited highest IL-2 production after stimulation with target cells.

C. Conclusion

As shown, cells engineered to express exemplary CARs by targeted integration of the nucleic acid sequences into the endogenous TRAC locus exhibited similar or improved expression and antigen-specific activity and more uniform expression, including in the context of multiple rounds of stimulation.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | atatccagaaccctgaccctgccgtgtaccagctgagagactctaaatccagtgac | Human TCR alpha |
| | aagtctgtctgcctattcaccgattttgattctcaaacaaatgtgtcacaaagtaa | constant (TRAC) |
| | ggattctgatgtgtatatcacagacaaaactgtgctagacatgaggtctatggact | NCBI Reference |
| | tcaagagcaacagtgctgtggcctggagcaacaaatctgactttgcatgtgcaaac | Sequence: |
| | gccttcaacaacagcattattccagaagacaccttcttccccagcccaggtaaggg | NG_001332.3, TRAC |
| | cagctttggtgccttcgcaggctgtttccttgcttcaggaatggccaggttctgcc | |
| | cagagctctggtcaatgatgtctaaaactcctctgattggtggtctcggccttatc | |
| | cattgccaccaaaaccctctttttactaagaaacagtgagccttgttctggcagtc | |
| | cagagaatgacacgggaaaaaagcagatgaagagaaggtggcaggagagggcacgt | |
| | ggcccagcctcagtctctccaactgagttcctgcctgcctgcctttgctcagactg | |
| | tttgccccttactgctcttctaggcctcattctaagccccttctccaagttgcctc | |
| | tccttatttctccctgtctgccaaaaaatctttcccagctcactaagtcagtctca | |
| | cgcagtcactcattaacccaccaatcactgattgtgccggcacatgaatgcaccag | |
| | gtgttgaagtggaggaattaaaaagtcagatgaggggtgtgcccagaggaagcacc | |
| | attctagttgggggagcccatctgtcagctgggaaaagtccaaataacttcagatt | |
| | ggaatgtgttttaactcagggttgagaaaacagctaccttcaggacaaaagtcagg | |
| | gaagggctctctgaagaaatgctacttgaagataccagccctaccaagggcaggga | |
| | gaggaccctatagaggcctgggacaggagctcaatgagaaaggagaagagcagcag | |
| | gcatgagttgaatgaaggaggcagggccgggtcacagggccttctaggccatgaga | |
| | gggtagacagtattctaaggacgccagaaagctgttgatcggcttcaagcagggga | |
| | gggacacctaatttgcttttcttttttttttttttttttttttttttttttgagat | |
| | ggagttttgctcttgttgcccaggctggagtgcaatggtgcatcttggctcactgc | |
| | aacctccgcctcccaggttcaagtgattctcctgcctcagcctcccgagtagctga | |
| | gattacaggcacccgccaccatgcctggctaattttttgtatttttagtagagaca | |
| | gggtttcactatgttggccaggctggtctcgaactcctgacctcaggtgatccacc | |
| | cgcttcagcctcccaaagtgctgggattacaggcgtgagccaccacacccggcctg | |
| | cttttcttaaagatcaatctgagtgctgtacggagagtgggttgtaagccaagagt | |
| | agaagcagaaagggagcagttgcagcagagagatgatggaggcctgggcagggtgg | |
| | tggcagggaggtaaccaacaccattcaggtttcaaaggtagaaccatgcagggatg | |
| | agaaagcaaagaggggatcaaggaaggcagctggattttggcctgagcagctgagt | |
| | caatgatagtgccgtttactaagaagaaaccaaggaaaaaatttggggtgcaggga | |
| | tcaaaactttttggaacatatgaaagtacgtgtttatactctttatggcccttgtc | |
| | actatgtatgcctcgctgcctccattggactctagaatgaagccaggcaagagcag | |
| | ggtctatgtgtgatggcacatgtggccagggtcatgcaacatgtactttgtacaaa | |
| | cagtgtatattgagtaaatagaaatggtgtccaggagccgaggtatcggtcctgcc | |
| | agggccaggggctctccctagcaggtgctcatatgctgtaagttccctccagatct | |
| | ctccacaaggaggcatggaaaggctgtagttgttcacctgcccaagaactaggagg | |
| | tctggggtgggagagtcagcctgctctggatgctgaaagaatgtctgtttttcctt | |
| | ttagaaagttcctgtgatgtcaagctggtcgagaaaagctttgaaacaggtaagac | |
| | aggggtctagcctgggtttgcacaggattgcggaagtgatgaacccgcaataaccc | |
| | tgcctggatgagggagtgggaagaaattagtagatgtgggaatgaatgatgaggaa | |
| | tggaaacagcggttcaagacctgcccagagctgggtggggtctctcctgaatccct | |
| | ctcaccatctctgactttccattctaagcactttgaggatgagtttctagcttcaa | |
| | tagaccaaggactctctcctaggcctctgtattcctttcaacagctccactgtcaa | |
| | gagagccagagagagcttctgggtggcccagctgtgaaatttctgagtcccttagg | |
| | gatagccctaaacgaaccagatcatcctgaggacagccaagaggttttgccttctt | |
| | tcaagacaagcaacagtactcacataggctgtgggcaatggtcctgtctctcaaga | |
| | atcccctgccactcctcacacccaccctgggcccatattcatttccatttgagttg | |
| | ttcttattgagtcatccttcctgtggtagcggaactcactaaggggcccatctgga | |
| | cccgaggtattgtgatgataaattctgagcacctaccccatccccagaagggctca | |
| | gaaataaaataagagccaagtctagtcggtgtttcctgtcttgaaacacaatactg | |
| | ttggccctggaagaatgcacagaatctgtttgtaaggggatatgcacagaagctgc | |
| | aagggacaggaggtgcaggagctgcaggcctcccccacccagcctgctctgccttg | |
| | gggaaaaccgtgggtgtgtcctgcaggccatgcaggcctgggacatgcaagcccat | |
| | aaccgctgtggcctcttggttttacagatacgaacctaaactttcaaaacctgtca | |
| | gtgattgggttccgaatcctcctcctgaaagtggccgggtttaatctgctcatgac | |
| | gctgcggctgtggtccagctgaggtgaggggccttgaagctgggagtggggtttag | |
| | ggacgcgggtctctgggtgcatcctaagctctgagagcaaacctccctgcagggtc | |
| | ttgcttttaagtccaaagcctgagcccaccaaactctcctacttcttcctgttaca | |
| | aattcctcttgtgcaataataatggcctgaaacgctgtaaaatatcctcatttcag | |
| | ccgcctcagttgcacttctcccctatgaggtaggaagaacagttgtttagaaacga | |
| | agaaactgaggccccacagctaatgagtggaggaagagagacacttgtgtacacca | |
| | catgccttgtgttgtacttctctcaccgtgtaacctcctcatgtcctctctcccca | |
| | gtacggctctcttagctcagtagaaagaagacattacactcatattacaccccaat | |
| | cctggctagagtctccgcaccctcctcccccagggtccccagtcgtcttgctgaca | |
| | actgcatcctgttccatcaccatcaaaaaaaaactccaggctgggtgcgggggctc | |
| | acacctgtaatcccagcactttgggaggcagaggcaggaggagcacaggagctgga | |
| | gaccagcctgggcaacacagggagaccccgcctctacaaaaagtgaaaaaattaac | |
| | caggtgtggtgctgcacacctgtagtcccagctacttaagaggctgagatgggagg | |
| | atcgcttgagccctggaatgttgaggctacaatgagctgtgattgcgtcactgcac | |
| | tccagcctggaagacaaagcaagatcctgtctcaaataataaaaaaaataagaact | |
| | ccagggtacatttgctcctagaactctaccacatagccccaaacagagccatcacc | |
| | atcacatccctaacagtcctgggtcttcctcagtgtccagcctgacttctgttctt | |
| | cctcattccagatctgcaagattgtaagacagcctgtgctccctcgctccttcctc | |
| | tgcattgcccctcttctccctctccaaacagagggaactctcctacccccaaggag | |
| | gtgaaagctgctaccacctctgtgcccccccggcaatgccaccaactggatcctac | |
| | ccgaatttatgattaagattgctgaagagctgccaaacactgctgccaccccctct | |
| | gttcccttattgctgcttgtcactgcctgacattcacggcagaggcaaggctgctg | |
| | cagcctcccctggctgtgcacattccctcctgctccccagagactgcctccgccat | |
| | cccacagatgatggatcttcagtgggttctcttgggctctaggtcctgcagaatgt | |
| | tgtgaggggtttatttttttttaatagtgttcataaagaaatacatagtattcttc | |
| | ttctcaagacgtggggggaaattatctcattatcgaggccctgctatgctgtgtat | |
| | ctgggcgtgttgtatgtcctgctgccgatgccttc | |
| 2 | aggacctgaacaaggtgttcccacccgaggtcgctgtgtttgagccatcagaagca | Human TCR beta |
| | gagatctcccacacccaaaaggccacactggtgtgcctggccacaggcttcttccc | constant 1 (TRBC1) |
| | cgaccacgtggagctgagctggtgggtgaatgggaaggaggtgcacagtggggtca | NCBI Reference |
| | gcacagacccgcagcccctcaaggagcagcccgccctcaatgactccagatactgc | Sequence: |
| | ctgagcagccgcctgagggtctcggccaccttctggcagaacccccgcaaccactt | NG_001333.2, |
| | ccgctgtcaagtccagttctacgggctctcggagaatgacgagtggacccaggata | TRBC1 |
| | gggccaaacccgtcacccagatcgtcagcgccgaggcctggggtagagcaggtgag | |
| | tggggcctggggagatgcctggaggagattaggtgagaccagctaccagggaaaat | |
| | ggaaagatccaggtagcagacaagactagatccaaaaagaaaggaaccagcgcaca | |
| | ccatgaaggagaattgggcacctgtggttcattcttctcccagattctcagcccaa | |
| | cagagccaagcagctgggtcccctttctatgtggcctgtgtaactctcatctgggt | |
| | ggtgccccccatccccctcagtgctgccacatgccatggattgcaaggacaatgtg | |
| | gctgacatctgcatggcagaagaaaggaggtgctgggctgtcagaggaagctggtc | |
| | tgggcctgggagtctgtgccaactgcaaatctgactttacttttaattgcctatga | |
| | aaataaggtctctcatttattttcctctccctgctttctttcagactgtggcttta | |
| | cctcgggtaagtaagcccttccttttcctctccctctctcatggttcttgacctag | |
| | aaccaaggcatgaagaactcacagacactggagggtggagggtgggagagaccaga | |
| | gctacctgtgcacaggtacccacctgtccttcctccgtgccaacagtgtcctacca | |
| | gcaaggggtcctgtctgccaccatcctctatgagatcctgctagggaaggccaccc | |
| | tgtatgctgtgctggtcagcgcccttgtgttgatggccatggtaagcaggagggca | |
| | ggatggggccagcaggctggaggtgacacactgacaccaagcacccagaagtatag | |
| | agtccctgccaggattggagctgggcagtagggagggaagagatttcattcaggtg | |
| | cctcagaagataacttgcacctctgtaggatcacagtggaagggtcatgctgggaa | |
| | ggagaagctggagtcaccagaaaacccaatggatgttgtgatgagccttactattt | |
| | gtgtggtcaatgggccctactactttctctcaatcctcacaactcctggctcttaa | |
| | taacccccaaaactttctcttctgcaggtcaagagaaaggatttctga | |
| 3 | aggacctgaaaaacgtgttcccacccgaggtcgctgtgtttgagccatcagaagca | Human TCR beta |
| | gagatctcccacacccaaaaggccacactggtatgcctggccacaggcttctaccc | constant 2 (TRBC2) |
| | cgaccacgtggagctgagctggtgggtgaatgggaaggaggtgcacagtggggtca | NCBI Reference |
| | gcacagacccgcagcccctcaaggagcagcccgccctcaatgactccagatactgc | Sequence: |
| | ctgagcagccgcctgagggtctcggccaccttctggcagaacccccgcaaccactt | NG_001333.2, |
| | ccgctgtcaagtccagttctacgggctctcggagaatgacgagtggacccaggata | TRBC2 |
| | gggccaaacccgtcacccagatcgtcagcgccgaggcctggggtagagcaggtgag | |
| | tggggcctggggagatgcctggaggagattaggtgagaccagctaccagggaaaat | |
| | ggaaagatccaggtagcggacaagactagatccagaagaaagccagagtggacaag | |
| | gtgggatgatcaaggttcacagggtcagcaaagcacggtgtgcacttcccccacca | |
| | agaagcatagaggctgaatggagcacctcaagctcattcttccttcagatcctgac | |
| | accttagagctaagctttcaagtctccctgaggaccagccatacagctcagcatct | |
| | gagtggtgtgcatcccattctcttctggggtcctggtttcctaagatcatagtgac | |
| | cacttcgctggcactggagcagcatgagggagacagaaccagggctatcaaaggag | |
| | gctgactttgtactatctgatatgcatgtgtttgtggcctgtgagtctgtgatgta | |
| | aggctcaatgtccttacaaagcagcattctctcatccatttttcttcccctgtttt | |
| | ctttcagactgtggcttcacctccggtaagtgagtctctcctttttctctctatct | |
| | ttcgccgtctctgctctcgaaccagggcatggagaatccacggacacaggggcgtg | |
| | agggaggccagagccacctgtgcacaggtgcctacatgctctgttcttgtcaacag | |
| | agtcttaccagcaaggggtcctgtctgccaccatcctctatgagatcttgctaggg | |
| | aaggccaccttgtatgccgtgctggtcagtgccctcgtgctgatggccatggtaag | |
| | gaggagggtgggatagggcagatgatgggggcaggggatggaacatcacacatggg | |
| | cataaaggaatctcagagccagagcacagcctaatatatcctatcacctcaatgaa | |
| | accataatgaagccagactggggagaaaatgcagggaatatcacagaatgcatcat | |
| | gggaggatggagacaaccagcgagccctactcaaattaggcctcagagcccgcctc | |
| | ccctgccctactcctgctgtgccatagcccctgaaaccctgaaaatgttctctctt | |
| | ccacaggtcaagagaaaggattccagaggctag | |
| 4 | cgtgaggctccggtgcccgtcagtgggcagagcgcacatcgcccacagtccccgag | EF1alpha promoter |
| | aagttggggggaggggtcggcaattgaaccggtgcctagagaaggtggcgcggggt | (GenBank: J04617.1) |
| | aaactgggaaagtgatgtcgtgtactggctccgcctttttcccgagggtgggggag | |
| | aaccgtatataagtgcagtagtcgccgtgaacgttctttttcgcaacgggtttgcc | |
| | gccagaacacaggtaagtgccgtgtgtggttcccgcgggcctggcctctttacggg | |
| | ttatggcccttgcgtgccttgaattacttccacgcccctggctgcagtacgtgatt | |
| | cttgatcccgagcttcgggttggaagtgggtgggagagttcgaggccttgcgctta | |
| | aggagccccttcgcctcgtgcttgagttgaggcctggcctgggcgctggggccgcc | |
| | gcgtgcgaatctggtggcaccttcgcgcctgtctcgctgctttcgataagtctcta | |
| | gccatttaaaatttttgatgacctgctgcgacgctttttttctggcaagatagtct | |
| | tgtaaatgcgggccaagatctgcacactggtatttcggtttttggggccgcgggcg | |
| | gcgacggggcccgtgcgtcccagcgcacatgttcggcgaggcggggcctgcgagcg | |
| | cggccaccgagaatcggacgggggtagtctcaagctggccggcctgctctggtgcc | |
| | tggcctcgcgccgccgtgtatcgccccgccctgggcggcaaggctggcccggtcgg | |
| | caccagttgcgtgagcggaaagatggccgcttcccggccctgctgcagggagctca | |
| | aaatggaggacgcggcgctcgggagagcgggcgggtgagtcacccacacaaaggaa | |
| | aagggcctttccgtcctcagccgtcgcttcatgtgactccacggagtaccgggcgc | |
| | cgtccaggcacctcgattagttctcgagcttttggagtacgtcgtctttaggttgg | |
| | ggggaggggttttatgcgatggagtttccccacactgagtgggtggagactgaagt | |
| | taggccagcttggcacttgatgtaattctccttggaatttgccctttttgagtttg | |
| | gatcttggttcattctcaagcctcagacagtggttcaaagtttttttcttccattt | |
| | caggtgtcgtgaa | |
| 5 | CGTGAGGCTCCGGTGCCCGTCAGTGGGCAGAGCGCACATCGCCCACAGTCCCCGAG | EF1alpha promoter |
| | AAGTTGGGGGGAGGGGTCGGCAATTGAACCGGTGCCTAGAGAAGGTGGCGCGGGGT | |
| | AAACTGGGAAAGTGATGTCGTGTACTGGCTCCGCCTTTTTCCCGAGGGTGGGGGAG | |
| | AACCGTATATAAGTGCACTAGTCGCCGTGAACGTTCTTTTTCGCAACGGGTTTGCC | |
| | GCCAGAACACAGGTAAGTGCCGTGTGTGGTTCCCGCGGGCCTGGCCTCTTTACGGG | |
| | TTATGGCCCTTGCGTGCCTTGAATTACTTCCACCTGGCTGCAGTACGTGATTCTTG | |
| | ATCCCGAGCTTCGGGTTGGAAGTGGGTGGGAGAGTTCGTGGCCTTGCGCTTAAGGA | |
| | GCCCCTTCGCCTCGTGCTTGAGTTGTGGCCTGGCCTGGGCGCTGGGGCCGCCGCGT | |
| | GCGAATCTGGTGGCACCTTCGCGCCTGTCTCGCTGCTTTCGATAAGTCTCTAGCCA | |
| | TTTAAAATTTTTGATGACCTGCTGCGACGCTTTTTTTCTGGCAAGATAGTCTTGTA | |
| | AATGCGGGCCAAGATCAGCACACTGGTATTTCGGTTTTTGGGGCCGCGGGCGGCGA | |
| | CGGGGCCCGTGCGTCCCAGCGCACATGTTCGGCGAGGCGGGGCCTGCGAGCGCGGC | |
| | CACCGAGAATCGGACGGGGGTAGTCTCAAGCTGCCCGGCCTGCTCTGGTGCCTGGC | |
| | CTCGCGCCGCCGTGTATCGCCCCGCCCTGGGCGGCAAGGCTGGCCCGGTCGGCACC | |
| | AGTTGCGTGAGCGGAAAGATGGCCGCTTCCCGGCCCTGCTGCAGGGAGCACAAAAT | |
| | GGAGGACGCGGCGCTCGGGAGAGCGGGCGGGTGAGTCACCCACACAAAGGAAAAGG | |
| | GCCTTTCCGTCCTCAGCCGTCGCTTCATGTGACTCCACGGAGTACCGGGCGCCGTC | |
| | CAGGCACCTCGATTAGTTCTCCAGCTTTTGGAGTACGTCGTCTTTAGGTTGGGGGG | |
| | AGGGGTTTTATGCGATGGAGTTTCCCCACACTGAGTGGGTGGAGACTGAAGTTAGG | |
| | CCAGCTTGGCACTTGATGTAATTCTCCTTGGAATTTGCCCTTTTTGAGTTTGGATC | |
| | TTGGTTCATTCTCAAGCCTCAGACAGTGGTTCAAAGTTTTTTTCTTCCATTTCAGG | |
| | TGTCGTGAAAACTACCCCTAAAAGCCAAA | |
| 6 | LEGGGEGRGSLLTCGDVEENPGPR | T2A |
| 7 | EGRGSLLTCGDVEENPGP | T2A |
| 8 | GSGATNFSLLKQAGDVEENPGP | P2A |
| 9 | ATNFSLLKQAGDVEENPGP | P2A |
| 10 | QCTNYALLKLAGDVESNPGP | E2A |
| 11 | VKQTLNFDLLKLAGDVESNPGP | F2A |
| 12 | MLLLVTSLLLCELPHPAFLLIPRKVCNGIGIGEFKDSLSINATNIKHFKNCTSISG | EGFRt |
| | DLHILPVAFRGDSFTHTPPLDPQELDILKTVKEITGFLLIQAWPENRTDLHAFENL | |
| | ElIRGRTKQHGQFSLAVVSLNITSLGLRSLKEISDGDVIISGNKNLCYANTINWKK | |
| | LFGTSGQKTKIISNRGENSCKATGQVCHALCSPEGCWGPEPRDCVSCRNVSRGREC | |
| | VDKCNLLEGEPREFVENSECIQCHPECLPQAMNITCTGRGPDNCIQCAHYIDGPHC | |
| | VKTCPAGVMGENNTLVWKYADAGHVCHLCHPNCTYGCTGPGLEGCPTNGPKIPSIA | |
| | TGMVGALLLLLVVALGIGLFM | |
| 13 | RKVCNGIGIGEFKDSLSINATNIKHFKNCTSISGDLHILPVAFRGDSFTHTPPLDP | EGFRt |
| | QELDILKTVKEITGFLLIQAWPENRTDLHAFENLEIIRGRTKQHGQFSLAVVSLNI | |
| | TSLGLRSLKEISDGDVIISGNKNLCYANTINWKKLFGTSGQKTKIISNRGENSCKA | |
| | TGQVCHALCSPEGCWGPEPRDCVSCRNVSRGRECVDKCNLLEGEPREFVENSECIQ | |
| | CHPECLPQAMNITCTGRGPDNCIQCAHYIDGPHCVKTCPAGVMGENNTLVWKYADA | |
| | GHVCHLCHPNCTYGCTGPGLEGCPTNGPKIPSIATGMVGALLLLLVVALGIGLFM | |
| 14 | DIQNPEPAVYQLKDPRSQDSTLCLFTDFDSQINVPKTMESGTFITDKTVLDMKAMD | Mouse TCR alpha |
| | SKSNGAIAWSNQTSFTCQDIFKETNATYPSSDVPCDATLTEKSFETDMNLNFQNLS | constant |
| | VMGLRILLLKVAGFNLLMTLRLWSS | |
| 15 | NIQNPEPAVYQLKDPRSQDSTLCLFTDFDSQINVPKTMESGTFITDKTVLDMKAMD | Mouse TCR alpha |
| | SKSNGAIAWSNQTSFTCQDIFKETNATYPSSDVPCDATLTEKSFETDMNLNFQNLS | constant |
| | VMGLRILLLKVAGFNLLMTLRLWSS | |
| 16 | EDLRNVTPPKVSLFEPSKAEIANKQKATLVCLARGFFPDHVELSWWVNGKEVHSGV | Mouse TCR beta |
| | STDPQAYKESNYSYCLSSRLRVSATFWHNPRNHFRCQVQFHGLSEEDKWPEGSPKP | constant (Uniprot |
| | VTQNISAEAWGRADCGITSASYQQGVLSATILYEILLGKATLYAVLVSTLVVMAMV | P01852) |
| | KRKNS | |
| 17 | DLRNVTPPKVSLFEPSKAEIANKQKATLVCLARGFFPDHVELSWWVNGKEVHSGVS | Mouse TCR beta |
| | TDPQAYKESNYSYCLSSRLRVSATFWHNPRNHFRCQVQFHGLSEEDKWPEGSPKPV | constant |
| | TQNISAEAWGRADCGITSASYHQGVLSATILYEILLGKATLYAVLVSGLVLMAMVK | |
| | RKNS | |
| 18 | gggtctctctggttagaccagatctgagcctgggagctctctggctaactagggaa | MND promoter |
| | cccactgcttaagcctcaataaagcttgccttgagtgcttcaagtagtgtgtgccc | |
| | gtctgttgtgtgactctggtaactagagatccctcagacccttttagtcagtgtgg | |
| | aaaatctctagca | |
| 19 | PNIQNPDPAVYQLRDSKSSDKSVCLFTDFDSQTNVSQSKDSDVYITDKTVLDMRSM | Human TCR alpha |
| | DFKSNSAVAWSNKSDFACANAFNNSIIPEDTFFPSPESSCDVKLVEKSFETDTNLN | constant (Uniprot |
| | FQNLSVIGFRILLLKVAGFNLLMTLRLWSS | P01848) |
| 20 | EDLNKVFPPEVAVFEPSEAEISHTQKATLVCLATGFFPDHVELSWWVNGKEVHSGV | Human TCR beta |
| | STDPQPLKEQPALNDSRYCLSSRLRVSATFWQNPRNHFRCQVQFYGLSENDEWTQD | constant 1 (Uniprot |
| | RAKPVTQIVSAEAWGRADCGFTSVSYQQGVLSATILYEILLGKATLYAVLVSALVL | P01850) |
| | MAMVKRKDF | |
| 21 | DLKNVFPPEVAVFEPSEAEISHTQKATLVCLATGFYPDHVELSWWVNGKEVHSGVS | Human TCR beta |
| | TDPQPLKEQPALNDSRYCLSSRLRVSATFWQNPRNHFRCQVQFYGLSENDEWTQDR | constant 2 (Uniprot |
| | AKPVTQIVSAEAWGRADCGFTSESYQQGVLSATILYEILLGKATLYAVLVSALVLM | A0A5B9) |
| | AMVKRKDSRG | |
| 22 | -PGGG-(SGGGG)n-P- wherein P is proline, G is glycine and | Linker |
| | S is serine | |
| 23 | GSADDAKKDAAKKDGKS | Linker |
| 24 | NIQNPDPAVYQLRDSKSSDKSVCLFTDFDSQTNVSQSKDSDVYITDKTVLDMRSMD | human TCR alpha |
| | FKSNSAVAWSNKSDFACANAFNNSIIPEDTFFPSPESSCDVKLVEKSFETDTNLNF | constant (Genbank |
| | QNLSVIGFRILLLKVAGFNLLMTLRLWSS | Accession No. |
| | | CAA26636.1) |
| 25 | EDLKNVFPPEVAVFEPSEAEISHTQKATLVCLATGFYPDHVELSWWVNGKEVHSGV | human TCR beta |
| | STDPQPLKEQPALNDSRYCLSSRLRVSATFWQNPRNHFRCQVQFYGLSENDEWTQD | constant (Uniprot |
| | RAKPVTQIVSAEAWGRADCGFTSESYQQGVLSATILYEILLGKATLYAVLVSALVL | Accession No. |
| | MAMVKRKDSRG | A0A0G2JNG9) |
| 26 | AGCGCTCTCGTACAGAGTTGGCATTATAATACGACTCACTATAGGGGAGAATCAAA | TRAC gRNA |
| | ATCGGTGAATGTTTTAGAGCTAGAAATAGCAAGTTAAAATAAGGCTAGTCCGTTAT | transcription |
| | CAACTTGAAAAAGTGGCACCGAGTCGGTGCTTTTTTT | sequence |
| 27 | GAGAAUCAAAAUCGGUGAAUGUUUUAGAGCUAGAAAUAGCAAGUUAAAAUAAGGCU | TRAC gRNA |
| | AGUCCGUUAUCAACUUGAAAAAGUGGCACCGAGUCGGUGCUUUU | sequence |
| 28 | UCUCUCAGCUGGUACACGGC | TRAC-10 gRNA |
| | | targeting domain |
| 29 | UGGAUUUAGAGUCUCUCAGC | TRAC-110 gRNA |
| | | targeting domain |
| 30 | ACACGGCAGGGUCAGGGUUC | TRAC-l16 gRNA |
| | | targeting domain |
| 31 | GAGAAUCAAAAUCGGUGAAU | TRAC-16 gRNA |
| | | targeting domain |
| 32 | GCUGGUACACGGCAGGGUCA | TRAC-4 gRNA |
| | | targeting domain |
| 33 | CUCAGCUGGUACACGGC | TRAC-49 gRNA |
| | | targeting domain |
| 34 | UGGUACACGGCAGGGUC | TRAC-2 gRNA |
| | | targeting domain |
| 35 | GCUAGACAUGAGGUCUA | TRAC-30 gRNA |
| | | targeting domain |
| 36 | GUCAGAUUUGUUGCUCC | TRAC-43 gRNA |
| | | targeting domain |
| 37 | UCAGCUGGUACACGGCA | TRAC-23 gRNA |
| | | targeting domain |
| 38 | GCAGACAGACUUGUCAC | TRAC-34 gRNA |
| | | targeting domain |
| 39 | GGUACACGGCAGGGUCA | TRAC-25 gRNA |
| | | targeting domain |
| 40 | CUUCAAGAGCAACAGUGCUG | TRAC-128 gRNA |
| | | targeting domain |
| 41 | AGAGCAACAGUGCUGUGGCC | TRAC-105 gRNA |
| | | targeting domain |
| 42 | AAAGUCAGAUUUGUUGCUCC | TRAC-106 gRNA |
| | | targeting domain |
| 43 | ACAAAACUGUGCUAGACAUG | TRAC-123 gRNA |
| | | targeting domain |
| 44 | AAACUGUGCUAGACAUG | TRAC-64 gRNA |
| | | targeting domain |
| 45 | UGUGCUAGACAUGAGGUCUA | TRAC-97 gRNA |
| | | targeting domain |
| 46 | GGCUGGGGAAGAAGGUGUCUUC | TRAC-148 gRNA |
| | | targeting domain |
| 47 | GCUGGGGAAGAAGGUGUCUUC | TRAC-147 gRNA |
| | | targeting domain |
| 48 | GGGGAAGAAGGUGUCUUC | TRAC-234 gRNA |
| | | targeting domain |
| 49 | GUUUUGUCUGUGAUAUACACAU | TRAC-167 gRNA |
| | | targeting domain |
| 50 | GGCAGACAGACUUGUCACUGGAUU | TRAC-177 gRNA |
| | | targeting domain |
| 51 | GCAGACAGACUUGUCACUGGAUU | TRAC-116 gRNA |
| | | targeting domain |
| 52 | GACAGACUUGUCACUGGAUU | TRAC-251 gRNA |
| | | targeting domain |
| 53 | GUGAAUAGGCAGACAGACUUGUCA | TRAC-233 gRNA |
| | | targeting domain |
| 54 | GAAUAGGCAGACAGACUUGUCA | TRAC-231 gRNA |
| | | targeting domain |
| 55 | GAGUCUCUCAGCUGGUACACGG | TRAC-163 gRNA |
| | | targeting domain |
| 56 | GUCUCUCAGCUGGUACACGG | TRAC-241 gRNA |
| | | targeting domain |
| 57 | GGUACACGGCAGGGUCAGGGUU | TRAC-119 gRNA |
| | | targeting domain |
| 58 | GUACACGGCAGGGUCAGGGUU | TRAC-m gRNA |
| | | targeting domain |
| 59 | CACCCAGAUCGUCAGCGCCG | TRBC-40 gRNA |
| | | targeting domain |
| 60 | CAAACACAGCGACCUCGGGU | TRBC-52 gRNA |
| | | targeting domain |
| 61 | UGACGAGUGGACCCAGGAUA | TRBC-25 gRNA |
| | | targeting domain |
| 62 | GGCUCUCGGAGAAUGACGAG | TRBC-35 gRNA |
| | | targeting domain |
| 63 | GGCCUCGGCGCUGACGAUCU | TRBC-50 gRNA |
| | | targeting domain |
| 64 | GAAAAACGUGUUCCCACCCG | TRBC-39 gRNA |
| | | targeting domain |
| 65 | AUGACGAGUGGACCCAGGAU | TRBC-49 gRNA |
| | | targeting domain |
| 66 | AGUCCAGUUCUACGGGCUCU | TRBC-51 gRNA |
| | | targeting domain |
| 67 | CGCUGUCAAGUCCAGUUCUA | TRBC-26 gRNA |
| | | targeting domain |
| 68 | AUCGUCAGCGCCGAGGCCUG | TRBC-41 gRNA |
| | | targeting domain |
| 69 | UCAAACACAGCGACCUCGGG | TRBC-45 gRNA |
| | | targeting domain |
| 70 | CGUAGAACUGGACUUGACAG | TRBC-34 gRNA |
| | | targeting domain |
| 71 | AGGCCUCGGCGCUGACGAUC | TRBC-221 gRNA |
| | | targeting domain |
| 72 | UGACAGCGGAAGUGGUUGCG | TRBC-41 gRNA |
| | | targeting domain |
| 73 | UUGACAGCGGAAGUGGUUGC | TRBC-30 gRNA |
| | | targeting domain |
| 74 | UCUCCGAGAGCCCGUAGAAC | TRBC-206 gRNA |
| | | targeting domain |
| 75 | CGGGUGGGAACACGUUUUUC | TRBC-32 gRNA |
| | | targeting domain |
| 76 | GACAGGUUUGGCCCUAUCCU | TRBC-216 gRNA |
| | | targeting domain |
| 77 | GAUCGUCAGCGCCGAGGCCU | TRBC-214 gRNA |
| | | targeting domain |
| 78 | GGCUCAAACACAGCGACCUC | TRBC-230 gRNA |
| | | targeting domain |
| 79 | UGAGGGUCUCGGCCACCUUC | TRBC-235 gRNA |
| | | targeting domain |
| 80 | AGGCUUCUACCCCGACCACG | TRBC-38 gRNA |
| | | targeting domain |
| 81 | CCGACCACGUGGAGCUGAGC | TRBC-223 gRNA |
| | | targeting domain |
| 82 | UGACAGGUUUGGCCCUAUCC | TRBC-221 gRNA |
| | | targeting domain |
| 83 | CUUGACAGCGGAAGUGGUUG | TRBC-48 gRNA |
| | | targeting domain |
| 84 | AGAUCGUCAGCGCCGAGGCC | TRBC-216 gRNA |
| | | targeting domain |
| 85 | GCGCUGACGAUCUGGGUGAC | TRBC-210 gRNA |
| | | targeting domain |
| 86 | UGAGGGCGGGCUGCUCCUUG | TRBC-268 gRNA |
| | | targeting domain |
| 87 | GUUGCGGGGGUUCUGCCAGA | TRBC-193 gRNA |
| | | targeting domain |
| 88 | AGCUCAGCUCCACGUGGUCG | TRBC-246 gRNA |
| | | targeting domain |
| 89 | GCGGCUGCUCAGGCAGUAUC | TRBC-228 |
| | | gRNA |
| | | targeting domain |
| 90 | GCGGGGGUUCUGCCAGAAGG | TRBC-43 gRNA |
| | | targeting domain |
| 91 | UGGCUCAAACACAGCGACCU | TRBC-272 gRNA |
| | | targeting domain |
| 92 | ACUGGACUUGACAGCGGAAG | TRBC-33 gRNA |
| | | targeting domain |
| 93 | GACAGCGGAAGUGGUUGCGG | TRBC-44 gRNA |
| | | targeting domain |
| 94 | GCUGUCAAGUCCAGUUCUAC | TRBC-211 gRNA |
| | | targeting domain |
| 95 | GUAUCUGGAGUCAUUGAGGG | TRBC-253 gRNA |
| | | targeting domain |
| 96 | CUCGGCGCUGACGAUCU | TRBC-18 gRNA |
| | | targeting domain |
| 97 | CCUCGGCGCUGACGAUC | TRBC-6 gRNA |
| | | targeting domain |
| 98 | CCGAGAGCCCGUAGAAC | TRBC-85 gRNA |
| | | targeting domain |
| 99 | CCAGAUCGUCAGCGCCG | TRBC-129 gRNA |
| | | targeting domain |
| 100 | GAAUGACGAGUGGACCC | TRBC-93 gRNA |
| | | targeting domain |
| 101 | GGGUGACAGGUUUGGCCCUAUC | TRBC-415 gRNA |
| | | targeting domain |
| 102 | GGUGACAGGUUUGGCCCUAUC | TRBC-414 gRNA |
| | | targeting domain |
| 103 | GUGACAGGUUUGGCCCUAUC | TRBC-310 gRNA |
| | | targeting domain |
| 104 | GACAGGUUUGGCCCUAUC | TRBC-308 gRNA |
| | | targeting domain |
| 105 | GAUACUGCCUGAGCAGCCGCCU | TRBC-401 gRNA |
| | | targeting domain |
| 106 | GACCACGUGGAGCUGAGCUGGUGG | TRBC-468 gRNA |
| | | targeting domain |
| 107 | GUGGAGCUGAGCUGGUGG | TRBC-462 gRNA |
| | | targeting domain |
| 108 | GGGCGGGCUGCUCCUUGAGGGGCU | TRBC-424 gRNA |
| | | targeting domain |
| 109 | GGCGGGCUGCUCCUUGAGGGGCU | TRBC-423 gRNA |
| | | targeting domain |
| 110 | GCGGGCUGCUCCUUGAGGGGCU | TRBC-422 gRNA |
| | | targeting domain |
| 111 | GGGCUGCUCCUUGAGGGGCU | TRBC-420 gRNA |
| | | targeting domain |
| 112 | GGCUGCUCCUUGAGGGGCLJ | TRBC-419 gRNA |
| | | targeting domain |
| 113 | GCUGCUCCUUGAGGGGCU | TRBC-418 gRNA |
| | | targeting domain |
| 114 | GGUGAAUGGGAAGGAGGUGCACAG | TRBC-445 gRNA |
| | | targeting domain |
| 115 | GUGAAUGGGAAGGAGGUGCACAG | TRBC-444 gRNA |
| | | targeting domain |
| 116 | GAAUGGGAAGGAGGUGCACAG | TRBC-442 gRNA |
| | | targeting domain |
| 117 | ATTCACCGATTTTGATTCTC | TRAC target sequence |
| | | 1 |
| 118 | AGATCGTCAGCGCCGAGGCC | TRBC target sequence |
| | | 2 |
| 119 | CTGACCTCTTCTCTTCCTCCCACAG | HBB splice site |
| | | acceptor |
| 120 | TTTCTCTCCACAG | IgG splice site |
| | | acceptor |
| 121 | PYIQNPEPAVYQLKDPRSQDSTLCLFTDFDSQINVPKTMESGTFITDKTVLDMKAM | Mouse TCR alpha |
| | DSKSNGAIAWSNQTSFTCQDIFKETNATYPSSDVPCDATLTEKSFETDMNLNFQNL | constant (Uniprot |
| | SVMGLRILLLKVAGFNLLMTLRLWSS | P01849) |
| 122 | IQNPEPAVYQLKDPRSQDSTLCLFTDFDSQINVPKTMESGTFITDKCVLDMKAMDS | Modified mouse TCR |
| | KSNGAIAWSNQTSFTCQDIFKETNATYPSSDVPCDATLTEKSFETDMNLNFQNLLV | alpha constant |
| | IVLRILLLKVAGFNLLMTLRLWSS | |
| 123 | EDLRNVTPPKVSLFEPSKAEIANKQKATLVCLARGFFPDHVELSWWVNGKEVHSGV | Modified mouse TCR |
| | CTDPQAYKESNYSYCLSSRLRVSATFWHNPRNHFRCQVQFHGLSEEDKWPEGSPKP | beta constant |
| | VTQNISAEAWGRADCGITSASYQQGVLSATILYEILLGKATLYAVLVSTLVVMAMV | |
| | KRKNS | |
| 124 | CTCTATCAATGAGAGAGCAATCTCCTGGTAATGTGATAGATTTCCCAACTTAATGC | TRAC 5′ homology |
| | CAACATACCATAAACCTCCCATTCTGCTAATGCCCAGCCTAAGTTGGGGAGACCAC | arm |
| | TCCAGATTCCAAGATGTACAGTTTGCTTTGCTGGGCCTTTTTCCCATGCCTGCCTT | |
| | TACTCTGCCAGAGTTATATTGCTGGGGTTTTGAAGAAGATCCTATTAAATAAAAGA | |
| | ATAAGCAGTATTATTAAGTAGCCCTGCATTTCAGGTTTCCTTGAGTGGCAGGCCAG | |
| | GCCTGGCCGTGAACGTTCACTGAAATCATGGCCTCTTGGCCAAGATTGATAGCTTG | |
| | TGCCTGTCCCTGAGTCCCAGTCCATCACGAGCAGCTGGTTTCTAAGATGCTATTTC | |
| | CCGTATAAAGCATGAGACCGTGACTTGCCAGCCCCACAGAGCCCCGCCCTTGTCCA | |
| | TCACTGGCATCTGGACTCCAGCCTGGGTTGGGGCAAAGAGGGAAATGAGATCATGT | |
| | CCTAACCCTGATCCTCTTGTCCCACAGATATCCAGAACCCTGACCCTGCCGTGTAC | |
| | CAGCTGAGAGACTCTAAATCCAGTGACAAGTCTGTCTGCCTATTCACCGAT | |
| 125 | TTTGATTCTCAAACAAATGTGTCACAAAGTAAGGATTCTGATGTGTATATCACAGA | TRAC 3′ homology |
| | CAAAACTGTGCTAGACATGAGGTCTATGGACTTCAAGAGCAACAGTGCTGTGGCCT | arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTCTAGG | |
| | CCTCATTCTAAGCCCCTTCTCCAAGTTGCCTCTCCTTATTTCTCCCTGTCTGCCAA | |
| | AAAATCTTTCCCAGCTCACTAAGTCAGTCTCACGCAGTCACTCATTAACCCACCAA | |
| | TCACTGATTGTG | |
| 126 | GAACAGAGAAACAGGAGAATATGGGCCAAACAGGATATCTGTGGTAAGCAGTTCCT | MND promoter |
| | GCCCCGGCTCAGGGCCAAGAACAGTTGGAACAGCAGAATATGGGCCAAACAGGATA | |
| | TCTGTGGTAAGCAGTTCCTGCCCCGGCTCAGGGCCAAGAACAGATGGTCCCCAGAT | |
| | GCGGTCCCGCCCTCAGCAGTTTCTAGAGAACCATCAGATGTTTCCAGGGTGCCCCA | |
| | AGGACCTGAAATGACCCTGTGCCTTATTTGAACTAACCAATCAGTTCGCTTCTCGC | |
| | TTCTGTTCGCGCGCTTCTGCTCCCCGAGCTCTATATAAGCAGAGCTCGTTTAGTGA | |
| | ACCGTCAGATC | |
| 127 | GGATCTGCGATCGCTCCGGTGCCCGTCAGTGGGCAGAGCGCACATCGCCCACAGTC | Ef1alpha promoter |
| | CCCGAGAAGTTGGGGGGAGGGGTCGGCAATTGAACCGGTGCCTAGAGAAGGTGGCG | with HTLV1 enhancer |
| | CGGGGTAAACTGGGAAAGTGATGTCGTGTACTGGCTCCGCCTTTTTCCCGAGGGTG | |
| | GGGGAGAACCGTATATAAGTGCAGTAGTCGCCGTGAACGTTCTTTTTCGCAACGGG | |
| | TTTGCCGCCAGAACACAGCTGAAGCTTCGAGGGGCTCGCATCTCTCCTTCACGCGC | |
| | CCGCCGCCCTACCTGAGGCCGCCATCCACGCCGGTTGAGTCGCGTTCTGCCGCCTC | |
| | CCGCCTGTGGTGCCTCCTGAACTGCGTCCGCCGTCTAGGTAAGTTTAAAGCTCAGG | |
| | TCGAGACCGGGCCTTTGTCCGGCGCTCCCTTGGAGCCTACCTAGACTCAGCCGGCT | |
| | CTCCACGCTTTGCCTGACCCTGCTTGCTCAACTCTACGTCTTTGTTTCGTTTTCTG | |
| | TXCTGCGCCGTTACAGATCCAAGCTGTGACCGGCGCCTAC | |
| 128 | GGATCTGGAGCGACGAATTTTAGTCTACTGAAACAAGCGGGAGACGTGGAGGAAAA | P2A nucleotide |
| | CCCTGGACCT | sequence |
| 129 | RVKFSRSAEPPAYQQGQNQLYNELNLGRREEYDVLDKRRGRDPEMGGKPRRKNPQE | CD3 zeta |
| | GLYNELQKDKMAEAYSEIGMKGERRRGKGHDGLYQGLSTATKDTYDALHMQALPPR | |
| 130 | RVKFSRSADAPAYKQGQNQLYNELNLGRREEYDVLDKRRGRDPEMGGKPRRKNPQE | CD3 zeta |
| | GLYNELQKDKMAEAYSEIGMKGERRRGKGHDGLYQGLSTATKDTYDALHMQALPPR | |
| 131 | ESKYGPPCPPCP | spacer (IgG4hinge) |
| 132 | GAATCTAAGTACGGACCGCCCTGCCCCCCTTGCCCT | spacer (IgG4hinge) |
| 133 | ESKYGPPCPPCPGQPREPQVYTLPPSQEEMTKNQVSLTCLVKGFYPSDIAVEWESN | Hinge-CH3 spacer |
| | GQPENNYKTTPPVLDSDGSFFLYSRLTVDKSRWQEGNVFSCSVMHEALHNHYTQKS | |
| | LSLSLGK | |
| 134 | ESKYGPPCPPCPAPEFLGGPSVFLFPPKPKDTLMISRTPEVTCVVVDVSQEDPEVQ | Hinge-CH2-CH3 |
| | FNWYVDGVEVHNAKTKPREEQFNSTYRVVSVLTVLHQDWLNGKEYKCKVSNKGLPS | spacer |
| | SIEKTISKAKGQPREPQVYTLPPSQEEMTKNQVSLTCLVKGFYPSDIAVEWESNGQ | |
| | PENNYKTTPPVLDSDGSFFLYSRLTVDKSRWQEGNVFSCSVMHEALHNHYTQKSLS | |
| | LSLGK | |
| 135 | RWPESPKAQASSVPTAQPQAEGSLAKATTAPATTRNTGRGGEEKKKEKEKEEQEER | IgD-hinge-Fc |
| | ETKTPECPSHTQPLGVYLLTPAVQDLWLRDKATFTCFVVGSDLKDAHLTWEVAGKV | |
| | PTGGVEEGLLERHSNGSQSQHSRLTLPRSLWNAGTSVTCTLNHPSLPPQRLMALRE | |
| | PAAQAPVKLSLNLLASSDPPEAASWLLCEVSGFSPPNILLMWLEDQREVNTSGFAP | |
| | ARPPPQPGSTTFWAWSVLRVPAPPSPQPATYTCVVSHEDSRTLLNASRSLEVSYVT | |
| | DH | |
| 136 | FWVLVVVGGVLACYSLLVTVAFIIFWV | CD28 (amino acids |
| | | 153-179 of Accession |
| | | No. P10747) |
| 137 | IEVMYPPPYLDNEKSNGTIIHVKGKHLCPSPLFPGPSKPFWVLVVVGGVLACYSLL | CD28 (amino acids |
| | VTVAFIIFWV | 114-179 of Accession |
| | | No. P10747) |
| 138 | RSKRSRLLHSDYMNMTPRRPGPTRKHYQPYAPPRDFAAYRS | CD28 (amino acids |
| | | 180-220 of P10747) |
| 139 | RSKRSRGGHSDYMNMTPRRPGPTRKHYQPYAPPRDFAAYRS | CD28 (LL to GG) |
| 140 | KRGRKKLLYIFKQPFMRPVQTTQEEDGCSCRFPEEEEGGCEL | 4-1BB (amino acids |
| | | 214-255 of Q07011.1) |
| 141 | RVKFSRSADAPAYQQGQNQLYNELNLGRREEYDVLDKRRGRDPEMGGKPRRKNPQE | CD3 zeta |
| | GLYNELQKDKMAEAYSEIGMKGERRRGKGHDGLYQGLSTATKDTYDALHMQALPPR | |
| 142 | NNNNNNNNNNNNNNNNNNNNGUUUUAGAGCUAGAAAUAGCAAGUUAAAAUAAGGCU | exemplary gRNA |
| | AGUCCGUUAUCAACUUGAAAAAGUGGCACCGAGUCGGUGC | complementary |
| | | domain |
| 143 | NNNNNNNNNNNNNNNNNNNNGUUUUAGAGCUAUGCUGAAAAGCAUAGCAAGUUAAA | exemplary gRNA |
| | AUAAGGCUAGUCCGUUAUCAACUUGAAAAAGUGGCACCGAGUCGGUGC | complementary |
| | | domain |
| 144 | NNNNNNNNNNNNNNNNNNNNGUUUUAGAGCUAUGCUGGAAACAGCAUAGCAAGUUA | exemplary gRNA |
| | AAAUAAGGCUAGUCCGUUAUCAACUUGAAAAAGUGGCACCGAGUCGGUGC | complementary |
| | | domain |
| 145 | NNNNNNNNNNNNNNNNNNNNGUUUUAGAGCUAUGCUGUUUUGGAAACAAAACAGCA | exemplary gRNA |
| | UAGCAAGUUAAAAUAAGGCUAGUCCGUUAUCAACUUGAAAAAGUGGCACCGAGUCG | complementary |
| | GUGC | domain |
| 146 | NNNNNNNNNNNNNNNNNNNNGUAUUAGAGCUAGAAAUAGCAAGUUAAUAUAAGGCU | exemplary gRNA |
| | AGUCCGUUAUCAACUUGAAAAAGUGGCACCGAGUCGGUGC | |
| 147 | NNNNNNNNNNNNNNNNNNNNGUUUAAGAGCUAGAAAUAGCAAGUUUAAAUAAGGCU | exemplary gRNA |
| | AGUCCGUUAUCAACUUGAAAAAGUGGCACCGAGUCGGUGC | |
| 148 | NNNNNNNNNNNNNNNNNNNNGUAUUAGAGCUAUGCUGUAUUGGAAACAAUACAGCA | exemplary gRNA |
| | UAGCAAGUUAAUAUAAGGCUAGUCCGUUAUCAACUUGAAAAAGUGGCACCGAGUCG | |
| | GUGC | |
| 149 | AAGGCUAGUCCGUUAUCAACUUGAAAAAGUGGCACCGAGUCGGUGCU | exemplary proximal |
| | | and tail domain |
| 150 | AAGGCUAGUCCGUUAUCAACUUGAAAAAGUGGCACCGAGUCGGUGGUGC | exemplary proximal |
| | | and tail domain |
| 151 | AAGGCUAGUCCGUUAUCAACUUGAAAAAGUGGCACCGAGUCGGUGCGGAUC | exemplary proximal |
| | | and tail domain |
| 152 | AAGGCUAGUCCGUUAUCAACUUGAAAAAGUG | exemplary proximal |
| | | and tail domain |
| 153 | AAGGCUAGUCCGUUAUCA | exemplary proximal |
| | | and tail domain |
| 154 | AAGGCUAGUCCG | exemplary proximal |
| | | and tail domain |
| 155 | NNNNNNNNNNNNNNNNNNNNGUUUUAGAGCUAGAAAUAGCAAGUUAAAAUAAGGCU | exemplary chimeric |
| | AGUCCGUUAUCAACUUGAAAAAGUGGCACCGAGUCGGUGCUUUUUU | gRNA |
| 156 | NNNNNNNNNNNNNNNNNNNNGUUUUAGUACUCUGGAAACAGAAUCUACUAAAACAA | exemplary chimeric |
| | GGCAAAAUGCCGUGUUUAUCUCGUCAACUUGUUGGCGAGAUUUUUU | gRNA |
| 157 | KKPYSIGLDIGTNSVGWAVVTDDYKVPAKKMKVLGNTDKSHIEKNLLGALLFDSGN | Streptococcus mutans |
| | TAEDRRLKRTARRRYTRRRNRILYLQEIFSEEMGKVDDSFFHRLEDSFLVTEDKRG | Cas9 |
| | ERHPIFGNLEEEVKYHENFPTIYHLRQYLADNPEKVDLRLVYLALAHIIKFRGHFL | |
| | IEGKFDTRNNDVQRLFQEFLAVYDNTFENSSLQEQNVQVEEILTDKISKSAKKDRV | |
| | LKLFPNEKSNGRFAEFLKLIVGNQADFKKHFELEEKAPLQFSKDTYEEELEVLLAQ | |
| | IGDNYAELFLSAKKLYDSILLSGILTVTDVGTKAPLSASMIQRYNEHQMDLAQLKQ | |
| | FIRQKLSDKYNEVFSDVSKDGYAGYIDGKTNQEAFYKYLKGLLNKIEGSGYFLDKI | |
| | EREDFLRKQRTFDNGSIPHQIHLQEMRAIIRRQAEFYPFLADNQDRIEKLLTFRIP | |
| | YYVGPLARGKSDFAWLSRKSADKITPWNFDEIVDKESSAEAFINRMTNYDLYLPNQ | |
| | KVLPKHSLLYEKFTVYNELTKVKYKTEQGKTAFFDANMKQEIFDGVFKVYRKVTKD | |
| | KLMDFLEKEFDEFRIVDLTGLDKENKVFNASYGTYHDLCKILDKDFLDNSKNEKIL | |
| | EDIVLTLTLFEDREMIRKRLENYSDLLTKEQVKKLERRHYTGWGRLSAELIHGIRN | |
| | KESRKTILDYLIDDGNSNRNFMQLINDDALSFKEEIAKAQVIGETDNLNQVVSDIA | |
| | GSPAIKKGILQSLKIVDELVKIMGHQPENIVVEMARENQFTNQGRRNSQQRLKGLT | |
| | DSXKEFGSQILKEHPVENSQLQNDRLFLYYLQNGRDMYTGEELDIDYLSQYDIDHI | |
| | IPQAFIKDNSIDNRVLTSSKENRGKSDDVPSKDVVRKMKSYWSKLLSAKLITQRKF | |
| | DNLTKAERGGLTDDDKAGFIKRQLVETRQITKHVARILDERFNTETDENNKKIRQV | |
| | KIVTLKSNLVSNFRKEFELYKVREINDYHHAHDAYLNAVIGKALLGVYPQLEPEFV | |
| | YGDYPHFHGHKENKATAKKFFYSNIMNFFKKDDVRTDKNGEIIWKKDEHISNIKKV | |
| | LSYPQVNIVKKVEEQTGGFSKESILPKGNSDKLIPRKTKKFYWDTKKYGGFDSPIV | |
| | AYSILVIADIEKGKSKKLKTVKALVGVTIMEKMTFERDPVAFLERKGYRNVQEENI | |
| | IKLPKYSLFKLENGRKRLLASARELQKGNEIVLPNHLGTLLYHAKNIHKVDEPKHL | |
| | DYVDKHKDEFKELLDVVSNFSKKYTLAEGNLEKIKELYAQNNGEDLKELASSFINL | |
| | LTFTAIGAPATFKFFDKNIDRKRYTSTTEILNATLIHQSITGLYETRIDLNKLGGD | |
| 158 | DKKYSIGLDIGTNSVGWAVITDEYKVPSKKFKVLGNTDRHSIKKNLIGALLFDSGE | Streptococcus |
| | TAEATRLKRTARRRYTRRKNRICYLQEIFSNEMAKVDDSFFHRLEESFLVEEDKKH | pyogenes Cas9 |
| | ERHPIFGNXVDEVAYHEKYPTIYHLRKKLVDSTDKADLRLIYLALAHMIKFRGHFL | |
| | IEGDLNPDNSDVDKLFIQLVQTYNQLFEENPINASGVDAKAILSARLSKSRRLENL | |
| | IAQLPGEKKNGLFGNLIALSLGLTPNFKSNFDLAEDAKLQLSKDTYDDDLDNLLAQ | |
| | IGDQYADLFLAAKNLSDAILLSDILRVNTEITKAPLSASMIKRYDEHHQDLTLLKA | |
| | LVRQQLPEKYKEIFFDQSKNGYAGYIDGGASQEEFYKFIKPILEKMDGTEELLVKL | |
| | NREDLLRKQRTFDNGSIPHQIHLGELHAILRRQEDFYPFLKDNREKIEKILTFRIP | |
| | YYVGPLARGNSRFAWMTRKSEETITPWNFEEVVDKGASAQSFIERMTNFDKNLPNE | |
| | KVLPKHSLLYEYFTVYNELTKVKYVTEGMRKPAFLSGEQKKAIVDLLFKTNRKVTV | |
| | KQLKEDYFKKIECFDSVEISGVEDRFNASLGTYHDLLKIIKDKDFLDNEENEDILE | |
| | DIVLTLTLFEDREMIEERLKTYAHLFDDKVMKQLKRRRYTGWGRLSRKLINGIRDK | |
| | QSGKTILDFLKSDGFANRNFMQLIHDDSLTFKEDIQKAQVSGQGDSLHEHIANLAG | |
| | SPAIKKGILQTVKVVDELVKVMGRHKPENIVIEMARENQTTQKGQKNSRERMKRIE | |
| | EGIKELGSQILKEHPVENTQLQNEKLYLYYLQNGRDMYVDQELDINRLSDYDVDHI | |
| | VPQSFLKDDSIDNKVLTRSDKNRGKSDNVPSEEVVKKMKNYWRQLLNAKLITQRKF | |
| | DNLTKAERGGLSELDKAGFIKRQLVETRQITKHVAQILDSRMNTKYDENDKLIREV | |
| | KVITLKSKLVSDFRKDFQFYKVREINNYHHAHDAYLNAVVGTALIKKYPKLESEFV | |
| | YGDYKVYDVRKMIAKSEQEIGKATAKYFFYSNIMNFFKTEITLANGEIRKRPLIET | |
| | NGETGEIVWDKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGITIMERSSFEKN | |
| | PIDFLEAKGYKEVKKDLIIKLPKYSLFELENGRKRMLASAGELQKGNELALPSKYV | |
| | NFLYLASHYEKLKGSPEDNEQKQLFVEQHKHYLDEIIEQISEFSKRVILADANLDK | |
| | VLSAYNKHRDKPIREQAENIIHLFTLTNLGAPAAFKYFDTTIDRKRYTSTKEVLDA | |
| | TLIHQSITGLYETRIDLSQLGGD | |
| 159 | TKPYSIGLDIGTNSVGWAVTTDNYKVPSKKMKVLGNTSKKYIKKNLLGVLLFDSGI | Streptococcus |
| | TAEGRRLKRTARRRYTRRRNRILYLQEIFSTEMATLDDAFFQRLDDSFLVPDDKRD | thermophilus Cas9 |
| | SKYPIFGNLVEEKAYHDEFPTIYHLRKYLADSTKKADLRLVYLALAHMIKYRGHFL | |
| | IEGEFNSKNNDIQKNFQDFLDTYNAIFESDLSLENSKQLEEIVKDKISKLEKKDRI | |
| | LKLFPGEKNSGIFSEFLKLIVGNQADFRKCFNLDEKASLHFSKESYDEDLETLLGY | |
| | IGDDYSDVFLKAKKLYDAILLSGFLTVTDNETEAPLSSAMIKRYNEHKEDLALLKE | |
| | YIRNISLKTYNEVFKDDTKNGYAGYIDGKTNQEDFYVYLKKLLAEFEGADYFLEKI | |
| | DREDFLRKQRTFDNGSIPYQIHLQEMRAILDKQAKFYPFLAKNKERIEKILTFRIP | |
| | YYVGPLARGNSDFAWSIRKRNEKITPWNFEDVIDKESSAEAFINRMTSFDLYLPEE | |
| | KVLPKHSLLYETFNVYNELTKVRFIAESMRDYQFLDSKQKKDIVRLYFKDKRKVTD | |
| | KDIIEYLHAIYGYDGIELKGIEKQFNSSLSTYHDLLNIINDKEFLDDSSNEAIIEE | |
| | IIHTLTIFEDREMIKQRLSKFENIFDKSVLKKLSRRHYTGWGKLSAKLINGIRDEK | |
| | SGNTILDYLIDDGISNRNFMQLIHDDALSFKKKIQKAQIIGDEDKGNIKEVVKSLP | |
| | GSPAIKKGILQSIKIVDELVKVMGGRKPESIVVEMARENQYTNQGKSNSQQRLKRL | |
| | EKSLKELGSKILKENIPAKLSKIDNNALQNDRLYLYYLQNGKDMYTGDDLDIDRLS | |
| | NYDIDHIIPQAFLKDNSIDNKVLVSSASNRGKSDDVPSLEVVKKRKTFWYQLLKSK | |
| | LISQRKFDNLTKAERGGLSPEDKAGFIQRQLVETRQITKHVARLLDEKFNNKKDEN | |
| | NRAVRTVKIITLKSTLVSQFRKDFELYKVREINDFHHAHDAYLNAVVASALLKKYP | |
| | KLEPEFVYGDYPKYNSFRERKSATEKVYFYSNIMNIFKKSISLADGRVIERPLIEV | |
| | NEETGESVWNKESDLATVRRVLSYPQVNVVKKVEEQNHGLDRGKPKGLFNANLSSK | |
| | PKPNSNENLVGAKEYLDPKKYGGYAGISNSFTVLVKGTIEKGAKKKITNVLEFQGI | |
| | SILDRINYRKDKLNFLLEKGYKDIELIIELPKYSLFELSDGSRRMLASILSTNNKR | |
| | GEIHKGNQIFLSQKFVKLLYHAKRISNTINENHRKYVENHKKEFEELFYYILEFNE | |
| | NYVGAKKNGKLLNSAFQSWQNHSIDELCSSFIGPTGSERKGLFELTSRGSAADFEF | |
| | LGVKIPRYRDYTPSSLLKDATLIHQSVTGLYETRIDLAKLGEG | |
| 160 | KKPYTIGLDIGTNSVGWAVLTDQYDLVKRKMKIAGDSEKKQIKKNFWGVRLFDEGQ | Listeria innocua Cas9 |
| | TAADRRMARTARRRIERRRNRISYLQGIFAEEMSKTDANFFCRLSDSFYVDNEKRN | |
| | SRHPFFATIEEEVEYHKNYPTIYHLREELVNSSEKADLRLVYLALAHIIKYRGNFL | |
| | IEGALDTQNTSVDGIYKQFIQTYNQVFASGIEDGSLKKLEDNKDVAKILVEKVTRK | |
| | EKLERILKLYPGEKSAGMFAQFISLIVGSKGNFQKPFDLIEKSDIECAKDSYEEDL | |
| | ESLLALIGDEYAELFVAAKNAYSAVVLSSIITVAETETNAKLSASMIERFDTHEED | |
| | LGELKAFIKLHLPKHYEEIFSNTEKHGYAGYIDGKTKQADFYKYMKMTLENIEGAD | |
| | YFIAKIEKENFLRKQRTFDNGAIPHQLHLEELEAILHQQAKYYPFLKENYDKIKSL | |
| | VTFRIPYFVGPLANGQSEFAWLTRKADGEIRPWNIEEKVDFGKSAVDFIEKMTNKD | |
| | TYLPKENVLPKHSLCYQKYLVYNELTKVRYINDQGKTSYFSGQEKEQIFNDLFKQK | |
| | RKVKKKDLELFLRNMSHVESPTIEGLEDSFNSSYSTYHDLLKVGIKQEILDNPVNT | |
| | EMLENIVKILTVFEDKRMIKEQLQQFSDVLDGVVLKKLERRHYTGWGRLSAKLLMG | |
| | IRDKQSHLTILDYLMNDDGLNRNLMQLINDSNLSFKSIIEKEQVTTADKDIQSIVA | |
| | DLAGSPAIKKGILQSLKIVDELVSVMGYPPQTIVVEMARENQTTGKGKNNSRPRYK | |
| | SLEKAIKEFGSQILKEHPTDNQELRNNRLYLYYLQNGKDMYTGQDLDIHNLSNYDI | |
| | DHIVPQSFITDNSIDNLVLTSSAGNREKGDDVPPLEIVRKRKVFWEKLYQGNLMSK | |
| | RKFDYLTKAERGGLTEADKARFIHRQLVETRQITKNVANILHQRFNYEKDDHGNTM | |
| | KQVRIVTLKSALVSQFRKQFQLYKVRDVNDYHHAHDAYLNGVVANTLLKVYPQLEP | |
| | EFVYGDYHQFDWFKANKATAKKQFYTNIMLFFAQKDRIIDENGEILWDKKYLDTVK | |
| | KVMSYRQMNIVKKTEIQKGEFSKATIKPKGNSSKLIPRKTNWDPMKYGGLDSPNMA | |
| | YAVVIEYAKGKNKLVFEKKIIRVTIMERKAFEKDEKAFLEEQGYRQPKVLAKLPKY | |
| | TLYECEEGRRRMLASANEAQKGNQQVLPNHLVTLLHHAANCEVSDGKSLDYIESNR | |
| | EMFAELLAHVSEFAKRYTLAEANLNKINQLFEQNKEGDIKAIAQSFVDLMAFNAMG | |
| | APASFKFFETTIERKRYNNLKELLNSTIIYQSITGLYESRKRLDD | |
| 161 | MAAFKPNSINYILGLDIGIASVGWAMVEIDEEENPIRLIDLGVRVFERAEVPKTGD | Neisseria meningitidis |
| | SLAMARRLARSVRRLTRRRAHRLLRTRRLLKREGVLQAANFDENGLIKSLPNTPWQ | Cas9 |
| | LRAAALDRKLTPLEWSAVLLHLIKHRGYLSQRKNEGETADKELGALLKGVAGNAHA | |
| | LQTGDFRTPAELALNKFEKESGHIRNQRSDYSHTFSRKDLQAELILLFEKQKEFGN | |
| | PHVSGGLKEGIETLLMTQRPALSGDAVQKMLGHCTFEPAEPKAAKNTYTAERFIWL | |
| | TKLNNLRILEQGSERPLTDTERATLMDEPYRKSKLTYAQARKLLGLEDTAFFKGLR | |
| | YGKDNAEASTLMEMKAYHAISRALEKEGLKDKKSPLNLSPELQDEIGTAFSLFKTD | |
| | EDITGRLKDRIQPEILEALLKHISFDKFVQISLKALRRIVPLMEQGKRYDEACAEI | |
| | YGDHYGKKNTEEKIYLPPIPADEIRNPVVLRALSQARKVINGVVRRYGSPARIHIE | |
| | TAREVGKSFKDRKEIEKRQEENRKDREKAAAKFREYFPNFVGEPKSKDILKLRLYE | |
| | QQHGKCLYSGKEINLGRLNEKGYVEIDHALPFSRTWDDSFNNKVLVLGSENQNKGN | |
| | QTPYEYFNGKDNSREWQEFKARVETSRFPRSKKQRILLQKFDEDGFKERNLNDTRY | |
| | VNRFLCQFVADRMRLTGKGKKRVFASNGQITNLLRGFWGLRKVRAENDRHHALDAV | |
| | VVACSTVAMQQKITRFVRYKEMNAFDGKTIDKETGEVLHQKTHFPQPWEFFAQEVM | |
| | IRVFGKPDGKPEFEEADTLEKLRTLLAEKLSSRPEAVHEYVTPLFVSRAPNRKMSG | |
| | QGHMETVKSAKRLDEGVSVLRVPLTQLKLKDLEKMVNREREPKLYEALKARLEAHK | |
| | DDPAKAFAEPFYKYDKAGNRTQQVKAVRVEQVQKTGVWVRNHNGIADNATMVRVDV | |
| | FEKGDKYYLVPIYSWQVAKGILPDRAVVQGKDEEDWQLIDDSFNFKFSLHPNDLVE | |
| | VITKKARMFGYFASCHRGTGNINIRIHDLDHKIGKNGILEGIGVKTALSFQKYQID | |
| | ELGKEIRPCRLKKRPPVR | |
| 162 | MDKKYSIGLDIGTNSVGWAVITDEYKVPSKKFKVLGNTDRHSIKKNLIGALLFDSG | Streptococcus |
| | ETAEATRLKRTARRRYTRRKNRICYLQEIFSNEMAKVDDSFFHRLEESFLVEEDKK | pyogenes Cas9 |
| | HERHPIFGNIVDEVAYHEKYPTIYHLRKKLVDSTDKADLRLIYLALAHMIKFRGHF | |
| | LIEGDLNPDNSDVDKLFIQLVQTYNQLFEENPINASGVDAKAILSARLSKSRRLEN | |
| | LIAQLPGEKKNGLFGNLIALSLGLTPNFKSNFDLAEDAKLQLSKDTYDDDLDNLLA | |
| | QIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVK | |
| | LNREDLLRKQRTFDNGSIPHQIHLGELHAILRRQEDFYPFLKDNREKIEKILTFRI | |
| | PYYVGPLARGNSRFAWMTRKSEETITPWNFEEVVDKGASAQSFIERMTNFDKNLPN | |
| | EKVLPKHSLLYEYFTVYNELTKVKYVTEGMRKPAFLSGEQKKAIVDLLFKTNRKVT | |
| | VKQLKEDYFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRLSRKLINGIRD | |
| | KQSGKTILDFLKSDGFANRNFMQLIHDDSLTFKEDIQKAQVSGQGDSLHEHIANLA | |
| | GSPAIKKGILQTVKVVDELVKVMGRHKPENIVIEMARENQTTQKGQKNSRERMKRI | |
| | EEGIKELGSQILKEHPVENTQLQNEKLYLYYLQNGRDMYVDQELDINRLSDYDVDH | |
| | IVPQSFLKDDSIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKYDENDKLIRE | |
| | VKVITLKSKLVSDFRKDFQFYKVREINNYHHAHDAYLNAVVGTALIKKYPKLESEF | |
| | VYGDYKVYDVRKMIAKSEQEIGKATAKYFFYSNIMNFFKTEITLANGEIRKRPLIE | |
| | TNGETGEIVWDKGRDFATVRKVLSMPQVNIVKKTEVQTGGFSKESILPKRNSDKLI | |
| | ARKKDWDPKKYGGFDSPTVAYSVLVVAKVEKGKSKKLKSVKELLGITIMERSSFEK | |
| | NPIDFLEAKGYKEVKKDLIIKLPKYSLFELENGRKRMLASAGELQKGNELALPSKY | |
| | VNFLYLASHYEKLKGSPEDNEQKQLFVEQHKHYLDEIIEQISEFSKRVILADANLD | |
| | KVLSAYNKHRDKPIREQAENIIHLFTLTNLGAPAAFKYFDTTIDRKRYTSTKEVLD | |
| | ATLIHQSITGLYETRIDLSQLGGD | |
| 163 | GAGAATCAAAATCGGTGAAT | TRAC target sequence |
| | | 2 |
| 164 | GGCCTCGGCGCTGACGATCT | TRBC target sequence |
| | | 2 |
| 165 | GAGAAT CAAAATCGGT GAATAGG | TRAC target sequence |
| | | with PAM 3 |
| 166 | TTCAAAACCTGTCAGTGATTGGG | TRAC target sequence |
| | | with PAM 4 |
| 167 | TGTGCTAGACATGAGGTCTATGG | TRAC target sequence |
| | | with PAM 5 |
| 168 | CGTCATGAGCAGATTAAACCCGG | TRAC target sequence |
| | | with PAM 6 |
| 169 | TCAGGGTTCTGGATATCTGTGGG | TRAC target sequence |
| | | with PAM 7 |
| 170 | GTCAGGGTTCTGGATATCTGTGG | TRAC target sequence |
| | | with PAM 8 |
| 171 | TTCGGAACCCAATCACTGACAGG | TRAC target sequence |
| | | with PAM 9 |
| 172 | TAAACCCGGCCACTTTCAGGAGG | TRAC target sequence |
| | | with PAM 10 |
| 173 | AAAGTCAGATTTGTTGCTCCAGG | TRAC target sequence |
| | | with PAM 11 |
| 174 | AACAAATGTGTCACAAAGTAAGG | TRAC target sequence |
| | | with PAM 12 |
| 175 | TGGATTTAGAGTCTCTCAGCTGG | TRAC target sequence |
| | | with PAM 13 |
| 176 | TAGGCAGACAGACTTGTCACTGG | TRAC target sequence |
| | | with PAM 14 |
| 177 | AGCTGGTACACGGCAGGGTCAGG | TRAC target sequence |
| | | with PAM 15 |
| 178 | GCTGGTACACGGCAGGGTCAGGG | TRAC target sequence |
| | | with PAM 16 |
| 179 | TCTCTCAGCTGGTACACGGCAGG | TRAC target sequence |
| | | with PAM 17 |
| 180 | TTTCAAAACCTGTCAGTGATTGG | TRAC target sequence |
| | | with PAM 18 |
| 181 | GATTAAACCCGGCCACTTTCAGG | TRAC target sequence |
| | | with PAM 19 |
| 182 | CTCGACCAGCTTGACATCACAGG | TRAC target sequence |
| | | with PAM 20 |
| 183 | AGAGTCTCTCAGCTGGTACACGG | TRAC target sequence |
| | | with PAM 21 |
| 184 | CTCTCAGCTGGTACACGGCAGGG | TRAC target sequence |
| | | with PAM 22 |
| 185 | AAGTTCCTGTGATGTCAAGCTGG | TRAC target sequence |
| | | with PAM 23 |
| 186 | ATCCTCCTCCTGAAAGTGGCCGG | TRAC target sequence |
| | | with PAM 24 |
| 187 | TGCTCATGACGCTGCGGCTGTGG | TRAC target sequence |
| | | with PAM 25 |
| 188 | ACAAAACTGTGCTAGACATGAGG | TRAC target sequence |
| | | with PAM 26 |
| 189 | ATTTGTTTGAGAATCAAAATCGG | TRAC target sequence |
| | | with PAM 27 |
| 190 | CATCACAGGAACTTTCTAAAAGG | TRAC target sequence |
| | | with PAM 28 |
| 191 | GTCGAGAAAAGCTTTGAAACAGG | TRAC target sequence |
| | | with PAM 29 |
| 192 | CCACTTTCAGGAGGAGGATTCGG | TRAC target sequence |
| | | with PAM 30 |
| 193 | CTGACAGGTTTTGAAAGTTTAGG | TRAC target sequence |
| | | with PAM 31 |
| 194 | AGCTTTGAAACAGGTAAGACAGG | TRAC target sequence |
| | | with PAM 32 |
| 195 | TGGAATAATGCTGTTGTTGAAGG | TRAC target sequence |
| | | with PAM 33 |
| 196 | AGAGCAACAGTGCTGTGGCCTGG | TRAC target sequence |
| | | with PAM 34 |
| 197 | CTGTGGTCCAGCTGAGGTGAGGG | TRAC target sequence |
| | | with PAM 35 |
| 198 | CTGCGGCTGTGGTCCAGCTGAGG | TRAC target sequence |
| | | with PAM 36 |
| 199 | TGTGGTCCAGCTGAGGTGAGGGG | TRAC target sequence |
| | | with PAM 37 |
| 200 | CTTCTTCCCCAGCCCAGGTAAGG | TRAC target sequence |
| | | with PAM 38 |
| 201 | ACACGGCAGGGTCAGGGTTCTGG | TRAC target sequence |
| | | with PAM 39 |
| 202 | CTTCAAGAGCAACAGTGCTGTGG | TRAC target sequence |
| | | with PAM 40 |
| 203 | CTGGGGAAGAAGGTGTCTTCTGG | TRAC target sequence |
| | | with PAM 41 |
| 204 | TCCTCCTCCTGAAAGTGGCCGGG | TRAC target sequence |
| | | with PAM 42 |
| 205 | TTAATCTGCTCATGACGCTGCGG | TRAC target sequence |
| | | with PAM 43 |
| 206 | ACCCGGCCACTTTCAGGAGGAGG | TRAC target sequence |
| | | with PAM 44 |
| 207 | TTCTTCCCCAGCCCAGGTAAGGG | TRAC target sequence |
| | | with PAM 45 |
| 208 | CTTACCTGGGCTGGGGAAGAAGG | TRAC target sequence |
| | | with PAM 46 |
| 209 | GACACCTTCTTCCCCAGCCCAGG | TRAC target sequence |
| | | with PAM 47 |
| 210 | GCTGTGGTCCAGCTGAGGTGAGG | TRAC target sequence |
| | | with PAM 48 |
| 211 | CCGAATCCTCCTCCTGAAAGTGG | TRAC target sequence |
| | | with PAM 49 |
| 212 | GCTGTCAAGTCCAGTTCTACGGG | TRBC target sequence |
| | | with PAM 3 |
| 213 | CTATGGACTTCAAGAGCAACAGTGCTGT | TRAC ZFN target |
| | | sequence 1 |
| 214 | CTCATGTCTAGCACAGTTTTGTCTGTGA | TRAC ZFN target |
| | | sequence 2 |
| 215 | GTGCTGTGGCCTGGAGCAACAAATCTGA | TRAC ZFN target |
| | | sequence 3 |
| 216 | TTGCTCTTGAAGTCCATAGACCTCATGT | TRAC ZFN target |
| | | sequence 4 |
| 217 | GCTGTGGCCTGGAGCAACAAATCTGACT | TRAC ZFN target |
| | | sequence 5 |
| 218 | CTGTTGCTCTTGAAGTCCATAGACCTCA | TRAC ZFN target |
| | | sequence 6 |
| 219 | CTGTGGCCTGGAGCAACAAATCTGACTT | TRAC ZFN target |
| | | sequence 7 |
| 220 | CTGACTTTGCATGTGCAAACGCCTTCAA | TRAC ZFN target |
| | | sequence 8 |
| 221 | TTGTTGCTCCAGGCCACAGCACTGTTGC | TRAC ZFN target |
| | | sequence 9 |
| 222 | TGAAAGTGGCCGGGTTTAATCTGCTCAT | TRAC ZFN target |
| | | sequence 10 |
| 223 | AGGAGGATTCGGAACCCAATCACTGACA | TRAC ZFN target |
| | | sequence 11 |
| 224 | GAGGAGGATTCGGAACCCAATCACTGAC | TRAC ZFN target |
| | | sequence 12 |
| 225 | CCGTAGAACTGGACTTGACAGCGGAAGT | TRBC ZFN target |
| | | sequence 1 |
| 226 | TCTCGGAGAATGACGAGTGGACCCAGGA | TRBC ZFN target |
| | | sequence 2 |
| 227 | AGCTGAGAGACTCTAAATCCAGTGACAAGTCTGTCTGCCTATTCACCGAT | TRAC 50 bp 5′ |
| | | Homology Arm |
| 228 | CTGATCCTCTTGTCCCACAGATATCCAGAACCCTGACCCTGCCGTGTACCAGCTGA | TRAC 100 bp 5′ |
| | GAGACTCTAAATCCAGTGACAAGTCTGTCTGCCTATTCACCGAT | Homology Arm |
| 229 | GTGACTTGCCAGCCCCACAGAGCCCCGCCCTTGTCCATCACTGGCATCTGGACTCC | TRAC 200 bp 5′ |
| | AGCCTGGGTTGGGGCAAAGAGGGAAATGAGATCATGTCCTAACCCTGATCCTCTTG | Homology Arm |
| | TCCCACAGATATCCAGAACCCTGACCCTGCCGTGTACCAGCTGAGAGACTCTAAAT | |
| | CCAGTGACAAGTCTGTCTGCCTATTCACCGAT | |
| 230 | CCTCTTGGCCAAGATTGATAGCTTGTGCCTGTCCCTGAGTCCCAGTCCATCACGAG | TRAC 300 bp 5′ |
| | CAGCTGGTTTCTAAGATGCTATTTCCCGTATAAAGCATGAGACCGTGACTTGCCAG | Homology Arm |
| | CCCCACAGAGCCCCGCCCTTGTCCATCACTGGCATCTGGACTCCAGCCTGGGTTGG | |
| | GGCAAAGAGGGAAATGAGATCATGTCCTAACCCTGATCCTCTTGTCCCACAGATAT | |
| | CCAGAACCCTGACCCTGCCGTGTACCAGCTGAGAGACTCTAAATCCAGTGACAAGT | |
| | CTGTCTGCCTATTCACCGAT | |
| 231 | ATTAAATAAAAGAATAAGCAGTATTATTAAGTAGCCCTGCATTTCAGGTTTCCTTG | TRAC 400 bp 5′ |
| | AGTGGCAGGCCAGGCCTGGCCGTGAACGTTCACTGAAATCATGGCCTCTTGGCCAA | Homology Arm |
| | GATTGATAGCTTGTGCCTGTCCCTGAGTCCCAGTCCATCACGAGCAGCTGGTTTCT | |
| | AAGATGCTATTTCCCGTATAAAGCATGAGACCGTGACTTGCCAGCCCCACAGAGCC | |
| | CCGCCCTTGTCCATCACTGGCATCTGGACTCCAGCCTGGGTTGGGGCAAAGAGGGA | |
| | AATGAGATCATGTCCTAACCCTGATCCTCTTGTCCCACAGATATCCAGAACCCTGA | |
| | CCCTGCCGTGTACCAGCTGAGAGACTCTAAATCCAGTGACAAGTCTGTCTGCCTAT | |
| | TCACCGAT | |
| 232 | CTCCAGATTCCAAGATGTACAGTTTGCTTTGCTGGGCCTTTTTCCCATGCCTGCCT | TRAC 500 bp 5′ |
| | TTACTCTGCCAGAGTTATATTGCTGGGGTTTTGAAGAAGATCCTATTAAATAAAAG | Homology Arm |
| | AATAAGCAGTATTATTAAGTAGCCCTGCATTTCAGGTTTCCTTGAGTGGCAGGCCA | |
| | GGCCTGGCCGTGAACGTTCACTGAAATCATGGCCTCTTGGCCAAGATTGATAGCTT | |
| | GTGCCTGTCCCTGAGTCCCAGTCCATCACGAGCAGCTGGTTTCTAAGATGCTATTT | |
| | CCCGTATAAAGCATGAGACCGTGACTTGCCAGCCCCACAGAGCCCCGCCCTTGTCC | |
| | ATCACTGGCATCTGGACTCCAGCCTGGGTTGGGGCAAAGAGGGAAATGAGATCATG | |
| | TCCTAACCCTGATCCTCTTGTCCCACAGATATCCAGAACCCTGACCCTGCCGTGTA | |
| | CCAGCTGAGAGACTCTAAATCCAGTGACAAGTCTGTCTGCCTATTCACCGAT | |
| 233 | AGAGAGCAATCTCCTGGTAATGTGATAGATTTCCCAACTTAATGCCAACATACCAT | TRAC 600 bp 5′ |
| | AAACCTCCCATTCTGCTAATGCCCAGCCTAAGTTGGGGAGACCACTCCAGATTCCA | Homology Arm |
| | AGATGTACAGTTTGCTTTGCTGGGCCTTTTTCCCATGCCTGCCTTTACTCTGCCAG | |
| | AGTTATATTGCTGGGGTTTTGAAGAAGATCCTATTAAATAAAAGAATAAGCAGTAT | |
| | TATTAAGTAGCCCTGCATTTCAGGTTTCCTTGAGTGGCAGGCCAGGCCTGGCCGTG | |
| | AACGTTCACTGAAATCATGGCCTCTTGGCCAAGATTGATAGCTTGTGCCTGTCCCT | |
| | GAGTCCCAGTCCATCACGAGCAGCTGGTTTCTAAGATGCTATTTCCCGTATAAAGC | |
| | ATGAGACCGTGACTTGCCAGCCCCACAGAGCCCCGCCCTTGTCCATCACTGGCATC | |
| | TGGACTCCAGCCTGGGTTGGGGCAAAGAGGGAAATGAGATCATGTCCTAACCCTGA | |
| | TCCTCTTGTCCCACAGATATCCAGAACCCTGACCCTGCCGTGTACCAGCTGAGAGA | |
| | CTCTAAATCCAGTGACAAGTCTGTCTGCCTATTCACCGAT | |
| 234 | TTTGATTCTCAAACAAATGTGTCACAAAGTAAGGATTCTGATGTGTATAT | TRAC 50 bp 3′ |
| | | Homology Arm |
| 235 | TTTGATTCTCAAACAAATGTGTCACAAAGTAAGGATTCTGATGTGTATATCACAGA | TRAC 100 bp 3′ |
| | CAAAACTGTGCTAGACATGAGGTCTATGGACTTCAAGAGCAACA | Homology Arm |
| 236 | TTTGATTCTCAAACAAATGTGTCACAAAGTAAGGATTCTGATGTGTATATCACAGA | TRAC 200 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGG | |
| 237 | TTTGATTCTCAAACAAATGTGTCACAAAGTAAGGATTCTGATGTGTATATCACAGA | TRAC 300 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGT | |
| 238 | TTTGATTCTCAAACAAATGTGTCACAAAGTAAGGATTCTGATGTGTATATCACAGA | TRAC 400 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAG | |
| 239 | TTTGATTCTCAAACAAATGTGTCACAAAGTAAGGATTCTGATGTGTATATCACAGA | TRAC 500 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTC | |
| 240 | TTTGATTCTCAAACAAATGTGTCACAAAGTAAGGATTCTGATGTGTATATCACAGA | TRAC 600 bp 3′ |
| | CAAAACTGTGCTAGACATGAGGTCTATGGACTTCAAGAGCAACAGTGCTGTGGCCT | Homology Arm |
| | GGAGCAACAAATCTGACTTTGCATGTGCAAACGCCTTCAACAACAGCATTATTCCA | |
| | GAAGACACCTTCTTCCCCAGCCCAGGTAAGGGCAGCTTTGGTGCCTTCGCAGGCTG | |
| | TTTCCTTGCTTCAGGAATGGCCAGGTTCTGCCCAGAGCTCTGGTCAATGATGTCTA | |
| | AAACTCCTCTGATTGGTGGTCTCGGCCTTATCCATTGCCACCAAAACCCTCTTTTT | |
| | ACTAAGAAACAGTGAGCCTTGTTCTGGCAGTCCAGAGAATGACACGGGAAAAAAGC | |
| | AGATGAAGAGAAGGTGGCAGGAGAGGGCACGTGGCCCAGCCTCAGTCTCTCCAACT | |
| | GAGTTCCTGCCTGCCTGCCTTTGCTCAGACTGTTTGCCCCTTACTGCTCTTCTAGG | |
| | CCTCATTCTAAGCCCCTTCTCCAAGTTGCCTCTCCTTATTTCTCCCTGTCTGCCAA | |
| | AAAATCTTTCCCAGCTCACTAAGTCAGTCTCACGCAGTCA | |
| 241 | NIQKPDPAVYQLRDSKSSDKSVCLFTDFDSQTNVSQSKDSDVYITDKTVLDMRSMD | Human TCR alpha |
| | FKSNSAVAWSNKSDFACANAFNNSIIPADTFFPSPESSCDVKLVEKSFETDTNLNF | constant |
| | QNLSVIGFRILLLKVAGFNLLMTLRLWSS | |
| 242 | HIQNPDPAVYQLRDSKSSDKSVCLFTDFDSQTNVSQSKDSDVYITDKTVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 243 | YIQNPDPAVYQLRDSKSSDKSVCLFTDFDSQTNVSQSKDSDVYITDKTVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 244 | DIQNPDPAVYQLRDSKSSDKSVCLFTDFDSQTNVSQSKDSDVYITDKTVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 245 | PNIQKPDPAVYQLRDSKSSDKSVCLFTDFDSQTNVSQSKDSDVYITDKTVLDMRSM | Human TCR alpha |
| | DFKSNSAVAWSNKSDFACANAFNNSIIPADTFFPSPESSCDVKLVEKSFETDTNLN | constant |
| | FQNLSVIGFRILLLKVAGFNLLMTLRLWSS | |
| 246 | NIQKPDPAVYQLRDSKSSDKSVCLFTDFDSQTNVSQSKDSDVYITDKCVLDMRSMD | Human TCR alpha |
| | FKSNSAVAWSNKSDFACANAFNNSIIPADTFFPSPESSCDVKLVEKSFETDTNLNF | constant |
| | QNLSVIGFRILLLKVAGFNLLMTLRLWSS | |
| 247 | HIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 248 | YIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 249 | NIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 250 | DIQNPDPAVYQLRDSKSSDKSVCLFTDFDSQTNVSQSKDSDVYITDKCVLDMRSMD | Human TCR alpha |
| | FKSNSAVAWSNKSDFACANAFNNSIIPEDTFFPSPESSCDVKLVEKSFETDTNLNF | constant |
| | QNLSVIGFRILLLKVAGFNLLMTLRLWSS | |
| 251 | EDLNKVFPPEVAVFEPSEAEISHTQKATLVCLATGFFPDHVELSWWVNGKEVHSGV | Human TCR beta |
| | CTDPQPLKEQPALNDSRYCLSSRLRVSATFWQNPRNHFRCQVQFYGLSENDEWTQD | constant |
| | RAKPVTQIVSAEAWGRADCGFTSVSYQQGVLSATILYEILLGKATLYAVLVSALVL | |
| | MAMVKRKDF | |
| 252 | TEDLKNVFPPEVAVFEPSEAEISHTQKATLVCLATGFYPDHVELSWWVNGKEVHSG | Human TCR beta |
| | VSTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |
| 253 | LEDLKNVFPPEVAVFEPSEAEISHTQKATLVCLATGFYPDHVELSWWVNGKEVHSG | Human TCR beta |
| | VSTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |
| 254 | EDLKNVFPPEVAVFEPSEAEISHTQKATLVCLATGFYPDHVELSWWVNGKEVHSGV | Human TCR beta |
| | CTDPQPLKEQPALNDSRYCLSSRLRVSATFWQNPRNHFRCQVQFYGLSENDEWTQD | constant |
| | RAKPVTQIVSAEAWGRADCGFTSESYQQGVLSATILYEILLGKATLYAVLVSALVL | |
| | MAMVKRKDSRG | |
| 255 | TEDLKNVFPPEVAVFEPSEAEISHTQKATLVCLATGFYPDHVELSWWVNGKEVHSG | Human TCR beta |
| | VCTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |
| 256 | LEDLKNVFPPEVAVFEPSEAEISHTQKATLVCLATGFYPDHVELSWWVNGKEVHSG | Human TCR beta |
| | VCTDPQPLKEQPALNDSRYCLSSRLRVSATFWQNPRNHFRCQVQFYGLSENDEWTQ | constant |
| | DRAKPVTQIVSAEAWGRADCGFTSESYQQGVLSATILYEILLGKATLYAVLVSALV | |
| | LMAMVKRKDSRG | |

What is claimed:

1. A composition, comprising a plurality of engineered T cells comprising a recombinant receptor or an antigen-binding fragment or chain thereof encoded by a transgene and a genetic disruption of at least one target site within a T cell receptor alpha constant (TRAC) gene and/or a T cell receptor beta constant (TRBC) gene, wherein the recombinant receptor is capable of binding to an antigen that is associated with, specific to, and/or expressed on a cell or tissue of a disease, disorder or condition, and wherein:
at least or greater than 35%, 40%, 45%, 50%, 55%, 60%, 65%, 70%, 75%, 80%, or 90% of the cells in the composition comprise a genetic disruption of at least one target site within a TRAC gene and/or a TRBC gene; and/or at least or greater than 35%, 40%, 45%, 50%, 55%, 60%, 65%, 70%, 75%, 80%, or 90% of the cells in the composition express the recombinant receptor or antigen-binding fragment or chain thereof and/or exhibits binding to the antigen; and
the transgene encoding the recombinant receptor or antigen-binding fragment or chain thereof is integrated at or near one of the at least one target site via homology directed repair (HDR).

2. A composition, comprising a plurality of engineered T cells comprising a recombinant receptor or an antigen-binding fragment or α chain thereof encoded by a transgene and a genetic disruption of at least one target site within a T cell receptor alpha constant (TRAC) gene and/or a T cell receptor beta constant (TRBC) gene, wherein the recombinant receptor is capable of binding to an antigen that is associated with, specific to, and/or expressed on a cell or tissue of a disease, disorder or condition and wherein:
the coefficient of variation of expression and/or antigen binding of the recombinant receptor among the plurality of cells is lower than 0.70, 0.65, 0.60, 0.55, 0.50, 0.45, 0.40, 0.35 or 0.30 or less; and/or
the coefficient of variation of expression and/or antigen binding of the recombinant receptor among the plurality of cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

3. The composition of claim 1 or claim 2, wherein engineered T cells comprise at least one genetic disruption of a target site in a TRAC gene.

4. The composition of any of claims 1-3, wherein engineered T cells comprise at least one genetic disruption of a target site in a TRBC gene.

5. The composition of any of claims 1-4, wherein engineered T cells comprise at least one genetic disruption of a target site in a TRAC gene and at least one genetic disruption of a target site in a TRBC gene.

6. The composition of any of claims 1-5, wherein the TRBC gene is one or both of a T cell receptor beta constant 1 (TRBC1) or T cell receptor beta constant 2 (TRBC2) gene.

7. The composition of any of claims 1-6, wherein the genetic disruption is by a zinc finger nuclease (ZFN), a TAL-effector nuclease (TALEN), or a CRISPR-Cas9 combination that specifically binds to, recognizes, or hybridizes to the target site.

8. The composition of any of claims 1-7, wherein the genetic disruption is by a CRISPR-Cas9 combination and the CRISPR-Cas9 combination comprises a guide RNA (gRNA) having a targeting domain that is complementary to the at least one target site.

9. The composition of claim 8, wherein the CRISPR-Cas9 combination is a ribonucleoprotein (RNP) complex comprising the gRNA and a Cas9 protein.

10. The composition of claim 9, wherein the genetic disruption of each of the plurality of engineered T cells is effected by the RNP introduced into a plurality of T cells via electroporation.

11. The composition of any of claims 1-10, wherein the at least one target site is within an exon of the TRAC, TRBC1 and/or TRBC2 gene.

12. The composition of any of claims 8-11, wherein the gRNA has a targeting domain that is complementary to a target site in a TRAC gene and comprises a sequence selected from the group consisting of UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

13. The composition of any of claims 8-12, wherein the gRNA has a targeting domain comprising the sequence GAGAAUCAAAAUCGGUGAAU (SEQ ID NO:31).

14. The composition of any of claims 8-11, wherein the gRNA has a targeting domain that is complementary to a target site in one or both of a TRBC1 and a TRBC2 gene and comprises a sequence selected from the group consisting of CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

15. The composition of any of claims 8-11 and 14, wherein the gRNA has a targeting domain comprising the sequence GGCCUCGGCGCUGACGAUCU (SEQ ID NO:63).

16. The composition of any of claims 1-15, wherein the integration of the transgene is by a template polynucleotide introduced into each of the plurality of T cells, said template polynucleotide comprising the structure [5′ homology arm]-[transgene]-[3′ homology arm].

17. The composition of claim 16, wherein the 5′ homology arm and 3′ homology arm comprises nucleic acid sequences homologous to nucleic acid sequences surrounding the at least one target site.

18. The composition of claim 16 or claim 17, wherein the 5′ homology arm and 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

19. The composition of any of claims 16-18, wherein the 5′ homology arm and 3′ homology arm independently are from at or about 100 to at or about 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides in length.

20. The composition of any of claims 16-19, wherein the 5′ homology arm and 3′ homology arm independently are at or about 200, 300, 400, 500, 600, 700 or 800 nucleotides in length, or any value between any of the foregoing.

21. The composition of any of claims 16-20, wherein the 5′ homology arm and 3′ homology arm independently are greater than at or about 300 nucleotides in length, optionally wherein the 5′ homology arm and 3′ homology arm independently are at or about 400, 500 or 600 nucleotides in length or any value between any of the foregoing, optionally wherein the 5′ homology arm and 3′ homology arm independently are between at or about 500 and at or about 600 nucleotides in length.

22. The composition of any of claims 16-21, wherein the 5′ homology arm and 3′ homology arm independently are greater than at or about 300 nucleotides in length.

23. The composition of any of claims 1-22, wherein the transgene encoding the recombinant receptor or antigen-binding fragment or chain thereof is integrated at or near the target site in the TRAC gene.

24. The composition of any of claims 1-22, wherein the transgene encoding the recombinant receptor or antigen-binding fragment or chain thereof is integrated at or near the target site in one or both of the TRBC1 and the TRBC2 gene.

25. The composition of any of claims 1-24, wherein the recombinant receptor is a chimeric antigen receptor (CAR).

26. The method of claim 25, wherein the CAR comprises an extracellular domain comprising an antigen binding domain specific for the antigen, optionally wherein the antigen binding domain is an scFv; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the antigen-binding domain.

27. The composition of any of claims 1-24, wherein the recombinant receptor is a recombinant TCR or antigen-binding fragment or α chain thereof.

28. The composition of claim 27, wherein the recombinant receptor is a recombinant TCR comprising an alpha (TCRα) chain and a beta (TCRβ) chain and the transgene encoding the recombinant TCR or antigen-binding fragment or chain thereof comprises a nucleic acid sequence encoding the TCRα chain and a nucleic acid sequence encoding the TCRβ chain.

29. The composition of claim 28, wherein the transgene further comprises one or more multicistronic element(s) and the multicistronic element(s) is positioned between the nucleic acid sequence encoding the TCRα or a portion thereof and the nucleic acid sequence encoding the TCRβ or a portion thereof.

30. The composition of claim 29, wherein the multicistronic element(s) comprises a sequence encoding a ribosome skip element selected from among a T2A, a P2A, a E2A or a F2A or an internal ribosome entry site (IRES).

31. The composition of any of claims 1-24, wherein the engineered cells further comprises one or more second transgene(s), wherein the second transgene is integrated at or near one of the at least one target site via homology directed repair (HDR).

32. The composition of claim 31, wherein the recombinant receptor is a recombinant TCR and the transgene encoding the recombinant TCR or antigen-binding fragment or chain thereof comprises a nucleic acid sequence encoding one chain of the recombinant TCR and the second transgene comprises a nucleic acid sequence encoding a different chain of the recombinant TCR.

33. The composition of claim 32, wherein the transgene encoding the recombinant TCR or antigen-binding fragment or chain thereof comprises the nucleic acid sequence encoding the TCRα chain and the second transgene comprises the nucleic acid sequence encoding the TCRβ chain or a portion thereof.

34. The composition of any of claims 31-33, wherein the integration of the second transgene is by a second template polynucleotide introduced into each of the plurality of T cells, said second template polynucleotide comprising the structure [second 5′ homology arm]-[one or more second transgene]-[second 3′ homology arm].

35. The composition of any of claims 31-34, wherein the transgene encoding the recombinant receptor or antigen-binding fragment or chain thereof is integrated at or near a target site in the TRAC gene, the TRBC1 gene or the TRBC2 gene, and the one or more second transgene is integrated at or near one or more other target site among the TRAC gene, the TRBC1 gene or the TRBC2 gene and that is not integrated by the transgene encoding the recombinant receptor or antigen-binding fragment or chain thereof.

36. The composition of any of claims 31-35, wherein the transgene encoding the recombinant receptor or antigen-binding fragment or chain thereof is integrated at or near a target site in the TRAC gene, and the one or more second transgene is integrated at or near one or more target site in the TRBC1 gene and/or the TRBC2 gene.

37. The composition of any of claims 31 and 34-36, wherein the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor.

38. The composition of any of claims 1-37, wherein the transgene encoding the recombinant receptor or antigen-binding fragment or chain thereof further comprises a heterologous regulatory or control element.

39. The composition of any of claims 31-37, wherein the transgene encoding the recombinant receptor or antigen-binding fragment or chain thereof and/or the one or more second transgene independently further comprises a heterologous regulatory or control element.

40. The composition of claim 38 or claim 39, wherein the heterologous regulatory or control element comprises a heterologous promoter.

41. The composition of claim 40, wherein the heterologous promoter is or comprises a human elongation factor 1 alpha (EF1α) promoter or an MND promoter or a variant thereof.

42. The composition of claim 40, wherein the heterologous promoter is an inducible promoter or a repressible promoter.

43. The composition of any of claims 28-42, wherein the TCRα chain comprises a constant (Cα) region comprising introduction of one or more cysteine residues and/or the TCRβ chain comprises aCβ region comprising introduction of one or more cysteine residues, wherein the one or more introduced cysteine residues are capable of forming one or more non-native disulfide bridges between the alpha chain and beta chain.

44. The composition of claim 43, wherein the introduction of the one or more cysteine residues comprises replacement of a non-cysteine residue with a cysteine residue.

45. The composition of any of claims 28-44, wherein the Cα region comprises a cysteine at a position corresponding to position 48 with numbering as set forth in any of SEQ ID NO: 24; and/or the Cβ region comprises a cysteine at a position corresponding to position 57 with numbering as set forth in SEQ ID NO: 20.

46. The composition of any of claims 1-45, wherein the disease, disorder or condition is an infectious disease or disorder, an autoimmune disease, an inflammatory disease, or a tumor or a cancer.

47. The composition of any of claims 1-46, wherein T cells comprise CD8+ T cell and/or CD4+ T cells or subtypes thereof.

48. The composition of any of claims 1-47, wherein the T cells are autologous to the subject.

49. The composition of any of claims 1-48, wherein the T cells are allogeneic to the subject.

50. The composition of any of claims 1-49, further comprising a pharmaceutically acceptable carrier.

51. A method of producing a genetically engineered immune cell, comprising:
(a) introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and
(b) introducing into the immune cell a template polynucleotide comprising a transgene encoding a recombinant receptor or an antigen-binding fragment thereof or α chain thereof, said recombinant receptor being capable of binding to an antigen that is associated with, specific to, and/or expressed on a cell or tissue of a disease, disorder or condition, wherein the transgene encoding the recombinant receptor or antigen-binding fragment or chain thereof is targeted for integration at or near one of the at least one target site via homology directed repair (HDR),
wherein the introduction of the template polynucleotide is performed after the introduction of the one or more agent capable of inducing a genetic disruption.

52. A method of producing a genetically engineered immune cell, comprising:
introducing into an immune cell having a genetic disruption of at least one target site within a T cell receptor alpha constant (TRAC) gene and/or a T cell receptor beta constant (TRBC) gene a template polynucleotide comprising a transgene encoding a recombinant receptor or an antigen-binding fragment thereof or α chain thereof, said recombinant receptor being capable of binding to an antigen that is associated with, specific to, and/or expressed on a cell or tissue of a disease, disorder or condition, wherein the genetic disruption has been induced by one or more agent, wherein each of the one or more agent is independently capable of inducing a genetic disruption, and the transgene encoding the recombinant receptor or antigen-binding fragment or chain thereof is targeted for integration at or near one of the at least one target site via homology directed repair (HDR).

53. The method of claim 51 or claim 52, wherein the template polynucleotide is introduced immediately after, or within at or about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption, optionally at or about 2 hours after the introduction of the one or more agents.

54. The method of any of claims 1-53, wherein the one or more immune cells comprises T cells.

55. The method of claim 54, wherein the T cells comprise CD4+ T cells, CD8+ T cells or CD4+ and CD8+ T cells.

56. The method of claim 55, wherein the T cells comprise CD4+ and CD8+ T cells and the ratio of CD4+ to CD8+ T cells is at or about 1:3 to at or about 3:1, optionally at or about 1:2 to at or about 2:1, optionally at or about 1:1.

57. The method of any of claims 51-56, wherein the each of the one or more agent comprises a CRISPR-Cas9 combination and the CRISPR-Cas9 combination comprises a guide RNA (gRNA) having a targeting domain that is complementary to the at least one target site.

58. The method of claim 57, wherein the CRISPR-Cas9 combination is a ribonucleoprotein (RNP) complex comprising the gRNA and a Cas9 protein.

59. The method of claim 58, wherein the concentration of the RNP is or is about 1 μM to at or about 5 μM, optionally wherein the concentration of the RNP is or is about 2 μM.

60. The method of any of claims 51-58, wherein the introduction of the one or more agent is by electroporation.

61. The method of any of claims 51-60, wherein the template polynucleotide is comprised in a viral vector(s) and the introduction of the template polynucleotide is by transduction.

62. The method of claim 61, wherein the vector is an AAV vector

63. The method of any of claims 51-62, wherein prior to the introducing of the one or more agent, the method comprises incubating the cells, in vitro with a stimulatory agent(s) under conditions to stimulate or activate the one or more immune cells.

64. The method of claim 63, wherein the stimulatory agent (s) comprises and anti-CD3 and/or anti-CD28 antibodies, optionally anti-CD3/anti-CD28 beads, optionally wherein the bead to cell ratio is or is about 1:1.

65. The method of claim 63 or claim 64, comprising removing the stimulatory agent(s) from the one or more immune cells prior to the introducing with the one or more agents.

66. The method of any of claims 51-65, wherein the method further comprises incubating the cells prior to, during or subsequent to the introducing of the one or more agents and/or the introducing of the template polynucleotide with one or more recombinant cytokines, optionally wherein the one or more recombinant cytokines are selected from the group consisting of IL-2, IL-7, and IL-15.

67. The method of claim 66, wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.

68. The method of claim 66 or claim 67, wherein the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

69. The method of any of claims 51-68, wherein the recombinant receptor is a chimeric antigen receptor (CAR).

70. The method of claim 69, wherein the CAR comprises an extracellular domain comprising an antigen binding domain specific for the antigen, optionally wherein the antigen binding domain is an scFv; a transmembrane domain; a cytoplasmic signaling domain derived from a costimulatory molecule, which optionally is or comprises a 4-1BB, optionally human 4-1BB; and a cytoplasmic signaling domain derived from a primary signaling ITAM-containing molecule, which optionally is or comprises a CD3zeta signaling domain, optionally a human CD3zeta signaling domain; and optionally wherein the CAR further comprises a spacer between the transmembrane domain and the antigen-binding domain.

71. The method of any of claims 51-68, wherein the recombinant receptor is a recombinant TCR or antigen-binding fragment or α chain thereof.

72. The composition of claim 71, wherein the recombinant receptor is a recombinant TCR comprising an alpha (TCRα) chain and a beta (TCRβ) chain and the transgene encoding the recombinant TCR or antigen-binding fragment or chain thereof comprises a nucleic acid sequence encoding the TCRα chain and a nucleic acid sequence encoding the TCRβ chain.

73. A method of producing a genetically engineered immune cell, comprising:
(a) introducing into an immune cell one or more agent, wherein each of the one or more agent is independently capable of inducing a genetic disruption of a target site within a T cell receptor alpha constant (TRAC) gene and/or a T cell receptor beta constant (TRBC) gene, thereby inducing a genetic disruption of at least one target site; and
(b) introducing into the immune cell a template polynucleotide comprising a transgene encoding a recombinant receptor that is a recombinant T cell receptor (TCR) or an antigen-binding fragment thereof or α chain thereof, said transgene comprising a heterologous promoter and wherein the transgene is targeted for integration at or near one of the at least one target site via homology directed repair (HDR).

74. A method of producing a genetically engineered immune cell, comprising:
introducing into an immune cell having a genetic disruption of at least one target site within a T cell receptor alpha constant (TRAC) gene and/or a T cell receptor beta constant (TRBC) gene a template polynucleotide comprising a transgene encoding a recombinant receptor that is a recombinant T cell receptor (TCR) or an antigen-binding fragment thereof or α chain thereof, said transgene comprising a heterologous promoter, wherein the genetic disruption has been induced by one or more agent wherein each of the one or more agent is independently capable of inducing a genetic disruption, and the transgene encoding the recombinant receptor or antigen-binding fragment or chain thereof is targeted for integration at or near one of the at least one target site via homology directed repair (HDR).

75. The method of any of claims 51-74, wherein at least one of the one or more agent is capable of inducing a genetic disruption of a target site in a TRAC gene.

76. The method of any of claims 51-74, wherein at least one of the one or more agent is capable of inducing a genetic disruption of a target site in a TRBC gene.

77. The method of any of claims 51-74, wherein the one or more agents comprises at least one agent that capable of inducing a genetic disruption of a target site in a TRAC gene and at least one agent that is capable of inducing a genetic disruption of a target site in a TRBC gene.

78. A method of producing a genetically engineered immune cell, comprising:
(a) introducing into an immune cell at least one agent that is capable of inducing a genetic disruption of a target site within a T cell receptor alpha constant (TRAC) gene and at least one agent that is capable of inducing a genetic disruption of a target site within a T cell receptor beta constant (TRBC) gene, thereby inducing a genetic disruption of the target sites; and
(b) introducing into the immune cell a template polynucleotide comprising a transgene encoding a recombinant receptor that is a recombinant T cell receptor (TCR) or an antigen-binding fragment thereof or α chain thereof, wherein the transgene encoding the recombinant receptor or antigen-binding fragment or chain thereof is targeted for integration at or near one of the at least one of the target site via homology directed repair (HDR).

79. A method of producing a genetically engineered immune cell, comprising:
introducing into an immune cell having a genetic disruption of at least one target site within a T cell receptor alpha constant (TRAC) gene and a genetic disruption of at least one target site within a T cell receptor beta constant (TRBC) gene a template polynucleotide comprising a transgene encoding a recombinant receptor that is a recombinant T cell receptor (TCR) or an antigen-binding fragment thereof or α chain thereof, wherein the genetic disruptions have been induced by at least one agent that is capable of inducing a genetic disruption of a target site within the TRAC gene and at least one agent that is capable of inducing a genetic disruption with the TRBC gene,—and the transgene encoding the recombinant receptor or antigen-binding fragment or chain thereof is targeted for integration at or near one of the at least one target site via homology directed repair (HDR).

80. The method of any of claims 51-79, wherein the TRBC gene is one or both of a T cell receptor beta constant 1 (TRBC1) or T cell receptor beta constant 2 (TRBC2) gene.

81. The method of any of claims 51-56 and 59-80, wherein the one or more agent comprises a zinc finger nuclease (ZFN), a TAL-effector nuclease (TALEN), or and a CRISPR-Cas9 combination that specifically binds to, recognizes, or hybridizes to the target site.

82. The method of any of claims 51-56 and 59-81, wherein the each of the one or more agent comprises a CRISPR-Cas9 combination and the CRISPR-Cas9 combination comprises a guide RNA (gRNA) having a targeting domain that is complementary to the at least one target site.

83. The method of claim 82, wherein the CRISPR-Cas9 combination is a ribonucleoprotein (RNP) complex comprising the gRNA and a Cas9 protein.

84. The method of claim 83, wherein the concentration of the RNP is or is about 1 μM to at or about 5 μM, optionally wherein the concentration of the RNP is or is about 2 μM.

85. The method of claim 83 or claim 84, wherein the RNP is introduced via electroporation.

86. The method of any of claims 51-76 and 80-85, wherein the at least one target site is within an exon of the TRAC, TRBC1 and/or TRBC2 gene.

87. The method of any of claims 77-85, wherein the at least one target site is within an exon of the TRAC and an exon with the TRBC1 or TRBC2 gene.

88. The method of any of claims 57-72 and 82-87, wherein the gRNA has a targeting domain that is complementary to a target site in a TRAC gene and comprises a sequence selected from the group consisting of UCUCUCAGCUGGUACACGGC (SEQ ID NO:28), UGGAUUUAGAGUCUCUCAGC (SEQ ID NO:29), ACACGGCAGGGUCAGGGUUC (SEQ ID NO:30), GAGAAUCAAAAUCGGUGAAU (SEQ ID NO:31), GCUGGUACACGGCAGGGUCA (SEQ ID NO:32), CUCAGCUGGUACACGGC (SEQ ID NO:33), UGGUACACGGCAGGGUC (SEQ ID NO:34), GCUAGACAUGAGGUCUA (SEQ ID NO:35), GUCAGAUUUGUUGCUCC (SEQ ID NO:36), UCAGCUGGUACACGGCA (SEQ ID NO:37), GCAGACAGACUUGUCAC (SEQ ID NO:38), GGUACACGGCAGGGUCA (SEQ ID NO:39), CUUCAAGAGCAACAGUGCUG (SEQ ID NO:40), AGAGCAACAGUGCUGUGGCC (SEQ ID NO:41), AAAGUCAGAUUUGUUGCUCC (SEQ ID NO:42), ACAAAACUGUGCUAGACAUG (SEQ ID NO:43), AAACUGUGCUAGACAUG (SEQ ID NO:44), UGUGCUAGACAUGAGGUCUA (SEQ ID NO:45), GGCUGGGGAAGAAGGUGUCUUC (SEQ ID NO:46), GCUGGGGAAGAAGGUGUCUUC (SEQ ID NO:47), GGGGAAGAAGGUGUCUUC (SEQ ID NO:48), GUUUUGUCUGUGAUAUACACAU (SEQ ID NO:49), GGCAGACAGACUUGUCACUGGAUU (SEQ ID NO:50), GCAGACAGACUUGUCACUGGAUU (SEQ ID NO:51), GACAGACUUGUCACUGGAUU (SEQ ID NO:52), GUGAAUAGGCAGACAGACUUGUCA (SEQ ID NO:53), GAAUAGGCAGACAGACUUGUCA (SEQ ID NO:54), GAGUCUCUCAGCUGGUACACGG (SEQ ID NO:55), GUCUCUCAGCUGGUACACGG (SEQ ID NO:56), GGUACACGGCAGGGUCAGGGUU (SEQ ID NO:57) and GUACACGGCAGGGUCAGGGUU (SEQ ID NO:58).

89. The method of claim any of claims 57-72 and 82-88, wherein the gRNA has a targeting domain comprising the sequence GAGAAUCAAAAUCGGUGAAU (SEQ ID NO:31).

90. The method of any of claims any of claims 57-72 and 82-87, wherein the gRNA has a targeting domain that is complementary to a target site in one or both of a TRBC1 and a TRBC2 gene and comprises a sequence selected from the group consisting of CACCCAGAUCGUCAGCGCCG (SEQ ID NO:59), CAAACACAGCGACCUCGGGU (SEQ ID NO:60), UGACGAGUGGACCCAGGAUA (SEQ ID NO:61), GGCUCUCGGAGAAUGACGAG (SEQ ID NO:62), GGCCUCGGCGCUGACGAUCU (SEQ ID NO:63), GAAAAACGUGUUCCCACCCG (SEQ ID NO:64), AUGACGAGUGGACCCAGGAU (SEQ ID NO:65), AGUCCAGUUCUACGGGCUCU (SEQ ID NO:66), CGCUGUCAAGUCCAGUUCUA (SEQ ID NO:67), AUCGUCAGCGCCGAGGCCUG (SEQ ID NO:68), UCAAACACAGCGACCUCGGG (SEQ ID NO:69), CGUAGAACUGGACUUGACAG (SEQ ID NO:70), AGGCCUCGGCGCUGACGAUC (SEQ ID NO:71), UGACAGCGGAAGUGGUUGCG (SEQ ID NO:72), UUGACAGCGGAAGUGGUUGC (SEQ ID NO:73), UCUCCGAGAGCCCGUAGAAC (SEQ ID NO:74), CGGGUGGGAACACGUUUUUC (SEQ ID NO:75), GACAGGUUUGGCCCUAUCCU (SEQ ID NO:76), GAUCGUCAGCGCCGAGGCCU (SEQ ID NO:77), GGCUCAAACACAGCGACCUC (SEQ ID NO:78), UGAGGGUCUCGGCCACCUUC (SEQ ID NO:79), AGGCUUCUACCCCGACCACG (SEQ ID NO:80), CCGACCACGUGGAGCUGAGC (SEQ ID NO:81), UGACAGGUUUGGCCCUAUCC (SEQ ID NO:82), CUUGACAGCGGAAGUGGUUG (SEQ ID NO:83), AGAUCGUCAGCGCCGAGGCC (SEQ ID NO:84), GCGCUGACGAUCUGGGUGAC (SEQ ID NO:85), UGAGGGCGGGCUGCUCCUUG (SEQ ID NO:86), GUUGCGGGGGUUCUGCCAGA (SEQ ID NO:87), AGCUCAGCUCCACGUGGUCG (SEQ ID NO:88), GCGGCUGCUCAGGCAGUAUC (SEQ ID NO:89), GCGGGGGUUCUGCCAGAAGG (SEQ ID NO:90), UGGCUCAAACACAGCGACCU (SEQ ID NO:91), ACUGGACUUGACAGCGGAAG (SEQ ID NO:92), GACAGCGGAAGUGGUUGCGG (SEQ ID NO:93), GCUGUCAAGUCCAGUUCUAC (SEQ ID NO:94), GUAUCUGGAGUCAUUGAGGG (SEQ ID NO:95), CUCGGCGCUGACGAUCU (SEQ ID NO:96), CCUCGGCGCUGACGAUC (SEQ ID NO:97), CCGAGAGCCCGUAGAAC (SEQ ID NO:98), CCAGAUCGUCAGCGCCG (SEQ ID NO:99), GAAUGACGAGUGGACCC (SEQ ID NO:100), GGGUGACAGGUUUGGCCCUAUC (SEQ ID NO:101), GGUGACAGGUUUGGCCCUAUC (SEQ ID NO:102), GUGACAGGUUUGGCCCUAUC (SEQ ID NO:103), GACAGGUUUGGCCCUAUC (SEQ ID NO:104), GAUACUGCCUGAGCAGCCGCCU (SEQ ID NO:105), GACCACGUGGAGCUGAGCUGGUGG (SEQ ID NO:106), GUGGAGCUGAGCUGGUGG (SEQ ID NO:107), GGGCGGGCUGCUCCUUGAGGGGCU (SEQ ID NO:108), GGCGGGCUGCUCCUUGAGGGGCU (SEQ ID NO:109), GCGGGCUGCUCCUUGAGGGGCU (SEQ ID NO:110), GGGCUGCUCCUUGAGGGGCU (SEQ ID NO:111), GGCUGCUCCUUGAGGGGCU (SEQ ID NO:112), GCUGCUCCUUGAGGGGCU (SEQ ID NO:113), GGUGAAUGGGAAGGAGGUGCACAG (SEQ ID NO:114), GUGAAUGGGAAGGAGGUGCACAG (SEQ ID NO:115) and GAAUGGGAAGGAGGUGCACAG (SEQ ID NO:116).

91. The method of any of claims 57-72 and 82-87 and 90, wherein the gRNA has a targeting domain comprising the sequence GGCCUCGGCGCUGACGAUCU (SEQ ID NO:63).

92. The method of any of claims 51-91, wherein the template polynucleotide comprises the structure [5′ homology arm]-[transgene]-[3′ homology arm].

93. The method of claim 92, wherein the 5′ homology arm and 3′ homology arm comprises nucleic acid sequences homologous to nucleic acid sequences surrounding the at least one target site.

94. The method of claim 92 or claim 93, wherein the 5′ homology arm and 3′ homology arm independently are between at or about 50 and at or about 100 nucleotides in length, at or about 100 and at or about 250 nucleotides in length, at or about 250 and at or about 500 nucleotides in length, at or about 500 and at or about 750 nucleotides in length, at or about 750 and at or about 1000 nucleotides in length, or at or about 1000 and at or about 2000 nucleotides in length.

95. The method of any of claims 92-94, wherein the 5′ homology arm and 3′ homology arm independently are from at or about 100 to at or about 1000 nucleotides, 100 to 750 nucleotides, 100 to 600 nucleotides, 100 to 400 nucleotides, 100 to 300 nucleotides, 100 to 200 nucleotides, 200 to 1000 nucleotides, 200 to 750 nucleotides, 200 to 600 nucleotides, 200 to 400 nucleotides, 200 to 300 nucleotides, 300 to 1000 nucleotides, 300 to 750 nucleotides, 300 to 600 nucleotides, 300 to 400 nucleotides, 400 to 1000 nucleotides, 400 to 750 nucleotides, 400 to 600 nucleotides, 600 to 1000 nucleotides, 600 to 750 nucleotides or 750 to 1000 nucleotides in length.

96. The method of any of claims 92-95, wherein the 5′ homology arm and 3′ homology arm independently are at or about 200, 300, 400, 500, 600, 700 or 800 nucleotides in length, or any value between any of the foregoing.

97. The method of any of claims 92-96, wherein the 5′ homology arm and 3′ homology arm independently are greater than at or about 300 nucleotides in length, optionally wherein the 5′ homology arm and 3′ homology arm independently are at or about 400, 500 or 600 nucleotides in length or any value between any of the foregoing.

98. The method of any of claims 92-97, wherein the 5′ homology arm and 3′ homology arm independently are greater than at or about 300 nucleotides in length.

99. The method of any of claims 51-98, wherein the transgene encoding the recombinant receptor or antigen-binding fragment or chain thereof is targeted for integration at or near the target site in the TRAC gene.

100. The method of any of claims 51-99, wherein the transgene encoding the recombinant receptor or antigen-binding fragment or chain thereof is targeted for integration at or near the target site in one or both of the TRBC1 and the TRBC2 gene.

101. The method of any of claims 73-100, wherein the recombinant receptor is a recombinant TCR comprising an alpha (TCRα) chain and a beta (TCRβ) chain and the transgene encoding the recombinant TCR or antigen-binding fragment or chain thereof comprises a nucleic acid sequence encoding the TCRα chain and a nucleic acid sequence encoding the TCRβ chain.

102. The method of claim 72 or claim 101, wherein the transgene further comprises one or more multicistronic element(s) and the multicistronic element(s) is positioned between the nucleic acid sequence encoding the TCRα or a portion thereof and the nucleic acid sequence encoding the TCRβ or a portion thereof.

103. The method of claim 102, wherein the multicistronic element(s) comprises a sequence encoding a ribosome skip element selected from among a T2A, a P2A, a E2A or a F2A or an internal ribosome entry site (IRES).

104. The method of any of claims 51-103, wherein the method further comprises introducing into the immune cell one or more second template polynucleotide comprising one or more second transgene(s), wherein the second transgene is targeted for integration at or near one of the at least one target site via homology directed repair (HDR).

105. The method of claim 104, wherein the recombinant receptor is a recombinant TCR and the transgene encoding the recombinant TCR or antigen-binding fragment or chain thereof comprises a nucleic acid sequence encoding one chain of the recombinant TCR and the second transgene comprises a nucleic acid sequence encoding a different chain of the recombinant TCR.

106. The method of claim 105, wherein the transgene encoding the recombinant TCR or antigen-binding fragment or chain thereof comprises the nucleic acid sequence encoding the TCRα chain and the second transgene comprises the nucleic acid sequence encoding the TCRβ chain or a portion thereof.

107. The method of any of claims 104-106, wherein the second template polynucleotide comprises the structure [second 5′ homology arm]-[one or more second transgene]-[second 3′ homology arm].

108. The method of any of claims 104-107, wherein the transgene encoding the recombinant receptor or antigen-binding fragment or chain thereof is targeted for integration at or near a target site in the TRAC gene, the TRBC1 gene or the TRBC2 gene, and the one or more second transgene is targeted for integration at or near one or more other target site among the TRAC gene, the TRBC1 gene or the TRBC2 gene and that is not targeted by the transgene encoding the recombinant receptor or antigen-binding fragment or chain thereof.

109. The method of any of claims 104-108, wherein the transgene encoding the recombinant receptor or antigen-binding fragment or chain thereof is targeted for integration at or near a target site in the TRAC gene, and the one or more second transgene is targeted for integration at or near one or more target site in the TRBC1 gene and/or the TRBC2 gene.

110. The method of any of claims 104-109, wherein the one or more second transgene encodes a molecule selected from a co-stimulatory ligand, a cytokine, a soluble single-chain variable fragment (scFv), an immunomodulatory fusion protein, a chimeric switch receptor (CSR) or a co-receptor.

111. The method of any of claims 51-110, wherein the transgene encoding the recombinant receptor or antigen-binding fragment or chain thereof further comprises a regulatory or control element.

112. The method of any of claims 104-111, wherein the transgene encoding the recombinant receptor or antigen-binding fragment or chain thereof and/or the one or more second transgene independently further comprises a heterologous regulatory or control element.

113. The method of claim 111 or claim 112, wherein the heterologous regulatory or control element comprises a heterologous promoter.

114. The method of claim 73, claim 74 or claim 113, wherein the heterologous promoter is or comprises a human elongation factor 1 alpha (EF1α) promoter or an MND promoter or a variant thereof.

115. The method of claim 73, claim 74 or claim 113, wherein the heterologous promoter is an inducible promoter or a repressible promoter.

116. The method of any of claims 51-115, wherein the TCRα chain comprises a constant (Ca) region comprising introduction of one or more cysteine residues and/or the TCRβ chain comprises a Cβ region comprising introduction of one or more cysteine residues, wherein the one or more introduced cysteine residues are capable of forming one or more non-native disulfide bridges between the alpha chain and beta chain.

117. The method of claim 116, wherein the introduction of the one or more cysteine residues comprises replacement of a non-cysteine residue with a cysteine residue.

118. The method of claim 116 or 117, wherein the Cα region comprises a cysteine at a position corresponding to position 48 with numbering as set forth in any of SEQ ID NO: 24; and/or the Cβ region comprises a cysteine at a position corresponding to position 57 with numbering as set forth in SEQ ID NO: 20.

119. The method of any of claims 51-118, wherein the disease, disorder or condition is an infectious disease or disorder, an autoimmune disease, an inflammatory disease, or a tumor or a cancer.

120. The method of any of claims 51-119, wherein the immune cells comprise or are enriched in T cells.

121. The method of claim 120, wherein the T cells comprise a CD8+ T cells or subtypes thereof.

122. The method of claim 120, wherein the T cells comprise a CD4+ T cell or subtypes thereof.

123. The method of claim 120, wherein the T cells comprise CD4+ T cell or subtypes thereof and CD8+ T cells or subtypes thereof.

124. The method of claim 123, wherein the T cells comprise CD4+ and CD8+ T cells and the ratio of CD4+ to CD8+ T cells is at or about 1:3 to at or about 3:1, optionally at or about 1:2 to at or about 2:1, optionally at or about 1:1.

125. The method of any of claims 51-53 and 57-119, wherein the immune cell is derived from a multipotent or pluripotent cell, which optionally is an iPSC.

126. The method of any of claims 51-125, wherein the immune cell is a primary cell from a subject.

127. The method of claim 126, wherein the subject has or is suspected of having the disease, or disorder condition.

128. The method of claim 126, wherein the subject is or is suspected of being healthy.

129. The method of claim 126 or claim 127, wherein the immune cell is autologous to the subject.

130. The method of any of claims 126-128, wherein the immune cell is allogeneic to the subject.

131. The method of any of claims 73-130, wherein the template polynucleotide is comprised in one or more vector(s), which optionally is a viral vector(s).

132. The method of claim 131, wherein the vector is a viral vector and the viral vector is an AAV vector.

133. The method of claim 62 or claim 132, wherein the AAV vector is selected from the group consisting of AAV1, AAV2, AAV3, AAV4, AAV5, AAV6, AAV7 and AAV8 vector.

134. The method of claim 62, claim 132 or claim 133, wherein the AAV vector is an AAV2 or AAV6 vector.

135. The method of claim 61 or claim 131, wherein vector is a viral vector and the viral vector is a retroviral vector, optionally a lentiviral vector.

136. The method of any of claims 51-135, wherein the template polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing.

137. The method of any of claims 51-136, wherein the polynucleotide is between at or about 2500 and at or about 5000 nucleotides, at or about 3500 and at or about 4500 nucleotides, or at or about 3750 nucleotides and at or about 4250 nucleotides in length.

138. The method of any of claims 73-137, wherein the introduction of the one or more agent capable of inducing a genetic disruption and the introduction of the template polynucleotide are performed simultaneously or sequentially, in any order.

139. The method of any of claims 73-138, wherein the introduction of the template polynucleotide is performed after the introduction of the one or more agent capable of inducing a genetic disruption.

140. The method of claim 139, wherein the template polynucleotide is introduced immediately after, or within at or about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after the introduction of one or more agents capable of inducing a genetic disruption, optionally at or about 2 hours after the introduction of the one or more agents.

141. The method of any of claims 73-138, wherein introduction of the one or more agent capable of inducing a genetic disruption and the introduction of the template polynucleotide are performed in one experimental reaction.

142. The method of any of claims 73-141, wherein prior to the introducing of the one or more agent, the method comprises incubating the cells, in vitro with a stimulatory agent(s) under conditions to stimulate or activate the one or more immune cells.

143. The method of claim 142, wherein the stimulatory agent (s) comprises and anti-CD3 and/or anti-CD28 antibodies, optionally anti-CD3/anti-CD28 beads, optionally wherein the bead to cell ratio is or is about 1:1.

144. The method of claim 142 or claim 143, comprising removing the stimulatory agent(s) from the one or more immune cells prior to the introducing with the one or more agents.

145. The method of any of claims 73-144, wherein the method further comprises incubating the cells prior to, during or subsequent to the introducing of the one or more agents and/or the introducing of the template polynucleotide with one or more recombinant cytokines, optionally wherein the one or more recombinant cytokines are selected from the group consisting of IL-2, IL-7, and IL-15.

146. The method of claim 145, wherein the one or more recombinant cytokine is added at a concentration selected from a concentration of IL-2 from at or about 10 U/mL to at or about 200 U/mL, optionally at or about 50 IU/mL to at or about 100 U/mL; IL-7 at a concentration of 0.5 ng/mL to 50 ng/mL, optionally at or about 5 ng/mL to at or about 10 ng/mL and/or IL-15 at a concentration of 0.1 ng/mL to 20 ng/mL, optionally at or about 0.5 ng/mL to at or about 5 ng/mL.

147. The method of claim 145 or claim 146, wherein the incubation is carried out subsequent to the introducing of the one or more agents and the introducing of the template polynucleotide for up to or approximately 24 hours, 36 hours, 48 hours, 3, 4, 5, 6, 7, 8, 9, 10, 11, 12, 13, 14, 15, 16, 17, 18, 19, 20 or 21 days, optionally up to or about 7 days.

148. The method of any of claims 51-147, wherein at least or greater than 35%, 40%, 45%, 50%, 55%, 60%, 65%, 70%, 75%, 80%, or 90% of the cells in a plurality of engineered cells comprise a genetic disruption of at least one target site within a gene encoding a domain or region of T cell receptor alpha constant (TRAC) gene and/or a T cell receptor beta constant (TRBC) gene.

149. The method of any of claims 51-148, wherein at least or greater than 35%, 40%, 45%, 50%, 55%, 60%, 65%, 70%, 75%, 80%, or 90% of the cells in a plurality of engineered cells express the recombinant receptor or antigen-binding fragment thereof and/or exhibit binding to the antigen.

150. The method of any of claims 51-149, wherein the coefficient of variation of expression and/or antigen binding of the recombinant receptor or antigen-binding fragment thereof among a plurality of engineered cells is lower than 0.70, 0.65, 0.60, 0.55, 0.50, 0.45, 0.40, 0.35 or 0.30 or less.

151. The method of any of claims 51-150, wherein the coefficient of variation of expression and/or antigen binding of the recombinant receptor or antigen-binding fragment thereof among a plurality of engineered cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

152. An engineered cell or a plurality of engineered cells, generated using the method of any of claims 51-151.

153. A composition, comprising the engineered cell or plurality of engineered cells of claim 152.

154. The composition of claim 153, wherein at least or greater than 35%, 40%, 45%, 50%, 55%, 60%, 65%, 70%, 75%, 80%, or 90% of the cells in the composition comprise a genetic disruption of at least one target site within a gene encoding a domain or region of T cell receptor alpha constant (TRAC) gene and/or a T cell receptor beta constant (TRBC) gene.

155. The composition of claim 153 or claim 154, wherein at least or greater than 35%, 40%, 45%, 50%, 55%, 60%, 65%, 70%, 75%, 80%, or 90% of the cells in the composition express the recombinant receptor or antigen-binding fragment thereof and/or exhibit binding to the antigen.

156. The composition of any of claims 153-155, wherein the coefficient of variation of expression and/or antigen binding of the recombinant receptor or antigen-binding fragment or a chain thereof among the plurality of cells is lower than 0.70, 0.65, 0.60, 0.55, 0.50, 0.45, 0.40, 0.35 or 0.30 or less.

157. The composition of any of claims 153-156, wherein the coefficient of variation of expression and/or antigen binding of the recombinant receptor or antigen-binding fragment or a chain thereof among the plurality of cells is at least 100%, 95%, 90%, 80%, 70%, 60%, 50%, 40%, 30%, 20% or 10% lower than the coefficient of variation of expression and/or antigen binding of the same recombinant receptor that is integrated into the genome by random integration.

158. The composition of any of claims 153-157, further comprising a pharmaceutically acceptable carrier.

159. A method of treatment comprising administering the engineered cell, plurality of engineered cells of claim 152 or the composition of any of claims 1-50 and 153-158 to a subject in need thereof, optionally wherein the subject has the disease, disorder or condition, optionally wherein the disease, disorder or condition is a cancer.

160. Use of the engineered cell, plurality of engineered cells of claim 152 or composition of any of claims 1-50 and 153-158 for treating cancer disease, disorder or condition, optionally wherein the disease, disorder or condition is a cancer.

161. Use of the engineered cell, plurality of engineered cells of claim 152 or composition of any of claims 1-50 and 153-158 in the manufacture of a medicament for treating a disease, disorder or condition, optionally wherein the disease, disorder or condition is a cancer.

162. The engineered cell or plurality of engineered cells of claim 152 or the composition of any of claims 1-50 and 153-158 for use in treating cancer disease disorder or condition, optionally wherein the disease, disorder or condition is a cancer.

163. A kit, comprising:
one or more agent, wherein each of the one or more agent is independently capable of inducing a genetic disruption of a target site within a T cell receptor alpha constant (TRAC) gene and/or a T cell receptor beta constant (TRBC) gene; and
a template polynucleotide comprising a transgene encoding a recombinant receptor or an antigen-binding fragment or α chain thereof, wherein the transgene encoding the recombinant receptor or antigen-binding fragment or chain thereof is targeted for integration at or near the target site via homology directed repair (HDR)
and instructions for carrying out the method of any of claims 51-151.


SEQUENCE LISTING (256 sequences)

SEQ ID NO: 1 - DNA, 4627 bp, Homo sapiens
  Human TCR alpha constant (TRAC)
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 2 - DNA, 1448 bp, Homo sapiens
  Human TCR beta constant 1 (TRBC1)
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 3 - DNA, 1489 bp, Homo sapiens
  Human TCR beta constant 2 (TRBC2)
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 4 - DNA, 1189 bp, Artificial Sequence
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 5 - DNA, 1205 bp, Artificial Sequence
  EF1alpha promoter
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaa

SEQ ID NO: 6 - protein, 24 aa, Artificial Sequence
  T2A
LEGGGEGRGSLLTCGDVEENPGPR

SEQ ID NO: 7 - protein, 18 aa, Artificial Sequence
  T2A
EGRGSLLTCGDVEENPGP

SEQ ID NO: 8 - protein, 22 aa, Artificial Sequence
  P2A
GSGATNFSLLKQAGDVEENPGP

SEQ ID NO: 9 - protein, 19 aa, Artificial Sequence
  P2A
ATNFSLLKQAGDVEENPGP

SEQ ID NO: 10 - protein, 20 aa, Artificial Sequence
  E2A
QCTNYALLKLAGDVESNPGP

SEQ ID NO: 11 - protein, 22 aa, Artificial Sequence
  F2A
VKQTLNFDLLKLAGDVESNPGP

SEQ ID NO: 12 - protein, 357 aa, Artificial Sequence
  EGFRt
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 13 - protein, 335 aa, Artificial Sequence
  EGFRt
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 14 - protein, 137 aa, Mus musculus
  Mouse TCR alpha constant
DIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 15 - protein, 137 aa, Mus musculus
  Mouse TCR alpha constant
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 16 - protein, 173 aa, Mus musculus
  Mouse TCR beta constant
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 17 - protein, 172 aa, Mus musculus
  Mouse TCR beta constant
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 18 - DNA, 181 bp, Artificial Sequence
  MND promoter
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 19 - protein, 142 aa, Homo sapiens
  Human TCR alpha constant
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 177 aa, Homo sapiens
  Human TCR beta constant 1
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 21 - protein, 178 aa, Homo sapiens
  Human TCR beta constant 2
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 22 - protein, 10 aa, Artificial Sequence
  Linker
  REPEAT (0)...(0): SGGGG is repeated 5 or 6 times
PGGGSGGGGP

SEQ ID NO: 23 - protein, 17 aa, Artificial Sequence
  Linker
GSADDAKKDAAKKDGKS

SEQ ID NO: 24 - protein, 141 aa, Homo sapiens
  human TCR alpha constant
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 25 - protein, 179 aa, Homo sapiens
  human TCR beta constant
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 26 - DNA, 149 bp, Artificial Sequence
  TRAC gRNA transcription sequence
agcgctctcgtacagagttggcattataatacgactcactataggggagaatcaaaatcggtgaatgttttagagctagaaatagcaagttaaaataaggctagtccgttatcaacttgaaaaagtggcaccgagtcggtgcttttttt

SEQ ID NO: 27 - RNA, 100 nt, Artificial Sequence
  TRAC gRNA sequence
gagaaucaaaaucggugaauguuuuagagcuagaaauagcaaguuaaaauaaggcuaguccguuaucaacuugaaaaaguggcaccgagucggugcuuuu

SEQ ID NO: 28 - RNA, 20 nt, Artificial Sequence
  TRAC-10 gRNA targeting domain
ucucucagcugguacacggc

SEQ ID NO: 29 - RNA, 20 nt, Artificial Sequence
  TRAC-110 gRNA targeting domain
uggauuuagagucucucagc

SEQ ID NO: 30 - RNA, 20 nt, Artificial Sequence
  TRAC-116 gRNA targeting domain
acacggcagggucaggguuc

SEQ ID NO: 31 - RNA, 20 nt, Artificial Sequence
  TRAC-16 gRNA targeting domain
gagaaucaaaaucggugaau

SEQ ID NO: 32 - RNA, 20 nt, Artificial Sequence
  TRAC-4 gRNA targeting domain
gcugguacacggcaggguca

SEQ ID NO: 33 - RNA, 17 nt, Artificial Sequence
  TRAC-49 gRNA targeting domain
cucagcugguacacggc

SEQ ID NO: 34 - RNA, 17 nt, Artificial Sequence
  TRAC-2 gRNA targeting domain
ugguacacggcaggguc

SEQ ID NO: 35 - RNA, 17 nt, Artificial Sequence
  TRAC-30 gRNA targeting domain
gcuagacaugaggucua

SEQ ID NO: 36 - RNA, 17 nt, Artificial Sequence
  TRAC-43 gRNA targeting domain
gucagauuuguugcucc

SEQ ID NO: 37 - RNA, 17 nt, Artificial Sequence
  TRAC-23 gRNA targeting domain
ucagcugguacacggca

SEQ ID NO: 38 - RNA, 17 nt, Artificial Sequence
  TRAC-34 gRNA targeting domain
gcagacagacuugucac

SEQ ID NO: 39 - RNA, 17 nt, Artificial Sequence
  TRAC-25 gRNA targeting domain
gguacacggcaggguca

SEQ ID NO: 40 - RNA, 20 nt, Artificial Sequence
  TRAC-128 gRNA targeting domain
cuucaagagcaacagugcug

SEQ ID NO: 41 - RNA, 20 nt, Artificial Sequence
  TRAC-105 gRNA targeting domain
agagcaacagugcuguggcc

SEQ ID NO: 42 - RNA, 20 nt, Artificial Sequence
  TRAC-106 gRNA targeting domain
aaagucagauuuguugcucc

SEQ ID NO: 43 - RNA, 20 nt, Artificial Sequence
  TRAC-123 gRNA targeting domain
acaaaacugugcuagacaug

SEQ ID NO: 44 - RNA, 17 nt, Artificial Sequence
  TRAC-64 gRNA targeting domain
aaacugugcuagacaug

SEQ ID NO: 45 - RNA, 20 nt, Artificial Sequence
  TRAC-97 gRNA targeting domain
ugugcuagacaugaggucua

SEQ ID NO: 46 - RNA, 22 nt, Artificial Sequence
  TRAC-148 gRNA targeting domain
ggcuggggaagaaggugucuuc

SEQ ID NO: 47 - RNA, 21 nt, Artificial Sequence
  TRAC-147 gRNA targeting domain
gcuggggaagaaggugucuuc

SEQ ID NO: 48 - RNA, 18 nt, Artificial Sequence
  TRAC-234 gRNA targeting domain
ggggaagaaggugucuuc

SEQ ID NO: 49 - RNA, 22 nt, Artificial Sequence
  TRAC-167 gRNA targeting domain
guuuugucugugauauacacau

SEQ ID NO: 50 - RNA, 24 nt, Artificial Sequence
  TRAC-177 gRNA targeting domain
ggcagacagacuugucacuggauu

SEQ ID NO: 51 - RNA, 23 nt, Artificial Sequence
  TRAC-176 gRNA targeting domain
gcagacagacuugucacuggauu

SEQ ID NO: 52 - RNA, 20 nt, Artificial Sequence
  TRAC-257 gRNA targeting domain
gacagacuugucacuggauu

SEQ ID NO: 53 - RNA, 24 nt, Artificial Sequence
  TRAC-233 gRNA targeting domain
gugaauaggcagacagacuuguca

SEQ ID NO: 54 - RNA, 22 nt, Artificial Sequence
  TRAC-231 gRNA targeting domain
gaauaggcagacagacuuguca

SEQ ID NO: 55 - RNA, 22 nt, Artificial Sequence
  TRAC-163 gRNA targeting domain
gagucucucagcugguacacgg

SEQ ID NO: 56 - RNA, 20 nt, Artificial Sequence
  TRAC-241 gRNA targeting domain
gucucucagcugguacacgg

SEQ ID NO: 57 - RNA, 22 nt, Artificial Sequence
  TRAC-179 gRNA targeting domain
gguacacggcagggucaggguu

SEQ ID NO: 58 - RNA, 21 nt, Artificial Sequence
  TRAC-178 gRNA targeting domain
guacacggcagggucaggguu

SEQ ID NO: 59 - RNA, 20 nt, Artificial Sequence
  TRBC-40 gRNA targeting domain
cacccagaucgucagcgccg

SEQ ID NO: 60 - RNA, 20 nt, Artificial Sequence
  TRBC-52 gRNA targeting domain
caaacacagcgaccucgggu

SEQ ID NO: 61 - RNA, 20 nt, Artificial Sequence
  TRBC-25 gRNA targeting domain
ugacgaguggacccaggaua

SEQ ID NO: 62 - RNA, 20 nt, Artificial Sequence
  TRBC-35 gRNA targeting domain
ggcucucggagaaugacgag

SEQ ID NO: 63 - RNA, 20 nt, Artificial Sequence
  TRBC-50 gRNA targeting domain
ggccucggcgcugacgaucu

SEQ ID NO: 64 - RNA, 20 nt, Artificial Sequence
  TRBC-39 gRNA targeting domain
gaaaaacguguucccacccg

SEQ ID NO: 65 - RNA, 20 nt, Artificial Sequence
  TRBC-49 gRNA targeting domain
augacgaguggacccaggau

SEQ ID NO: 66 - RNA, 20 nt, Artificial Sequence
  TRBC-51 gRNA targeting domain
aguccaguucuacgggcucu

SEQ ID NO: 67 - RNA, 20 nt, Artificial Sequence
  TRBC-26 gRNA targeting domain
cgcugucaaguccaguucua

SEQ ID NO: 68 - RNA, 20 nt, Artificial Sequence
  TRBC-47 gRNA targeting domain
aucgucagcgccgaggccug

SEQ ID NO: 69 - RNA, 20 nt, Artificial Sequence
  TRBC-45 gRNA targeting domain
ucaaacacagcgaccucggg

SEQ ID NO: 70 - RNA, 20 nt, Artificial Sequence
  TRBC-34 gRNA targeting domain
cguagaacuggacuugacag

SEQ ID NO: 71 - RNA, 20 nt, Artificial Sequence
  TRBC-227 gRNA targeting domain
aggccucggcgcugacgauc

SEQ ID NO: 72 - RNA, 20 nt, Artificial Sequence
  TRBC-41 gRNA targeting domain
ugacagcggaagugguugcg

SEQ ID NO: 73 - RNA, 20 nt, Artificial Sequence
  TRBC-30 gRNA targeting domain
uugacagcggaagugguugc

SEQ ID NO: 74 - RNA, 20 nt, Artificial Sequence
  TRBC-206 gRNA targeting domain
ucuccgagagcccguagaac

SEQ ID NO: 75 - RNA, 20 nt, Artificial Sequence
  TRBC-32 gRNA targeting domain
cgggugggaacacguuuuuc

SEQ ID NO: 76 - RNA, 20 nt, Artificial Sequence
  TRBC-276 gRNA targeting domain
gacagguuuggcccuauccu

SEQ ID NO: 77 - RNA, 20 nt, Artificial Sequence
  TRBC-274 gRNA targeting domain
gaucgucagcgccgaggccu

SEQ ID NO: 78 - RNA, 20 nt, Artificial Sequence
  TRBC-230 gRNA targeting domain
ggcucaaacacagcgaccuc

SEQ ID NO: 79 - RNA, 20 nt, Artificial Sequence
  TRBC-235 gRNA targeting domain
ugagggucucggccaccuuc

SEQ ID NO: 80 - RNA, 20 nt, Artificial Sequence
  TRBC-38 gRNA targeting domain
aggcuucuaccccgaccacg

SEQ ID NO: 81 - RNA, 20 nt, Artificial Sequence
  TRBC-223 gRNA targeting domain
ccgaccacguggagcugagc

SEQ ID NO: 82 - RNA, 20 nt, Artificial Sequence
  TRBC-221 gRNA targeting domain
ugacagguuuggcccuaucc

SEQ ID NO: 83 - RNA, 20 nt, Artificial Sequence
  TRBC-48 gRNA targeting domain
cuugacagcggaagugguug

SEQ ID NO: 84 - RNA, 20 nt, Artificial Sequence
  TRBC-216 gRNA targeting domain
agaucgucagcgccgaggcc

SEQ ID NO: 85 - RNA, 20 nt, Artificial Sequence
  TRBC-210 gRNA targeting domain
gcgcugacgaucugggugac

SEQ ID NO: 86 - RNA, 20 nt, Artificial Sequence
  TRBC-268 gRNA targeting domain
ugagggcgggcugcuccuug

SEQ ID NO: 87 - RNA, 20 nt, Artificial Sequence
  TRBC-193 gRNA targeting domain
guugcggggguucugccaga

SEQ ID NO: 88 - RNA, 20 nt, Artificial Sequence
  TRBC-246 gRNA targeting domain
agcucagcuccacguggucg

SEQ ID NO: 89 - RNA, 20 nt, Artificial Sequence
  TRBC-228 gRNA targeting domain
gcggcugcucaggcaguauc

SEQ ID NO: 90 - RNA, 20 nt, Artificial Sequence
  TRBC-43 gRNA targeting domain
gcggggguucugccagaagg

SEQ ID NO: 91 - RNA, 20 nt, Artificial Sequence
  TRBC-272 gRNA targeting domain
uggcucaaacacagcgaccu

SEQ ID NO: 92 - RNA, 20 nt, Artificial Sequence
  TRBC-33 gRNA targeting domain
acuggacuugacagcggaag

SEQ ID NO: 93 - RNA, 20 nt, Artificial Sequence
  TRBC-44 gRNA targeting domain
gacagcggaagugguugcgg

SEQ ID NO: 94 - RNA, 20 nt, Artificial Sequence
  TRBC-211 gRNA targeting domain
gcugucaaguccaguucuac

SEQ ID NO: 95 - RNA, 20 nt, Artificial Sequence
  TRBC-253 gRNA targeting domain
guaucuggagucauugaggg

SEQ ID NO: 96 - RNA, 17 nt, Artificial Sequence
  TRBC-18 gRNA targeting domain
cucggcgcugacgaucu

SEQ ID NO: 97 - RNA, 17 nt, Artificial Sequence
  TRBC-6 gRNA targeting domain
ccucggcgcugacgauc

SEQ ID NO: 98 - RNA, 17 nt, Artificial Sequence
  TRBC-85 gRNA targeting domain
ccgagagcccguagaac

SEQ ID NO: 99 - RNA, 17 nt, Artificial Sequence
  TRBC-129 gRNA targeting domain
ccagaucgucagcgccg

SEQ ID NO: 100 - RNA, 17 nt, Artificial Sequence
  TRBC-93 gRNA targeting domain
gaaugacgaguggaccc

SEQ ID NO: 101 - RNA, 22 nt, Artificial Sequence
  TRBC-415 gRNA targeting domain
gggugacagguuuggcccuauc

SEQ ID NO: 102 - RNA, 21 nt, Artificial Sequence
  TRBC-414 gRNA targeting domain
ggugacagguuuggcccuauc

SEQ ID NO: 103 - RNA, 20 nt, Artificial Sequence
  TRBC-310 gRNA targeting domain
gugacagguuuggcccuauc

SEQ ID NO: 104 - RNA, 18 nt, Artificial Sequence
  TRBC-308 gRNA targeting domain
gacagguuuggcccuauc

SEQ ID NO: 105 - RNA, 22 nt, Artificial Sequence
  TRBC-401 gRNA targeting domain
gauacugccugagcagccgccu

SEQ ID NO: 106 - RNA, 24 nt, Artificial Sequence
  TRBC-468 gRNA targeting domain
gaccacguggagcugagcuggugg

SEQ ID NO: 107 - RNA, 18 nt, Artificial Sequence
  TRBC-462 gRNA targeting domain
guggagcugagcuggugg

SEQ ID NO: 108 - RNA, 24 nt, Artificial Sequence
  TRBC-424 gRNA targeting domain
gggcgggcugcuccuugaggggcu

SEQ ID NO: 109 - RNA, 23 nt, Artificial Sequence
  TRBC-423 gRNA targeting domain
ggcgggcugcuccuugaggggcu

SEQ ID NO: 110 - RNA, 22 nt, Artificial Sequence
  TRBC-422 gRNA targeting domain
gcgggcugcuccuugaggggcu

SEQ ID NO: 111 - RNA, 20 nt, Artificial Sequence
  TRBC-420 gRNA targeting domain
gggcugcuccuugaggggcu

SEQ ID NO: 112 - RNA, 19 nt, Artificial Sequence
  TRBC-419 gRNA targeting domain
ggcugcuccuugaggggcu

SEQ ID NO: 113 - RNA, 18 nt, Artificial Sequence
  TRBC-418 gRNA targeting domain
gcugcuccuugaggggcu

SEQ ID NO: 114 - RNA, 24 nt, Artificial Sequence
  TRBC-445 gRNA targeting domain
ggugaaugggaaggaggugcacag

SEQ ID NO: 115 - RNA, 23 nt, Artificial Sequence
  TRBC-444 gRNA targeting domain
gugaaugggaaggaggugcacag

SEQ ID NO: 116 - RNA, 21 nt, Artificial Sequence
  TRBC-442 gRNA targeting domain
gaaugggaaggaggugcacag

SEQ ID NO: 117 - DNA, 20 bp, Artificial Sequence
  TRAC target sequence 1
attcaccgattttgattctc

SEQ ID NO: 118 - DNA, 20 bp, Artificial Sequence
  TRBC target sequence 2
agatcgtcagcgccgaggcc

SEQ ID NO: 119 - DNA, 25 bp, Artificial Sequence
  HBB splice site acceptor
ctgacctcttctcttcctcccacag

SEQ ID NO: 120 - DNA, 13 bp, Artificial Sequence
  IgG splice site acceptor
tttctctccacag

SEQ ID NO: 121 - protein, 138 aa, mus musculus
  Mouse TCR alpha constant
PYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 122 - protein, 136 aa, Artificial Sequence
  Modified mouse TCR alpha constant
IQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 123 - protein, 173 aa, Artificial Sequence
  Modified mouse TCR beta constant
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 124 - DNA, 611 bp, Artificial Sequence
  TRAC 5′ homology arm
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 125 - DNA, 628 bp, Artificial Sequence
  TRAC 3′ homology arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg

SEQ ID NO: 126 - DNA, 347 bp, Artificial Sequence
  MND promoter
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 127 - DNA, 544 bp, Artificial Sequence
  Ef1alpha promoter with HTLV1 enhancer
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 128 - DNA, 66 bp, Artificial Sequence
  P2A nucleotide sequence
ggatctggagcgacgaattttagtctactgaaacaagcgggagacgtggaggaaaaccctggacct

SEQ ID NO: 129 - protein, 112 aa, homo sapiens
  CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 112 aa, homo sapiens
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 12 aa, homo sapiens
  spacer (IgG4hinge)
ESKYGPPCPPCP

SEQ ID NO: 132 - DNA, 36 bp, homo sapiens
  spacer (IgG4hinge)
gaatctaagtacggaccgccctgccccccttgccct

SEQ ID NO: 133 - protein, 119 aa, homo sapiens
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 229 aa, homo sapiens
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 135 - protein, 282 aa, homo sapiens
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 136 - protein, 27 aa, homo sapiens
  CD28
FWVLVVVGGVLACYSLLVTVAFIIFWV

SEQ ID NO: 137 - protein, 66 aa, homo sapiens
  CD28
IEVMYPPPYLDNEKSNGTIIHVKGKHLCPSPLFPGPSKPFWVLVVVGGVLACYSLLVTVAFIIFWV

SEQ ID NO: 138 - protein, 41 aa, homo sapiens
  CD28
RSKRSRLLHSDYMNMTPRRPGPTRKHYQPYAPPRDFAAYRS

SEQ ID NO: 139 - protein, 41 aa, homo sapiens
  CD28 (LL to GG)
RSKRSRGGHSDYMNMTPRRPGPTRKHYQPYAPPRDFAAYRS

SEQ ID NO: 140 - protein, 42 aa, homo sapiens
  4-1BB
KRGRKKLLYIFKQPFMRPVQTTQEEDGCSCRFPEEEEGGCEL

SEQ ID NO: 141 - protein, 112 aa, homo sapiens
  CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - RNA, 96 nt, Artificial Sequence
  exemplary gRNA complementary domain
  modified_base (1)...(20): a, c, u, g, unknown or other
  misc_feature (1)...(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 143 - RNA, 104 nt, Artificial Sequence
  exemplary gRNA complementary domain
  modified_base (1)...(20): a, c, u, g, unknown or other
  misc_feature (1)...(20): n is a, c, g, or u
nnnnnnnnnnnnnnnnnnnnguuuuagagcuaugcugaaaagcauagcaaguuaaaauaaggcuaguccguuaucaacuugaaaaaguggcaccgagucggugc

SEQ ID NO: 144 - RNA, 106 nt, Artificial Sequence
  exemplary gRNA complementary domain
  modified_base (1)...(20): a, c, u, g, unknown or other
  misc_feature (1)...(20): n is a, c, g, or u
nnnnnnnnnnnnnnnnnnnnguuuuagagcuaugcuggaaacagcauagcaaguuaaaauaaggcuaguccguuaucaacuugaaaaaguggcaccgagucggugc

SEQ ID NO: 145 - RNA, 116 nt, Artificial Sequence
  exemplary gRNA complementary domain
  modified_base (1)...(20): a, c, u, g, unknown or other
  misc_feature (1)...(20): n is a, c, g, or u
nnnnnnnnnnnnnnnnnnnnguuuuagagcuaugcuguuuuggaaacaaaacagcauagcaaguuaaaauaaggcuaguccguuaucaacuugaaaaaguggcaccgagucggugc

SEQ ID NO: 146 - RNA, 96 nt, Artificial Sequence
  exemplary gRNA
  modified_base (1)...(20): a, c, u, g, unknown or other
  misc_feature (1)...(20): n is a, c, g, or u
nnnnnnnnnnnnnnnnnnnnguauuagagcuagaaauagcaaguuaauauaaggcuaguccguuaucaacuugaaaaaguggcaccgagucggugc

SEQ ID NO: 147 - RNA, 96 nt, Artificial Sequence
  exemplary gRNA
  modified_base (1)...(20): a, c, u, g, unknown or other
  misc_feature (1)...(20): n is a, c, g, or u
nnnnnnnnnnnnnnnnnnnnguuuaagagcuagaaauagcaaguuuaaauaaggcuaguccguuaucaacuugaaaaaguggcaccgagucggugc

SEQ ID NO: 148 - RNA, 116 nt, Artificial Sequence
  exemplary gRNA
  modified_base (1)...(20): a, c, u, g, unknown or other
  misc_feature (1)...(20): n is a, c, g, or u
nnnnnnnnnnnnnnnnnnnnguauuagagcuaugcuguauuggaaacaauacagcauagcaaguuaauauaaggcuaguccguuaucaacuugaaaaaguggcaccgagucggugc

SEQ ID NO: 149 - RNA, 47 nt, Artificial Sequence
  exemplary proximal and tail domain
aaggcuaguccguuaucaacuugaaaaaguggcaccgagucggugcu

SEQ ID NO: 150 - RNA, 49 nt, Artificial Sequence
  exemplary proximal and tail domain
aaggcuaguccguuaucaacuugaaaaaguggcaccgagucgguggugc

SEQ ID NO: 151 - RNA, 51 nt, Artificial Sequence
  exemplary proximal and tail domain
aaggcuaguccguuaucaacuugaaaaaguggcaccgagucggugcggauc

SEQ ID NO: 152 - RNA, 31 nt, Artificial Sequence
  exemplary proximal and tail domain
aaggcuaguccguuaucaacuugaaaaagug

SEQ ID NO: 153 - RNA, 18 nt, Artificial Sequence
  exemplary proximal and tail domain
aaggcuaguccguuauca

SEQ ID NO: 154 - RNA, 12 nt, Artificial Sequence
  exemplary proximal and tail domain
aaggcuaguccg

SEQ ID NO: 155 - RNA, 102 nt, Artificial Sequence
  exemplary chimeric gRNA
  modified_base (1)...(20): a, c, u, g, unknown or other
  misc_feature (1)...(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuuuu

SEQ ID NO: 156 - RNA, 102 nt, Artificial Sequence
  exemplary chimeric gRNA
  modified_base (1)...(20): a, c, u, g, unknown or other
  misc_feature (1)...(20): n is a, c, g, or u
nnnnnnnnnnnnnnnnnnnnguuuuaguacucuggaaacagaaucuacuaaaacaaggcaaaaugccguguuuaucucgucaacuuguuggcgagauuuuuu

SEQ ID NO: 157 - protein, 1344 aa, Artificial Sequence
  Streptococcus mutans Cas9
KKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 158 - protein, 1367 aa, Artificial Sequence
  Streptococcus pyogenes Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 159 - protein, 1387 aa, Artificial Sequence
  Streptococcus thermophilus Cas9
TKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 160 - protein, 1333 aa, Artificial Sequence
  Listeria innocua Cas9
KKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 161 - protein, 1082 aa, Artificial Sequence
  Neisseria meningitidis Cas9
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 162 - protein, 1368 aa, Artificial Sequence [sequence omitted]

SEQ ID NO: 163 - DNA, 20 bp, Artificial Sequence
  TRAC target sequence 2
gagaatcaaaatcggtgaat

SEQ ID NO: 164 - DNA, 20 bp, Artificial Sequence
  TRBC target sequence 2
ggcctcggcgctgacgatct

SEQ ID NO: 165 - DNA, 23 bp, Artificial Sequence
  TRAC target sequence with PAM 3
gagaatcaaaatcggtgaatagg

SEQ ID NO: 166 - DNA, 23 bp, Artificial Sequence
  TRAC target sequence with PAM 4
ttcaaaacctgtcagtgattggg

SEQ ID NO: 167 - DNA, 23 bp, Artificial Sequence
  TRAC target sequence with PAM 5
tgtgctagacatgaggtctatgg

SEQ ID NO: 168 - DNA, 23 bp, Artificial Sequence
  TRAC target sequence with PAM 6
cgtcatgagcagattaaacccgg

SEQ ID NO: 169 - DNA, 23 bp, Artificial Sequence
  TRAC target sequence with PAM 7
tcagggttctggatatctgtggg

SEQ ID NO: 170 - DNA, 23 bp, Artificial Sequence
  TRAC target sequence with PAM 8
gtcagggttctggatatctgtgg

SEQ ID NO: 171 - DNA, 23 bp, Artificial Sequence
  TRAC target sequence with PAM 9
ttcggaacccaatcactgacagg

SEQ ID NO: 172 - DNA, 23 bp, Artificial Sequence
  TRAC target sequence with PAM 10
taaacccggccactttcaggagg

SEQ ID NO: 173 - DNA, 23 bp, Artificial Sequence
  TRAC target sequence with PAM 11
aaagtcagatttgttgctccagg

SEQ ID NO: 174 - DNA, 23 bp, Artificial Sequence
  TRAC target sequence with PAM 12
aacaaatgtgtcacaaagtaagg

SEQ ID NO: 175 - DNA, 23 bp, Artificial Sequence
  TRAC target sequence with PAM 13
tggatttagagtctctcagctgg

SEQ ID NO: 176 - DNA, 23 bp, Artificial Sequence
  TRAC target sequence with PAM 14
taggcagacagacttgtcactgg

SEQ ID NO: 177 - DNA, 23 bp, Artificial Sequence
  TRAC target sequence with PAM 15
agctggtacacggcagggtcagg

SEQ ID NO: 178 - DNA, 23 bp, Artificial Sequence
  TRAC target sequence with PAM 16
gctggtacacggcagggtcaggg

SEQ ID NO: 179 - DNA, 23 bp, Artificial Sequence
  TRAC target sequence with PAM 17
tctctcagctggtacacggcagg

SEQ ID NO: 180 - DNA, 23 bp, Artificial Sequence
  TRAC target sequence with PAM 18
tttcaaaacctgtcagtgattgg

SEQ ID NO: 181 - DNA, 23 bp, Artificial Sequence
  TRAC target sequence with PAM 19
gattaaacccggccactttcagg

SEQ ID NO: 182 - DNA, 23 bp, Artificial Sequence
  TRAC target sequence with PAM 20
ctcgaccagcttgacatcacagg

SEQ ID NO: 183 - DNA, 23 bp, Artificial Sequence
  TRAC target sequence with PAM 21
agagtctctcagctggtacacgg

SEQ ID NO: 184 - DNA, 23 bp, Artificial Sequence
  TRAC target sequence with PAM 22
ctctcagctggtacacggcaggg

SEQ ID NO: 185 - DNA, 23 bp, Artificial Sequence
  TRAC target sequence with PAM 23
aagttcctgtgatgtcaagctgg

SEQ ID NO: 186 - DNA, 23 bp, Artificial Sequence
  TRAC target sequence with PAM 24
atcctcctcctgaaagtggccgg

SEQ ID NO: 187 - DNA, 23 bp, Artificial Sequence
  TRAC target sequence with PAM 25
tgctcatgacgctgcggctgtgg

SEQ ID NO: 188 - DNA, 23 bp, Artificial Sequence
  TRAC target sequence with PAM 26
acaaaactgtgctagacatgagg

SEQ ID NO: 189 - DNA, 23 bp, Artificial Sequence
  TRAC target sequence with PAM 27
atttgtttgagaatcaaaatcgg

SEQ ID NO: 190 - DNA, 23 bp, Artificial Sequence
  TRAC target sequence with PAM 28
catcacaggaactttctaaaagg

SEQ ID NO: 191 - DNA, 23 bp, Artificial Sequence
  TRAC target sequence with PAM 29
gtcgagaaaagctttgaaacagg

SEQ ID NO: 192 - DNA, 23 bp, Artificial Sequence
  TRAC target sequence with PAM 30
ccactttcaggaggaggattcgg

SEQ ID NO: 193 - DNA, 23 bp, Artificial Sequence
  TRAC target sequence with PAM 31
ctgacaggttttgaaagtttagg

SEQ ID NO: 194 - DNA, 23 bp, Artificial Sequence
  TRAC target sequence with PAM 32
agctttgaaacaggtaagacagg

SEQ ID NO: 195 - DNA, 23 bp, Artificial Sequence
  TRAC target sequence with PAM 33
tggaataatgctgttgttgaagg

SEQ ID NO: 196 - DNA, 23 bp, Artificial Sequence
  TRAC target sequence with PAM 34
agagcaacagtgctgtggcctgg

SEQ ID NO: 197 - DNA, 23 bp, Artificial Sequence
  TRAC target sequence with PAM 35
ctgtggtccagctgaggtgaggg

SEQ ID NO: 198 - DNA, 23 bp, Artificial Sequence
  TRAC target sequence with PAM 36
ctgcggctgtggtccagctgagg

SEQ ID NO: 199 - DNA, 23 bp, Artificial Sequence
  TRAC target sequence with PAM 37
tgtggtccagctgaggtgagggg

SEQ ID NO: 200 - DNA, 23 bp, Artificial Sequence
  TRAC target sequence with PAM 38
cttcttccccagcccaggtaagg

SEQ ID NO: 201 - DNA, 23 bp, Artificial Sequence
  TRAC target sequence with PAM 39
acacggcagggtcagggttctgg

SEQ ID NO: 202 - DNA, 23 bp, Artificial Sequence
  TRAC target sequence with PAM 40
cttcaagagcaacagtgctgtgg

SEQ ID NO: 203 - DNA, 23 bp, Artificial Sequence
  TRAC target sequence with PAM 41
ctggggaagaaggtgtcttctgg

SEQ ID NO: 204 - DNA, 23 bp, Artificial Sequence
  TRAC target sequence with PAM 42
tcctcctcctgaaagtggccggg

SEQ ID NO: 205 - DNA, 23 bp, Artificial Sequence
  TRAC target sequence with PAM 43
ttaatctgctcatgacgctgcgg

SEQ ID NO: 206 - DNA, 23 bp, Artificial Sequence
  TRAC target sequence with PAM 44
acccggccactttcaggaggagg

SEQ ID NO: 207 - DNA, 23 bp, Artificial Sequence
  TRAC target sequence with PAM 45
ttcttccccagcccaggtaaggg

SEQ ID NO: 208 - DNA, 23 bp, Artificial Sequence
  TRAC target sequence with PAM 46
cttacctgggctggggaagaagg

SEQ ID NO: 209 - DNA, 23 bp, Artificial Sequence
  TRAC target sequence with PAM 47
gacaccttcttccccagcccagg

SEQ ID NO: 210 - DNA, 23 bp, Artificial Sequence
  TRAC target sequence with PAM 48
gctgtggtccagctgaggtgagg

SEQ ID NO: 211 - DNA, 23 bp, Artificial Sequence
  TRAC target sequence with PAM 49
ccgaatcctcctcctgaaagtgg

SEQ ID NO: 212 - DNA, 23 bp, Artificial Sequence
  TRBC target sequence with PAM 3
gctgtcaagtccagttctacggg

SEQ ID NO: 213 - DNA, 28 bp, Artificial Sequence
  TRAC ZFN target sequence 1
ctatggacttcaagagcaacagtgctgt

SEQ ID NO: 214 - DNA, 28 bp, Artificial Sequence
  TRAC ZFN target sequence 2
ctcatgtctagcacagttttgtctgtga

SEQ ID NO: 215 - DNA, 28 bp, Artificial Sequence
  TRAC ZFN target sequence 3
gtgctgtggcctggagcaacaaatctga

SEQ ID NO: 216 - DNA, 28 bp, Artificial Sequence
  TRAC ZFN target sequence 4
ttgctcttgaagtccatagacctcatgt

SEQ ID NO: 217 - DNA, 27 bp, Artificial Sequence
  TRAC ZFN target sequence 5
ctgtggcctggagcaacaaatctgact

SEQ ID NO: 218 - DNA, 28 bp, Artificial Sequence
  TRAC ZFN target sequence 6
ctgttgctcttgaagtccatagacctca

SEQ ID NO: 219 - DNA, 28 bp, Artificial Sequence
  TRAC ZFN target sequence 7
ctgtggcctggagcaacaaatctgactt

SEQ ID NO: 220 - DNA, 28 bp, Artificial Sequence
  TRAC ZFN target sequence 8
ctgactttgcatgtgcaaacgccttcaa

SEQ ID NO: 221 - DNA, 28 bp, Artificial Sequence
  TRAC ZFN target sequence 9
ttgttgctccaggccacagcactgttgc

SEQ ID NO: 222 - DNA, 28 bp, Artificial Sequence
  TRAC ZFN target sequence 10
tgaaagtggccgggtttaatctgctcat

SEQ ID NO: 223 - DNA, 28 bp, Artificial Sequence
  TRAC ZFN target sequence 11
aggaggattcggaacccaatcactgaca

SEQ ID NO: 224 - DNA, 28 bp, Artificial Sequence
  TRAC ZFN target sequence 12
gaggaggattcggaacccaatcactgac

SEQ ID NO: 225 - DNA, 28 bp, Artificial Sequence
  TRBC ZFN target sequence 1
ccgtagaactggacttgacagcggaagt

SEQ ID NO: 226 - DNA, 28 bp, Artificial Sequence
  TRBC ZFN target sequence 2
tctcggagaatgacgagtggacccagga

SEQ ID NO: 227 - DNA, 50 bp, Artificial Sequence
  TRAC 50 bp 5′ Homology Arm
agctgagagactctaaatccagtgacaagtctgtctgcctattcaccgat

SEQ ID NO: 228 - DNA, 100 bp, Artificial Sequence
  TRAC 100 bp 5′ Homology Arm
ctgatcctcttgtcccacagatatccagaaccctgaccctgccgtgtaccagctgagagactctaaatccagtgacaagtctgtctgcctattcaccgat

SEQ ID NO: 229 - DNA, 200 bp, Artificial Sequence
  TRAC 200 bp 5′ Homology Arm
gtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 230 - DNA, 300 bp, Artificial Sequence
  TRAC 300 bp 5′ Homology Arm
cctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 231 - DNA, 400 bp, Artificial Sequence
  TRAC 400 bp 5′ Homology Arm
attaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 232 - DNA, 500 bp, Artificial Sequence
  TRAC 500 bp 5′ Homology Arm
ctccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 233 - DNA, 600 bp, Artificial Sequence
  TRAC 600 bp 5′ Homology Arm
agagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 234 - DNA, 50 bp, Artificial Sequence
  TRAC 50 bp 3′ Homology Arm
tttgattctcaaacaaatgtgtcacaaagtaaggattctgatgtgtatat

SEQ ID NO: 235 - DNA, 100 bp, Artificial Sequence
  TRAC 100 bp 3′ Homology Arm
tttgattctcaaacaaatgtgtcacaaagtaaggattctgatgtgtatatcacagacaaaactgtgctagacatgaggtctatggacttcaagagcaaca

SEQ ID NO: 236 - DNA, 200 bp, Artificial Sequence
  TRAC 200 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaaggg

SEQ ID NO: 237 - DNA, 300 bp, Artificial Sequence
  TRAC 300 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggt

SEQ ID NO: 238 - DNA, 400 bp, Artificial Sequence
  TRAC 400 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaag

SEQ ID NO: 239 - DNA, 500 bp, Artificial Sequence
  TRAC 500 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttc

SEQ ID NO: 240 - DNA, 600 bp, Artificial Sequence
  TRAC 600 bp 3′ Homology Arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtca

SEQ ID NO: 241 - protein, 141 aa, Artificial Sequence
  Human TCR alpha constant
NIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 242 - protein, 141 aa, Artificial Sequence
  Human TCR alpha constant
HIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 243 - protein, 141 aa, Artificial Sequence
  Human TCR alpha constant
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 244 - protein, 141 aa, Artificial Sequence
  Human TCR alpha constant
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 245 - protein, 142 aa, Artificial Sequence
  Human TCR alpha constant
PNIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 246 - protein, 141 aa, Artificial Sequence
  Human TCR alpha constant
NIQKPDPAVYQLRDSKSSDKSVCLFTDFDSQTNVSQSKDSDVYITDKCVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 247 - protein, 141 aa, Artificial Sequence
  Human TCR alpha constant
HIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 248 - protein, 140 aa, Artificial Sequence
  Human TCR alpha constant
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 249 - protein, 141 aa, Artificial Sequence
  Human TCR alpha constant
NIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 250 - protein, 141 aa, Artificial Sequence
  Human TCR alpha constant
DIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 251 - protein, 177 aa, Artificial Sequence
  Human TCR beta constant
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 252 - protein, 180 aa, Artificial Sequence
  Human TCR beta constant
TEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 253 - protein, 180 aa, Artificial Sequence
  Human TCR beta constant
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 254 - protein, 179 aa, Artificial Sequence
  Human TCR beta constant
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 255 - protein, 180 aa, Artificial Sequence
  Human TCR beta constant
TEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 256 - protein, 180 aa, Artificial Sequence
  Human TCR beta constant
LEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG